(19) United States
(12) Patent Application Publication
(10) Pub. No.: US 2025/0032611 A1
(43) Pub. Date: Jan. 30, 2025

(54) GENE EDITING AND ENGINEERING STEM CELLS FOR DRUG DELIVERY

(71) Applicant: THE BRIGHAM AND WOMEN'S HOSPITAL, INC., Boston, MA (US)

(72) Inventor: Khalid SHAH, Andover, MA (US)

(73) Assignee: THE BRIGHAM AND WOMEN'S HOSPITAL, INC., Boston, MA (US)

(21) Appl. No.: 18/709,327

(22) PCT Filed: Nov. 10, 2022
(86) PCT No.: PCT/US2022/049523

§ 371 Date: May 10, 2024

Related U.S. Application Data
(60) Provisional application No. 63/278,872, filed on Nov. 12, 2021.

(51) Int. Cl. A61K 39/00; A61K 35/763; A61P 35/04

(52) U.S. Cl. CPC A61K 39/461; A61K 35/763; A61K 39/464439; A61K 39/464441; A61P 35/04; A61K 2239/31; A61K 2239/57

(57) ABSTRACT

Technology provided herein relates to methods of treating cancer comprising administering to a subject in need thereof a first stem cell (SC) modified to release an oncolytic virus and a second SC which is gene edited to inactivate a receptor for the oncolytic virus, thereby generating a stem cell resistant to the virus, wherein the second SC is also engineered to express an immunomodulatory polypeptide agent. Compositions comprising a first and second SC, and uses thereof, are also provided herein.

### CROSS-REFERENCE TO RELATED APPLICATIONS

This application claims benefit under 35 U.S.C. § 119(e) of U.S. Provisional Application No. 63/278,872 filed Nov. 12, 2021, the contents of which are incorporated herein by reference in their entirety.

### TECHNICAL FIELD

The technology described herein relates to the treatment of cancer, and more particularly to the treatment of cancer using stem cell-delivered therapeutics.

### BACKGROUND

Virotherapy is a promising therapeutic approach for cancer treatment. In particular, oncolytic viruses (OV) have been the subject of active research for tumor therapies. OVs are reported to induce immunogenic cell death, and can also be combined, for example with various chemotherapeutics such as oxaliplatin and cyclophosphamide, and/or with radiation, and are candidates for use in combination with immune checkpoint modulators, such as immune checkpoint inhibitors.

Talimogene laherparepvec (T-VEC), the first FDA approved oncolytic herpes simplex virus (oHSV), exhibited therapeutic benefits against melanoma as a monotherapy in a phase III clinical trial and recently demonstrated promising efficacy and acceptable safety in combination with pembrolizumab for patients with advanced melanoma in a phase Ib clinical trial. T-VEC introduces granulocyte-macrophage colony-stimulating factor (GM-CSF) into tumors by expression from the oHSV during viral replication, and local GM-CSF enhances migration and maturation of dendritic cells which recognize tumor antigens, and promotes activation of the anti-tumor immune system. While T-VEC can be effective against melanoma when injected directly into tumors, T-VEC has not been shown to improve overall survival for patients with brain, liver, or lung metastasis. The amount of GM-CSF expression depends on viral replication, and virus neutralization is a significant barrier to the effective delivery of oHSV to target metastatic tumor lesions.

Mesenchymal stem cells have a marked tendency to home to tumors, including metastases. Allogeneic MSCs and neuronal stem cells (NSCs) can home, for example, to brain tumors and act as carriers for oncolytic viruses. Other approaches use stem cells to deliver other factors, such as cytotoxic cytokines or other immunomodulators to the tumor microenvironment to stimulate or enhance anti-tumor immune activities.

### SUMMARY

The compositions and methods described herein are based, in part, on the discovery that stem cell-delivered therapies tend to be limited by susceptibility of the stem cells to the agents they deliver. This is particularly relevant in the situation in which stem cells modified to deliver a receptor-targeted cytotoxic agent, alone or in combination with stem cell-mediated delivery of one or more additional polypeptides, such as polypeptides that stimulate or enhance an anti-tumor immune response, are themselves susceptible to the receptor-targeted cytotoxic agent. Where, as a non-limiting example, an MSC that expresses an immunomodulatory polypeptide is to be combined with an MSC loaded with an oncolytic virus, susceptibility of the MSC that expresses the immunomodulatory polypeptide to the oncolytic virus (or other receptor-targeted cytotoxic agent), can sharply limit the anticipated benefit of the combined therapy. It is demonstrated in the Examples herein that modification of stem cells used to deliver agents that promote anti-tumor effects to inactivate the receptor(s) for a receptor-targeted cytotoxic agent can dramatically improve the efficacy of stem cell-delivered therapeutics. Targeted gene editing of stem cells, e.g., via CRISPR/Cas or similar approaches, can limit sensitivity of the stem cells to the agent(s) they or other populations of modified stem cells deliver, while preserving the ability of the stem cells to home to tumors, including metastatic tumors, and thereby dramatically improve the efficacy of stem cell-mediated anti-tumor approaches.

One aspect provided herein describes a method of treating cancer comprising administering to a subject in need thereof a first stem cell (SC) modified to release an oncolytic virus and a second SC modified to express a first therapeutic polypeptide, wherein the second SC is further engineered to inactivate a receptor for the oncolytic virus. In one embodiment of this and all other aspects described herein, the therapeutic polypeptide is an immunomodulatory polypeptide that promotes or enhances an anti-tumor immune response.

In one embodiment of this aspect and all other aspects described herein, the method comprises administering to a subject in need thereof a first stem cell (SC) modified to release an oncolytic virus and a second SC which is gene edited to inactivate a receptor for the oncolytic virus to create a SC resistant to the virus, and which is subsequently engineered to express one or more immunomodulatory polypeptide agents.

In one embodiment of this and all other aspects described herein, the first and/or second stem cell is a mesenchymal stem cell (MSC) or a neuronal stem cell (NSC). In one embodiment of this and all other aspects described herein the second cell is alternatively an NK cell, a macrophage, or a T cell. Any or all of such cells (MSC, NSC, NK cell, macrophage, T cell, etc.) can be derived from an induced pluripotent stem (iPS) cell. Methods of generating iPS cells are known in the art, as are methods of differentiating them to MSCs, NSCs, NK cells, macrophages, T cells, etc.).

In one embodiment of this and all other aspects described herein, the first and/or second stem cell is autologous to the subject.

In one embodiment of this and all other aspects described herein, the first and/or second stem cell is allogeneic to the subject.

In one embodiment of this and all other aspects described herein, the oncolytic virus is, for example, an oncolytic adenovirus, herpes simplex virus, Newcastle Disease Virus, or other oncolytic virus; see, e.g., Lawler et al., JAMA Oncol. 3: 841-849 (2017) or Zeyaullah et al., Pathol. Oncol. Res. (2012) for reviews regarding oncolytic viruses. In one embodiment of this and all other aspects described herein, the oncolytic virus is an oncolytic herpes simplex virus (oHSV). In one embodiment of this and all other aspects described herein, the oHSV is or is derived from G47Δ oHSV. In one embodiment of this and all other aspects described herein, the receptor for the oncolytic virus is nectin-1.

In one embodiment of this and all other aspects described herein, the oncolytic virus encodes a heterologous polypeptide. In one embodiment of this and all other aspects described herein, the heterologous polypeptide is a tumor necrosis factor related apoptosis-inducing ligand (TRAIL) polypeptide or a cytokine that promotes an anti-tumor immune response. In one embodiment of this and all other aspects described herein, the cytokine that promotes an anti-tumor immune response is one or more of GM-CSF, IL-2, IL-12, Flt3L, IL-5 and IL-15.

In one embodiment of this and all other aspects described herein, the heterologous polypeptide comprises an inhibitor of IL-6, an inhibitor of CSF1R, and/or an inhibitor of CD47. In one embodiment of this and all other aspects described herein, the inhibitor of IL-6, CSF1R and/or CD47 comprises an antigen-binding domain of an antibody that specifically binds the relevant factor, and includes, but is not limited to a nanobody or an scFv.

In one embodiment of this and all other aspects described herein, the first therapeutic polypeptide comprises an immunomodulator polypeptide. In one embodiment of this and all other aspects described herein, the immunomodulator polypeptide is a cytokine that promotes an anti-tumor immune response. In one embodiment of this and all other aspects described herein, the cytokine is one or more of GM-CSF, IL-2, IL-12, Flt3L, IL-5 and IL-15. In one embodiment of this and all other aspects described herein, the first therapeutic polypeptide comprises an inhibitor of IL-6, an inhibitor of CSF1R, and/or an inhibitor of CD47. In one embodiment of this and all other aspects described herein, the cytokine is a GM-CSF polypeptide.

In one embodiment of this and all other aspects described herein, the immunomodulator polypeptide comprises a modulator of an immune checkpoint molecule. In one embodiment of this and all other aspects described herein, the immunomodulator polypeptide produced by the second SC comprises a modulator of an immune checkpoint molecule. In one embodiment of this and all other aspects described herein, the modulator of an immune checkpoint molecule is an agonist of a stimulatory checkpoint molecule or an antagonist of an inhibitory checkpoint molecule. In one embodiment of this and all other aspects described herein, the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40. In one embodiment of this and all other aspects described herein, the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3. In one embodiment of this and all other aspects described herein, the modulator of an immune checkpoint molecule comprises an antibody or an antigen-binding fragment thereof that specifically binds the immune checkpoint molecule.

In one embodiment of this and all other aspects described herein, the second SC further expresses a second therapeutic polypeptide. In one embodiment of this and all other aspects described herein, the second therapeutic polypeptide comprises a cytokine or a modulator of an immune checkpoint molecule. In one embodiment of this and all other aspects described herein, the cytokine is one or more of GM-CSF, IL-2, IL-12, Flt3L, IL-5 and IL-15. In one embodiment of this and all other aspects described herein, the second therapeutic polypeptide comprises an inhibitor of IL-6, an inhibitor of CSF1R, and/or an inhibitor of CD47. In one embodiment of this and all other aspects described herein, the modulator of an immune checkpoint molecule is an agonist of a stimulatory checkpoint molecule or an antagonist of an inhibitory checkpoint molecule. In one embodiment of this and all other aspects described herein, the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40. In one embodiment of this and all other aspects described herein, the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3. In one embodiment of this and all other aspects described herein, the modulator of an immune checkpoint molecule comprises an antibody or an antigen-binding fragment thereof that specifically binds the immune checkpoint molecule.

Another aspect provided herein describes a method of treating cancer comprising administering to a subject in need thereof a stem cell (SC) modified to express and secrete a receptor-targeted cytotoxic agent, wherein the SC is gene edited to inactivate a receptor for the receptor-targeted cytotoxic agent. In one embodiment of this and all other aspects described herein, the gene editing to inactivate a receptor is performed before the stem cell is modified to express and secrete a receptor-targeted cytotoxic agent.

In one embodiment of this and all other aspects described herein, the receptor-targeted cytotoxic agent is a cytokine or a death receptor-targeted pro-apoptotic factor. In one embodiment of this and all other aspects described herein, the cytokine is IFNβ. In one embodiment of this and all other aspects described herein, the receptor for the receptor-targeted cytotoxic agent is IFNaR1 or IFNaR2.

In one embodiment of this and all other aspects described herein, the death receptor-targeted pro-apoptotic factor is tumor necrosis factor related apoptosis-inducing ligand (TRAIL). In one embodiment of this and all other aspects described herein, the receptor for the receptor-targeted cytotoxic agent is death receptor (DR) 4 or DR5.

In one embodiment of this and all other aspects described herein, the receptor for the oncolytic virus or the receptor-targeted cytotoxic agent is inactivated by targeted gene editing. In one embodiment of this and all other aspects described herein, the targeted gene editing involves use of a guide RNA-directed endonuclease, as for example, CRISPR/Cas9 mediated gene editing.

In one embodiment of this and all other aspects described herein, the stem cell is further engineered to express an immunomodulator polypeptide. In one embodiment of this and all other aspects described herein, the immunomodulator polypeptide is a cytokine that promotes an anti-tumor immune response. In one embodiment of this and all other aspects described herein, the cytokine is one or more of GM-CSF, IL-12, IL-2, IL-12, Flt3L, IL-5 and IL-15. In one embodiment of this and all other aspects described herein, the cytokine is a GM-CSF polypeptide. In one embodiment of this and all other aspects described herein, the immunomodulator comprises an inhibitor of IL-6, an inhibitor of CSF1R, and/or an inhibitor of CD47.

In one embodiment of this and all other aspects described herein, the immunomodulator polypeptide comprises a modulator of an immune checkpoint molecule. In one embodiment of this and all other aspects described herein, the modulator of an immune checkpoint molecule is an agonist of a stimulatory checkpoint molecule or an antagonist of an inhibitory checkpoint molecule. In one embodiment of this and all other aspects described herein, the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40. In one embodiment of this and all other aspects described herein, the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3. In one embodiment of this and all other aspects described herein, the modulator of an immune checkpoint molecule comprises an antibody or an antigen-binding fragment thereof that specifically binds the immune checkpoint molecule.

In one embodiment of this and all other aspects described herein, the method further comprises administering a second stem cell engineered to express an immunomodulator polypeptide. In one embodiment of this aspect and all other aspects described herein, the immunomodulator polypeptide is different from that expressed by the first stem cell. In one embodiment of this and all other aspects described herein, the additional or second immunomodulator polypeptide is an immunomodulator polypeptide as described herein.

In one embodiment of this aspect and all other aspects described herein, the cancer comprises a solid tumor cancer. In one embodiment of this aspect and all other aspects described herein, the cancer is selected from melanoma, lung cancer, breast cancer, and glioblastoma.

In one embodiment of this aspect and all other aspects described herein, the cancer comprises a primary tumor or a metastatic tumor. In one embodiment of this aspect and all other aspects described herein, the metastatic tumor comprises a metastasis to the brain.

In one embodiment of this aspect and all other aspects described herein, the cancer is PTEN-deficient.

In one embodiment of this aspect and all other aspects described herein, the administering comprises intratumor administration.

In one embodiment of this aspect and all other aspects described herein, the administering comprises systemic administration.

In one embodiment of this aspect and all other aspects described herein, the administering comprises administration of the first and/or second stem cells to a tumor resection cavity.

Another aspect provided herein describes a composition comprising a) a first stem cell (SC) modified to release an oncolytic virus, and b) a second SC which is gene edited to inactivate a receptor for the oncolytic virus, thereby generating a SC resistant to the virus, wherein the second SC is also engineered to express an immunomodulatory polypeptide agent.

Another aspect provided herein describes a composition comprising a stem cell (SC) modified to express and secrete a receptor-targeted cytotoxic agent, wherein the SC is gene edited to inactivate a receptor for the receptor-targeted cytotoxic agent.

Yet another aspect provided herein describes a use of any composition described herein for the treatment of cancer in a subject in need thereof.

In one embodiment of any one of the aspects described herein, the immunomodulatory polypeptide agent produced by the first and second SC are the same. In one embodiment of any one of the aspects described herein, the immunomodulatory polypeptide agents produced by the first and second SC are different.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “decrease”, “reduce”, “inhibit”, or other grammatical forms thereof are used herein to mean a decrease by a statistically significant amount. In some embodiments, “reduce” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “inhibition” does not encompass a complete inhibition as compared to a reference level. “Complete inhibition” is a 100% inhibition as compared to a reference level. Where applicable, a decrease can be preferably down to a level accepted as within the range of normal for an subject without a given disease (e.g., cancer).

The terms “increased”, “increase”, “enhance”, or grammatical forms thereof are used herein to mean an increase by a statically significant amount. In some embodiments, the terms “increased”, “increase”, or “enhance”, can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. glioblastoma or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

As used herein, “genetically modified” refers to a cell (e.g., an MSC or other stem cell) that has been altered to introduce changes to its genetic composition. A cell can be genetically modified to contain and/or express a gene product from one or more exogenous nucleic acid sequences not found in its genome (e.g., a MSC genetically modified to express a gene product from a heterologous nucleic acid sequence). Alternatively, or in addition, a cell can be genetically modified to either overexpress or inactivate or disrupt the expression of one or more genes or polypeptides. One skilled in the art will know how to introduce changes to the cell's genome using gene editing approaches.

As used herein, the term “modified to release an oncolytic virus” means that a stem cell has been loaded with and will release the oncolytic virus. Viral loading is described in the Examples herein, but briefly, comprises infecting the stem cell, e.g., a mesenchymal stem cell, neuronal stem cell or other stem cell that exhibits tumor-homing activity, at a relatively low multiplicity of infection such that the stem cell survives long enough to be delivered to or home to the location of a tumor and to deliver oncolytic viral particles to the tumor before dying.

As used herein, the term “engineered” and its grammatical equivalents can refer to one or more human-designed alterations of a nucleic acid, e.g., the nucleic acid within an organism's genome or genetic composition. The term can refer to alterations, additions, and/or deletion of genes. An “engineered cell” can refer to a cell with an added, deleted and/or altered gene. The term “cell” or “engineered cell” and their grammatical equivalents as used herein can refer to a cell of human or non-human animal origin.

As used herein, the term “inactivate” or “inactivated,” when used in reference to the activity or expression of a receptor, means that the gene from which the receptor is normally expressed has been modified such that the receptor polypeptide is either not expressed, or if expression remains, the polypeptide is not functional. Inactivation can be, for example, via targeted gene editing, e.g., CRISPR/Cas gene editing or the like, and can include wholesale deletion of the gene, or mutation such that the protein is either not expressed, or the form expressed lacks one or more elements necessary for function. Where, for example, function is as a receptor for a virus, inactivation renders the receptor incapable of mediating viral entry. Where, for example, function is transduction of a signal by the receptor polypeptide, inactivation knocks out expression of the receptor polypeptide or renders the receptor polypeptide that is expressed incapable of binding its target ligand and/or incapable of transducing a signal in response to ligand binding.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

A “receptor-targeted cytotoxic agent” as used herein refers to a polypeptide that binds to and activates signaling by a receptor in or on a cell that induces a cell death program in the cell. In one embodiment, the cell death program is an apoptotic cell death program. Non-limiting examples include: tumor necrosis factor-related apoptosis-inducing ligand (TRAIL), which binds death receptors DR4 and DR5 to activate FADD (Fas-associated protein with death domain)-dependent apoptosis; and Interferon β (IFNβ), which binds and signals through the Interferon α/β receptor (IFNAR).

As used herein, a “checkpoint molecule” or an “immune checkpoint molecule” is a member of a ligand/receptor pair that exerts an inhibitory or stimulatory effect on the innate immune system. An immune checkpoint molecule is directly or indirectly involved in an immune pathway that under normal physiological conditions is important for preventing uncontrolled immune reactions and thus for the maintenance of self-tolerance and/or tissue protection. Immune checkpoint molecules are important in modulating the length and magnitude of immune responses. Tumor expression of, for example, inhibitory checkpoint molecules is a common component of tumor immune evasion and provides a target for overcoming such immune evasion to promote immune attack of the tumor.

Immune checkpoint molecules can be described based on their ability to inhibit or stimulate the innate immune system. For example, immune checkpoint molecules that naturally inhibit innate immunity are referred to herein as “inhibitory checkpoint molecules.” In order to induce an anti-tumor immune response, one of skill in the art will recognize that inhibitors of such inhibitory checkpoint molecules can release the inhibition of the innate immune system mediated by the inhibitory checkpoint molecule. Exemplary inhibitory checkpoint molecules include, but are not limited to, PD-1, PD-L1, CTLA4, B7-H3, B7-H4, VISTA, TMIGD2, B7-H7, BTLA, HVEM, CD160, LAG3, TIGIT, CD96, CD155 and TIM-3. Non-limiting examples of checkpoint inhibitors include pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).

In contrast, an immune checkpoint molecule that can induce or increase an innate immune response is referred to herein as a “stimulatory checkpoint molecule.” As will be appreciated by those of skill in the art, an agonist of a stimulatory checkpoint molecule can induce or increase the innate immune response. Exemplary stimulatory checkpoint molecules include, but are not limited to, OX40, 4-1BB, GITR, CD28, ICOS, LIGHT, CD27, DNAM-1, 2B4, DC-SIGN, DR3, and CD40.

The term “immune checkpoint modulator” refers to a molecule capable of modulating the function of an immune checkpoint molecule or protein in a positive or negative way. The immune checkpoint modulator(s) applicable to the methods and compositions described herein can independently act at any step of T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that fine tune the response. The term “immune checkpoint modulator” encompasses immune checkpoint modulator(s) capable of down-regulating at least partially the function of an inhibitory immune checkpoint (antagonist) and/or immune checkpoint modulator(s) capable of upregulating at least partially the function of a stimulatory immune checkpoint (agonist). For example, the term “immune checkpoint modulator” can refer to molecules that can totally or partially reduce, inhibit, interfere with or modulate one or more inhibitory checkpoint proteins, which in turn regulates T-cell activation or function. Similarly, the term “immune checkpoint modulator” can also refer to molecules that can increase or induce the expression or activity of one or more stimulatory checkpoint proteins, which in turn regulates T-cell activation or function. Immune checkpoint modulators include small molecules, peptides, peptidomimetics, and/or antibodies or antigen binding fragments thereof (e.g., a construct employing the antigen-binding domain of an antibody) that bind a checkpoint protein.

As used herein, the term “anti-tumor immune response” refers to an increase in immune cell-related activity within a tumor, lesion or tissue. Examples of an increased anti-tumor response include, but are not limited to, an increase in recruitment, number or activity of inflammatory cells (e.g., CD8+ T cells, among others) within the tumor or tissue, increased processing and presentation of released tumor antigens by antigen-presenting cells (APCs; such as CD103+ dendritic cells), increased immune or T-cell activation within the tumor or the tumor microenvironment, trafficking of antigen-specific effector cells to the tumor, or engagement of a target tumor cell by an activated effector T cell.

In some embodiments, an anti-tumor immune response is assessed by measuring the number of CD8+ T cells within the tumor, with an increase as the term is defined herein following treatment as described herein providing evidence of effective treatment.

As used herein, “antibodies” or “antigen-binding fragments” thereof include monoclonal, human, humanized or chimeric antibodies, single chain antibodies, Fab fragments, F(ab′) fragments, fragments produced by a Fab expression library, and/or antigen-binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

The terms “specificity” or “specific for” refers to the number of different types of antigens or antigenic determinants to which a binding protein, antibody or antibody fragment, or antigen-binding portion thereof as described herein can bind. The specificity of a binding protein, antibody or antibody fragment, or antigen-binding portion thereof can be determined based on affinity and/or avidity. The affinity, represented by the equilibrium constant for the dissociation (K_{D}) of an antigen with an antigen-binding protein, is a measure of the binding strength between an antigenic determinant and an antigen-binding site on the antigen-binding protein, such as a binding protein, antibody or antibody fragment, or antigen-binding portion thereof: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, a binding protein, antibody or antibody fragment, or antigen-binding portion thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10000 times or more better than the affinity with which said amino acid sequence or polypeptide binds to another target or polypeptide.

Accordingly, as used herein, “selectively binds” or “specifically binds” or “specific binding” in reference to the interaction of an antibody, or antibody fragment thereof, or a binding protein described herein, means that the interaction is dependent upon the presence of a particular structure (e.g., an antigenic determinant or epitope or target) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In certain embodiments, a binding protein or antibody or antigen-binding fragment thereof that specifically binds to an antigen binds to that antigen with a K_{D} greater than 10⁻⁶ M, 10⁻⁷ M, 10⁻¹ M, 10⁻⁹ M, 10⁻¹⁰ M, 10⁻¹ M, 10⁻¹² M, 10⁻¹³ M, 10⁻¹⁴ M. In other embodiments, a binding protein or antibody or antigen binding fragment thereof that specifically binds to an antigen binds to that antigen with a K_{D} between 10⁻⁶ and 10⁻⁷M, 10⁻⁶ and 10⁻⁸ M, 10⁻⁶ and 10⁻⁹ M, 10⁻⁶ and 10⁻¹⁰ M, 10⁻⁶ and 10⁻¹¹M, 10⁻⁶ and 10⁻¹²M, 10⁻⁶ and 10⁻¹³ M, 10⁻⁶ and 10⁻¹⁴ M, 10⁻⁹ and 10⁻¹⁰ M, 10⁻⁹ and 10⁻¹¹ M, 10⁻⁹ and 10⁻¹² M, 10⁻⁹ and 10⁻¹³ M, 10⁹ and 10⁻¹⁴ M. In some embodiments, a binding protein or antibody or antigen-binding fragment thereof binds to an epitope, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. In certain embodiments, a binding protein or antibody or antigen-binding fragment thereof is said to “specifically bind” an antigen when it preferentially recognizes its target antigen in a complex mixture of proteins and/or macromolecules. Binding proteins, antibodies or antigen-binding fragments that bind to the same or similar epitopes will likely cross-compete (one prevents the binding or modulating effect of the other). Cross-competition, however, can occur even without epitope overlap, e.g., if epitopes are adjacent in three-dimensional space and/or due to steric hindrance.

As used herein, an “antigen-binding fragment” refers to a protein fragment that comprises at least an antigen binding site of the intact antibody and thus retains the ability to bind a given target antigen or epitope. Non-limiting examples of antibody fragments encompassed by the term antigen-binding fragment include: (i) the Fab fragment, having V_{L}, C_{L}, V_{H} and C_{H}1 domains; (ii) the Fab′ fragment, which is a Fab fragment having one or more cysteine residues at the C-terminus of the C_{H} i domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) the dAb fragment (Ward et al., Nature 341, 544-546 (1989)) which consists of a V_{H} domain; (vii) isolated CDR regions; (viii) F(ab′)₂ fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}—C_{H}1-V_{H}—C_{H}1) which, together with complementary light chain polypeptides, form a pair of antigen binding regions (Zapata et al. Protein Eng. 8(10):1057-1062 (1995); and U.S. Pat. No. 5,641,870).

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

The “Fab” fragment contains a variable and constant domain of the light chain and a variable domain and the first constant domain (C_{H}1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “monoclonal antibody” or “mAb” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A monoclonal antibody can be of any species, including, but not limited to, mouse, rat, goat, rabbit, and human monoclonal antibodies. Various methods for making monoclonal antibodies specific for TIGIT, PD-1, TIM-3, LAG-3, or CTLA-4 as described herein are available in the art. For example, the monoclonal antibodies can be made using the hybridoma method first described by Kohler et al., Nature, 256:495 (1975), or by recombinant DNA methods (U.S. Pat. No. 4,816,567). “Monoclonal antibodies” can also be isolated from or produced using phage antibody libraries using the techniques originally described in Clackson et al., Nature 352:624-628 (1991), Marks et al., J. Mol. Biol. 222:581-597 (1991), McCafferty et al., Nature, 348:552-554 (1990), Marks et al., Bio/Technology, 10:779-783 (1992)), Waterhouse et al., Nuc. Acids. Res., 21:2265-2266 (1993), and techniques known to those of ordinary skill in the art.

The term “human antibody” includes antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies that comprise heavy and light chain variable domain sequences from one species and constant region sequences from another species, such as antibodies having murine heavy and light chain variable domains linked to human constant regions.

The term “CDR-grafted antibody” refers to antibodies that comprise heavy and light chain variable domain sequences from one species but in which the sequences of one or more of the CDR regions of VH and/or VL are replaced with CDR sequences of another species, such as antibodies having murine heavy and light chain variable domains in which one or more of the murine CDRs (e.g., CDR3) has been replaced with human CDR sequences.

The term “CDR” refers to the complementarity determining region within antibody variable sequences. There are three CDRs in each of the variable domains of the heavy chain and the light chain, which are designated CDR1, CDR2 and CDR3, for each of the variable domains. The term “CDR set” as used herein refers to a group of three CDRs that occur in a single variable domain capable of binding the antigen. The exact boundaries of these CDRs have been defined differently according to different systems. The system described by Kabat (Kabat et al., Sequences of Proteins of Immunological Interest, National Institutes of Health, Bethesda, Md. (1987) and (1991)) not only provides an unambiguous residue numbering system applicable to any variable domain of an antibody, but also provides precise residue boundaries defining the three CDRs. These CDRs may be referred to as Kabat CDRs. Chothia and coworkers (Chothia et al. (1987) J. Mol. Biol. 196: 901-917; and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. ((1995) FASEB J. 9:133-139) and MacCallum et al. ((1996) J. Mol. Biol. 262(5):732-745). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although exemplary embodiments use Kabat or Chothia defined CDRs.

The terms “Kabat numbering”, “Kabat definitions”, and “Kabat labeling” are used interchangeably herein. These terms, which are recognized in the art, refer to a system of numbering amino acid residues which are more variable (i.e., hypervariable) than other amino acid residues in the heavy and light chain variable domains of an antibody, or an antigen binding portion thereof (Kabat et al. (1971) Ann. NY Acad. Sci. 190:382-391; and Kabat et al. (1991) Sequences of Proteins of Immunological Interest, Fifth Edition, U.S. Department of Health and Human Services, NIH Publication No. 91-3242, which is also available on the world wide web, and is expressly incorporated herein in its entirety by reference. The “EU index as in Kabat” refers to the residue numbering of the human IgG1 EU antibody. As used herein, “Kabat sequence numbering” refers to numbering of the sequence encoding a variable region according to the EU index as in Kabat. In some embodiments, IMGT (INTERNATIONAL IMMUNOGENETICS INFORMATION SYSTEM) numbering of variable regions can also be used, which is the numbering of the residues in an immunoglobulin variable heavy or light chain according to the methods of the IIMGT, as described in Lefranc, M.-P., “The IMGT unique numbering for immunoglobulins, T cell Receptors and Ig-like domains”, The Immunologist, 7, 132-136 (1999), and is expressly incorporated herein in its entirety by reference. As used herein, “IMGT sequence numbering” refers to numbering of the sequence encoding a variable region according to the IMGT. For the heavy chain variable domain, the hypervariable region ranges from amino acid positions 31 to 35 for CDR1, amino acid positions 50 to 65 for CDR2, and amino acid positions 95 to 102 for CDR3. For the light chain variable domain, the hypervariable region ranges from amino acid positions 24 to 34 for CDR1, amino acid positions 50 to 56 for CDR2, and amino acid positions 89 to 97 for CDR3.

As used herein, the term “canonical” residue refers to a residue in a CDR or framework that defines a particular canonical CDR structure as defined by Chothia et al. ((1987) J. Mol. Biol. 196: 901-917); and Chothia et al. ((1992) J. Mol. Biol. 227: 799-817), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). Each heavy chain is composed of a variable region of the heavy chain (V_{H} refers to the variable domain of the heavy chain) and a constant region of said heavy chain. The heavy chain constant region consists of three domains CH1, CH2 and CH3. Each light chain is composed of a variable region of said light chain (V_{L} refers to the variable domain of the light chain) and a constant region of the light chain. The light chain constant region consists of a CL domain. The VH and VL regions can be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, FR4. This structure is well known to those skilled in the art. According to the methods used herein, the amino acid positions assigned to CDRs and FRs can be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

The term “therapeutically effective amount” therefore refers to an amount of the inhibitors or potentiators described herein, using the methods as disclosed herein, that is sufficient to provide a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign tumors and malignant cancers, as well as dormant tumors or micrometastases. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

As used herein, the term “promotes an anti-tumor immune response” means that a given treatment induces one or more activities of the innate or adaptive immune system that either directly or indirectly kills or suppresses the establishment or growth of a tumor, including but not limited to a metastatic tumor. In methods and compositions as described herein, an anti-tumor immune response can be in addition to an anti-tumor effect mediated by a receptor-targeted cytotoxic agent. In one embodiment, an anti-tumor immune response can include killing or suppression of the growth or establishment of new tumors, including metastases. In another embodiment, an anti-tumor immune response and can include killing or suppression of the growth or establishment of new primary tumors. An anti-tumor immune response can be detected or measured in a manner as known in the art or, for example, as described herein.

According to the methods described herein, including, without limitation, methods of treating cancer, methods of inhibiting metastases and methods of inducing an anti-tumor immune response, it should be understood that in one embodiment, the engineered or modified stem cells can be autologous to the individual to be treated. In another embodiment, the engineered or modified stem cells can be syngeneic or allogeneic to the individual to be treated.

In some embodiments, because stem cells such as MSCs tend to have characteristics of immune privilege or immune indifference, the genetically modified stem cells can be used “off the shelf” to treat individuals that are not necessarily closely matched in terms of MHC expression.

The term “pharmaceutically acceptable” as used herein is consistent with the art and means compatible with the other ingredients of the pharmaceutical composition and not deleterious to the recipient thereof.

The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an agent as described herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. glioblastoma or other solid tumor cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic or pharmaceutical composition or agent as described herein into a subject by a method or route which results in at least partial delivery of the agent at a desired site. Pharmaceutical compositions comprising agents as described herein can be administered by any appropriate route which results in an effective treatment in the subject.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology of the following.

### BRIEF DESCRIPTION OF THE DRAWINGS

FIGS. 1A-1M show PTEN deficiency is correlated with melanoma brain metastasis and immune suppression. Development and characterization of primary and metastatic mouse tumor models of PTEN-deficient melanoma. (FIG. 1A) Heatmap of mRNA levels of PTEN, BRAF, TP53 and KRAS in patient samples of metastatic stage from TCGA database (n=337). mRNA expression z-scores relative to all samples (≥1.5) was defined as positive expression. (FIG. 1B) Comparison of PTEN levels. (FIG. 1C) Kaplan-Meier curves of overall survival for high PTEN melanoma and low PTEN melanoma. (FIG. 1D) Immune profiles analysis of high PTEN melanoma and low PTEN melanoma. (FIG. 1E) Western blotting of PTEN and Tubulin in six murine melanoma cell lines. (FIG. 1F) Schematic of primary melanoma. BLI signal curve of primary UV2-GFl (n=4) or Y1.1-GFl (n=3)-bearing mice. (FIG. 1G) Schematic of leptomeningeal metastasis. BLI signal curve of flank and IT-injected UV2-GFl (n=3) or Y1.1-GFl (n=3)-bearing mice. (FIG. 1H) HE staining of primary melanoma (edge and central area) and LM model (Lateral ventricle and cerebellum area). Scale bar, 100 mm. (FIG. 1I) Immunofluorescence analysis of CD11c, CD3, CD4, CD8, and CD68 in UV2-GFl primary (n=4) and LM (n=3) model. Scale bar, 6 mm. (FIG. 1J) Mean number of TILs expressing CD11c, CD3, CD4, CD8, CD68, and IBA1 was statistically assessed from three selected fields. IBA1 was compared UV2-GFl LM to GBM (CT2A-mCherry-Fluc) bearing mice (n=3 per group). (FIG. 1K) Flowcytometry showing difference of immune profiles between primary (n=4) and LM (n=3) model. (FIG. 1L) Heatmap of differential expression of genes associated with immune cell types in flank and LM melanoma plotted as z-score of normalized gene expression for each gene. (FIG. 1M) GO analysis for Down-regulated Immune-related Pathway enrichment (Flank versus LM). *: p<0.05.

FIGS. 2A-2K show utility of SCs as carrier for oHSV. (FIG. 2A) Cell viability assay of six melanoma cells was assessed 3 days after oHSV treatment at the indicated doses (MOI) (n=5 per group, technical replicates). Data are represented as mean±SD. (FIG. 2B) Whole-cell lysates of Y1.1, Y2.1 and UV2 cells collected 3 days after oHSV treatment (0, 1, 2, 5, 10, and 20 MOI) were subjected to western blot analysis of p-AKT, p-mTOR, PI3K and R-actin expression (technical replicates). (FIG. 2C) Extracellular ATP secreted from Y1.1, Y2.1 and UV2 cells was measured using a luminescence assay 24 h and 48 h after oHSV treatment (0, 2, and 5 MOI). Data are represented as mean±SD. (FIG. 2D) Concept of SC-oHSV. oHSV can infect into SCs via Nectin-1 receptors. (FIG. 2E) SCs were was assessed using cell viability assay 0-3 days after oHSV treatment infected at the indicated doses (MOI). (n=5 per group, technical replicates). Data are represented as mean±SD. (FIG. 2F) SC-oHSV therapy was effective for Y1.1-GFl cells in vitro. Y1.1-GFl cells were co-cultured with SC-oHSV cells infected at either 2 or 5 MOI oHSV (n=5 per group, technical replicates). Plot showing quantified differences in Y1.1-GFl cell viabilities between co-cultures with SC-oHSV at 2 and 5 MOI 72 h (n=5 per group, technical replicates). Data are represented as mean±SD. (FIG. 2G) PTEN mutant melanoma cells were investigated by cell viability assay 3 days after SC-oHSV treatment in vitro (n=5 per group, technical replicates). Data are represented as mean±SD. (FIG. 2H) Imaging for co-culture with melanoma cells and SC-oHSV for 12 h, 24 h, 48 h and 72 h, respectively. Scale bar, 100 μm. (FIG. 2I) Plots showing subcutaneous UV2-GFl tumor growth in mice treated with oHSV (2×10⁵ PFU per mouse, n=5) or SC-oHSV (2×10⁵ cells per mouse, n=5). Plot showing quantified differences in these expressions between oHSV and SC-oHSV. Data are represented as mean±SEM. (FIGS. 2J and 2K) Stealth effect of SC-oHSV providing protection from the immune system in vivo. PBS, SC-oHSV-FmC, or oHSV-FmC were systemically injected to C57BL/6 mice twice every week (day 1 and 8) and the blood was collected from each mouse at day 14. Vero cells were infected with oHSV-FmC for 2 days with the serum collected from the mice. mCherry spots were observed on fluorescence microscopy and intensity of mCherry spots is measured by ImageJ. Data are represented as mean±SD. Scale bar, 100 μm. *: p<0.05. **: p<0.01, ***: p<0.001, ****: p<0.0001.

FIGS. 3A-3L show establishment of oHSV-resistant SC secreting GM-CSF and the influence against dendritic cells and Macrophages. (FIG. 3A) Scheme showing creation of SC^{N1KO}-immunomodulator. (FIG. 3B) Flowcytometry (FCM) and western blotting showing expression of Nectin-1 in SC (technical replicates). (FIG. 3C). Cell viability assays showing SC^{N1KO} resistance to oHSV compared to SC (n=5 per group, technical replicates). Data are represented as mean±SD. (FIG. 3D) Expression of immunomodulators in SC^{N1KO} by western blotting (technical replicates). (FIG. 3E) SC^{N1KO} mediated GM-CSF expression enhances the antitumor activity of oHSV-loading SC in vivo primary melanoma model (n=5 per group). (FIG. 3F) Cell viability assays showing influence of SC^{N1KO}-G and SC-Rluc-mCherry (RmC) on murine macrophage (RAW264.7) and melanoma (Y1.1-GFl, Y2.1-GFl and UV2-GFl). GM-CSF induced macrophage growth but not murine melanoma cells in vitro (n=5 per group, technical replicates). Data are represented as mean±SD. (FIG. 3G) SC^{N1KO}-G conditioned medium (CM) increased TNFα positive RAW264.7 cells by FCM after incubation for 4 days (n=3 per group). Data are represented as mean±SD. (FIG. 3H) SC^{N1KO}-G conditioned medium (CM) induces differentiation to dendritic cells (DCs) and mature DCs from murine bone marrow cells by FCM after incubation for 4 days (n=3-4 per group). Data are represented as mean±SD. Scale bar, 100 μm. (FIG. 3I) Experimental design. In brief, in UV2-GFl subcutaneous tumor model, tumor was treated with SC-oHSV and SC^{N1KO}-G intratumorally twice. Then, tumor volumes were measured every 3-4 days post-implantation. Plots showing subcutaneous UV2-GFl tumor growth in mice treated with control SC-RmC (n=6), oHSV-GM-CSF (n=6), SC-oHSV-GM-CSF (n=7), or SC-oHSV and SC^{N1KO}-G (n=7). Data are represented as mean SEM. (FIG. 3J) Representative images of immunofluorescence analysis for CD3+ TILs in tumor tissues harvested 30 days after UV2-GFl inoculation. Scale bar, 6 μm. (FIG. 3K) Mean number of TILs expressing CD3 was statistically assessed from three selected fields (n=5 per group). Data are represented as mean±SD. (FIG. 3L) In vivo levels of GM-CSF after treatment with SC-RmC (n=3), oHSV-GM-CSF (n=3-4), SC-oHSV-GM-CSF (n=3), or SC-oHSV and SC^{N1KO}-G (n=4). The tumors were collected on day 2 and 5 aftertreatment were isolated. Data are represented as mean±SD. *: p<0.05. **: p<0.01, ***: p<0.001, ****: p<0.0001.

FIGS. 4A-4I show TSC-G therapy generates systemic immunity against bilateral flank PTEN-deficient melanoma in vivo. (FIG. 4A) Experimental design. In brief, in the bilateral Y1.1-GFl and UV2-GFl subcutaneous tumor model, one side was treated with SC-oHSV and SC^{N1KO}-GM-CSF (SC^{N1KO}-G) intratumorally twice, and the other side was left untreated. Tumor volumes were measured every 3-5 days post-implantation. (FIG. 4B) Plots showing subcutaneous Y1.1-GFl tumor growth in mice treated with control SC-Rluc-mCherry (RmC) (n=7), oHSV-GM-CSF (n=7), SC-oHSV (n=8), or TSC-G (n=8). (Left) Subcutaneous tumor growth in treated tumors. (Right) Subcutaneous tumor growth in untreated tumors. Data are represented as mean±SEM. (FIG. 4C) Plots showing subcutaneous UV2-GFl tumor growth in mice treated with control SC-RmC (n=6), oHSV-GM-CSF (n=6), SC-oHSV (n=6), or TSC-G (n=6). (Left) Subcutaneous tumor growth in treated tumors. (Right) Subcutaneous tumor growth in untreated tumors. Data are represented as mean±SEM. (FIG. 4D) Y1.1-GFl bearing mice after treatment (n=4 per group) were re-challenged into the brain. Plot showed brain metastatic tumor growth measured by in vivo Flue bioluminescence. Data are represented as mean±SEM. (FIG. 4E) Splenocytes from mice after treatment (n=3 per group) were incubated at 37° C. with either Y1.1-GFl melanoma cells or TC-1-GFl lung at effector cell:target cell ratios (8:1). Data are represented as mean±SD. (FIG. 4F) Immunofluorescence analysis of CD11c, CD3, CD4, and CD8 in UV2-GFl tumors on 14 days after treatment (n=4-5 per group). Scale bar, 6 μm. (FIG. 4G) Mean number of TILs expressing CD11c, CD3, CD4, and CD8 was statistically assessed from three selected fields. (FIGS. 4H and 4I) Flowcytometry showing central memory CD8 positive T cells and effecter memory CD8 positive T cells on splenocytes aftertreatment. Data are represented as mean±SD. *: p<0.05. **: p<0.01, ***: p<0.001.

FIGS. 5A-5J show SC secreting dual immunomodulators with SC-oHSV (TSC) against immunosuppressive leptomeningeal metastasis. (FIG. 5A) Scheme showing creation of SC^{N1KO}-GM-CSF/scFvPD-1 (G/P). (FIG. 5B) Expression of GM-CSF and scFvPD-1 in supernatant from SC^{N1KO}-G/P by western blotting. (technical replicates). (FIG. 5C) BLI signal and photographs of IT-injected SC^{N1KO}-G/P-Fluc-bearing mice (n=3). Data are represented as mean±SEM. (FIG. 5D) Experimental design. In brief, in the UV2-GFl leptomeningeal metastasis tumor model, SCs were intrathecally administrated one time 5 days after implant tumors. Tumor volumes were measured every 2-3 days by BLI imaging. (FIG. 5E) Flue signal curves and representative BLI images of mice bearing UV2-GFl tumors treated with SC-RmC (n=6), SC-oHSV (n=6), TSC-G or TSC-G/P (n=7). (FIG. 5F) Kaplan-Meier curves of overall survival of mice. Data are represented as mean±SEM. (FIG. 5G) Kaplan-Meier curves of overall survival of NOD/SCID mice after treatment with SC-RmC (n=4), TSC-G (n=4), or TSC-G/P (n=4). (FIGS. 5H and 5I) Flowcytometric analysis of TILs collected from UV2-GFl-bearing leptomeningeal metastasis tumor 7 days after treatment. Data are represented as mean±SD. (FIG. 5J) Heatmap of differential expression of genes associated with immune cell types after treatment in leptomeningeal metastasis tumor model. *: p<0.05. ***: p<0.001.

FIGS. 6A-6N show establishment of patient derived PTEN-deficient melanoma brain metastasis BLT mouse model and allogeneic SC releasing human GM-CSF/scFvPD-1-TK and SC-oHSV (hTSC-G/P-TK) against brain metastasis. (FIG. 6A) Scheme showing intracranial injection (IC) and intrathecal injection as leptomeningeal metastasis (LM) with patient derived melanoma brain metastasis (M12-GFl) cells. (FIG. 6B) BLI signal curve of intracranially or intrathecal injected M12-GFl-bearing BLT humanized mice (n=3-4 per each group). Data are represented as mean±SEM. (FIG. 6C) HE staining of brain M12-GFl tumor model. Below. Immunofluorescence analysis of CD11c, and CD3 in brain M12-GFl tumor BLT humanized model. Scale bar, 6 μm. (FIG. 6D) Flowcytometric (FCM) analysis of immune profiling of brain M12-GFl tumor model (IC) and leptomeningeal metastasis model (LM) (n=4 per group). Data are represented as mean±SD. (FIG. 6E) Scheme showing creation of human SC^{N1KO} (hSC^{N1KO})-human GM-CSF/scFvPD-1 (hG/P)-TK. (FIG. 6F) Cell viability assays showing hSC^{N1KO}-hG/P-TK resistance to oHSV compared to human SC (hSC) (n=5 per group, technical replicates). Data are represented as mean±SD. (FIG. 6G) Expression of human GM-CSF and scFvPD-1 in supernatant from hSC^{N1KO}-G/P-TK by western blotting. (FIG. 6H) Cell viability assay of SC^{N1KO}-hG/P-TK in the absence or presence of GCV for 2 days (n=5 per group, technical replicates). Data are represented as mean±SD. (FIG. 6I) M12-GFl cells were investigated by cell viability assay 3 days after hSC-oHSV treatment in vitro (n=5 per group, technical replicates). Data are represented as mean±SD. (FIG. 6J) Experimental design. In brief, in the M12-GFl leptomeningeal metastasis tumor model, SCs were intrathecally administered one time 5 days after implant tumors. Tumor volumes were measured every 2-4 days by BLI imaging. (FIG. 6K) Flue signal curves and representative BLI images of BLT humanized mice bearing M12-GFl tumors treated with hSC (n=4), or hTSC-G/P-TK (n=4). Data are represented as mean±SEM. (FIG. 6L) Kaplan-Meier curves of overall survival of mice. (FIGS. 6M and 6N) FCM analysis of immune cells collected from M12-GFl leptomeningeal metastasis tumor 10 days after treatment (n=3 per group). *: p<0.05.

FIGS. 7A-7C show PTEN-deficient is correlated with melanoma brain metastasis and immune suppression. (FIG. 7A) mRNA levels of BRAF, TP53 and KRAS in patient samples of metastatic stage from TCGA database (n=337). (FIG. 7B) Immune profiles analysis of high and low PI3K/AKT pathway in melanoma. (FIG. 7C) Immune profiles analysis in primary melanoma and metastatic melanoma.

FIG. 8 shows characterizing engineered murine melanoma cells. Correlation between Flue signals in vitro and the number of cells engineered to express Flue that were used in this study (n=5, technical replicates). Scar bar, 100 μm.

FIGS. 9A-9F show Utility of SCs as carrier for oHSV. (FIG. 9A) Cell viability assay of human melanoma (Mewo and M12) cells was assessed 3 days after oHSV treatment at the indicated doses (MOI) (n=5, technical replicates). Data are represented as mean±SD. (FIG. 9B) Cell viability assay of murine melanoma cells (Y1.1, Y2.1 and UV2) was assessed 3 days after Temozolomide (TMZ) at the indicated doses (n=5, technical replicates). Data are represented as mean±SD. (FIG. 9C) Cell viability assay of melanoma cells was assessed 3 days after oHSV or oHSV-GM-CSF treatment at the indicated doses (MOI) (n=5, technical replicates). Data are represented as mean±SD. (FIG. 9D) Whole-cell lysates of Y1.1 and UV2 cells collected 3 days after oHSV treatment (0, 1, 2, 5, 10, and 20 MOI) were subjected to western blot analysis of PARP, pRIP3, HMGB1, and Vinculin expression (technical replicates). (FIG. 9E) Representative light microscopy and fluorescence microscopy images of SCs infected with 2 and 5 MOI oHSV-FmC at 12 h and 24 h after infection. (FIG. 9F) Representative plots of multi-cytokine and chemokine assays 24 h after oHSV infection (0, 2MOI). Cytokines and chemokines secreted from SC-oHSV cells were measured using multi-cytokine and chemokine assays 24 h after oHSV infection (0, 2MOI) and ratio of 2 to 0 MOI was plotted for each cytokine or chemokine by ImageJ.

FIGS. 10A-10I show establishment of oHSV-resistant SC secreting GM-CSF and the influence against dendritic cells and Macrophages. (FIG. 10A) Cell viability assays showing SC^{N1KO}-GM-CSF (SC^{N1KO}-G) resistance to oHSV compared to SC (n=5 per group, technical replicates). (FIG. 10B) Expression of GM-CSF Ra on SCs, RAW264.7 cells and murine melanoma cells by flowcytometry (FCM). (FIG. 10C) Cell viability assays showing influence of SC^{N1KO}-GM-CSF (SC^{N1KO}-G) and SC-Rluc-mCherry (RmC) on murine macrophage (RAW264.7) and melanoma (Y1.1-GFl, Y2.1-GFl and UV2-GFl). GM-CSF induced macrophage growth but not melanoma in vitro (n=5 per group, technical replicates). Data are represented as mean±SD. (FIG. 10D) Plots showing subcutaneous Y1.1-GFl tumor growth in mice treated with control SC-RmC (n=8) or SC^{N1KO}-G (n=7). Data are represented as mean SEM. (FIG. 10E) Representative plots of TNFα positive RAW264.7 cells by FCM after incubation with SC^{N1KO}-G conditioned medium (CM) for 4 days (n=3 per group). (FIG. 10F) Representative plots of M1 macrophages (CD45+CD11b+F4/80+CD86+) and M2 macrophages (CD45+CD11b+F4/80+CD206+) by FCM. **: p<0.01, ***: p<0.001, ****: p<0.0001. (FIG. 10G) SC^{N1KO}-G conditioned medium (CM) induces differentiation to M1 and M2 macrophages from murine bone marrow derived macrophages by FCM after incubation for 3 days (n=3 per group). Data are represented as mean±SD. (FIG. 10H) Representative plots of dendritic cells (CD45+CD11b+CD11c+) and mature dendritic cells (CD45+CD11b+CD11c+MHC II I-A/I-E+) by FCM. (FIG. 10I) Western blot analysis showing GM-CSF release from in vitro co-culture with Y1.1-GFl cells and SC^{N1KO}-G or SC-RmC and oHSV-GM-CSF.

FIGS. 11A-11E show TSC-G therapy generates systemic immunity against bilateral flank PTEN-deficient melanoma in vivo. (FIG. 11A) Tumor volume (mm³) at day 20 after treatment in Y1.1-GFl bilateral flank model and (FIG. 11B) at day 18 after treatment in bilateral UV2-GFl subcutaneous model. Data are represented as mean±SEM. (FIG. 11C) Splenocytes from mice after treatment (n=3 per group) in bilateral Y1.1-GFl subcutaneous tumor model were incubated at 37° C. with either Y1.1-GFl melanoma cells and TC-1-GFl lung cancer cells at increasing effector cell:target cell ratios (1:1-8:1). Data are represented as mean±SD. (FIG. 11D) Representative plots of central memory and effecter memory CD8+ cells on splenocytes at 30 days after treatment. (FIG. 11E) H&E staining showing toxicity in major organs after treatment. Scale bar 100 μm. f. Variation in body weights of mice after treatment.

FIGS. 12A-12K show SC secreting dual immunomodulators with SC-oHSV (TSC) against immunosuppressive leptomeningeal metastasis. (FIG. 12A) Expression of PD-L1 on murine melanoma cells by flowcytometry (FCM). (FIG. 12B) FCM showing decreased PD-1 expression on CD3+spenocytes after incubation with condition medium of SC^{N1KO}-scFvPD-1. (FIG. 12C) Flue signal curves and representative BLI images of mice bearing UV2-GFl tumors treated with SC-RmC (n=6), SC-oHSV (n=6) or SC-oHSV+SC^{N1KO}-scFvPD-1 (n=6). Data are represented as mean±SEM. (FIG. 12D) Kaplan-Meier curves of overall survival of mice. (FIG. 12E) Flue signal curves and representative BLI images of NOD/SCID mice after treatment with SC-RmC (n=4), TSC-G (n=4), or TSC-G/P (n=4). Data are represented as mean±SEM. (FIG. 12F) Representative plots of gating for immune profiling. (FIG. 12G) GO analysis of immune associated pathways (Upregulated gene) after the treatment (TSC-G or TSC-G/P versus SC-RmC). (FIG. 12H) GO analysis of JAK-STAT pathways (Downregulated genes) after the treatment (TSC-G or TSC-G/P versus SC-RmC). (FIG. 12I) KEGG analysis of PI3K-AKT and JAK-STAT pathways (Downregulated genes) after the treatment (TSC-G or TSC-G/P versus SC-RmC). (FIG. 12J) Flue signal curves and representative BLI images of mice bearing Y2.1-GFl tumors treated with SC-RmC (n=4) or TSC-G or TSC-G/P (n=4). Data are represented as mean±SEM. (FIG. 12K) Kaplan-Meier curves of overall survival of mice bearing Y2.1-GFI tumors.

FIGS. 13A-13C show safety of SC secreting dual immunomodulators with SC-oHSV (TSC) against metastatic melanoma. (FIG. 13A) Representative photomicrographs of Hematoxylin and eosin stain and immunohistochemistry of NeuN, GFAP and IBA1 in brain from intracranially injected UV2-GFl-bearing mice treated with SC-RmC, oHSV-GM-CSF and TSC-G (n=3, Scale bars, 100 μm). (FIG. 13B) H&E staining showing toxicity in major organs after treatment of TSC-G or TSC-G/P. Scale bar 100 μm. (FIG. 13C) Variation in body weights of mice after intrathecal injection of TSC-G or TSC-G/P.

FIG. 14A-14G show establishment of patient derived PTEN-deficient melanoma brain metastasis BLT mouse model and allogeneic SC releasing human GM-CSF/scFvPD-1-TK and SC-oHSV (hTSC-G/P-TK) against brain metastasis. (FIG. 14A) BLI signal curve of intracranially injected M12-GFl-bearing NOD/SCID mice (n=5). Data are represented as mean±SEM. (FIG. 14B) Representative plots of immune profiling in UV2-GFl brain tumor BLT mouse model and leptomeningeal metastasis BLT mouse model. (FIG. 14C) Flowcytometric (FCM) analysis of immune profiling of brain M12-GFl tumor model (IC) and leptomeningeal metastasis model (LM) at the bone marrow cells (BM), the splenocytes (Sp) and the cervical lymph node (Ly) (n=4). Data are represented as mean±SD. (FIG. 14D) Cell viability assays showing human SC^{N1KO} resistance to oHSV compared to human SC (n=5). Data are represented as mean±SD. (FIG. 14E) Representative plots of gating for immune profiling after treatment by FCM (n=3 per group). (FIG. 14F) FCM analysis of immune cells collected from M12-GFl leptomeningeal metastasis tumor 10 days after treatment (n=3 per group). *: p<0.05. Data are represented as mean±SD. (FIG. 14G) Variation in body weights of mice after treatment in patient derived melanoma LM BLT mouse model (each group: n=4). Data are represented as mean±SD.

FIGS. 15A-15C show the concept of the study and screening of different human and mouse stem cell lines for sensitivity to receptor-targeted anticancer therapeutics (FIG. 15A) Stem cells (SCs) inherently sensitive (sSC) to surface receptor-targeted ligands (RTL), such as oHSV, IFNb, and TRAIL, can be engineered with CRISPR/Cas9 to knock out RTL-targeted surface receptors (RT). RT knockout stem (rSCs) cells gain resistance to RTL, which allows engineering them to secrete RTL (IFNb or TRAIL). When used in cancer these rSCs can now have anticancer effects by secreting cancer targeted RTL without inflicting autocrine toxicity. Moreover, rSCs could be used in admixtures with sSCs loaded with oHSV, allowing continued secretion of RTL from rSCs without being susceptible to oHSV infection from neighboring oHSV-releasing sSCs thereby potentially increasing anticancer efficacy in combined therapy models. (FIGS. 15B and 15C) Sensitivity screening of different mouse (FIG. 15B) and human (FIG. 15C) stem cell lines to receptor-targeted therapeutics oHSV, TRAIL and mIFNb (IFNb) identifies resistant and sensitive lines.

FIGS. 16A-16C show TCGA analysis identifies Nectin1, IFNaR1 and DR4/5 as promising RTL targets in a variety of cancers and screening of a panel of mouse and human cancer cell lines confirms broad applicability of oHSV, IFNb and TRAIL for anticancer therapy (FIG. 16A) TCGA analysis of commonly targeted receptors for different cancers as well as receptors targeted in this study (IFNaR1, DR4/5, Nectin1). (FIGS. 16B and 16C) A panel of mouse (FIG. 16B) and human (FIG. 16C) cancer cell lines were titrated with oHSV, TRAIL (human cancer lines) and IFNb (mouse cancer lines). Graphs show cell viability in comparison to control 72 h post treatment (oHSV and TRAIL) or 120 h post treatment (IFNb).

FIGS. 17A-17E show CRISPR/Cas9 engineering of therapy-sensitive stem cells allows targeted knockout of surface receptors (FIG. 17A) Stem cells (maMSC, hNSC and haMSC) identified as sensitive to potentially secretable therapeutics were engineered with lentivirus and blotted for Cas9-FLAG. (FIG. 17B) hNSCs expressing Cas9 and SgRNAs targeting DR4, DR5 or both receptors were subcloned and single clones where blotted for DR expression against non-SgRNA engineered control. (FIG. 17C) maMSCs expressing Cas9 and SgRNAs targeting Nectin1 were subcloned and single clones where blotted for Nectin1 expression against non-SgRNA engineered control. (FIG. 17D) maMSCs expressing Cas9 and SgRNA targeting IFNaR1 were subcloned followed by treatment of wild-type and IFNaR1 KO cells with and without recombinant IFNb (50 ng/ml, 6 h treatment). (FIG. 17E) Genomic DNA of single CRISPR-modified stem cell clones was analyzed to confirm indel mutations at Cas9-SgRNA targeted exonic gene sites.

FIGS. 18A-18E show DR knockout hNSCs are resistant to TRAIL-induced apoptosis, can be engineered to secrete TRAIL and show in vitro anticancer efficacy against a broad panel of cancers. (FIG. 18A) DR wild type hNSCs and DR4/5 knockout hNSCs (hNSC^{DR4/5}) were titrated with TRAIL and viability was measured 72 h post TRAIL treatment. Photomicrograph shows hNSC wild type control (hNSC) and hNSC^{DR4/5} treated with 50 ng/ml TRAIL 8 h post trail treatment. (FIG. 18B) hNSCDR4/5 were transduced with LV encoding secretable TRAIL and GFP (ST) followed by harvesting of cell lysates and conditioned media (CM) 5 days post transduction. Photomicrograph shows GFP expression following transduction. Western blot shows expression of S-TRAIL from cell lysates and CM in comparison to control. (FIG. 18C) hNSC or hNSC^{DR4/5} were transduced with LV encoding a fusion variant of S-TRAIL with the optical reporter Renilla luciferase (Rluc(o), RI). Following transduction S-TRAIL secretion was monitored using bioluminescent imaging over time. (FIG. 18D) Representative photomicrographs (top) and viability assessment (bottom) of LV-Fluc-mCherry engineered established (Gli36Δ and U251) and primary (GBM8) glioblastoma cell lines cocultured with increasing number of either GFP-transduced hNSC (control) or hNSC^{DR4/5}-ST. (FIG. 18E) Representative photomicrographs (top) and viability assessment (bottom) of LV-Fluc-mCherry engineered Lung, Breast and Colon cancer cell lines (H2170, MDA-231, HCT116) cocultured with an increasing number of either GFP-transduced hNSC (control) or hNSC^{DR4/5}-ST.Black/white scale bar indicates 200 μm.

FIGS. 19A-19C show IFNaR1 knockout maMSCs are resistant to IFNb, can be engineered to secrete IFNb and show in vitro anticancer efficacy against syngeneic glioblastoma and breast cancer cell lines. (FIG. 19A) IFNaR1 wild type maMSCs and IFNaR1 knockout maMSCs (maMSC^{IFNaR1}) were titrated with recombinant IFNb and viability was measured 72 h post TRAIL treatment. Photomicrograph shows maMSC wild type control (maMSC) and maMSC^{INFaR1} treated with 120 h post treatment with IFNß (250 IU/ml). (FIG. 19B) Photomicrographs show maMSC^{INFaR1} transduced with retrovirus (RV) encoding secretable IFNß and GFP (IFNß). In addition CM of two different IFNaR1 maMSC clones (10H4 & 10H7) transduced with IFNß was collected 24 h after plating followed by 25× concentration and blotting against mouse-specific IFNß antibody with recombinant IFNß used as control. (FIG. 19C) Representative photomicrographs (top) and viability assessment (bottom) of LV-Fluc-mCherry engineered glioblastoma (G1261 and CT2a) and breast cancer (4T1) cell lines cocultured with increasing number of either GFP-transduced maMSCs control (maMSC-GFP) or maMSC^{IFNaR1}-IFNß. Black/white scale bar indicates 200 μm.

FIGS. 20A-20C show Nectin1 knockout confers resistance to oHSV infection (FIG. 20A) haMSCs expressing Cas9 and different SgRNA targeting human Nectin1 (hNectin1) were titrated with oHSV. Photomicrographs show representative brightfield images 72 h after oHSV-infection (MOI 5). Bar graph indicates haMSC viability of hNectin1 wild type control versus SgRNA1 or SgRNA4 transduced controls depending on oHSV MOI. (FIG. 20B) maMSCs expressing Cas9 and different SgRNA targeting mouse Nectin1 (mNectin1) were titrated with oHSV. Photomicrographs show representative brightfield images 72 h after oHSV-infection (MOI 5). Bar graph indicates maMSC cell viability of mNectin1 wild type control versus SgRNA1 or SgRNA3 transduced controls depending on oHSV MOI. (FIG. 20C) maMSC mNectin1 wild type cells were transduced with LV encoding either Rluc-mCherry or GFP-Fluc (maMSC-RmC and maMSC-GFl respectively) and maMSC mNectin1 knockout cells (maMSC^{Nectin1}) were transduced with LV encoding GFP-Fluc (maMSC^{Nectin1}-GFl). Stably transduced maMSC-RmC (mNectin1 wild type) were cocultured overtime in a 1:1 ratio with either maMSC-GFl (mNectin1 wild type, above horizontal black line) or maMSCNectin1-GFl (below horizontal black line) in the presence or absence (control) of oHSV. Bar graphs on the right indicates increase/decrease of cell viability of individual cell populations over time. Black/white scale bar indicates 200 μm.

FIGS. 21A-21D show in vivo anticancer efficacy of IFNß and ST secreting CRISPR-enhanced stem cells. (FIG. 21A) CT2a-FmC were co-injected intracranially in 1:1 ratio with either maMSC-GFP or maMSC^{IFNaR1}-IFNß (1.5×10\hat{ }5 each) and CT2a-FmC tumor growth was followed by BLI overtime (n=5 each). Table shows representative mice images at indicated time points. Kaplan-Meier Survival graph shows survival of Control (CT2a-FmC vs. maMSC-GFP) versus IFNß group (CT2a-FmC vs. maMSC^{IFNaR1}-IFNß), p<0.0023. (FIG. 21B) 4T1-FmC were orthotopically co-injected in 1:1 ratio with either maMSC-GFP or maMSC^{IFNaR1}-IFNß (5×10\hat{ }5 each) and 4T1-FmC tumor growth was followed by BLI over time (n=5 each). Table shows representative mice images at indicated time points. (FIG. 21C) GBM8-FmC were co-injected intracranially in 1:1 ratio with either hNSC-GFP or hNSC^{DR4/5}-ST (1.5×10\hat{ }5 each) and GBM8-FmC tumor growth was followed by BLI overtime (GBM8-FmC vs. hNSC-GFP n=3, GBM8-FmC vs. hNSC^{DR4/5}-ST n=4). Table shows representative mice images at indicated time points. Kaplan-Meier Survival graph shows survival of Control (GBM8-FmC vs. hNSC-GFP) versus S-TRAIL group (GBM8-FmC vs. hNSC^{DR4/5}-ST) p<0.014. Photomicrographs show representative HE and fluorescence of Control and S-TRAIL groups at day 40 post implantation. (FIG. 21D) HCT116-FmC were co-injected into the flank of SCID mice (n=2 injections per mouse) in 1:1 ratio with either hNSC-GFP or hNSC^{DR4/5}-ST (5×10\hat{ }5 each) and HCT116-FmC tumor volume was followed by calipeter measurements over time (Control n=2 mice, S-TRAIL n=3 mice). In addition BLI-signal was evaluated at day 15 post implantation. Tumors were harvested at day 31 post implantation and tumor weight was compared using t test (p<0.005). In 2 of the 6 injection sites in the S-TRAIL group there was no tumor development.

FIGS. 22A-22B show Nectin1 knockout increases in vivo viability of maMSCs when co-injected with oHSV-infected Nectin1 wild type maMSCs. (FIG. 22A) Experimental outline for testing in vivo viability of wild type and Nectin1 KO maMSCs admixed with oHSV-infected maMSCs (maMSC-RmC). Nectin1 wild type maMSCs engineered with Rluc-mCherry (maMSC-RmC) were incubated for 6 h with oHSV (MOI 10) followed by co-injected into the flanks of C57/BL6 mice in 1:1 ratio with either maMSC-GFl (Nectin1 wild type) or maMSC^{Nectin1}-GFl (2 injections per mouse; maMSC-RmC/oHSV vs. maMSC-GFl n=2 mice, maMSC-RmC/oHSV vs. maMSC^{Nectin1}-GFl n=3 mice). (FIG. 22B) Top: Bar graph shows in vivo fate of GFl engineered maMSCs and representative mouse images from different time points post injection. Bottom: Bar graph shows in vivo fate of RmC engineered oHSV-infected maMSCs and representative mouse images from different time points post injection.

FIGS. 23A-23F shows TCGA analysis including heat maps for commonly targeted receptors in GBM cells (FIG. 23A), colon adenocarcinoma cells (FIG. 23B), prostate adenocarcinoma cells (FIG. 23C), breast cancer cells (FIG. 23D), lung adenocarcinoma cells (FIG. 23E), or melanoma cells (FIG. 23F).

FIG. 24 shows engineering of color and bioluminescent cell lines. Graphs show correlation of Flue or Rluc bioluminescent signal intensity with cell number. Black/white scale bar indicates 200 μm.

FIGS. 25A-25B show in vitro efficacy of oHSV and S-TRAIL against metastatic prostate cancer cell line PC3 (FIG. 25A) PC3 cells were titrated with oHSV (left) and TRAIL (right). (FIG. 25B) Viability assessment (left) and representative photomicrographs (right) of LV-Fluc-mCherry engineered PC3 cells (PC3-FmC) cocultured with increasing number of either GFP-transduced hNSC (control) or hNSC^{DR4/5}-ST. White scale bar indicates 200 μm.

FIG. 26 shows photomicrographs from in vitro time-laps video of coculture of Nectin1 wild type and knockout maMSCs after oHSV-mCherry infection: maMSC-RmC (Nectin1 wild type) were plated in 1:10 ratio with either maMSC-GFl (Nectin1 wild type) or maMSCNectin1-GFl (Nectin1 knockout) followed by infection with oHSV encoding an imageable fusion variant (oHSV-mCherry) to allow live-monitoring of oHSV-infection overtime. Photomicrographs show representative field of views at indicated time points post oHSV-mCherry infection (MOI 5).

FIGS. 27A-27B show CRISPR-engineered maMSCs and haMSCs retain their potential for in vitro osteogenic differentiation. (FIG. 27A) Wild type, IFNaR1 knockout and Nectin1 knockout maMSCs were plated into 12 well plates followed by induction of osteogenic differentiation and staining with alizarin red to visualize extracellular calcium deposits (red stain). Macroscopic photos (top) and photomicrographs (bottom) showing undifferentiated control in comparison to differentiated maMSCs post alizarin red staining. Graph on the right shows degree of differentiated cells based on microscopic evaluation of 6 fields of view, rated 0 to 10. (FIG. 27B) Wild type and Nectin1 knockout haMSCs (two different SgRNAs, as indicated) were plated into 12 well plates followed by induction of osteogenic differentiation and staining with alizarin red to visualize extracellular calcium deposits (red stain). Macroscopic photos (top) and representative photomicrograph (bottom) showing undifferentiated control in comparison to differentiated haMSCs post alizarin red staining. Graph on the right shows degree of differentiated cells based on microscopic evaluation of 6 fields of view, rated 0 to 10. Bar in 12-well plate wells indicates 5 mm, Black scale bar on photomicrographs indicates 200 μm.

FIG. 28 shows CRISPR-engineered hNSC retain neural stem cell markers Nestin and SOX2 Left panel: hNSC DR4/5 wild type Right panel: hNSC DR4/5 KOWhite scale bar on photomicrographs indicates 100 μm unless otherwise indicated.

FIGS. 29A-29D show creating and characterizing nectin-1 knock out MSC using CRISPR/Cas9 system. (FIGS. 29A-29B) oHSV-sensitive mouse MSC were engineered to co-express Cas9 and different SgRNAs targeting Nectin1 receptor gene. Western blot analysis showing expression of Cas-9 (FIG. 29A) and single clone sequencing of genomic DNA from wild type and Nectin1-targeting SgRNA clones showing indel mutation at SgRNA-targeted exonic Nectin1 gene segments (FIG. 29B). (FIGS. 29C-29D) oHSV-selected mixed populations expressing SgRNA1 were expanded and titrated with oHSV in comparison to wild type control. Fluorescent images (FIG. 29C) and plot (FIG. 29D) showing viability of wt MSC and nectin-1 knockout MSC post oHSV treatment.

FIG. 30 shows MSC-N1^{KO} mediated GMCSF expression enhances the efficacy of MSC-oHSV in vivo in melanomas. Y1.1 expressing GFP-firefly luciferase fusion protein were admixed with MSC-oHSV/MSC-oHSV-GMCSF (5:1 ratio) or with MSC-N1^{KO} expressing GMCSF or IL-12 or GMCSF or 41BBL or IL15 and MSC-oHSV or MSC and implanted in the flanks of C57BL6 mice. Plot showing the changes in Y1.1 tumor cells viability over time. Inset: Flue images showing tumor volumes on day 7.

FIG. 31 shows MSC-N1^{KO} mediated IL-12 expression enhances the efficacy of MSC-oHSV in vivo. Upper: Table showing different immunomodulator functions. Lower: GBM cells CT2A-GFP-Fluc were admixed with MSC-N1^{KO} expressing GMCSF, IL-12, 41BBL or IL15 and MSC-oHSV (10:1:1 ratio) and implanted in the flanks of C57BL6 mice. Plot showing the changes in CT2A-GFP-Fluc viability over time. Inset: Flue images showing tumor volumes on day 6.

FIGS. 32A-32E show MSC-N1^{KO} mediated IL-12 expression induces CD4 and CD8 T cell response in vitro (FIG. 32A) Photomicrograph of mouse MSC-N1^{KO} expressing IL-12 (FIG. 32B) Western blot analysis showing expression of IL-12 in cells lysates and conditioned medium. (FIG. 32C) Plot showing sorted T cells activated with anti-CD3 and anti-CD28 and cultured in presence of 1 μL of conc. supernatant from 1×10⁵ MSC-IL-12 or MSC-GFP. (FIGS. 32D-32E) Activated T cells in presence of titrating volumes of conc. IL-12 supernatant were followed by intracellular staining for IFNg. Plots showing % IFNg+ cells in CD4 (FIG. 32D) and CD8 (FIG. 32E) T cells. Insets D&E: representative flow cytometry plots.

FIG. 33 shows regulatable system Mice bearing established intracranial CT2A-FmC tumors were treated with intratumorally injections of MSC+MSC^{N1KO}, MSC-oHSV+MSC^{N1KO} and MSC-oHSV+MSC^{N1KO}-Tet-IL-12. Mice were administered 9 TB-dox (10 mg/Kg) i.p. daily after treatment as described. (B) Plot showing changes in tumor growth monitored overtime. *P<0.05 MSC-oHSV+MSC^{N1KO}-Tet-IL-12 (Dox Day 3-9) versus MSC+MSC^{N1KO} control.

FIGS. 34A-34I show the establishment and characterization of MSC^{N1KO}-GMCSF and influence for melanoma and macrophage (FIG. 34A). Scheme showing creation of MSC^{N1KO}-GMCSF, (FIG. 34B) Western blotting showing expression of Nectin-1 in MSC and MSC^{N1KO} (FIG. 34C). Western blotting showing expression of GMCSF in MSC^{N1KO}-GMCSF. (FIG. 34D) Cell viability assays showing MSC^{N1KO}-GMCSF resistance to oHSV compared to MSC (FIG. 34E) MSC^{N1KO} mediated GMCSF expression enhances the antitumor activity of oHSV-loading MSC in vivo as compared to MSC^{N1KO} releasing other immunomodulators, like IL-12, $1BBL and IL-15. (FIG. 34F). Flow cytometry showing expression of GMCSF receptorα in murine macrophage cell line, RAW264.7 and melanoma Y1.1-GFP-Fluc cell line (FIG. 34G) Cell viability assays showing influence of GM-CSF on murine macrophage RAW264.7 and melanoma Y1.1-GFP-Fluc. GM-CSF induced macrophage growth but not melanoma Y1.1-GFP-Fluc in vitro. (FIG. 34H) Western blot analysis showing TNF-α release from RAW264.7 in vitro post incubation with MSC^{N1KO}-GMCSF conditioned medium. (FIG. 34I) Influence of MSC^{N1KO}-GMCSF on. 1-GFP-Fluc tumors in vivo. Mice were treated with MSC-Rluc-mCherry (MSC-RmC), or MSC^{N1KO}-GM-CSF intratumorally two times. Tumor volumes were measured every 5 days post-implantation.

FIGS. 35A-35D show MSC-oHSV and MSC^{N1KO}-GM-CSF therapy generates systemic immunity against bilateral flank UV2-GFP-Fluc melanoma in vivo. (FIG. 35A) Experimental design. In brief, in the bilateral UV2-GFP-Fluc subcutaneous tumor model, one side was treated with MSC-oHSV and MSCN1KO-GM-CSF intratumorally two times, and the other side was left untreated. Tumor volumes were measured every 3-5 days post-implantation. (FIG. 35B) Plots showing subcutaneous UV2-GFP-Fluc tumor growth in mice treated with control MSC-RmC (n=6), oHSV-GM-CSF (n=6), MSC-oHSV (n=6), or MSC-oHSV and MSCN1KO-GM-CSF (n=6). (Left) Subcutaneous tumor growth in treated tumors. (Right) Subcutaneous tumor growth in untreated tumors. (FIG. 35C) Immunofluorescence analysis of CD11c, CD3, CD4, and CD8 in UV2-GFP-Fluc tumors on 14 days after treatment (n=4-5 per a group). Scale bar, 100 μm. (FIG. 35D) Mean number of TILs expressing CD11c, CD3, CD4, and CD8 was statistically assessed from three selected fields. *: p<0.05. **: p<0.01,

FIGS. 36A-36F show MSC secreting dual immunomodulators and MSC-oHSV against immune-suppressive leptomeningeal metastasis. (FIG. 36A) Scheme showing creation of MSCN1KO-GM-CSF/scFvPD-1 (G/PD-1). (FIG. 36B) Expression of GM-CSF and scFvPD-1 in supernatant from MSCN1KO-G/PD-1 by Western blotting. (FIG. 36C) Experimental design. In brief, in the UV2-GFP-Fluc leptomeningeal metastasis tumor model, MSCs were intrathecally administered one time 5 days after implant tumors. Tumor volumes were measured every 2-3 days by BLI imaging. (FIG. 36D) Flue signal curves and representative BLI images of mice bearing UV2-GFP-Fluc tumors treated with MSC-RmC (n=6), MSC-oHSV (n=6), MSC-oHSV+MSCN1KO-G (n=6), or MSC-oHSV+MSCN1KO-G (n=7). (FIG. 36E) Kaplan-Meier curves of overall survival of mice. (FIG. 36F) Flowcytometric analysis of TILs collected from UV2-GFP-Fluc tumor 7 days after treatment. *: p<0.05. ***: p<0.001.

FIGS. 37A-37D show MSC releasing human GM-CSF and MSC-oHSV against patient derived PTEN-deficient melanoma brain metastasis (M12-GFP-Fluc). (FIG. 37A) Scheme showing creation of human MSCN1KO-human GM-CSF. (FIG. 37B) Cell viability assays showing MSC^{N1KO} resistance to oHSV compared to human MSC. (FIG. 37C) Cell viability assay of MSCN1KO-G-TK in the absence or presence of GCV for 2 days (n=4 per group). (FIG. 37D) M12-GFP-Fluc cells were treated with oHSV (0-10MOI) for 3 days, and cell viability was assessed by CellTiterGlo assay (n=5).

FIGS. 38A and 38B show therapeutic efficacy of MSC-oHSV and MSC-MSCN1KO-IL-12 in resected GBMs in vivo. (FIGS. 38A and 38B) Mice bearing established intracranial CT2A-FmC tumors were resected and treated with intracavitary injections of synthetic extracellular matrix (sECM)-encapsulated MSC, oHSV-infected MSC (MSC-oHSV) and MSC-oHSV plus MSCN1KO-IL-12. (FIG. 38A) Plot showing changes in Flue activity as a measure of tumor growth monitored over time. Representative bioluminescence images from each group at day 12 after resection are shown. *P<0.05 MSC-oHSV and MSC-oHSV plus MSCN1KO-IL-12 versus MSC control at day 12; MSC-oHSV plus MSCN1KO-IL-12 versus MSC-oHSV at day 14 (t-test, two-sided). (FIG. 38B) Kaplan-Meier survival curves of mice bearing resected CT2A-FmC tumors in the three treatment groups. Median survival of MSC-oHSV plus MSCN1KO-IL-12-treated mice (56.5 days; n=6) was compared with that of MSC (22.5 days; n=4) and MSC-oHSV (28 days; n=6) by log-rank analysis (p<0.0001).

FIGS. 39A and 39B show characterization of MSC-N1KO mediated IL-12 expression. (FIG. 39A) GBM-bearing mice were sacrificed 2 and 6 days post-intratumoral injection of 2×105 MSC-N1KO-IL-12 or MSC-N1KO-GFP. Plot showing ELISA data for mouse IL-12p70 in brain homogenates and serum (FIG. 39B) Representative images of IHC of NeuN and H & E staining in brain from mice obtained 2 weeks post-treatment with MSCN1KO or MSCN1KO-IL-12.

### DETAILED DESCRIPTION

Described herein are methods of treating cancer that use the tumor-homing capacity of stem cells to deliver one or more therapeutic agents directly to a cancer cell or tumor. Various aspects involve the gene editing of stem cells to inactivate one or more receptors for cytotoxic factors to be expressed by the same or other populations of therapeutic stem cells. By rendering the stem cells insensitive or resistant to the effects of agents that they or another population of stem cells delivers to a tumor or the tumor microenvironment, the range of factors deliverable and the duration and effects of those factors delivered can be dramatically increased. The following describes modified stem cells and methods of use for the treatment of cancer, as well as considerations for one of skill in the art to prepare and use such cells in the methods described.

### Stem Cells

In some embodiments of the methods described herein, the cell type that is loaded with oncolytic virus or engineered to express a therapeutic or immunomodulatory polypeptide agent is a stem cell. Stem cells are undifferentiated cells defined by their ability at the single cell level to both self-renew and differentiate to produce progeny cells, including self-renewing progenitors, non-renewing progenitors, and terminally differentiated cells. Some stem cells, depending on their level of differentiation, are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts. (See, e.g., Potten et al., Development 110: 1001 (1990); U.S. Pat. Nos. 5,750,376, 5,851,832, 5,753,506, 5,589,376, 5,824,489, 5,654,183, 5,693,482, 5,672,499, and 5,849,553, all herein incorporated in their entireties by reference). Somatic or adult stem cells have certain advantages, for example, as using somatic stem cells allows a patient's own cells to be expanded in culture and then re-introduced into the patient. Natural somatic stem cells have been isolated from a wide variety of adult tissues including blood, bone marrow, brain, olfactory epithelium, skin, pancreas, skeletal muscle, and cardiac muscle. Each of these somatic stem cells can be characterized based on gene expression, factor responsiveness, and morphology in culture. Exemplary naturally occurring somatic stem cells include, but are not limited to, neural stem cells, neural crest stem cells, mesenchymal stem cells, hematopoietic stem cells, and pancreatic stem cells. In addition, iPS cells generated from a patient provide a source of cells that can be engineered to express a heterologous therapeutic polypeptide, including but not limited to a receptor-targeted cytotoxic polypeptide or an immunomodulatory polypeptide, expanded, and re-introduced to the patient, before or after stimulation to differentiate to a desired lineage or phenotype, such as a neural or neuronal stem cell. Certain stem cells, including but not limited to mesenchymal stem cells, exhibit at least a degree of immune privilege or immune indifference, such that they need not necessarily be MHC-matched with the recipient, providing an option for “off the shelf” preparations of genetically modified stem cells for use in the methods and compositions as described herein.

The stem cells for use with the compositions and methods described herein can be naturally occurring stem cells or “induced” stem cells, such as “induced pluripotent stem cells” (iPS cells) generated using any method or composition known to one of skill in the art. Stem cells can be obtained or generated from any mammalian species, e.g. human, primate, equine, bovine, porcine, canine, feline, rodent, e.g. mice, rats, hamster, etc. In some embodiments of the aspects described herein, a stem cell is a human stem cell.

Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and the cell types and elements (e.g., platelets) that are normal components of the blood); (4) oligopotent, meaning able to give rise to a more restricted subset of cell lineages than multipotent stem cells; and (5) unipotent, meaning able to give rise to a single cell lineage (e.g., spermatogenic stem cells).

Any stem cell type that migrates or homes to a tumor or tumor microenvironment can be used in the methods and compositions described herein. The stem cells can be multipotent, pluripotent or totipotent. In one embodiment, the adult stem cells are mesenchymal stem cells (MSCs). In one embodiment, the adult stem cells are tissue or organ specific stem cells such as neuronal stem cells, vascular stem cells, or epidermal stem cells.

MSCs can be obtained from a variety of sources such as bone marrow, umbilical cord blood, and adipose tissue. Common sources of stem cells are human umbilical vein endothelial cells (HUVEC), and primary human cutaneous microvascular endothelial cells (HCMEC). Analagous non-human stem cells can be obtained from similar non-human sources as well. The stem cells described herein are not considered to be cancer stem cells as the term is typically used in the art. Stem cells for use in the methods and compositions described herein can be primary or cells that have been maintained in cell culture for an extended period. The stem cells may be obtained from any animal type (e.g., human).

In one embodiment, the stem cells are obtained or derived from a subject who is in need of therapeutic treatment for a cell proliferative disorder, e.g., cancer. In one embodiment, the stem cells are obtained or derived from a subject who is in need of therapeutic treatment for a cell proliferative disorder in the brain (e.g., brain tumor or cancer). The subject can have the cell proliferative disorder or be at risk for the disorder.

A mesenchymal stem cell is a self-renewing, multipotent stem cell that comprises the capacity to differentiate into various cell types including, but not limited to, white adipocytes, brown adipocytes, myoblast, skeletal muscle, cardiac muscle, smooth muscle, chondrocytes, and a mature osteoblast upon introduction of proper differentiation cues. An MSC can be produced using techniques known in the art, for example, by a process comprising obtaining a cell by dispersing an embryonic stem (ES) cell colony and culturing the cell with MSC conditioned media. A population of MSCs can be confirmed by assessing the surface markers of the MSC population. For example, at a minimum, 95% or more of an MSC cell population expresses CD73/5′-Nucleotidase, CD90/Thy1, and CD105/Endoglin, and 2% or less of an MSC cell population expresses CD34, CD45, CD11b/Integrin alpha M or CD14, CD79 alpha or CD19, and HLA Class II. The expression of these surface markers can be assessed using techniques known in the art, e.g., FACS analysis.

MSCs can be easily extracted and, given their propensity to move to the site of tumors, are useful for the delivery of therapeutics to said tumors and tumor microenvironments. While not wishing to be bound by theory, MSCs tumor tropism (movement to the site of a tumor) is thought to be driven by paracrine signaling between the tumor microenvironment and the corresponding receptors on the cell surface of the MSC. Further discussion of MSCs, their preparation and therapeutic uses is found, for example, in Sage et al., Cytotherapy 18: 1435-1445 (2016).

MSCs recruit monocytes, T cells and dendritic cells to sites of inflammation following an infection or injury (e.g., tumor resection) via expression of chemokine (C-C motif) ligand 2 (CCL2, as known as MCP-1 and small inducible cytokine A2). CCL2 sequences are known for a number of species, e.g., human CD28 (NCBI Gene ID: 6347). It is contemplated that an MSC genetically modified to express increased levels of CCL2 (compared to wild-type CCL2 levels) will have a greater capacity to recruit T cells to the site of injury (e.g., tumor resection) compared to a wild-type MSC.

In one embodiment, the stem cells can be neuronal stem cells. As used herein, “neural stem cells” or “neuronal stem cells” or “NSCs” refer to a subset of multipotent cells which have partially differentiated along a neural cell pathway and express some neural markers including, for example, nestin. Neuronal stem cells, the markers they express, and their differentiation from human ESCs and iPS cells are described by Yuan et al. (PLOS One, 6(3): e17540 (2011), which is incorporated herein by reference in its entirety. In some embodiments of the methods described herein, NSCs are marked by the cell-surface expression profile of CD184+/CD271−/CD44−/CD24+. Neural stem cells can differentiate into neurons or glial cells (e.g., astrocytes and oligodendrocytes). Thus, “neural stem cells derived or differentiated from iPS cells” refers to cells that are multipotent but have partially differentiated along a neural cell pathway (i.e., express some neural cell markers), and themselves are the result of in vitro or in vivo differentiation iPS cells.

In one embodiment, a cell (e.g., an MSC, NSC or other stem cell) is genetically modified to express a polypeptide comprising an immunomodulatory agent. These can include, for example, GM-CSF, IL-2, IL-12, Flt3L, IL-5 and/or IL-15, among others. In one embodiment, a stem cell is engineered to deliver a heterologous polypeptide comprising a cytokine, (e.g., Interleukin (IL)-12B (NCBI Gene ID: 3593), IL-2 (NCBI Gene ID: 3558), IL-5 (NCBI Gene ID: 3567), IL-15 (NCBI Gene ID: 3600), or an interferon (e.g., interferon α-1 (NCBI Gene ID: 3439), interferon β-1 (NCBI Gene ID: 3456), or interferon γ (NCBI Gene ID: 3458), TNF-related apoptosis-inducing ligand (TRAIL; also known as TNF superfamily member 10, TL2, CD253, or TNLG6A; NCBI Gene ID: 8743), an EGFR nanobody-TRAIL fusion, Thrombospondin (THBS)-1 (NCBI Gene ID: 7057).

In some embodiments, cord blood cells are used as a source of stem cells. Accordingly, in some aspects, cells to be modified or engineered to deliver an oncolytic virus or an immunomodulatory polypeptide agent can also be derived from human umbilical cord blood cells (HUCBC), which are recognized as a rich source of hematopoietic and mesenchymal stem cells (Broxmeyer et al., 1992 Proc. Natl. Acad. Sci. USA 89:4109-4113). One advantage of HUCBC for use with the methods and compositions described herein is the immature immunity of these cells, which is very similar to fetal cells, and thus significantly reduces the risk for rejection by the host (Taylor & Bryson, 1985 J. Immunol. 134:1493-1497).

In some embodiments of the aspects described herein, iPS cells are engineered to express or secrete an immunomodulatory polypeptide agent. In some embodiments of the aspects described herein, iPS cells are engineered to express or secrete an immunomodulatory polypeptide agent prior to being differentiated into another desired cell type. In some embodiments of the aspects described herein, iPS cells are engineered to express or secrete an immunomodulatory polypeptide agent after differentiation into another desired cell type. This can be accomplished, for example, by placing the immunomodulatory polypeptide agent-expressing construct under control of a promoter only active in the more differentiated (stem) cell phenotype.

In some embodiments, wherein the stem cell therapies described herein comprise a first and second SC, the immunomodulatory polypeptide agents produced by the first and second SC are the same.

In some embodiments, wherein the stem cell therapies described herein comprise a first and second SC, the immunomodulatory polypeptide agents produced by the first and second SC are the different.

Studies have shown that NSCs injected into the parenchyma of the brain or intracranially migrate towards injured or pathological central nervous system (CNS) sites. This chemotropic property of NSCs has been utilized for cell-based therapies to treat diverse neurological diseases as described herein and in T. Bagci-Onder et al., Cancer Research 2011, 71:154-163; Hingtgen S. et al., Stem Cells 2010, 28(4):832-41; Hingtgen S. et al., Mol Cancer Ther. 2008, 7(11): 3575-85; Brustle O. et al., 6 Current Opinion in Neurobiology. 688 (1996); Flax J. D., et al., 16 Nature Biotechnology. 1033. (1998); Kim S. U., 24. Neuropathology. 159 (2004); Lindvall O et al., 10 (suppl) Nature Medicine. S42 (2004); Goldman S., 7. Nature Biotechnology. 862 (2005); Muller F. et al., 7 Nature Reviews Neuroscience. 75 (2006); Lee, J. P., et al. 13 Nature Medicine 439 (2007), and Kim S. U. et al., 87 Journal of Neuroscience Research 2183 (2009), the contents of each of which are herein incorporated in their entireties by reference.

Systemic or intra-arterial administration of engineered neuronal stem cells permits the cells to track to multifocal metastatic lesions for delivery of therapeutic polypeptides. The stem cells can cross the blood-brain barrier and become established at multifocal tumor sites and inhibit tumor growth and viability.

Accordingly, in some embodiments of the compositions and methods described herein, a pharmaceutically acceptable composition comprising a neural stem cell modified to express a receptor-targeted cytotoxic polypeptide, immunomodulatory polypeptide or other therapeutic agent can be administered to a subject. Because NSCs can be engineered to package and release oncolytic virus (e.g., oncolytic herpes virus) vectors which, in turn, can serve as vectors for the transfer of sequences to CNS cells, neural progenitor/stem cells can serve to magnify the efficacy of viral-mediated gene delivery to large regions in the brain. Additional vectors that can be used in the embodiments described herein include herpes simplex virus vectors, SV 40 vectors, polyoma virus vectors, papilloma virus vectors, picornavirus vectors, vaccinia virus vectors, and a helper-dependent or gutless adenovirus. In one embodiment, the vector can be a lentivirus. Methods for preparing genetically engineered neural stem cells and compositions thereof for therapeutic treatment have been described in U.S. Pat. Nos. 7,393,526 and 7,655,224, the contents of which are incorporated herein by reference in their entirety.

In various embodiments of the compositions and methods described herein, the neural stem cells that can be used include, but are not limited to, human neural stem cells, mouse neural stem cells HSN-1 cells, fetal pig cells and neural crest cells, bone marrow derived neural stem cells, and hNT cells. HSN-1 cells can be prepared, for example, as described in, e.g., Ronnett et al. (Science 248, 603-605, 1990). The preparation of neural crest cells is described in U.S. Pat. No. 5,654,183. hNT cells can be prepared as described in, e.g, Konubu et al. (Cell Transplant 7, 549-558, 1998). In some embodiments of the compositions and methods described herein, the neural stem cells that can be used are neural stem cells derived or differentiated from a precursor stem cell, such as a human embryonic stem cell or an induced pluripotent stem (iPS) cell. Such neural stem cells can be generated from or differentiated from human embryonic stem cells, using, for example, compositions and methods described in Nature Biotechnology 27, 275-280 (2009), “Highly efficient neural conversion of human ES and iPS cells by dual inhibition of SMAD signaling,” the contents of which are herein incorporated by reference in their entireties. Such neural stem cells can be generated from or differentiated from iPS cells, using, for example, the compositions and methods described in US Patent Publication US 2010/0021437 A1, “NEURAL STEM CELLS DERIVED FROM INDUCED PLURIPOTENT STEM CELLS,” the contents of which are herein incorporated by reference in their entireties.

Neural selection factors that can be used to differentiate pluripotent stem cells, such as embryonic stem cells or iPS cells into neural stem cells, include, for example, sonic hedgehog (SHH), fibroblast growth factor-2 (FGF-2), and fibroblast growth factor-8 (FGF-8), which can be used alone or in pairwise combination, or all three factors may be used together. In some embodiments, iPS cells are cultured in the presence of at least SHH and FGF-8. In other embodiments, FGF-2 is omitted. Preferably, the neural stem cells derived from iPS cells express nestin. In some embodiments, the pluripotent stem cells are cultured in the presence of the one or more neural selection factors for 4, 5, 6, 7, 8, 9, 10, 12, 14, 16, 18, 20 days or more. Preferably, the population of neural stem cells is characterized in that at least 50%, at least 75%, at least 85%, at least 90%, at least 95%, or at least 99% of the cells of the population express nestin. Preferably, the nestin-expressing cells further express at least one of En-1, Pitx3, and Nurr-1. In other embodiments, the population of neural stem cells has been depleted of at least 50%, 75%, 85%, 95%, or 99% of the cells expressing surface markers of immature embryonic stem cells including, for example, SSEA-1, SSEA-3, SSEA-4, Tra-1-81, and Tra-1-60. Preferably, the population of neural stem cells contains less than 10%, less than 5%, less than 2.5%, less than 1%, or less than 0.1% of cells that express the selected marker (e.g., SSEA-4).

In some embodiments, it can be beneficial to include a kill switch in a stem cell as described herein. This provides a mechanism to remove or kill the therapeutic cells, e.g., after they have treated the subject's cancer. In one embodiment, cells are rendered susceptible to an agent that can be delivered as a pro-drug which is only active upon cells carrying a construct encoding an enzyme that activates the pro-drug to a toxic form. As used herein, the term “pro-drug” refers to a drug, e.g., a small molecule drug, that is not active for its intended indication until acted upon by one or more systems in the cell or body of a patient. For example, the 2′-deoxyguanosine analogue ganciclovir is not efficiently metabolized to its active DNA synthesis-inhibiting form in cells lacking certain viral thymidine kinase (TK) enzymes (e.g., HSV-TK, CMV-TK), but in cells expressing such thymidine kinase enzymes, the ganciclovir pro-drug is efficiently metabolized to ganciclovir triphosphate, which is a competitive inhibitor of dGTP incorporation into DNA, leading to cell death. Thus, cells engineered to express a thymidine kinase that promotes the conversion of ganciclovir to ganciclovir triphosphate (e.g., HSV-TK) will be susceptible to selective killing by administering or contacting with the ganciclovir pro-drug. Other pro-drugs and agents or enzymes that promote their conversion to active form are known in the art. Non-limiting, exemplary prodrug converting enzymes with their prodrug partners include, but are not limited to, herpes simplex virus thymidine kinase/gancyclovir, varicella zoster thymidine kinase/gancyclovir, cytosine deaminase/5-fluorouracil, purine nucleoside phosphorylase/6-methylpurine deoxyriboside, beta lactamase/cephalosporin-doxorubicin, carboxypeptidase G2/4-[(2-chloroethyl)(2-mesuloxyethyl)amino]benzoyl-L-glutamic acid, cytochrome P450/acetominophen, horseradish peroxidase/indole-3-acetic acid, nitroreductase/CB 1954, rabbit carboxylesterase/7-ethyl-10-[4-(1-piperidino)-1-piperidino]carbonyloxycam-potothecin, mushroom tyrosinase/bis-(2-chloroethyl)amino-4-hydroxyphenylaminomethanone 28, beta galactosidase/1-chloromethyl-5-hydroxy-1,2-dihyro-3H-benz[e]indole, beta glucuronidase/epirubicin-glucoronide, thymidine phosphorylase/5′-deoxy-5-fluorouridine, deoxycytidine kinase/cytosine arabinoside, beta-lactamase and linamerase/linamarin. Coding sequences for the various prodrug converting enzymes are known in the art.

Such pro-drug systems can provide a heterologous inducible cell suicide system. As used herein, a “heterologous inducible cell suicide system” is a system for selectively killing engineered cancer cells as described herein. Such systems involve the introduction of one or more heterologous nucleic acid sequences to the cancer cell that render the cell responsive to a cell death-inducing agent. The system is maintained in an inactive state until the inducing agent, e.g., a small molecule or other drug, is administered to the patient. Different configurations of heterologous inducible cell suicide systems include, but are not limited to one in which the cell is modified to express an enzyme that converts a non-toxic pro-drug to a toxic form, and one in which the cell is modified to contain a nucleic acid construct encoding a cell death inducing polypeptide under control of a genetic element inducible by a small molecule or other drug. Various embodiments of such systems are known in the art and/or described further herein below. As used herein, the term “inducible” refers to a system that is substantially inactive until an inducing agent is provided. The term can refer, for example, to a gene or genetic element the expression of which is inducible by addition of a drug, such as a tetracycline- or doxycycline-inducible construct, or to a heterologous cell suicide system in which cell suicide is induced by the addition of a drug. By “substantially inactive” in the context of a heterologous inducible cell suicide system is meant that in the absence of the inducing drug, the inducible system maintains expression of the cell killing machinery at a level that permits the cell to remain viable, home to a tumor, and produce one or more therapeutic agents or polypeptides.

In one embodiment, an immune cell is used in place of a stem cell described herein. For example, in some embodiments of the methods described herein, the cell type that is loaded with oncolytic virus or engineered to express a therapeutic or immunomodulatory polypeptide agent is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the cell is a T cell; a NK cell; a NKT cell; lymphocytes, such as B cells and T cells; and myeloid cells, such as monocytes, macrophages, eosinophils, mast cells, basophils, and granulocytes. One skilled in the art will be able to isolate and engineer an immune cells using standard techniques in the art and described herein.

### Oncolytic Viruses

Any of a variety of oncolytic viruses can be incorporated into the stem cells described herein. Numerous oncolytic viruses are known in the art and are described, for example, in Kim et al. (1999, In: Gene Therapy of Cancer, Academic Press, San Diego, Calif., pp. 235-248), any of which is envisioned for use herein. By way of example, appropriate oncolytic viruses include type 1 herpes simplex viruses, type 2 herpes simplex viruses, vesicular stomatitis viruses, oncolytic adenovirus (U.S. Pat. No. 8,216,819), Newcastle disease viruses, vaccinia viruses, and mutant strains of these viruses. In some embodiments, the oncolytic virus is replication-selective or replication-competent. In some embodiments, the oncolytic virus is replication incompetent.

In some embodiments, the oncolytic virus is an oncolytic herpes simplex virus. Oncolytic herpes simplex viruses (oHSV) are known in the art and are described, for example, in Kim et al. (1999, In: Gene Therapy of Cancer, Academic Press, San Diego, Calif., pp. 235-248). In one embodiment, the oHSV used in the methods and compositions described herein is replication-selective or replication-competent. In one embodiment, the oHSV is replication-incompetent.

Herpes simplex 1 type viruses are among the preferred viruses, for example the variant of HSV-1 viruses that do not express functional ICP34.5 and thus exhibit significantly less neurotoxicity than their wild type counterparts. Such variants include, without limitation, oHSV-R3616, one of the HSV-1 viruses described in Coukos et al., Gene Ther. Mol. Biol. 3:79-89 (1998), and Varghese and Rabkin, Cancer Gene Therapy 9:967-978 (2002). Other exemplary HSV-1 viruses include 1716, R3616, and R4009. Other replication selective HSV-1 virus strains that can be used include, e.g., R47A (wherein genes encoding proteins ICP34.5 and ICP47 are deleted), G207 (genes encoding ICP34.5 and ribonucleotide reductase are deleted), NV1020 (genes encoding UL24, UL56 and the internal repeat are deleted), NV1023 (genes encoding UL24, UL56, ICP47 and the internal repeat are deleted), 3616-UB (genes encoding ICP34.5 and uracil DNA glycosylase are deleted), G92A (in which the albumin promoter drives transcription of the essential ICP4 gene), hrR3 (the gene encoding ribonucleotide reductase is deleted), and R7041 (Us3 gene is deleted) and HSV strains that do not express functional ICP34.5.

oHSV for use in the methods and compositions described herein is not limited to one of the HSV-1 mutant strains described herein. Any replication-selective strain of a herpes simplex virus can be used. In addition to the HSV-1 mutant strains described herein, other HSV-1 mutant strains that are replication selective have been described in the art. Furthermore, HSV-2, mutant strains such as, by way of example, HSV-2 strains 2701 (RL gene deleted), Delta RR (ICP10PK gene is deleted), and FusOn-H2 (ICP10 PK gene deleted) can also be used in the methods and compositions described herein.

Non-laboratory strains of HSV can also be isolated and adapted for use in the invention (U.S. Pat. No. 7,063,835, the contents of which are herein incorporated by reference in their entirety). Furthermore, HSV-2 mutant strains such as, by way of example, HSV-2 strains HSV-2701, HSV-2616, and HSV-2604 may be used in the methods and compositions as described herein.

In one embodiment, the oHSV is G47Δ. G47Δ is a third generation virus, which contains 1) a mutation of ICP6, which targets viral deletion to tumor cells, 2) a deletion of γ34.5, which encodes ICP34.5 and blocks eIF2a phosphorylation and is the major viral determinant of neuropathogenicity, and 3) an additional deletion of the ICP47 gene and US 11 promoter, so that the late gene US 11 is now expressed as an immediate-early gene and able to suppress the growth inhibited properties of γ34.5 mutants. Deletion of ICP47 also abrogates HSV-1 inhibition of the transporter associated with antigen presentation and MHC class 1 downregulation (Todo et al., Proc. Natl. Acad. Sci. USA, 98:6396-6401(2001)).

In one embodiment, the oHSV will comprise one or more exogenous nucleic acids encoding for one or more of the polypeptides described herein. Methods of generating an oHSV comprising such an exogenous nucleic acid are known in the art. The specific position of insertion of the nucleic acid into the oHSV genome can be determined by the skilled practitioner.

In one embodiment, the oHSV is replication-selective or replication-competent. In one embodiment, the oHSV is replication-incompetent.

The oHSV useful in the present methods and compositions are, in some embodiments, replication-selective. It is understood that an oncolytic virus can be made replication-selective if replication of the virus is placed under the control of a regulator of gene expression such as, for example, the enhancer/promoter region derived from the 5′-flank of the albumin gene (e.g. see Miyatake et al., 1997, J. Virol. 71:5124-5132). By way of example, the main transcriptional unit of an HSV can be placed under transcriptional control of the tumor growth factor-beta (TGF-β) promoter by operably linking HSV genes to the TGF-β promoter. It is known that certain tumor cells overexpress TGF-β, relative to non-tumor cells of the same type. Thus, an oHSV wherein replication is subject to transcriptional control of the TGF-β promoter is replication-selective, in that it is more capable of replicating in the certain tumor cells than in non-tumor cells of the same type. Similar replication-selective oHSV may be made using any regulator of gene expression which is known to selectively cause overexpression in an affected cell. The replication-selective oHSV may, for example, be an HSV-1 mutant in which a gene encoding ICP34.5 is mutated or deleted.

An oHSV in accordance with the present invention can further comprise other modifications in its genome. For example, it can comprise additional DNA inserted into the UL44 gene. This insertion can produce functional inactivation of the UL44 gene and the resulting lytic phenotype, or it may be inserted into an already inactivated gene, or substituted for a deleted gene. In one embodiment, the oHSV for use in the methods and compositions described herein is under the control of an exogenously added regulator such as tetracycline (U.S. Pat. No. 8,236,941, the contents of which are herein incorporated by reference in their entirety), such as by engineering the virus to have a tetracycline inducible promoter driving expression of ICP27.

The oHSV may also have incorporated therein one or more promoters that impart to the virus an enhanced level of tumor cell specificity. In this way, the oHSV can be targeted to specific tumor types using tumor cell-specific promoters. The term “tumor cell-specific promoter” or “tumor cell-specific transcriptional regulatory sequence” or “tumor-specific promoter” or “tumor-specific transcriptional regulatory sequence” indicates a transcriptional regulatory sequence, promoter and/or enhancer that is present at a higher level in the target tumor cell than in a normal cell.

In one embodiment, the oHSV useful in the methods and compositions as described herein is engineered to place at least one viral protein necessary for viral replication under the control of a tumor-specific promoter. Or, alternatively a gene (a viral gene or exogenous gene) that encodes a cytotoxic agent can be put under the control of a tumor-specific promoter. By cytotoxic agent as used here is meant any protein that causes cell death. For example, such would include ricin toxin, diphtheria toxin, or truncated versions thereof. Also, included would be genes that encode prodrugs, cytokines, or chemokines. Such viral vectors may utilize promoters from genes that are highly expressed in the targeted tumor such as the epidermal growth factor receptor promoter (EGFR) or the basic fibroblast growth factor (bFGF) promoter, or other tumor associated promoters or enhancer elements.

### TRAIL

In various embodiments, a receptor-targeted cytotoxic agent is encoded and expressed, e.g., from an oncolytic virus released by a stem cell as described herein, or from a genetically modified stem cell. In some embodiments, the receptor-targeted cytotoxic agent is a “Tumor necrosis factor-Related Apoptosis-Inducing Ligand” or “TRAIL” polypeptide.

“Tumor necrosis factor-related apoptosis-inducing ligand” or “TRAIL” as used herein refers to the 281 amino acid polypeptide having the amino acid sequence of: MAMMEVQGGPSLGQTCVLIVIFTVLLQSLCVAVTYVYFTNELKQMQDKYSKSGIACFLKEDDS YWDPNDEESMNSPCWQVKWQLRQLVRKMILRTSEETISTVQEKQQNISPLVRERGPQRVAAHIT GTRGRSNTLSSPNSKNEKALGRKINSWESSRSGHSFLSNLHLRNGELVIHEKGFYYIYSQTYFRFQ EEIKENTKNDKQMVQYIYKYTSYPDPILLMKSARNSCWSKDAEYGLYSIYQGGIFELKENDRIFV SVTNEHLIDMDHEASFFGAFLVG (SEQ ID NO: 1), as described by, e.g., NP_003801.1, together with any naturally occurring allelic, splice variants, and processed forms thereof. Typically, TRAIL refers to human TRAIL. The term TRAIL, in some embodiments of the aspects described herein, is also used to refer to truncated forms or fragments of the TRAIL polypeptide, comprising, for example, specific TRAIL domains or residues thereof. The amino acid sequence of the human TRAIL molecule as presented above comprises an N-terminal cytoplasmic domain (amino acids 1-18), a transmembrane region (amino acids 19-38), and an extracellular domain (amino acids 39-281). The extracellular domain comprises the TRAIL receptor-binding region. TRAIL also has a spacer region between the C-terminus of the transmembrane domain and a portion of the extracellular domain This spacer region, located at the N-terminus of the extracellular domain, consists of amino acids 39 through 94 of the sequence above. Amino acids 138 through 153 of the sequence above correspond to a loop between the R sheets of the folded (three dimensional) human TRAIL protein.

In one embodiment, the TRAIL polypeptide comprises the extracellular domain of TRAIL (e.g., human trial). In one embodiment, the TRAIL is a fusion protein comprising one or more domains of TRAIL (e.g., the extracellular domain) fused to a heterologous sequence. In one embodiment, the TRAIL fusion protein further comprises a signal for secretion.

Preferably, the TRAIL protein and the nucleic acids encoding it, are derived from the same species as will be administered in the therapeutic methods described herein. In one embodiment, the nucleotide sequence encoding TRAIL and the TRAIL amino acid sequence is derived from a mammal. In one embodiment, the mammal is a human (human TRAIL). In one embodiment, the mammal is a non-human primate.

In some embodiments, an oncolytic virus, e.g., an oHSV, for use in the methods and compositions described herein, or a genetically modified or engineered stem cell for use in the methods and compositions described herein, can comprise a nucleic acid sequence that encodes TRAIL, or a biologically active fragment thereof, incorporated into the virus genome in expressible form. As such the oHSV can serve as a vector for delivery of TRAIL to the infected cells. Where a stem cell is The use of various forms of TRAIL are envisioned, such as those described herein, including without limitation, a secreted form of TRAIL or a functional domain thereof (e.g., a secreted form of the extracellular domain), multimodal TRAIL, or a therapeutic TRAIL module, therapeutic TRAIL domain (e.g., the extracellular domain) or therapeutic TRAIL variant (examples of each of which are described in WO2012/106281, the contents of which are herein incorporated by reference in their entirety), and also fragments, variants and derivatives of these, and fusion proteins comprising one of these TRAIL forms such as described herein.

TRAIL is normally expressed on both normal and tumor cells as a noncovalent homotrimeric type-II transmembrane protein (memTRAIL). In addition, a naturally occurring soluble form of TRAIL (solTRAIL) can be generated due to alternative mRNA splicing or proteolytic cleavage of the extracellular domain of memTRAIL and thereby still retaining tumor-selective pro-apoptotic activity. TRAIL utilizes an intricate receptor system comprising four distinct membrane receptors, designated TRAIL-R1, TRAIL-R2, TRAIL-R3 and TRAIL-R4. Of these receptors, only TRAIL-R1 and TRAIL-2 transmit an apoptotic signal. These two receptors belong to a subgroup of the TNF receptor family, the so-called death receptors (DRs), and contain the hallmark intracellular death domain (DD). This DD is critical for apoptotic signaling by death receptors (J. M. A. Kuijlen et al., Neuropathology and Applied Neurobiology, 2010 Vol. 36 (3), pp. 168-182).

Apoptosis is integral to normal, physiologic processes that regulate cell number and results in the removal of unnecessary or damaged cells. Apoptosis is frequently dysregulated in human cancers, and recent advancements in the understanding of the regulation of programmed cell death pathways has led to the development of agents to reactivate or activate apoptosis in malignant cells. This evolutionarily conserved pathway can be triggered in response to damage to key intracellular structures or the presence or absence of extracellular signals that provide normal cells within a multicellular organism with contextual information.

Without wishing to be bound by theory, TRAIL activates the “extrinsic pathway” to apoptosis by binding to TRAIL-R1 and/or TRAIL-R2, whereupon the adaptor protein Fas-associated death domain and initiator caspase-8 are recruited to the DD of these receptors. Assembly of this “death-inducing signaling complex” (DISC) leads to the sequential activation of initiator and effector caspases, and ultimately results in apoptotic cell death. The extrinsic apoptosis pathway triggers apoptosis independently of p53 in response to pro-apoptotic ligands, such as TRAIL. TRAIL-R1 can induce apoptosis after binding non-cross-linked and cross-linked sTRAIL. TRAIL-R2 can only be activated by cross-linked sTRAIL. Death receptor binding leads to the recruitment of the adaptor FADD and initiator procaspase-8 and 10 to rapidly form the DISC. Procaspase-8 and 10 are cleaved into its activated configuration caspase-8 and 10. Caspase-8 and 10 in turn activate the effector caspase-3, 6 and 7, thus triggering apoptosis.

In certain cells, the execution of apoptosis by TRAIL further relies on an amplification loop via the “intrinsic mitochondrial pathway” of apoptosis. The mitochondrial pathway of apoptosis is a stress-activated pathway, e.g., upon radiation, and hinges on the depolarization of the mitochondria, leading to release of a variety of pro-apoptotic factors into the cytosol. Ultimately, this also triggers effector caspase activation and apoptotic cell death. This mitochondrial release of pro-apoptotic factors is tightly controlled by the Bcl-2 family of pro- and anti-apoptotic proteins. In the case of TRAIL receptor signaling, the Bcl-2 homology (BH3) only protein ‘Bid’ is cleaved into a truncated form (tBid) by active caspase-8. Truncated Bid subsequently activates the mitochondrial pathway.

TRAIL-R3 is a glycosylphosphatidylinositol-linked receptor that lacks an intracellular domain, whereas TRAIL-R4 only has a truncated and non-functional DD. The latter two receptors are thought, without wishing to be bound or limited by theory, to function as decoy receptors that modulate TRAIL sensitivity. Evidence suggests that TRAIL-R3 binds and sequesters TRAIL in lipid membrane microdomains. TRAIL-R4 appears to form heterotrimers with TRAIL-R2, whereby TRAIL-R2-mediated apoptotic signaling is disrupted. TRAIL also interacts with the soluble protein osteoprotegerin.

Diffuse expression of TRAIL has been detected on liver cells, bile ducts, convoluted tubules of the kidney, cardiomyocytes, lung epithelia, Leydig cells, normal odontogenic epithelium, megakaryocytic cells and erythroid cells. In contrast, none or weak expression of TRAIL was observed in colon, glomeruli, Henle's loop, germ and Sertoli cells of the testis, endothelia in several organs, smooth muscle cells in lung, spleen and in follicular cells in the thyroid gland. TRAIL protein expression was demonstrated in glial cells of the cerebellum in one study. Vascular brain endothelium appears to be negative for TRAIL-R1 and weakly positive for TRAIL-R2. With regard to the decoy receptors, TRAIL-R4 and TRAIL-R3 have been detected on oligodendrocytes and neurons.

TRAIL-R1 and TRAIL-R2 are ubiquitously expressed on a variety of tumor types. In a study on 62 primary GBM tumor specimens, TRAIL-R1 and TRAIL-R2 were expressed in 75% and 95% of the tumors, respectively. Of note, a statistically significant positive association was identified between agonistic TRAIL receptor expression and survival. Highly malignant tumors express a higher amount of TRAIL receptors in comparison with less malignant tumors or normal tissue. In general TRAIL-R2 is more frequently expressed on tumor cells than TRAIL-R1.

### Fragments, Variants and Derivatives of TRAIL

Fragments, variants and derivatives of native TRAIL proteins for use in the invention that retain a desired biological activity of TRAIL, such as TRAIL apoptotic activity are also envisioned for delivery by the oncolytic virus vector. In one embodiment, the biological or apoptotic activity of a fragment, variant or derivative of TRAIL is essentially equivalent to the biological activity of the corresponding native TRAIL protein. In one embodiment, the biological activity for use in determining the activity is apoptotic activity. In one embodiment, 100% of the apoptotic activity is retained by the fragment, variant or derivative. In one embodiment less than 100%, activity is retained (e.g., 95%, 90%, 85%, 80%, 75%, 70%, 65%, 60%, 55%, 50%, 45%, 40%) as compared to the full length native TRAIL. Fragments, variants or derivatives which retain less activity (e.g., 34%, 30%, 25%, 20%, 10%, etc.) may also be of value in the therapeutic methods described herein and as such are also encompassed in the invention. One measurement of TRAIL apoptotic activity by a TRAIL variant or TRAIL domain is the ability to induce apoptotic death of Jurkat cells. Assay procedures for identifying biological activity of TRAIL variants by detecting apoptosis of target cells, such as Jurkat cells, are well known in the art. DNA laddering is among the characteristics of cell death via apoptosis, and is recognized as one of the observable phenomena that distinguish apoptotic cell death from necrotic cell death. Apoptotic cells can also be identified using markers specific for apoptotic cells, such as Annexin V, in combination with flow cytometric techniques, as known to one of skill in the art. Further examples of assay techniques suitable for detecting death or apoptosis of target cells include those described in WO2012/106281.

A variety of TRAIL fragments that retain the apoptotic activity of TRAIL are known in the art, and include biologically active domains and fragments disclosed in Wiley et al. (U.S. Patent Publication 20100323399), the contents of which are herein incorporated by reference in their entireties.

TRAIL variants can be obtained by mutations of native TRAIL nucleotide sequences, for example. A “TRAIL variant,” as referred to herein, is a polypeptide substantially homologous to a native TRAIL, but which has an amino acid sequence different from that of native TRAIL because of one or a plurality of deletions, insertions or substitutions. “TRAIL encoding DNA sequences” encompass sequences that comprise one or more additions, deletions, or substitutions of nucleotides when compared to a native TRAIL DNA sequence, but that encode a TRAIL protein or fragment thereof that is essentially biologically equivalent to a native TRAIL protein, i.e., has the same apoptosis inducing activity.

The variant amino acid or DNA sequence preferably is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to a native TRAIL sequence. The degree of homology or percent identity) between a native and a mutant sequence can be determined, for example, by comparing the two sequences using freely available computer programs commonly employed for this purpose on the world wide web.

Alterations of the native amino acid sequence can be accomplished by any of a number of known techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

TRAIL variants can, in some embodiments, comprise conservatively substituted sequences, meaning that one or more amino acid residues of a native TRAIL polypeptide are replaced by different residues, and that the conservatively substituted TRAIL polypeptide retains a desired biological activity, i.e., apoptosis inducing activity or TRAIL apoptotic activity, that is essentially equivalent to that of the native TRAIL polypeptide. Examples of conservative substitutions include substitution of amino acids that do not alter the secondary and/or tertiary structure of TRAIL.

In other embodiments, TRAIL variants can comprise substitution of amino acids that have not been evolutionarily conserved. Conserved amino acids located in the C-terminal portion of proteins in the TNF family, and believed to be important for biological activity, have been identified. These conserved sequences are discussed in Smith et al. (Cell, 73:1349, 1993); Suda et al. (Cell, 75:1169, 1993); Smith et al. (Cell, 76:959, 1994); and Goodwin et al. (Eur. J. Immunol., 23:2631, 1993). Advantageously, in some embodiments, these conserved amino acids are not altered when generating conservatively substituted sequences. In some embodiments, if altered, amino acids found at equivalent positions in other members of the TNF family are substituted. Among the amino acids in the human TRAIL protein of SEQ ID NO:1 that are conserved are those at positions 124-125 (AH), 136 (L), 154 (W), 169 (L), 174 (L), 180 (G), 182 (Y), 187 (Q), 190 (F), 193 (Q), and 275-276 (FG) of SEQ ID NO:1. Another structural feature of TRAIL is a spacer region (i.e., TRAIL (39-94)) between the C-terminus of the transmembrane region and the portion of the extracellular domain that is believed to be important for biological apoptotic activity. In some embodiments, when the desired biological activity of TRAIL domain is the ability to bind to a receptor on target cells and induce apoptosis of the target cells substitution of amino acids occurs outside of the receptor-binding domain.

A given amino acid of a TRAIL domain can, in some embodiments, be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. TRAIL polypeptides comprising conservative amino acid substitutions can be tested in any one of the assays described herein to confirm that a desired TRAIL apoptotic activity of a native TRAIL molecule is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Particularly preferred conservative substitutions for use in the TRAIL variants described herein are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any cysteine residue not involved in maintaining the proper conformation of the multimodal TRAIL agent also can be substituted, generally with serine, to improve the oxidative stability of the molecule and prevent aberrant crosslinking. Conversely, cysteine bond(s) can be added to the multimodal TRAIL agent to improve its stability or facilitate oligomerization.

### Secreted TRAIL

In one embodiment, a form of TRAIL that is secreted (secreted TRAIL or sTRAIL) is expressed by the oHSV described herein. Various forms of secreted TRAIL can be used in the methods and compositions described herein. In one embodiment, the secreted TRAIL is the naturally occurring soluble TRAIL. (Ashkenazi A. et al., J Clin Oncol 2008; 26: 3621-30, and Kelley S K et al., J Pharmacol Exp Ther 2001; 299: 31-8). In one embodiment the naturally occurring soluble TRAIL is fused with an antibody derivative, such as scFv245 (Bremer E. et al., J Mol Med 2008; 86: 909-24; Bremer E, et al., Cancer Res 2005; 65: 3380-88; Bremer E, et al., J Biol Chem 2005; 280: 10025-33, and Stieglmaier J, et al., Cancer Immunol Immunother 2008; 57: 233-46).

Alternatively, the endogenous secretion sequence of TRAIL present on the N terminus can be replaced with the signal sequence (otherwise referred to as the extracellular domain) from Flt3 ligand and an isoleucine zipper (Shah et al., Cancer Research 64: 3236-3242 (2004); WO 2012/106281; Shah et al. Mol Ther. 2005 June; 11(6):926-31). Other secretion signal sequences can be added to TRAIL in turn to generate a secreted TRAIL for use in the invention. For example, SEC2 signal sequence and SEC(CV) signal sequence can be fused to TRAIL (see for example U.S. Patent Publication 2002/0128438, the contents of which are herein incorporated by reference in their entirety). Other secretion signal sequences can also be used and nucleotides including restriction enzyme sites can be added to the 5′ or 3′ terminal of respective secretion signal sequence, to facilitate the incorporation of such sequences into the DNA cassette. Such secretion signal sequences can be fused to the N-terminus or to the C-terminus.

In some embodiments of the aspects described herein, a soluble TRAIL polypeptide comprises the extracellular domain of TRAIL, but lacks the transmembrane domain. In some embodiments of the aspects described herein, a soluble TRAIL polypeptide is a fusion protein comprising one or more domains of TRAIL (e.g., the extracellular domain) fused to a heterologous sequence. In some embodiments of the aspects described herein, a soluble TRAIL polypeptide further comprises a signal for secretion.

Additionally, a linker sequence can be inserted between heterologous sequence and the TRAIL in order to preserve function of either portion of a generated fusion protein. Such linker sequences known in the art include a linker domain having the 7 amino acids (EASGGPE; SEQ ID NO: 3), a linker domain having 18 amino acids (GSTGGSGKPGSGEGSTGG; SEQ ID NO: 4). As used herein, a “linker sequence” refers to a peptide, or a nucleotide sequence encoding such a peptide, of at least 8 amino acids in length. In some embodiments of the aspects described herein, the linker module comprises at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 30 amino acids, at least 35 amino acids, at least 40 amino acids, at least 45 amino acids, at least 50 amino acids, at least 55 amino acids, at least 56 amino acids, at least 60 amino acids, or least 65 amino acids. In some embodiments of the aspects described herein, a linker module comprises a peptide of 18 amino acids in length. In some embodiments of the aspects described herein, a linker module comprises a peptide of at least 8 amino acids in length but less than or equal to 56 amino acids in length, i.e., the length of the spacer sequence in the native TRAIL molecule of SEQ ID NO: 1. In some embodiments, the linker sequence comprises the spacer sequence of human TRAIL, i.e., amino acids 39-94 of SEQ ID NO: 1, or a sequence having at least 80%, at least 85%, at least 90%, at least 95%, at least 99% identity to amino acids 39-94 of SEQ ID NO: 1.

### Signal Sequences

Secreted TRAIL can be generated by incorporation of a secretion signal sequence into the TRAIL or TRAIL fragment or derivative. As used herein, the terms “secretion signal sequence,” “secretion sequence,” “secretion signal peptide,” or “signal sequence,” refer to a sequence that is usually about 3-60 amino acids long and that directs the transport of a propeptide to the endoplasmic reticulum and through the secretory pathway during protein translation. As used herein, a signal sequence, which can also be known as a signal peptide, a leader sequence, a prepro sequence or a pre sequence, does not refer to a sequence that targets a protein to the nucleus or other organelles, such as mitochondria, chloroplasts and apicoplasts. In one embodiment, the secretion signal sequence comprises 5 to 15 amino acids with hydrophobic side chains that are recognized by a cytosolic protein, SRP (Signal Recognition Particle), which stops translation and aids in the transport of an mRNA-ribosome complex to a translocon in the membrane of the endoplasmic reticulum. In one embodiment, the secretion signal peptide comprises at least three regions: an amino-terminal polar region (N region), where frequently positive charged amino acid residues are observed, a central hydrophobic region (H region) of 7-8 amino acid residues and a carboxy-terminal region (C region) that includes the cleavage site. Commonly, the signal peptide is cleaved from the mature protein with cleavage occurring at this cleavage site.

The secretory signal sequence is operably linked to the TRAIL or TRAIL fragment or derivative such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences can be positioned elsewhere in the nucleotide sequence of interest (see, e.g., Welch et al., U.S. Pat. No. 5,037,743; Holland et al., U.S. Pat. No. 5,143,830).

In one embodiment, the secretory sequence comprises amino acids 1-81 of the following Flt3L amino acid sequence: MTVLAPAWSP NSSLLLLLLL LSPCLRGTPD CYFSHSPISS NFKVKFRELT DHLLKDYPVT VAVNLQDEKH CKALWSLFLA QRWIEQLKTV AGSKMQTLLE DVNTEIHFVT SCTFQPLPEC LRFVQTNISH LLKDTCTQLL ALKPCIGKAC QNFSRCLEVQ CQPDSSTLLP PRSPIALEAT ELPEPRPRQL LLLLLLLLPL TLVLLAAAWG LRWQRARRRG ELHPGVPLPS HP (GenBank Accession P49772), or a functional fragment thereof. In one embodiment, the signal peptide comprises amino acids 1-81 of the sequence above. In one embodiment, the secretory signal sequence comprises a sequence having at least 90% identity to amino acids 1-81 of the sequence above. In one embodiment, the secretory signal sequence consists essentially of amino acids 1-81 of the sequence above. In one embodiment, the secretory signal sequence consists of amino acids 1-81 of the sequence above.

While the secretory signal sequence can be derived from Flt3L, in other embodiments a suitable signal sequence can also be derived from another secreted protein or synthesized de novo. Other secretory signal sequences which can be substituted for the Flt3L signal sequence for expression in eukaryotic cells include, for example, naturally-occurring or modified versions of the human IL-17RC signal sequence, otPA pre-pro signal sequence, human growth hormone signal sequence, human CD33 signal sequence Ecdysteroid Glucosyltransferase (EGT) signal sequence, honey bee Melittin (Invitrogen Corporation; Carlsbad, Calif), baculovirus gp67 (PharMingen: San Diego, Calif) (US Pub. No. 20110014656). Additional secretory sequences include secreted alkaline phosphatase signal sequence, interleukin-1 signal sequence, CD-14 signal sequence and variants thereof (US Pub. No. 20100305002) as well as the following peptides and derivatives thereof: Sandfly Yellow related protein signal peptide, silkworm friboin LC signal peptide, snake PLA2, Cyrpidina noctiluca luciferase signal peptide, and pinemoth fibroin LC signal peptide (US Pub. No. 20100240097). Further signal sequences can be selected from databases of protein domains, such as SPdb, a signal peptide database described in Choo et al., BMC Bioinformatics 2005, 6:249, LOCATE, a mammalian protein localization database described in Sprenger et al. Nuc Acids Res, 2008, 36:D230D233, or identified using computer modeling by those skilled in the art (Ladunga, Curr Opin Biotech 2000, 1:13-18).

Selection of appropriate signal sequences and optimization or engineering of signal sequences is known to those skilled in the art (Stem et al., Trends in Cell & Molecular Biology 2007 2:1-17; Barash et al., Biochem Biophys Res Comm 2002, 294:835-842). In one embodiment, a signal sequence can be used that comprise a protease cleavage site for a site-specific protease (e.g., Factor IX or Enterokinase). This cleavage site can be included between the pro sequence and the bioactive secreted peptide sequence, e.g., TRAIL domain, and the pro-peptide can be activated by the treatment of cells with the site-specific protease (US Pub. No. 20100305002).

### Immune Checkpoint Molecules and Immune Checkpoint Modulators Thereof

The immune system has multiple inhibitory pathways that are critical for maintaining self-tolerance and modulating immune responses. In T-cells, the amplitude and quality of response is initiated through antigen recognition by the T-cell receptor and is regulated by immune checkpoint proteins that balance co-stimulatory and inhibitory signals. In some embodiments, the immune checkpoint modulator is an inhibitor of a blocking checkpoint molecule. Exemplary blocking checkpoint molecules include, but are not limited to, PD-1, PD-L1, CTLA4, B7-H3, B7-H4, VISTA, TMIGD2, B7-H7, BTLA, HVEM, CD160, LAG3, TIGIT, CD96, CD155 and TIM-3. In other embodiments, the immune checkpoint modulator is an agonist of a stimulative checkpoint molecule. Exemplary stimulative checkpoint molecules include, but are not limited to, OX40, 4-1BB, GITR, CD28, ICOS, LIGHT, CD27, DNAM-1, 2B4, DC-SIGN, DR3, and CD40.

Further examples of checkpoint molecules that can be targeted for blocking or inhibition include, but are not limited to, PDL2, B7-H3, B7-H4, BTLA, HVEM, GAL9, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR, TIGIT, DD1-□, TIM-3, Lag-3, and various B-7 family ligands. B7 family ligands include, but are not limited to, B7-1, B7-2, B7-DC, B7-H1, B7-H2, B7-H3, B7-H4, B7-H5, B7-H6 and B7-H7.

Immune checkpoints and modulators thereof as well as methods of using such compounds are known to those of skill in the art and/or are described in the literature, thus they are not described in detail herein. A brief description of immune checkpoint molecules and modulators thereof is provided below.

In some embodiments, the one or more immune checkpoint modulator(s) can independently be a polypeptide or a polypeptide-encoding nucleic acid molecule; said polypeptide comprising a domain capable of binding the desired immune checkpoint molecule and/or inhibiting the binding of a ligand to the immune checkpoint molecule so as to exert an antagonist function (i.e. being capable of antagonizing an immune checkpoint-mediated inhibitory signal) or an agonist function (i.e. being capable of boosting an immune checkpoint-mediated stimulatory signal). Such one or more immune checkpoint modulator(s) can be independently selected from the group consisting of peptides (e.g. peptide ligands), soluble domains of natural receptors, RNAi, antisense molecules, antibodies and protein scaffolds.

In certain embodiments, the immune checkpoint modulator is an antibody. The term “antibody” (“Ab”) is used in the broadest sense and encompasses those naturally occurring and engineered by man as well as full length antibodies or functional fragments (e.g., an scFv) or analogs thereof that are capable of binding the target immune checkpoint molecule or epitope thereof (thus retaining the target-binding portion). Such antibodies can be of any origin, e.g. human, humanized, animal (e.g. rodent or camelid antibody) or chimeric. It may be of any isotype with a specific preference for an IgG1 or IgG4 isotype. In addition, it may be glycosylated or non-glycosylated. The term antibody also includes bispecific or multispecific antibodies so long as they exhibit the binding specificity described herein.

As a brief description for illustrative purposes, full length antibodies are glycoproteins comprising at least two heavy (H) chains and two light (L) chains inter-connected by disulfide bonds. Each heavy chain is comprised of a heavy chain variable region (VH) and a heavy chain constant region which is made of three CH1, CH2 and CH3 domains (eventually with a hinge between CH1 and CH2). Each light chain is comprised of a light chain variable region (VL) and a light chain constant region which comprises one CL domain. The VH and VL regions comprise hypervariable regions, named complementarity determining regions (CDR), interspersed with more conserved regions named framework regions (FR). Each VH and VL is composed of three CDRs and four FRs in the following order: FR1-CDR1-FR2-CDR2-FR3-CDR3-FR4. The CDR regions of the heavy and light chains are determinant for the binding specificity.

As used herein, the term “humanized antibody” refers to a non-human (e.g. murine, camel, rat, etc) antibody whose protein sequence has been modified to increase its similarity to a human antibody (i.e. produced naturally in humans). The process of humanization is well known in the art (see e.g. Presta et al., 1997, Cancer Res. 57(20): 4593-9; U.S. Pat. Nos. 5,225,539; 5,530,101; 6,180,370; WO2012/110360). For example, a monoclonal antibody developed for human use can be humanized by substituting one or more residue of the FR regions to look like human immunoglobulin sequence whereas the vast majority of the residues of the variable regions (especially the CDRs) are not modified and correspond to those of a non-human immunoglobulin. For general guidance, the number of these amino acid substitutions in the FR regions is typically no more than 20 in each variable region VH or VL.

As used herein, a “chimeric antibody” refers to an antibody comprising one or more element(s) of one species and one or more element(s) of another species, for example, a non-human antibody comprising at least a portion of a constant region (Fc) of a human immunoglobulin.

Many forms of antibody can be engineered for use in the combination of the invention. Representative examples include without limitation Fab, Fab′, F(ab′)2, dAb, Fd, Fv, scFv, di-scFv and diabody, etc. More specifically:
(i) a Fab fragment represented by a monovalent fragment consisting of the VL, VH, CL and CH1 domains;
(ii) a F(ab′)2 fragment represented by a bivalent fragment comprising two Fab fragments linked by at least one disulfide bridge at the hinge region;
(iii) a Fd fragment consisting of the VH and CH1 domains;
(iv) a Fv fragment consisting of the VL and VH domains of a single arm of an antibody,
(v) a dAb fragment consisting of a single variable domain fragment (VH or VL domain);
(vi) a single chain Fv (scFv) comprising the two domains of a Fv fragment, VL and VH, that are fused together, eventually with a linker to make a single protein chain (see e.g. Bird et al., 1988, Science 242: 423-6; Huston et al., 1988, Proc. Natl. Acad. Sci. USA 85: 5879-83; U.S. Pat. Nos. 4,946,778; 5,258,498); and
(vii) any other artificial antibody.

Methods for preparing antibodies, fragments and analogs thereof are known in the art (see e.g. Harlow and Lane, 1988, Antibodies-A laboratory manual; Cold Spring Harbor Laboratory, Cold Spring Harbor N.Y.). One may cite for example hybridoma technology (as described in Kohler and Milstein, 1975, Nature 256: 495-7; Cote et al., 1983, Proc. Natl. Acad. Sci. USA 80: 2026-30; Cole et al. in Monoclonal antibodies and Cancer Therapy; Alan Liss pp 77-96), recombinant techniques (e.g. using phage display methods), peptide synthesis and enzymatic cleavage. Antibody fragments can be produced by recombinant technique as described herein. They can also be produced by proteolytic cleavage with enzymes such as papain to produce Fab fragments or pepsin to produce F(ab′)2 fragments as described in the literature (see e.g. Wahl et al., 1983, J. Nucl. Med. 24: 316-25). Analogs (or fragment thereof) can be generated by conventional molecular biology methods (PCR, mutagenesis techniques). If needed, such fragments and analogs may be screened for functionality in the same manner as with intact antibodies (e.g. by standard ELISA assay).

In some embodiments, at least one of the immune checkpoint modulator(s) for use with the methods and systems described herein is a monoclonal antibody, with a specific preference for a human (in which both the framework regions are derived from human germline immunoglobin sequences) or a humanized antibody according to well-known humanization process.

Desirably, the one or more immune checkpoint modulator(s) in use in the methods and compositions described herein antagonizes at least partially (e.g. more than 50%) the activity of inhibitory immune checkpoint(s), in particular those mediated by any of the following non-limiting examples: PD-1, PD-L1, PD-L2, LAG3, Tim3, KIR, BTLA and CTLA4, with a specific preference for a monoclonal antibody that specifically binds to any of such target proteins. The term “specifically binds to” refers to the capacity to a binding specificity and affinity for a particular target or epitope even in the presence of a heterogeneous population of other proteins and biologics. Thus, under designated assay conditions, the antibody binds preferentially to its target and does not bind in a significant amount to other components present in a test sample or subject. Preferably, such an antibody shows high affinity binding to its target with an equilibrium dissociation constant equal to or below 1×10⁻⁶M (e.g. at least 0.5×10⁻⁶, 1×10⁻⁷, 1×10⁻¹, 1×10⁻⁹, 1×10⁻¹⁰, etc). Alternatively, an immune checkpoint modulator(s) in use with the methods described herein can exert an agonist function in the sense that it is capable of stimulating or reinforcing stimulatory signals, in particular those mediated by CD28 with a specific preference for any of e.g., ICOS, CD137 (or 4-1BB), OX40, CD27, CD40 and GITR immune checkpoint molecules. Standard assays to evaluate the binding ability of the antibodies toward immune checkpoints are known in the art, including for example, ELISAs, Western blots, RIAs and flow cytometry. The binding kinetics (e.g., binding affinity) of the antibodies also can be assessed by standard assays known in the art, such as by Biacore analysis.

In certain embodiments, at least one of the immune checkpoint modulator(s) for use as described herein comprises a human or a humanized antibody capable of antagonizing an immune checkpoint involved in a T cell-mediated response.

In one embodiment, the blocking checkpoint molecule or stimulative checkpoint molecule modulates the activity of the programmed cell death 1 (PD-1)/programmed cell death ligand (PD-L1) signaling pathway. A non-limiting example of an immune checkpoint modulator is represented by a modulator capable of antagonizing at least partially the protein Programmed Death 1 (PD-1), and especially an antibody that specifically binds to human PD-1. PD-1 is part of the immunoglobulin (Ig) gene superfamily and a member of the CD28 family. It is a 55 kDa type 1 transmembrane protein expressed on antigen-experienced cells (e.g. activated B cells, T cells, and myeloid cells) (Agata et al., 1996, Int. Immunol. 8: 765-72; Okazaki et al., 2002, Curr. Opin. Immunol. 14: 391779-82; Bennett et al., 2003, J. Immunol 170: 711-8). In normal context, it acts by limiting the activity of T cells at the time of inflammatory response, thereby protecting normal tissues from destruction (Topalian, 2012, Curr. Opin. Immunol. 24: 207-12). Two ligands have been identified for PD-1, respectively PD-L1 (programmed death ligand 1) and PD-L2 (programmed death ligand 2) (Freeman et al., 2000, J. Exp. Med. 192: 1027-34; Carter et al., 2002, Eur. J. Immunol. 32: 634-43). PD-L1 was identified in 20-50% of human cancers (Dong et al., 2002, Nat. Med. 8: 787-9). The interaction between PD-1 and PD-L1 resulted in a decrease in tumor infiltrating lymphocytes, a decrease in T-cell receptor mediated proliferation, and immune evasion by the cancerous cells (Dong et al., 2003, J. Mol. Med. 81: 281-7; Blank et al., 2005, Cancer Immunol. Immunother. 54: 307-314). The complete nucleotide and amino acid PD-1 sequences can be found under GenBank Accession No U64863 and NP_005009.2. A number of anti PD1 antibodies are available in the art (see e.g. those described in WO2004/004771; WO2004/056875; WO2006/121168; WO2008/156712; WO2009/014708; WO2009/114335; WO2013/043569; and WO2014/047350, the contents of each of which are incorporated herein by reference in their entirety). Preferred anti PD-1 antibodies for use with the methods described herein are FDA approved or under advanced clinical development and one may use in particular an anti-PD-1 antibody selected from the group consisting of Nivolumab (also termed BMS-936558 under development by Bristol Myer Squibb), Pembrolizumab (also termed Lanbrolizumab or MK-3475; under development by Merck), and Pidilizumab (also termed CT-0I under development by CureTech).

Another non-limiting example of an immune checkpoint modulator is represented by a modulator capable of antagonizing, at least partially, the PD-1 ligand termed PD-L1, and especially an antibody that recognizes human PD-L1. A number of anti PD-L1 antibodies are available in the art (see e.g. those described in EP1907000). Preferred anti PD-L1 antibodies are FDA approved or under advanced clinical development (e.g. MPDL3280A under development by Genentech/Roche and BMS-936559 under development by Bristol Myer Squibb).

Cytotoxic T-lymphocyte associated antigen 4 (CTLA-4) is an immune checkpoint protein that down-regulates pathways of T-cell activation (Fong et al., Cancer Res. 69(2):609-615, 2009; Weber Cancer Immunol. Immunother, 58:823-830, 2009). CTLA4 (for cytotoxic T-lymphocyte-associated antigen 4) also known as CD152 was identified in 1987 (Brunet et al., 1987, Nature 328: 267-70) and is encoded by the CTLA4 gene (Dariavach et al., Eur. J. Immunol. 18: 1901-5). CTLA4 is a member of the immunoglobulin superfamily of receptors. It is expressed on the surface of helper T cells where it primarily regulates the amplitude of the early stages of T cell activation. Recent work has suggested that CTLA-4 may function in vivo by capturing and removing B7-1 and B7-2 from the membranes of antigen-presenting cells, thus making these unavailable for triggering of CD28 (Qureshi et al., Science, 2011, 332: 600-3). The complete CTLA-4 nucleic acid sequence can be found under GenBank Accession No LI 5006. Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells, thereby blocking the negative down regulation of the immune responses elicited by the interaction of these molecules. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238, the contents of each of which are incorporated herein by reference in their entirety. A non-limiting example of an anti-CDLA-4 antibody is tremelimumab, (ticilimumab, CP-675,206). In one embodiment, the anti-CTLA-4 antibody is ipilimumab (also known as 10D1, MDX-D010) a fully human monoclonal IgG antibody that binds to CTLA-4. Ipilimumab is marketed under the name Yervoy™ and has been approved for the treatment of unresectable or metastatic melanoma.

In other embodiments, the CTLA4 inhibitor comprises an antibody that recognizes human CTLA-4. A number of anti CTLA-4 antibodies are available in the art (see e.g. those described in U.S. Pat. No. 8,491,895). Preferred anti CTLA-4 antibodies for use with the methods and systems described herein are FDA approved or under advanced clinical development. For example, a CTLA-4 antibody for use as described herein include ipilimumab marketed by Bristol Myer Squibb as Yervoy (see e.g. U.S. Pat. Nos. 6,984,720; 8,017,114), tremelimumab under development by—Pfizer (see e.g. U.S. Pat. Nos. 7,109,003 and 8,143,379) and single chain anti-CTLA4 antibodies (see e.g. WO97/20574 and WO2007/123737). The contents of each of the aforementioned patents and patent applications are incorporated by reference herein in their entirety.

Another non-limiting example of a checkpoint molecule is TIGIT (T cell Immunoreceptor with Ig and ITIM domains), also called WUCAM, VSIG9 or Vstm3, which is a co-inhibitory receptor preferentially expressed on NK, CD8+ and CD4+ T cells as well as on regulatory T cells (Treg cells, or simply “Tregs”). TIGIT is a transmembrane protein containing a known ITIM domain in its intracellular portion, a transmembrane domain and an immunoglobulin variable domain on the extracellular part of the receptor. Several ligands have been described to bind to the TIGIT receptor with CD155/PVR showing the best affinity followed by CD113/PVRL3 and CD112/PVRL2 (Yu et al. (2009) Nat. Immunol. 10:48). DNAM/CD226, a known co-stimulatory receptor also expressed on NK and T cells competes with TIGIT for CD155 and CD112 binding but with a lower affinity, which suggests a tight control of the activation of these effector cells to avoid uncontrolled cytotoxicity against normal cells expressing CD155 ligand. TIGIT expression is increased on tumor infiltrating lymphocytes (TILs) and in disease settings such as HIV infection. TIGIT expression marks exhausted T cells that have lower effector function as compared to TIGIT negative counterparts (Kurtulus et al. (2015) J. Clin. Invest. 276:112; Chew et al. (2016) Plos Pathogens. 12). Conversely, Treg cells that express TIGIT show enhanced immunosuppressive activity as compared to TIGIT negative Treg population (Joller et al. (2014) Immunity. 40:569). Exemplary TIGIT antibodies are known in the art and/or are described in e.g., U.S. Pat. Nos. 10,329,349; 10,537,633; 10,047,158; 11,021,537; 11,008,390, the contents of each of which are incorporated herein by reference in their entirety.

Another exemplary immune checkpoint molecule comprises V-region Immunoglobulin-containing Suppressor of T cell Activation (VISTA) or PD-L3, which is a hematopoietically-restricted, structurally-distinct, Ig-superfamily inhibitory ligand designated as. The extracellular domain bears homology to the B7 family ligand PD-L1, and like PD-L1, VISTA has a profound impact on immunity. However, unlike PD-L1, expression of VISTA is exclusively within the hematopoietic compartment. Expression is most prominent on myeloid antigen-presenting cells (APCs), although expression on CD4+ T cells and on a subset of Foxp3+ regulatory T cells (Treg) is also of significant interest. Exemplary VISTA antibodies are described in U.S. Pat. Nos. 9,631,018; and 10,745,467, the contents of each of which are incorporated herein by reference in their entirety.

Other exemplary immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94).

Immune checkpoint modulators for antagonizing the LAG3 receptor can also be used in the methods described herein and are described in e.g., U.S. Pat. No. 5,773,578, the contents of which are incorporated herein by reference in their entirety

Another example of an immune checkpoint modulator is represented by an OX40 agonist such as agonist ligand of OX40 (Ox40L) (see e.g. U.S. Pat. Nos. 5,457,035, 7,622,444; WO03/082919, the contents of each of which are incorporated herein by reference in their entirety) or an antibody directed to the OX40 receptor (see e.g. U.S. Pat. No. 7,291,331 and WO03/106498, the contents of each of which are incorporated herein by reference in their entirety).

In some embodiments, an immune checkpoint modulator is selected from the group consisting of: anti-PD-1 antibodies (e.g., Nivolumab, Cemiplimab (REGN-2810), Pembrolizumab (MK-3475), Spartalizumab (PDR-001), Tislelizumab (BGB-A317), AMP-514 (MEDI0680), Dostarlimab (ANB011/TSR-042), Toripalimab (JS001), Camrelizumab (SHR-1210), Genolimzumab (CBT-501), Sintilimab (1B1308), STI-A1110, ENUM 388D4, ENUM 244C8, GLS010, MGA012, AGEN2034, CS1003, HLX10, BAT-1306, AK105, AK103, BI754091, LZM009, CMAB819, Sym021, GB226, SSI-361, JY034, HX008, ISU106, ABBV181, BCD-100, PF-06801591, CX-188 and JNJ-63723283, etc.), anti-PD-L1 antibodies (e.g., Atezolizumab (RG7446/MPDL3280A), Avelumab (PF-06834635/MSB0010718C), Durvalumab (MED14736), BMS-936559, STI-1014, KN035, LY3300054, HLX20, SHR-1316, CS1001 (WBP3155), MSB2311, BGB-A333, KL-A167, CK-301, AK106, AK104, ZKAB001, FAZ053, CBT-502 (TQB2450), JS003 and CX-072, etc.), PD-1 antagonists (e.g., AUNP-12, the respective compounds such as BMS-M1 to BMS-M10 (see WO2014/151634, WO2016/039749, WO2016/057624, WO2016/077518, WO2016/100285, WO2016/100608, WO2016/126646, WO2016/149351, WO2017/151830 and WO2017/176608), BMS-1, BMS-2, BMS-3, BMS-8, BMS-37, BMS-200, BMS-202, BMS-230, BMS-242, BMS-1001, BMS-1166 (see WO2015/034820, WO2015/160641, WO2017/066227 and Oncotarget. 2017 Sep. 22; 8 (42): 72167-72181), the respective compounds of Incyte-1 to Incyte-6 (see WO 2017/070089, WO2017/087777, WO2017/106634, WO 2017/112730, WO 2017/192961 and WO2017/205464), CAMC-1 to CAMC-4 (see WO 2017/202273, WO2017/202274, WO2017/202275 and WO2017/202276), RG_1 (see WO2017/118762) ant DPPA-1 (see Angew. Chem. Int. Ed. 2015, 54, 11760-11764), etc.), PD-L1/VISTA antagonists (e.g., CA-170 etc.), PD-L1/TIM3 antagonists (e.g., CA-327 etc.), anti-PD-L2 antibodies, PD-L1 fusion proteins, PD-L2 fusion proteins (e.g., AMP-224 etc.), anti-CTLA-4 antibodies (e.g., Ipilimumab (MDX-010), AGEN1884 and Tremelimumab, etc.), anti-LAG-3 antibodies (e.g., Relatlimab (BMS-986016/ONO-4482), LAG525, REGN3767 and MK-4280, etc.), LAG-3 fusion proteins (e.g., IMP321 etc.), anti-Tim3 antibodies (e.g., MBG453 and TSR-022, etc.), anti-KIR antibodies (e.g., Lililumab (BMS-986015, ONO-4483), IPH2101, LY3321367 and MK-4280, etc.), anti-BTLA antibodies, anti-TIGIT antibodies (e.g., Tiragolumab (MTIG-7192A/RG-6058/RO-7092284) and BMS-986207 (ONO-4686), anti-VISTA antibody (e.g., JNJ-61610588) and anti-CSF-1R antibody or CSF-1R inhibitor (e.g., Cabiralizumab (FPA008/BMS-986227/ONO-4687), Emactuzumab (RG7155/RO5509554), LY3022855, MCS-110, IMC-CS4, AMG820, Pexidartinib, BLZ945 and ARRY-382, etc.).

Additional immune checkpoint inhibitors and modulators thereof are known to those of skill in the art and are not described herein, however any immune checkpoint modulator can be used with the methods and systems described herein.

### Administration and Delivery of Therapeutic Stem Cells

Local delivery of cells, e.g., virus loaded and/or genetically modified stem cells as described herein, can provide benefits for cancer therapy. In one aspect, local delivery can provide a high local concentration of the therapeutic polypeptide(s) or effector cells. However, one of the benefits of stem cell delivery of therapeutic agents is the natural tumor-homing activity of those cells. Thus, systemic administration, e.g., via intravenous delivery is specifically contemplated for stem cell-delivered therapies as described herein. However, benefits of systemic administration can be hampered for certain tumor types, notably brain tumors, where the blood-brain barrier can limit access of systemically administered cells to a tumor. For this reason, local delivery to the site of a tumor, and especially considering the immunostimulatory effects of tumor resection, local delivery of therapeutic cells to the site of tumor resection, can be of particular benefit for the treatment of brain tumors, including but not limited to GBM, which are notoriously difficult to treat.

In one embodiment, the stem cell-delivered therapies as described herein are delivered via intrathecal administration. In one embodiment, intrathecal administration is intrathecal administration at the Cisterna Magna. In one embodiment, the stem cell-delivered therapies as described herein are delivered via intrathecal administration to a subject having melanoma leptomeningeal metastasis (MLM). In one embodiment, the stem cell-delivered therapies as described herein are delivered via intrathecal administration to a subject having a melanoma brain metastasis.

In one embodiment, the genetically modified stem cells, e.g., MSCs or NSCs, among others, are encapsulated in a matrix. This can assist in retaining the stem cells in a given location, such as a tumor resection cavity. The matrix can minimize wash out of cells from the resection cavity, e.g., by CSF in the case of brain tumors.

A matrix useful in the methods and compositions described herein will permit stem cells to migrate away from the matrix, rather than containing the cells within the matrix permanently. As used herein, “matrix” refers to a biological material that comprises a “biocompatible substrate” that can be used as a material that is suitable for implantation into a subject or into which a cell population can be deposited. A biocompatible substrate does not cause toxic or injurious effects once implanted in the subject. The biocompatible substrate can but need not necessarily provide the supportive framework that allows cells to attach to it, and grow on it. Cultured populations of cells (e.g., genetically modified MSCs or other stem cells) can be prepared with the biocompatible substrate (i.e., the matrix), which provides the appropriate interstitial distances required, e.g., for cell-cell interaction. As used herein, “encapsulated” refers to a cell that is enclosed within the matrix.

A matrix can be used to aid in further controlling and directing a cell or population of genetically modified stem cells as described herein. A matrix can be designed or selected to provide environmental cues to control and direct the migration of cells to a site of injury or disease. A structure can be engineered from a nanometer to micrometer to millimeter to macroscopic length, and can further comprise or be based on factors such as, but not limited to, material mechanical properties, material solubility, spatial patterning of bioactive compounds, spatial patterning of topological features, soluble bioactive compounds, mechanical perturbation (cyclical or static strain, stress, shear, etc.), electrical stimulation, and thermal perturbation.

In one embodiment, the matrix comprises a synthetic matrix. In one embodiment, the matrix comprises a thiol-modified hyaluronic acid and a thiol-reactive cross-linker molecule. In one embodiment, the thiol-reactive cross-linker molecule is polyethylene glycol diacrylate. Further description of components useful in constructing a matrix, as well as instruction for making a matrix, can be found in U.S. patent application Ser. No. 15/225,202, which is incorporated herein in its entirety by reference.

Methods of encapsulation of stem cells are known in the art and can be found, for example, in Shah et al. Biomatter. 2013 and Kauer et al. Nature Neuroscience. 2013.

For example, the synthetic extracellular matrix (ECM) components, such as those from Hystem and Extralink (Glycosan Hystem-C, Biotime Inc.), can be reconstituted according to the manufacturer's protocols. Stem cells (e.g. 1×10⁵, 2×10⁵ or 4×10⁵ cells) can be re-suspended in Hystem (e.g. 14 μl) and the matrix is cross-linked by adding Extralink (e.g. 6 μl). After about 20 minutes (gelation time) at 25° C., the stem cell and ECM hydrogel can be placed in the center of different sizes (35 or 60 mm) of glass-bottomed dish. Bioluminescence imaging can be used to determine the viability of the MSCs expressing a detectable label. To assess the numbers of cells expressing immunomodulatory polypeptides and the amounts of such polypeptides expressed, methods known in the art can be used such as flow cytometry, Western blotting, immunohistochemistry, or enzyme-linked immunosorbent assay (ELISA).

Biopolymers useful in the generation of the matrices and scaffolds for the embodiments directed to cellular therapies using stem cells as described herein include, but are not limited to, a) extracellular matrix proteins to direct cell adhesion and function (e.g., collagen, fibronectin, laminin, etc.); (b) growth factors to direct cell function specific to cell type (e.g., nerve growth factor, bone morphogenic proteins, vascular endothelial growth factor, etc.); (c) lipids, fatty acids and steroids (e.g., glycerides, non-glycerides, saturated and unsaturated fatty acids, cholesterol, corticosteroids, sex steroids, etc.); (d) sugars and other biologically active carbohydrates (e.g., monosaccharides, oligosaccharides, sucrose, glucose, glycogen, etc.); (e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins [e.g., selectins, immunoglobulins, hormones such as human chorionic gonadotropin, Alpha-fetoprotein and Erythropoietin (EPO), etc.]; proteolipids (e.g., N-myristoylated, palmitoylated and prenylated proteins); and glycolipids (e.g., glycoglycerolipids, glycosphingolipids, glycophosphatidylinositols, etc.); (f) biologically derived homopolymers, such as polylactic and polyglycolic acids and poly-L-lysine; (g) nucleic acids (e.g., DNA, RNA, etc.); (h) hormones (e.g., anabolic steroids, sex hormones, insulin, angiotensin, etc.); (i) enzymes (types: oxidoreductases, transferases, hydrolases, lyases, isomerases, ligases; examples: trypsin, collegenases, matrix metallproteinases, etc.); (j) pharmaceuticals (e.g., beta blockers, vasodilators, vasoconstrictors, pain relievers, gene therapy, viral vectors, anti-inflammatories, etc.); (k) cell surface ligands and receptors (e.g., integrins, selectins, cadherins, etc.); (l) cytoskeletal filaments and/or motor proteins (e.g., intermediate filaments, microtubules, actin filaments, dynein, kinesin, myosin, etc.), or any combination thereof. For example, a biopolymer can be selected from the group consisting of fibronectin, vitronectin, laminin, collagen, fibrinogen, silk or silk fibroin.

It is contemplated that in addition to the administration of cells in suspension via, e.g., intraarterial or intravenous injection, in some embodiments of the methods described herein, stem cells modified to release a virus or engineered to express or secrete therapeutic polypeptides are encapsulated in an extracellular matrix comprising a thiol-modified hyaluronic acid and a thiol-reactive cross-linker, such as, for example, polyethylene glycol diacrylate for administration.

In one method of cell encapsulation, the isolated cells are mixed with sodium alginate and extruded into calcium chloride so as to form gel beads or droplets. The gel beads are incubated with a high molecular weight (e.g., MW 60-500 kDa) concentration (0.03-0.1% w/v) polyamino acid (e.g., poly-L-lysine) to form a membrane. The interior of the formed capsule is re-liquified using sodium citrate. This creates a single membrane around the cells that is highly permeable to relatively large molecules (MW .about.200-400 kDa), but retains the cells inside. The capsules are incubated in physiologically compatible carrier for several hours in order that the entrapped sodium alginate diffuses out and the capsules expand to an equilibrium state. The resulting alginate-depleted capsules is reacted with a low molecular weight polyamino acid which reduces the membrane permeability (MW cut-off 40-80 kDa).

In some embodiments, additional bioactive substances can be added to a biopolymer matrix or scaffold comprising the stem cells as described herein. The amounts of such optionally added bioactive substances can vary widely with optimum levels being readily determined in a specific case by routine experimentation.

### Administration

In some embodiments, the methods described herein relate to treating a subject having or diagnosed a solid tumor cancer by administering one or more stem cells as described herein.

Pharmaceutically acceptable carriers and diluents include saline, aqueous buffer solutions, solvents and/or dispersion media. The use of such carriers and diluents is well known in the art. The solution is preferably sterile and fluid. Preferably, prior to the introduction of cells as described herein, the solution is stable under the conditions of manufacture and storage and preserved against the contaminating action of microorganisms such as bacteria and fungi through the use of agents such as parabens, chlorobutanol, phenol, ascorbic acid, thimerosal, and the like.

Direct injection techniques for cell administration can also be used to stimulate transmigration of cells through the entire vasculature, or to a particular organ, such as for example the brain. This includes non-specific targeting of the vasculature. One can target any organ by selecting a specific injection site, e.g., the carotid artery for targeting the brain. Accordingly, in some embodiments of the methods described herein, stem cells, such as neural stem cells, modified to deliver an oncolytic virus and/or one or more therapeutic polypeptides are administered via direct injection into the carotid artery.

Alternatively, the injection can be performed systemically into any vessel in the body. Thus, in some embodiments of the methods described herein, stem cells, such as MSCs, neural stem cells, etc. loaded with virus or genetically modified to express one or more therapeutic polypeptides are administered systemically. In some embodiments of the methods described herein, such stem cells are not administered intravenously.

In one embodiment, the solid tumor has been resected prior to administration. Subjects having a condition (e.g., glioblastoma) can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g. the condition, but are not limited to, blood screening and imaging (e.g., PET scan), and are known in the art for a given condition. A family history for a condition, or exposure to risk factors for a condition can also aid in determining if a subject is likely to have the condition or in making a diagnosis of the condition.

A variety of approaches for administering cells to subjects are known to those of skill in the art. In some embodiments, and particularly where delivery to the brain is desired, an advantage provided by the use of neural stem cells to deliver therapeutic proteins is that the cells need not be administered directly to the brain—that is, intracranial administration and administration directly to the parenchyma of the brain are not required. The ability to administer systemically, e.g., by intraarterial injection, permits a less invasive approach and facilitates delivery throughout the brain, rather than at just one or several focal points of injection to the brain parenchyma. This approach permits delivery of therapeutic cells via the brain's circulation is much the same way that the multifocal metastatic tumor cells arrived in the brain. Such methods can include systemic injection, for example, injection directly into the carotid artery. In some embodiments of the methods described herein, administration does not include or comprise implantation of cells directly into a tumor target site in a subject, such as a surgical site. Cells can be inserted into a delivery device which facilitates introduction by injection or implantation into the subject. Such delivery devices can include tubes, e.g., catheters, for injecting cells and fluids into the body of a recipient subject. In some embodiments, the tubes additionally have a needle, e.g., through which the cells can be introduced into the subject at a desired location. The cells can be prepared for delivery in a variety of different forms. Cells can be mixed with a pharmaceutically acceptable carrier or diluent in which the cells remain viable.

In one embodiment, the modified MSCs described herein are administered directly into the cavity formed by resection of a tumor.

The compositions described herein can be administered to a subject having or diagnosed as having a condition. In some embodiments, the methods described herein comprise administering an effective amount of stem cells as described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In one embodiment, the compositions described herein are administered systemically or locally.

The term “effective amount” as used herein refers to the amount of stem cells as described herein needed to alleviate at least one or more symptom of the disease (e.g., glioblatoma), and relates to a sufficient amount of the cell preparation or composition to provide the desired effect. The term “therapeutically effective amount” therefore refers to an amount of stem cells that is sufficient to provide a particular anti-condition effect when administered to atypical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized.

Pharmaceutically acceptable carriers for cell-based therapeutic formulation include saline and aqueous buffer solutions, Ringer's solution, and serum component, such as serum albumin, HDL and LDL. The terms such as “excipient”, “carrier”, “pharmaceutically acceptable carrier” or the like are used interchangeably herein.

In some embodiments, the pharmaceutical composition comprising stem cells as described herein can be a parenteral dose form. Since administration of parenteral dosage forms typically bypasses the patient's natural defenses against contaminants, the components apart from the stem cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the preparation of stem cells prior to administration.

Suitable vehicles that can be used to provide parenteral dosage forms of stem cells as described herein are well known to those skilled in the art. Examples include, without limitation: saline solution; glucose solution; aqueous vehicles including but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Dosage

The dosage of the stem cell-based treatments to be administered to a patient will vary with the precise nature of the condition being treated and the recipient of the treatment. The scaling of dosages for human administration can be performed according to art-accepted practices. The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

The stem cells as described herein can be formulated in unit dosage form for delivery. “Unit dosage form” as the term is used herein refers to a dosage suitable for one administration. By way of example a unit dosage form can be an amount of therapeutic disposed in a delivery device, e.g., a syringe or intravenous drip bag. In one embodiment, a unit dosage form is administered in a single administration. In another embodiment, more than one unit dosage form can be administered simultaneously.

A pharmaceutical composition comprising the stem cells as described herein can generally be administered at a dosage of 10⁴ to 10⁹ cells/kg body weight, in some instances 10⁵ to 10⁶ cells/kg body weight, including all integer values within those ranges. If necessary, stem cells as described herein can also be administered multiple times at these dosages. The cells can be administered by using infusion techniques that are commonly known in immunotherapy (see, e.g., Rosenberg et al., New Eng. J. of Med. 319:1676, 1988).

As discussed above, modes of administration can include, for example intravenous (i.v.) injection or infusion. The compositions described herein can be administered to a patient transarterially, intratumorally, intranodally, or intramedullary. In some embodiments, the stem cells can be injected directly into a tumor, lymph node, or site of infection. In one embodiment, the compositions described herein are administered into a body cavity or body fluid (e.g., ascites, pleural fluid, peritoneal fluid, or cerebrospinal fluid).

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The technology provided herein can further be described in any of the following numbered paragraphs:
1. A method of treating cancer, the method comprising administering to a subject in need thereof a first stem cell (SC) modified to release an oncolytic virus and a second SC which is gene edited to inactivate a receptor for the oncolytic virus, thereby generating a SC resistant to the virus, wherein the second SC is also engineered to express an immunomodulatory polypeptide agent.
2. The method of paragraph 1, wherein the second SC is gene edited to inactivate the receptor for the oncolytic virus before the second SC is engineered to express the immunomodulatory polypeptide agent.
3. The method of any preceding paragraph, wherein the first and/or second SC is a mesenchymal stem cell (MSC) or a neuronal stem cell (NSC).
4. The method of any preceding paragraph, wherein the first and/or second SC is autologous to the subject.
5. The method of any preceding paragraph, wherein the first and/or second SC is allogeneic to the subject.
6. The method of any preceding paragraph, wherein the oncolytic virus is an oncolytic herpes simplex virus (oHSV).
7. The method of any preceding paragraph, wherein the oHSV is or is derived from G47Δ oHSV.
8. The method of any preceding paragraph, wherein the receptor is nectin-1.
9. The method of any preceding paragraph, wherein the oncolytic virus encodes a heterologous polypeptide.
10. The method of any preceding paragraph, wherein the heterologous polypeptide is a tumor necrosis factor related apoptosis-inducing ligand (TRAIL) polypeptide or a cytokine that promotes an anti-tumor immune response.
11. The method of any preceding paragraph, wherein the cytokine that promotes an anti-tumor immune response is one or more of GM-CSF, IL-2, IL-12, Flt3L, IL-5 and IL-15.
12. The method of any preceding paragraph, wherein the immunomodulatory polypeptide agent expressed by the second SC comprises a cytokine that promotes an anti-tumor immune response.
13. The method of any preceding paragraph, wherein the cytokine is one or more of GM-CSF, IL-2, IL-12, Flt3L, IL-5 and IL-15.
14. The method of any preceding paragraph, wherein the cytokine is a GM-CSF polypeptide.
15. The method of any preceding paragraph, wherein the immunomodulatory polypeptide agent comprises a modulator of an immune checkpoint molecule.
16. The method of any preceding paragraph, wherein the modulator of an immune checkpoint molecule is an agonist of a stimulatory checkpoint molecule or an antagonist of an inhibitory checkpoint molecule.
17. The method of any preceding paragraph, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.
18. The method of any preceding paragraph, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.
19. The method of any preceding paragraph, wherein the modulator of an immune checkpoint molecule comprises an antibody or an antigen-binding fragment thereof that specifically binds the immune checkpoint molecule.
20. The method of any preceding paragraph, wherein the second SC further expresses a second therapeutic polypeptide.
21. The method of any preceding paragraph, wherein the second therapeutic polypeptide comprises a cytokine or a modulator of an immune checkpoint molecule.
22. The method of any preceding paragraph, wherein the cytokine is one or more of GM-CSF, IL-2, IL-12, Flt3L, IL-5 and IL-15.
23. The method of any preceding paragraph, wherein the modulator of an immune checkpoint molecule is an agonist of a stimulatory checkpoint molecule or an antagonist of an inhibitory checkpoint molecule.
24. The method of any preceding paragraph, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.
25. The method of any preceding paragraph, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.
26. The method of any preceding paragraph, wherein the modulator of an immune checkpoint molecule comprises an antibody or an antigen-binding fragment thereof that specifically binds the immune checkpoint molecule.
27. A method of treating cancer, the method comprising administering to a subject in need thereof a stem cell (SC) modified to express and secrete a receptor-targeted cytotoxic agent, wherein the SC is gene edited to inactivate a receptor for the receptor-targeted cytotoxic agent.
28. The method of any preceding paragraph, wherein the receptor-targeted cytotoxic agent is a cytokine or a death receptor-targeted pro-apoptotic factor.
29. The method of any preceding paragraph, wherein the cytokine is IFNβ.
30. The method of any preceding paragraph, wherein the receptor for the receptor-targeted cytotoxic agent is IFNaR1 or IFNaR2.
31. The method of any preceding paragraph, wherein the death receptor-targeted pro-apoptotic factor is tumor necrosis factor related apoptosis-inducing ligand (TRAIL).
32. The method of any preceding paragraph, wherein the receptor for the receptor-targeted cytotoxic agent is death receptor (DR) 4 or DR5.
33. The method of any preceding paragraph, wherein the receptor for the oncolytic virus or the receptor-targeted cytotoxic agent is inactivated by targeted gene editing.
34. The method of any preceding paragraph, wherein the SC is further engineered to express an immunomodulator polypeptide.
35. The method of any preceding paragraph, wherein the immunomodulator polypeptide is a cytokine that promotes an anti-tumor immune response.
36. The method of any preceding paragraph, wherein the cytokine is one or more of GM-CSF, IL-12, IL-2, IL-12, Flt3L, IL-5 and IL-15.
37. The method of any preceding paragraph, wherein the cytokine is a GM-CSF polypeptide.
38. The method of any preceding paragraph, wherein the immunomodulator polypeptide comprises a modulator of an immune checkpoint molecule.
39. The method of any preceding paragraph, wherein the modulator of an immune checkpoint molecule is an agonist of a stimulatory checkpoint molecule or an antagonist of an inhibitory checkpoint molecule.
40. The method of any preceding paragraph, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.
41. The method of any preceding paragraph, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.
42. The method of any preceding paragraph, wherein the modulator of an immune checkpoint molecule comprises an antibody or an antigen-binding fragment thereof that specifically binds the immune checkpoint molecule.
43. The method of any preceding paragraph, further comprising administering a second SC engineered to express an immunomodulatory polypeptide agent.
44. The method of any preceding paragraph, wherein the cancer comprises a solid tumor cancer.
45. The method of any preceding paragraph, wherein the cancer is selected from melanoma, lung cancer, breast cancer and glioblastoma.
46. The method of any preceding paragraph, wherein the cancer comprises a primary tumor or a metastatic tumor.
47. The method of any preceding paragraph, wherein the metastatic tumor comprises a metastasis to the brain.
48. The method of any preceding paragraph, wherein the cancer is PTEN-deficient.
49. The method of any preceding paragraph, wherein the administering comprises intratumor administration.
50. The method of any preceding paragraph, wherein the administering comprises systemic administration.
51. The method of any preceding paragraph, wherein the administering comprises administration of any or all of the SCs to a tumor resection cavity.
52. A composition comprising a) a first stem cell (SC) modified to release an oncolytic virus, and b) a second SC which is gene edited to inactivate a receptor for the oncolytic virus, thereby generating a SC resistant to the virus, wherein the second SC is also engineered to express an immunomodulatory polypeptide agent.
53. The composition of any preceding paragraph, wherein the second SC is gene edited to inactivate the receptor for the oncolytic virus before the second SC is engineered to express the immunomodulatory polypeptide agent.
54. The composition of any preceding paragraph, wherein the first and/or second SC is a mesenchymal stem cell (MSC) or a neuronal stem cell (NSC).
55. The composition of any preceding paragraph, wherein the first and/or second SC is autologous to the subject.
56. The composition of any preceding paragraph, wherein the first and/or second SC is allogeneic to the subject.
57. The composition of any preceding paragraph, wherein the oncolytic virus is an oncolytic herpes simplex virus (oHSV).
58. The composition of any preceding paragraph, wherein the oHSV is or is derived from G47Δ oHSV.
59. The composition of any preceding paragraph, wherein the receptor is nectin-1.
60. The composition of any preceding paragraph, wherein the oncolytic virus encodes a heterologous polypeptide.
61. The composition of any preceding paragraph, wherein the heterologous polypeptide is a tumor necrosis factor related apoptosis-inducing ligand (TRAIL) polypeptide or a cytokine that promotes an anti-tumor immune response.
62. The composition of any preceding paragraph, wherein the cytokine that promotes an anti-tumor immune response is one or more of GM-CSF, IL-2, IL-12, Flt3L, IL-5 and IL-15.
63. The composition of any preceding paragraph, wherein the immunomodulatory polypeptide agent expressed by the second SC comprises a cytokine that promotes an anti-tumor immune response.
64. The composition of any preceding paragraph, wherein the cytokine is one or more of GM-CSF, IL-2, IL-12, Flt3L, IL-5 and IL-15.
65. The composition of any preceding paragraph, wherein the cytokine is a GM-CSF polypeptide.
66. The composition of any preceding paragraph, wherein the immunomodulatory polypeptide agent comprises a modulator of an immune checkpoint molecule.
67. The composition of any preceding paragraph, wherein the modulator of an immune checkpoint molecule is an agonist of a stimulatory checkpoint molecule or an antagonist of an inhibitory checkpoint molecule.
68. The composition of any preceding paragraph, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.
69. The composition of any preceding paragraph, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.
70. The composition of any preceding paragraph, wherein the modulator of an immune checkpoint molecule comprises an antibody or an antigen-binding fragment thereof that specifically binds the immune checkpoint molecule.
71. The composition of any preceding paragraph, wherein the second SC further expresses a second therapeutic polypeptide.
72. The composition of any preceding paragraph, wherein the second therapeutic polypeptide comprises a cytokine or a modulator of an immune checkpoint molecule.
73. The composition of any preceding paragraph, wherein the cytokine is one or more of GM-CSF, IL-2, IL-12, Flt3L, IL-5 and IL-15.
74. The composition of any preceding paragraph, wherein the modulator of an immune checkpoint molecule is an agonist of a stimulatory checkpoint molecule or an antagonist of an inhibitory checkpoint molecule.
75. The composition of any preceding paragraph, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.
76. The composition of any preceding paragraph, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.
77. The composition of any preceding paragraph, wherein the modulator of an immune checkpoint molecule comprises an antibody or an antigen-binding fragment thereof that specifically binds the immune checkpoint molecule.
78. A composition comprising a stem cell (SC) modified to express and secrete a receptor-targeted cytotoxic agent, wherein the SC is gene edited to inactivate a receptor for the receptor-targeted cytotoxic agent.
79. The composition of any preceding paragraph, wherein the receptor-targeted cytotoxic agent is a cytokine or a death receptor-targeted pro-apoptotic factor.
80. The composition of any preceding paragraph, wherein the cytokine is IFNβ.
81. The composition of any preceding paragraph, wherein the receptor for the receptor-targeted cytotoxic agent is IFNaR1 or IFNaR2.
82. The composition of any preceding paragraph, wherein the death receptor-targeted pro-apoptotic factor is tumor necrosis factor related apoptosis-inducing ligand (TRAIL).
83. The composition of any preceding paragraph, wherein the receptor for the receptor-targeted cytotoxic agent is death receptor (DR) 4 or DR5.
84. The composition of any preceding paragraph, wherein the receptor for the oncolytic virus or the receptor-targeted cytotoxic agent is inactivated by targeted gene editing.
85. The composition of any preceding paragraph, wherein the SC is further engineered to express an immunomodulator polypeptide.
86. The composition of any preceding paragraph, wherein the immunomodulator polypeptide is a cytokine that promotes an anti-tumor immune response.
87. The composition of any preceding paragraph, wherein the cytokine is one or more of GM-CSF, IL-12, IL-2, IL-12, Flt3L, IL-5 and IL-15.
88. The composition of any preceding paragraph, wherein the cytokine is a GM-CSF polypeptide.
89. The composition of any preceding paragraph, wherein the immunomodulator polypeptide comprises a modulator of an immune checkpoint molecule.
90. The composition of any preceding paragraph, wherein the modulator of an immune checkpoint molecule is an agonist of a stimulatory checkpoint molecule or an antagonist of an inhibitory checkpoint molecule.
91. The composition of any preceding paragraph, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.
92. The composition of any preceding paragraph, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.
93. The composition of any preceding paragraph, wherein the modulator of an immune checkpoint molecule comprises an antibody or an antigen-binding fragment thereof that specifically binds the immune checkpoint molecule.
94. The composition of any preceding paragraph, further comprising at a least a second SC, wherein the second SC is different from the first SC.
95. The composition of any preceding paragraph, further comprising a pharmaceutically acceptable carrier.
96. Use of a composition of any preceding paragraph for the treatment of cancer in a subject in need thereof.
97. The use of any preceding paragraph, wherein the cancer comprises a solid tumor cancer.
98. The use of any preceding paragraph, wherein the cancer is selected from melanoma, lung cancer, breast cancer and glioblastoma.
99. The use of any preceding paragraph, wherein the cancer comprises a primary tumor or a metastatic tumor.
100. The use of any preceding paragraph, wherein the metastatic tumor comprises a metastasis to the brain.
101. The use of any preceding paragraph, wherein the cancer is PTEN-deficient.
102. The method of any preceding paragraph, wherein the immunomodulatory polypeptide agent of the second SC comprises a modulator of an immune checkpoint molecule.
103. The method of any preceding paragraph, wherein the modulator of an immune checkpoint molecule is an agonist of a stimulatory checkpoint molecule or an antagonist of an inhibitory checkpoint molecule.
104. The method of any preceding paragraph, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.
105. The method of any preceding paragraph, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.
106. The method of any preceding paragraph, wherein the immunomodulatory polypeptide agent of the first and second SC are the same.
107. The method of any preceding paragraph, wherein the immunomodulatory polypeptide agent of the first and second SC are different.
108. The method of any preceding paragraph, wherein the immunomodulatory polypeptide agent of the first and second SC are the same.
109. The method of any preceding paragraph, wherein the immunomodulatory polypeptide agent of the first and second SC are different.

### EXAMPLES

### Example 1. Gene Edited and Engineered Allogeneic Twin Stem Cell Mediated Immunotherapy for Brain Metastatic Melanomas

Oncolytic viral therapy has shown promising results in treating primary melanomas, however its efficacy in brain metastases remains challenging and is hampered by premature elimination of viral particles, the need to bypass the blood-brain barrier (BBB), and the immunosuppressive-nature of tumors in the brain. Work described herein shows the development of a multimodal approach by creating tumor-tropic allogeneic twin (T) stem cells (SC): one for producing oncolytic herpes simplex virus (oHSV) and the other one engineered with CRISPR/Cas9 mediated knockout of Nectin 1 (N1) receptor (NIKO) to acquire resistance to oHSV and releasing immunomodulators. An in vivo screening of SC^{N1KO} released cytokines identified GM-CSF as the most potent immunomodulator to augment SC-oHSV therapy for primary and metastatic PTEN-deficient melanomas. Utilizing mouse models of primary and orthotopic brain metastatic BRAF^{V600E}/PTEN^{-/-} and BRAF^{V600E}/wt/PTEN^{-/-} mutant melanomas, it was shown that locoregional delivery of twin stem cells releasing oHSV and GM-CSF (TSC-G) activated dendritic cell and T cell-mediated immune responses in both syngeneic and patient-derived humanized mouse models. Importantly, this strategy exhibited greater therapeutic efficacy when compared with the existing oncolytic viral therapeutic approaches. Moreover, the use of SC-oHSV with SC^{N1KO} releasing both GM-CSF and single-chain variable fragment (scFv) anti-PD-1 (TSC-G/P) effectively suppressed immunosuppressive PTEN-deficient leptomeningeal metastasis. Findings presented herein in this Example provide a novel allogeneic SC-based immunotherapeutic strategy against melanomas and a roadmap towards clinical application for leptomeningeal disease, among others.

### Introduction

Despite recent improvements in therapeutic approaches, patients with advanced melanoma still have limited survival prognosis, with brain metastases contributing to half of all melanoma-related deaths (1). Advanced stage melanomas have a high propensity to metastasize to the brain with 60% of patients developing brain metastases at some point (2, 3). Although multidisciplinary therapies such as radiotherapy, targeted therapy and surgeries have been used to treat melanoma brain metastasis (MBM), the overall survival is only 4-6 months (4). Given that the majority of patients with metastatic tumors in the brain do not undergo surgery and treatment with numerous systemic therapies has shown sub-optimal response (5), alternative therapeutic agents/strategies are urgently needed.

Oncolytic viruses (OV) selectively replicate in and kill neoplastic cells (6, 7) and are among the latest therapies that have progressed to the clinic (8-12). Intralesional injection of FDA-approved talimogene laherparepvec (T-VEC; recombinant oncolytic herpes simplex virus, oHSV) (13) induces anti-tumor immune responses for distant un-injected tumor lesions, but has not improved overall patient survival of stage IVM1b and IVM1c disease that has metastatic lesions to the brain, bone, liver, lungs or other internal organs (14). oHSV delivery is shown to be hampered by the issues such as virus neutralization, sequestration and inefficient extravasation (15). To counter these issues, the inventors have previously shown that mesenchymal stem cells (SC) loaded with oHSV home extensively to multiple metastatic tumor deposits in the brain and have therapeutic efficacy in imageable mouse models of MBM (16). However, the immunosuppressive tumor microenvironment of MBM (17) which prevents efficient anti-tumor immune responses is yet to be addressed. PTEN loss is associated with significantly shorter time to MBM and correlates with shorter overall survival (18). PTEN dysfunction drives the selectivity and spread of OVs via inhibition of interferon responses (19, 20). Therefore, PTEN intact and mutant melanoma cell lines have been utilized here to develop and characterize syngeneic mouse models of PTEN mutant MBM.

Cytokines are potent immunomodulatory molecules and have been successfully used as adjuvants in OV therapy for cancer. Numerous pre-clinical and clinical studies have investigated the therapeutic efficacy of cytokine-expressing OVs such as oHSV (such as T-VEC) (13), adenovirus (21), and vaccinia virus (22) in different cancer types. However, OV mediated cytokine expression is dependent on OV infection of cancer cells, and infected cells are expected to die, making the levels and duration of cytokine secretion in the tumor microenvironment unpredictable (23, 24). It was specifically reasoned that SC delivery of immunomodulator cytokines could address such problems. However, co-delivery of oHSV and immunomodulators from the same mesenchymal SCs is not feasible as they are susceptible to oHSV-mediated oncolysis (25). The inventors have previously shown that both mouse and human SC have high expression of Nectin-1 (CD111), the most efficient entry receptor for oHSV (26) (16). In this study, Nectin-1 receptor knockout SC (SC^{N1KO}) were first created using CRISPR/Cas-9 technology and the SC^{N1KO} cells were further engineered to release various immunomodulators. Together with oHSV-releasing SC (SC-oHSV), a multimodal therapeutic approach that can eliminate tumor cells as well as induce active and long-term immunity in an aggressive PTEN-deficient primary and metastatic melanoma is demonstrated.

Immune checkpoint inhibitors (ICI) are one of the major advances in recent cancer therapy, especially for the treatment of metastatic melanoma (27), which is typically immunogenic (28). Immune checkpoint inhibition can be rationally combined with oHSV therapy since virally infected dying tumor cells release tumor antigens into the tumor microenvironment to attract innate and adaptive immune cells, including tumor-specific CD4⁺ and CD8⁺ T cells, offering the potential to achieve a more durable response and outcome (29-31). Indeed, recent studies have shown that oHSV in combination with anti-PD-1, and/or CTLA-4 antibody leads to abscopal effects via the increases in CD3+, CD8+ T-cells, and M1-like polarization, and decreases in Tregs (32)-(33). Conversely, some clinical studies with systemic administration of ICIs also suggest that the intracranial responses are poorer than the extracranial responses due to the limited penetration of anti-PD-1 antibodies into the brain and cerebrospinal fluid (CSF) (34-37). Since oHSV therapy results in upregulation of PD-L1 via IFNγ production (38); GM-CSF increases PD-L1 expression in MDSC (39), and PTEN loss increases PD-L1 expression on tumor cells (40) and promotes immune resistance in melanomas (41), SC^{N1KO}-GM-CSF were further engineered to express single-chain variable fragment anti-PD-1 (scFvPD-1).

The presence of an adaptive immune system in syngeneic mice provides an excellent platform for studies of novel treatments modulating anti-tumor immune responses, however, the lack of a wide range of tumor models carrying clinically relevant oncogenic molecular alterations limits a thorough investigation of novel therapeutics in such models. Furthermore, the discrepancy in immune system activation and responses in humans and animals might preclude preclinical studies from accurately predicting clinical trial results. To overcome this gap, humanized mice, which were designed to have a human immune system, offer an appropriate immune microenvironment and can facilitate cancer immunotherapy development (42). Surgical transplantation of human fetal liver and thymus tissue fragments into immunodeficient mice generates Bone marrow-Liver-Thymus (BLT) humanized model that portrays a more robust and closer representation of the human immune system and response. Therefore, a patient-derived MBM BLT humanized mouse model was developed for use in testing the efficacy of human SC-oHSV and SC^{N1KO} releasing GM-CSF and scFv-PD-1 (hTSC-G/P).

### Results

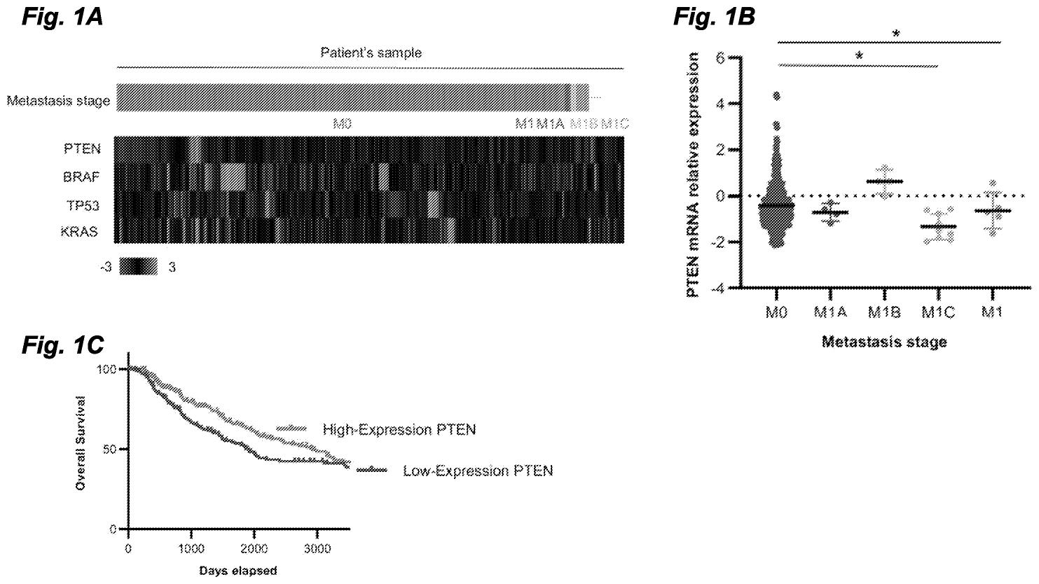

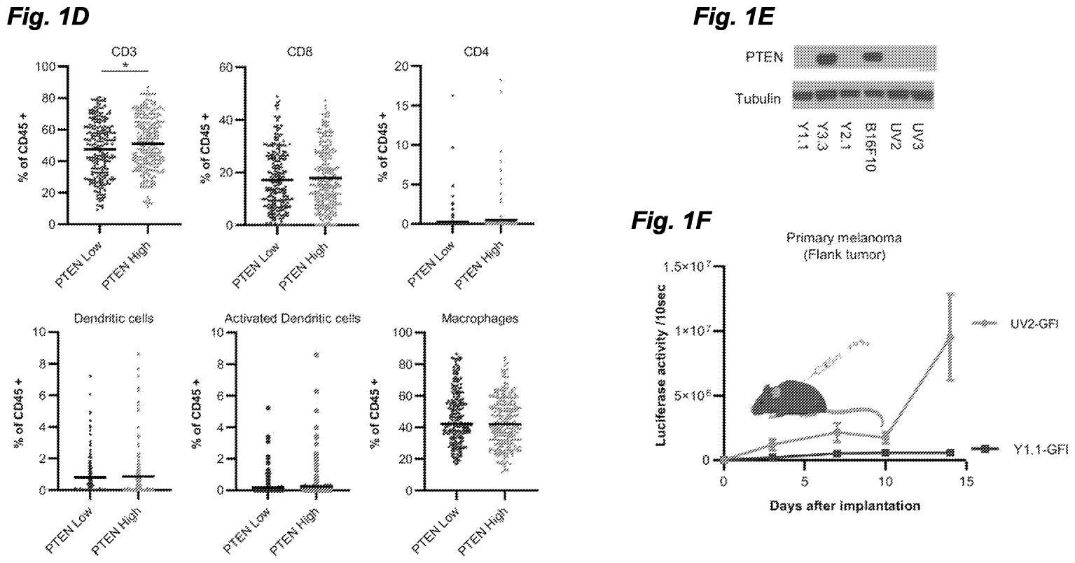

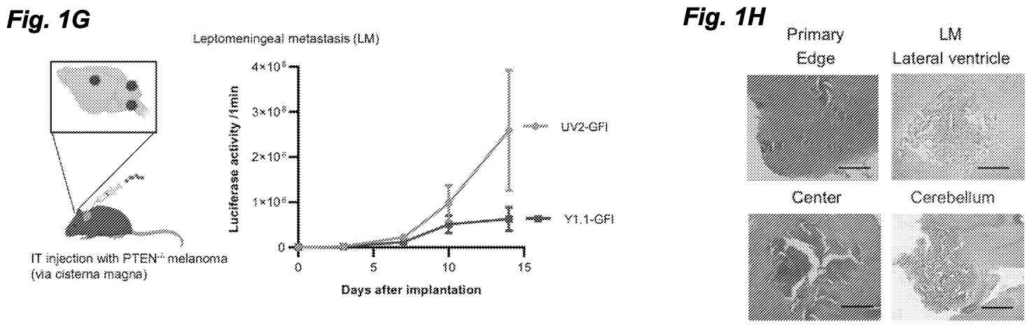

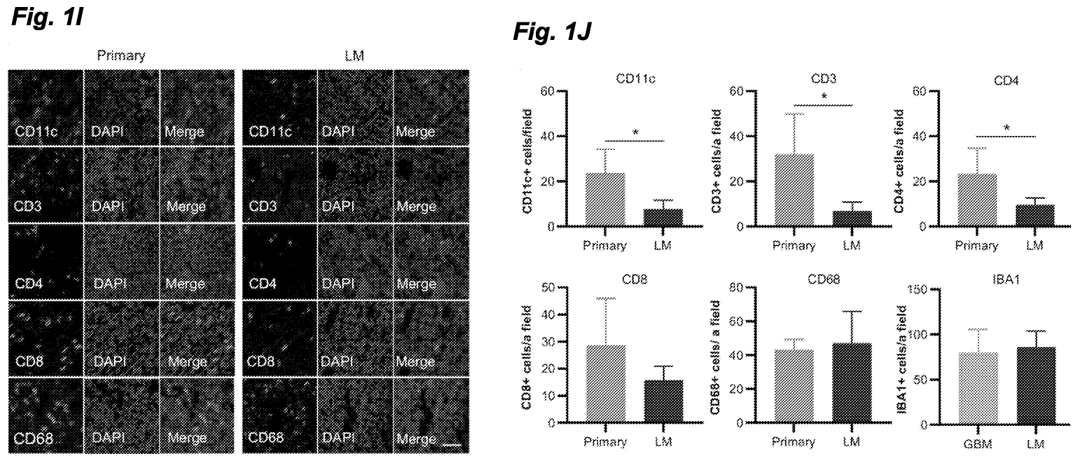

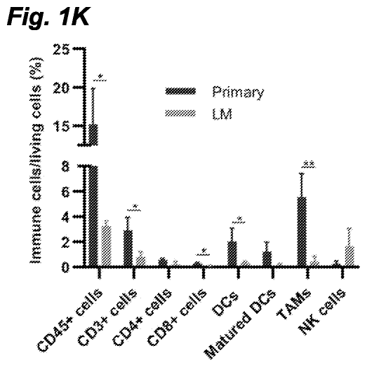

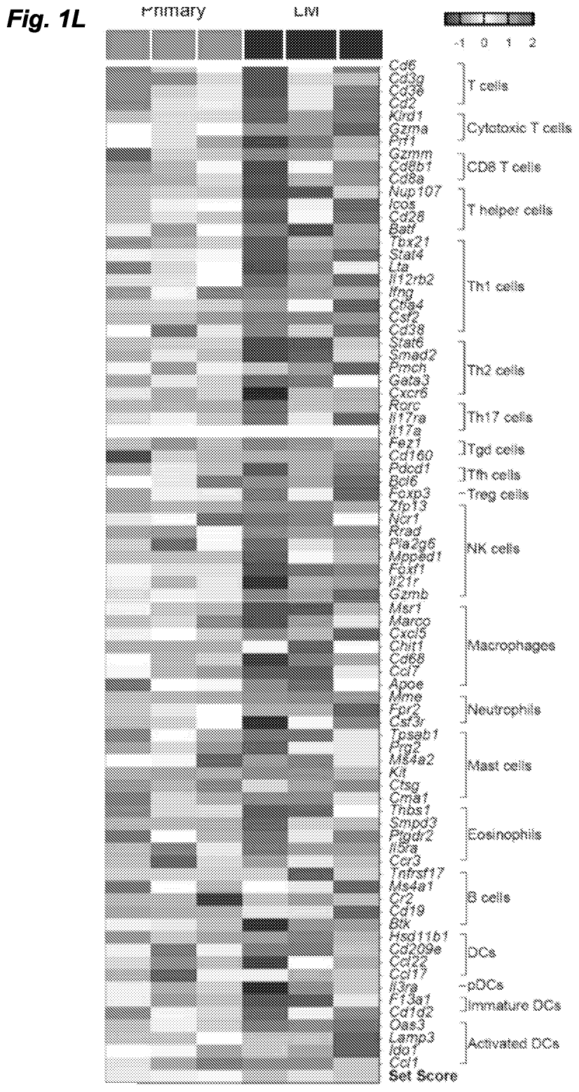

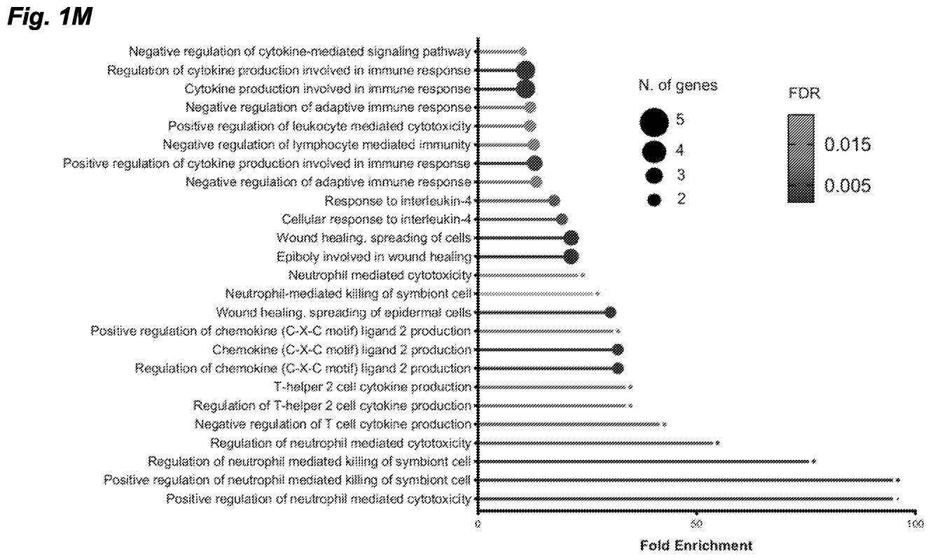

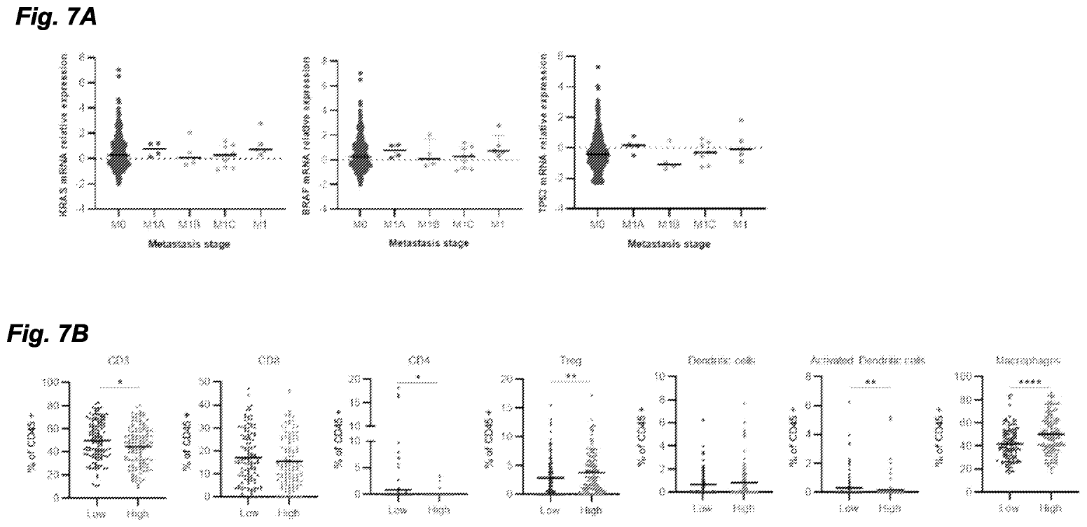

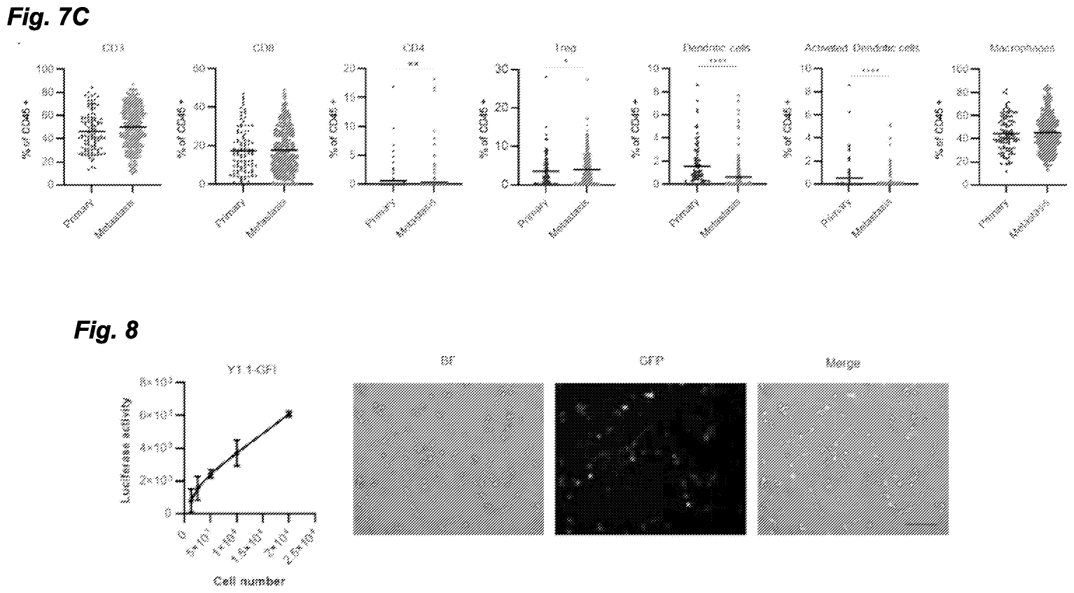

PTEN deficiency is associated with melanoma brain metastasis and immune suppression: PTEN is a phosphatase that is involved in the negative regulation of cell survival signaling through the PI3K/AKT pathway (43). First, the correlation between advanced melanoma and PTEN expression was explored. Overall, TCGA data shows that 20% of melanomas express PTEN (FIG. 1A). PTEN expression is significantly lost in late-stage metastatic melanoma, compared to other critical genes including BRAF and TP53 (FIG. 1B, FIG. 7A). A high proportion of clinical MBM (M1c: NCCN 6^{th} edition) cases are characterized by the loss of PTEN expression in the metastatic melanoma cells (FIG. 1B). Interestingly, melanoma patients with higher PTEN expression showed longer overall survival compared to patients with lower PTEN expression (FIG. 1C). To further explore this, immune cell infiltration in melanomas was analyzed. PTEN-low melanoma had significantly fewer T cells and a tendency of less dendritic cells (DCs) (FIG. 1D). Furthermore, patients with higher PI3K/AKT pathway expression had significantly less CD3+T cells, CD4+ T cells and activated DCs and more regulatory T cells and macrophages (FIG. 7B). Comparing primary melanoma to metastatic melanoma, the latter had significantly fewer CD4+ T cells and DCs (FIG. 7C). These results indicate that PTEN loss is associated with brain metastasis and poor prognosis through immunosuppressive mechanisms. Therefore, it is critical to develop a novel immunotherapy against advanced PTEN-deficient melanoma.

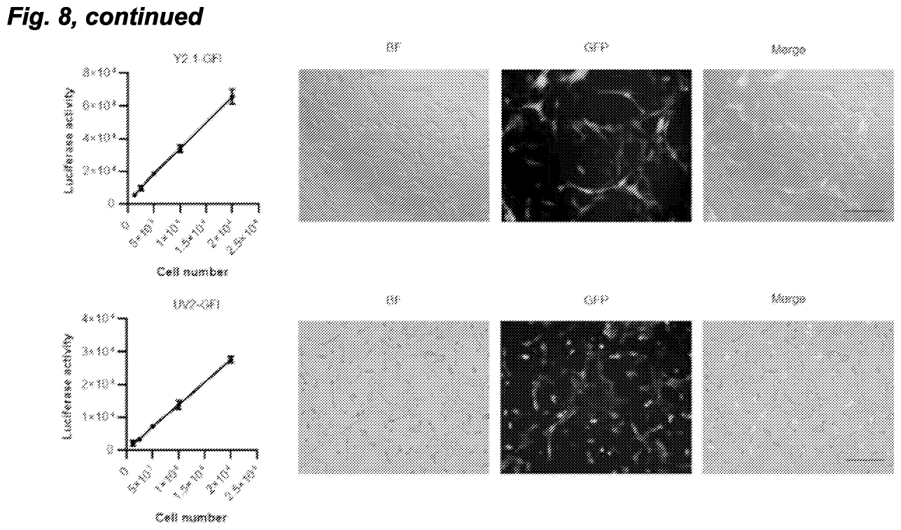

Development and characterization of primary and metastatic mouse tumor models of PTEN-deficient melanoma. The expression of PTEN in different murine melanoma cell lines, which were previously generated, was explored (44, 45). Western blot analysis revealed that several murine cell lines, YUMM1.1 (Y1.1), YUMM2.1 (Y2.1), D4MUV2 (UV2) and D4MUV3 (UV3) cells did not express PTEN (FIG. 1E). In previous studies, Y1.1 and Y2.1 cells have been used as models of BRAF^{V600E/wt}/PTEN^{-/-} melanomas, while UV2 and UV3 cells represent BRAF^{V600E/PTEN-/-} melanomas (45-47). On the other hand, YUMM3.3 (Y3.3) and B16F10 (B16) cells simulate BRAF^{V600E/wt/PTEN+/+} and BRAF^{wt}/PTEN^{+/+} melanomas, respectively (46, 47). Y1.1 and UV2 were selected to generate mouse PTEN-deficient melanoma cell lines expressing a bimodal GFP-firefly luciferase (Fluc) fusion protein (FIG. 8) and these cells were used to develop mouse models of both primary tumors and leptomeningeal metastasis (LM) tumor (FIG. 1F-1H). UV2-GFP-Fluc (UV2-GFI) cells grew efficiently as a LM model, whereas Y1.1-GFP-Fluc (Y1.1-GFI) cells often grew as extracranial tumors. To assess the composition of immune cells in the tumor microenvironment of primary melanoma and LM in these mouse models, immunofluorescence (IF) analysis was performed for dendritic cells (DCs, CD11c), T cells (CD3, CD4, and CD8), and macrophages (CD68, and IBA1). IF analysis showed that the LM model had less CD11c, CD3, CD4, and CD8 positive cells than the primary tumor model (FIGS. 1I and 1J). CD68+ cells were found in comparable levels between primary and LM models. To explore the difference of microglia in brain, IBA1+ cells were compared in the LM model to PTEN-deficient glioblastoma (CT2A) model. Then, no significant difference in IBA1+ cells was observed between mouse LM model and murine CT2A model (FIG. 1J). Furthermore, flowcytometry (FCM) analysis revealed that the LM model had less dendritic cells, CD4+, CD8+ T cells than the primary melanoma model (FIG. 1K). Moreover, transcriptome analysis also revealed that two out of three LM samples had downregulation of these immune cells compared to primary melanoma (FIG. 1L). Furthermore, LM samples downregulated several immunological pathways including cytokine related pathways (FIG. 1M). These results indicated that LM represents more immunosuppressive tumors compared to primary melanoma in the mouse models tested.

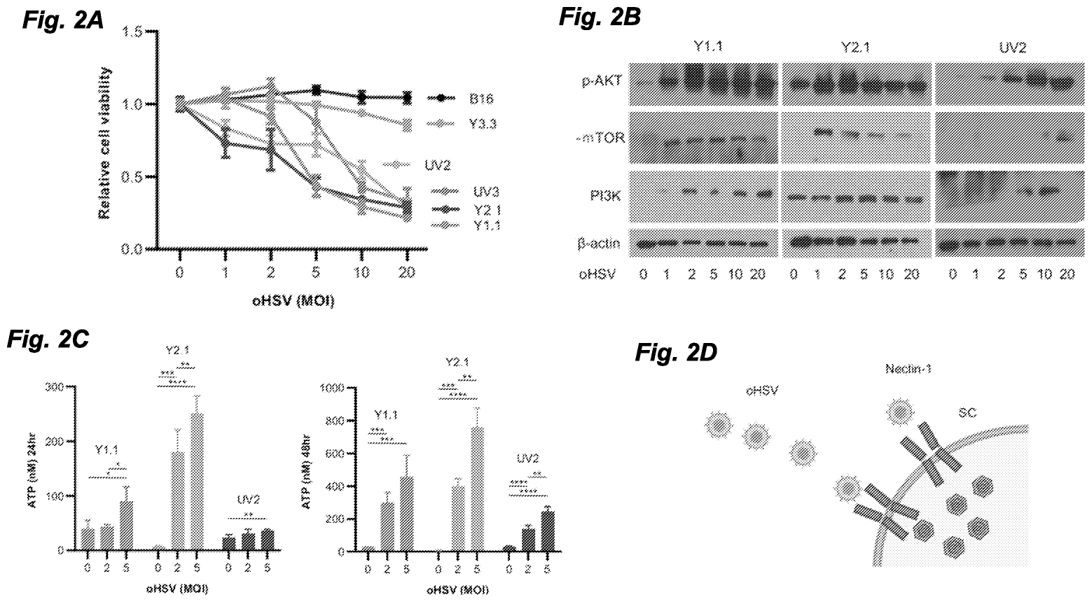

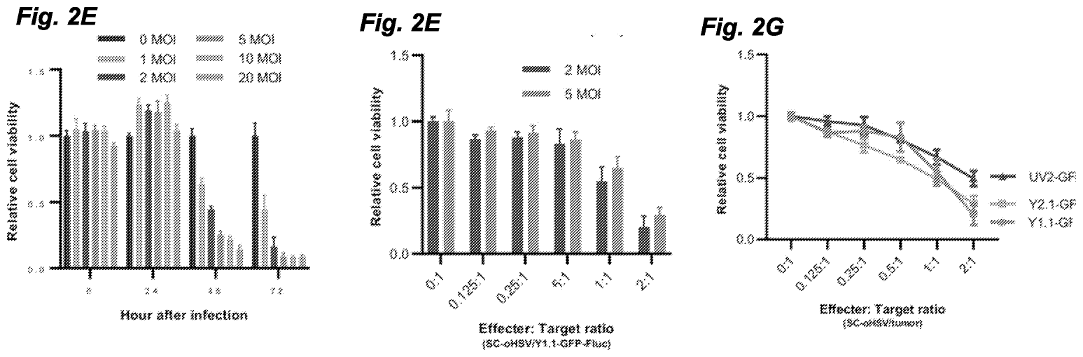

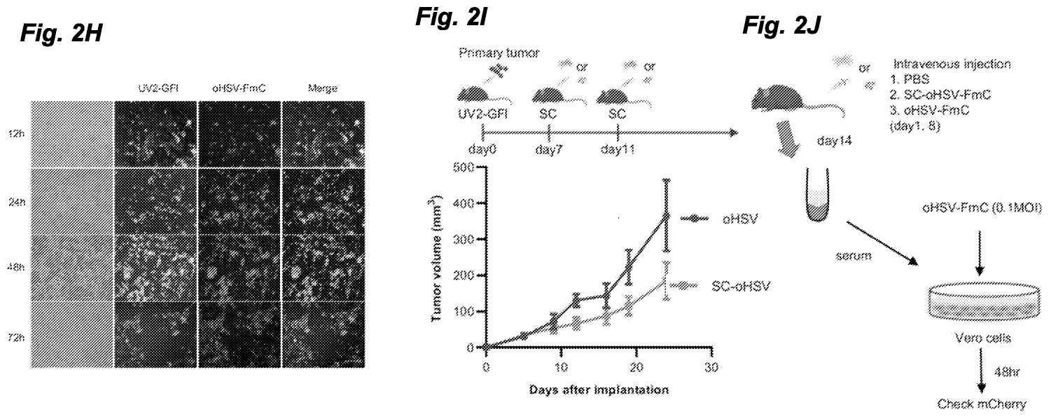

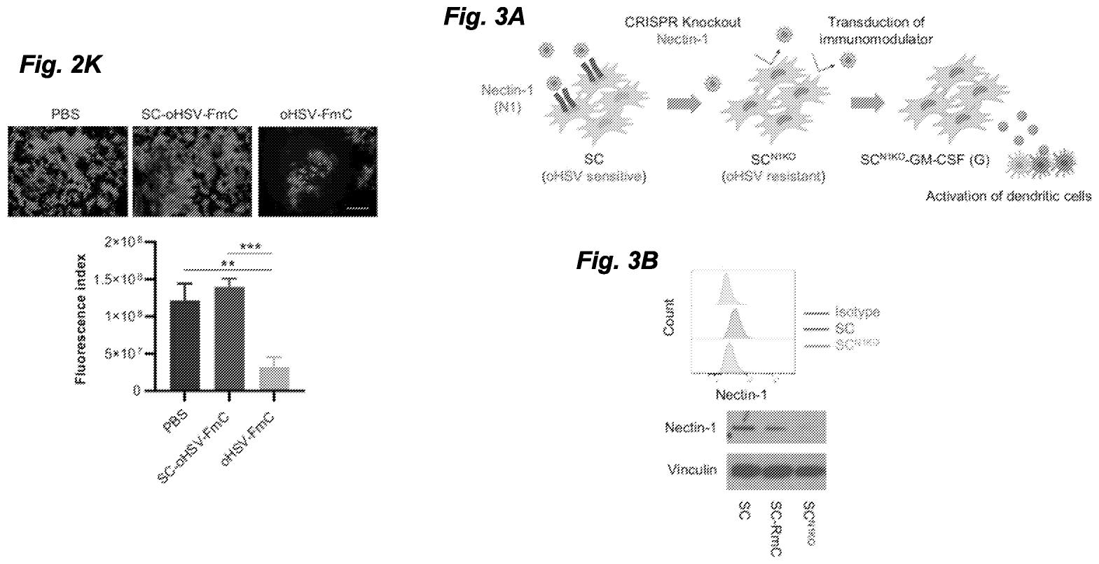

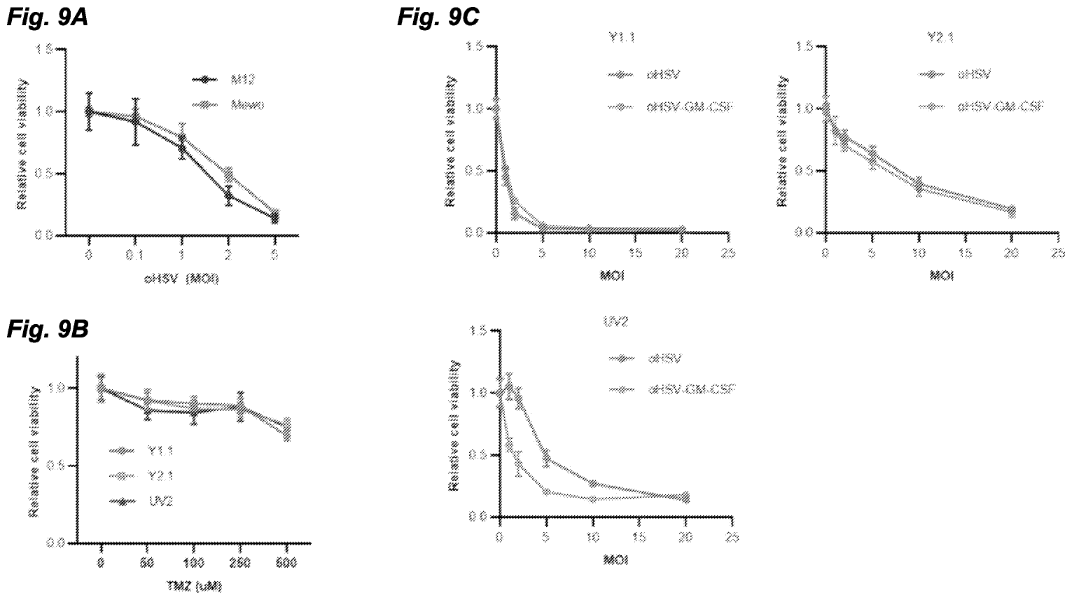

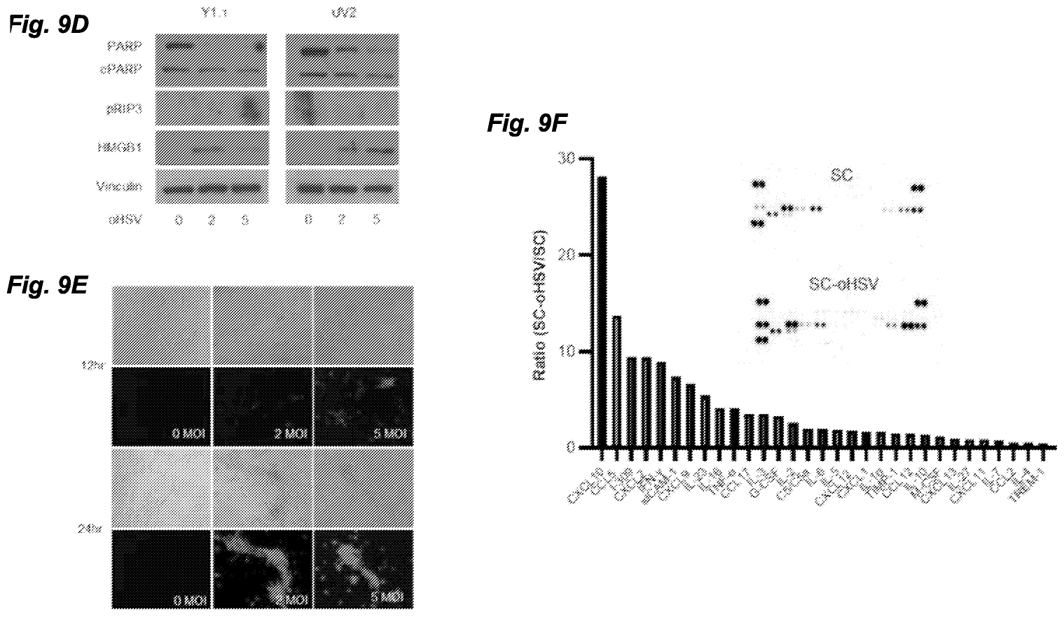

SC loaded oHSV have therapeutic effects in vitro and in vivo. The efficacy of recombinant oHSVs based on G47Δ, a third-generation oHSV type 1, against melanoma cells was explored. To characterize the oncolytic activity of oHSV, its cytopathic effects in human melanoma cell lines (Mewo and M12) (FIG. 9A) as well as murine melanoma cells was studied in vitro (FIG. 2A). Cell viability assay revealed that oHSV treatment resulted in the robust killing of Y1.1, Y2.1, UV2 and UV3 cells, while B16 and Y3.3 cells were more resistant (FIG. 2A). On the other hand, one of the standard chemotherapies for melanoma, temozolomide, did not induce any cell death (FIG. S3B). Therefore, Y1.1, Y2.1, and UV2 cells were chosen as both oHSV-sensitive and PTEN-deficient melanoma cells in this study. Since clinically approved oHSV, T-VEC expresses GM-CSF, oHSV that encodes mouse GM-CSF cDNA (oHSV-GM-CSF) were also created. oHSV-GM-CSF also induced cell death in Y1.1, Y2.1, and UV2 cells (FIG. 9C). Next, the influence of oHSV on the PI3K/AKT pathway in melanoma cells was explored. Western blot analysis showed that oHSV increased p-AKT, p-mTOR and PI3K in Y1.1, Y2.1 and UV2 cells (FIG. 2B). It was confirmed that oHSV-mediated tumor cell death was associated with induction of apoptosis while it wasn't associated with necroptosis, as indicated by changes in upregulation of cleaved PARP/PARP, but no change in pRIP3 expression (FIG. 9D). Next, it was determined whether oHSV could induce immunogenic cell death (ICD) via the release of Damage-associated molecular pattern (DAMPs) factors such as ATP and high-mobility group box 1 protein (HMGB1). oHSV significantly induced ATP release from Y1.1, Y2.1 and UV2 cells 24 and 48 h after oHSV infection (FIG. 2C). Further, western blot analysis also showed oHSV increased HMGB1 from Y1.1 and UV2 cells 24 h after oHSV infection (FIG. 9D). These results show that oHSV induces immunogenic cell death as well as oncolysis cell death in melanoma cells.

The use of adipose derived mesenchymal SC as delivery carriers for oHSV was explored (FIG. 2D). SCs were incubated with oHSV at different multiplicity of infection (MOIs) and their survival was measured in vitro. SCs infected at 1, 2, 5, 10, and 20 MOI survived for 24 h followed by a rapid decrease in viable populations by 48 h after infection (FIG. 2E). SCs infected at 1 and 2 MOI retained ˜50% of the original population at 48 h, while SCs infected at MOIs of 5 or higher retained 25% or fewer of the original population at the same timepoint. Furthermore, fluorescence microscopy showed that almost all SCs infected at 2 and 5 MOI expressed reporter mCherry 24 h after infection (FIG. 9E). Infection with 5 MOI led to a higher infection rate but a larger decline in SC viability at the 24 h timepoint, posing a challenge for their use in in vivo treatment. When co-cultured with murine melanoma cells, SC-oHSV at 2 and 5 MOI comparably decreased the cell viability of Y1.1-GFl cells (FIG. 2F). Therefore, oHSV at 2 MOI was chosen for treatment studies. SC-oHSV (2 MOI) effectivity killed Y1.1, Y2.1 and UV2-GFl (FIG. 2G). Imaging of co-culture with melanoma cells and SC-oHSV-FmC showed oHSV-FmC infection of melanoma cells overtime (FIG. 2H).

In vivo, in the primary tumor model of UV2-GFl, intra-tumoral administration of SC-oHSV led to a decrease in tumor growth relative to oHSV alone, although the difference was not statistically significant (FIG. 2I). Additionally, in vitro multi-cytokine and chemokine assays showed that SC-oHSV induced the release of several chemokines such as CCL5/RANTES, CXCL10/IP-10, IFN-γ and TNFα, which are known to play important roles in recruiting tumor infiltrating lymphocytes (TILs) (48, 49) (FIG. 9F). One of the clinical limitations of oHSV is that it cannot be given systemically because of neutralizing antibody-mediated immune elimination of the viruses (50). Thus, the production of anti-oHSV antibodies (Abs) after intravenous injections of SC-oHSV-FmC or oHSV-FmC was examined in mice. Vero cells were infected with oHSV-FmC in vitro for 2 days in the presence of serum collected from the mice (FIG. 2J). The serum collected from the mice treated systemically twice with SC-oHSV-FmC did not impede oHSV infection, while the serum collected from the mice treated twice with oHSV-FmC did reduce infection (FIG. 2K), indicating that SC loading of oHSV reduced the production of anti-oHSV Abs in immune-competent mice. These results indicated that SC-oHSV has the potential to induce ICD via the active release of immunogenic molecules and chemokines, leading to activation of the host immune response against tumors, while minimizing the clearance of the oHSV by the host's immune system. Taken together, these data show that SCs are suitable oHSV carriers for virotherapy.

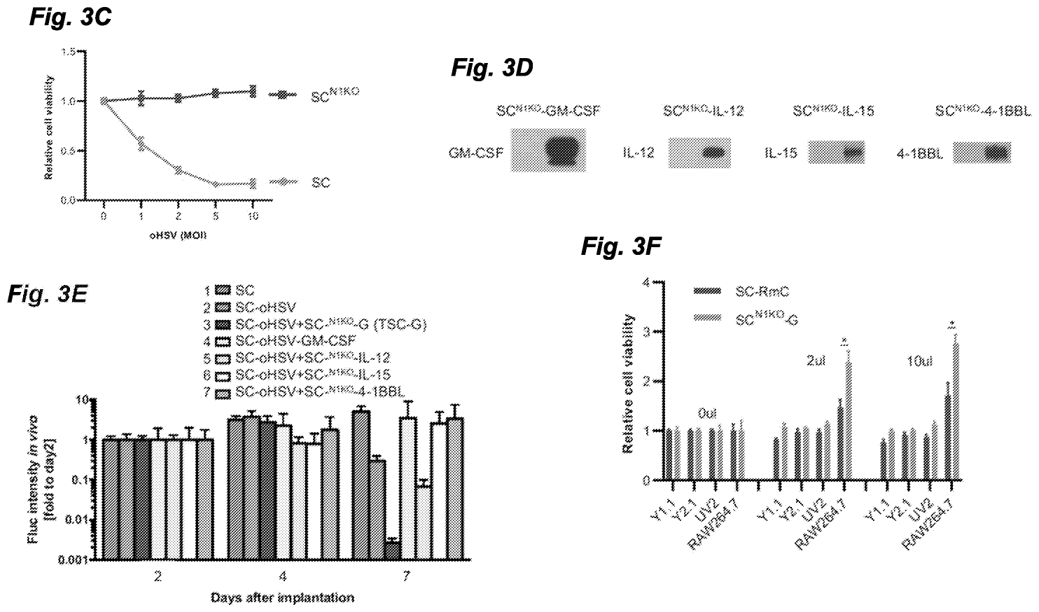

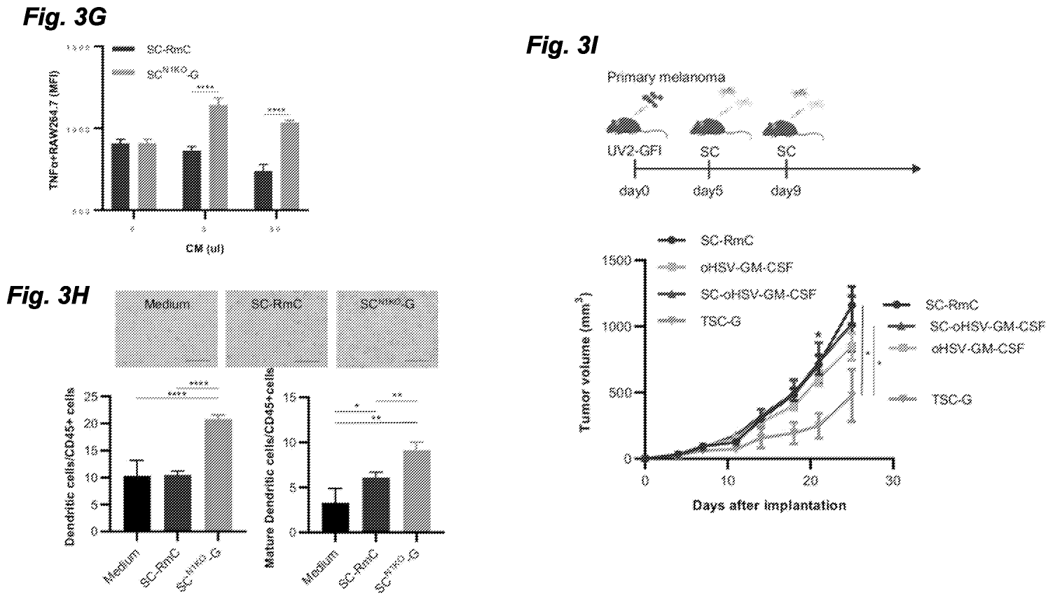

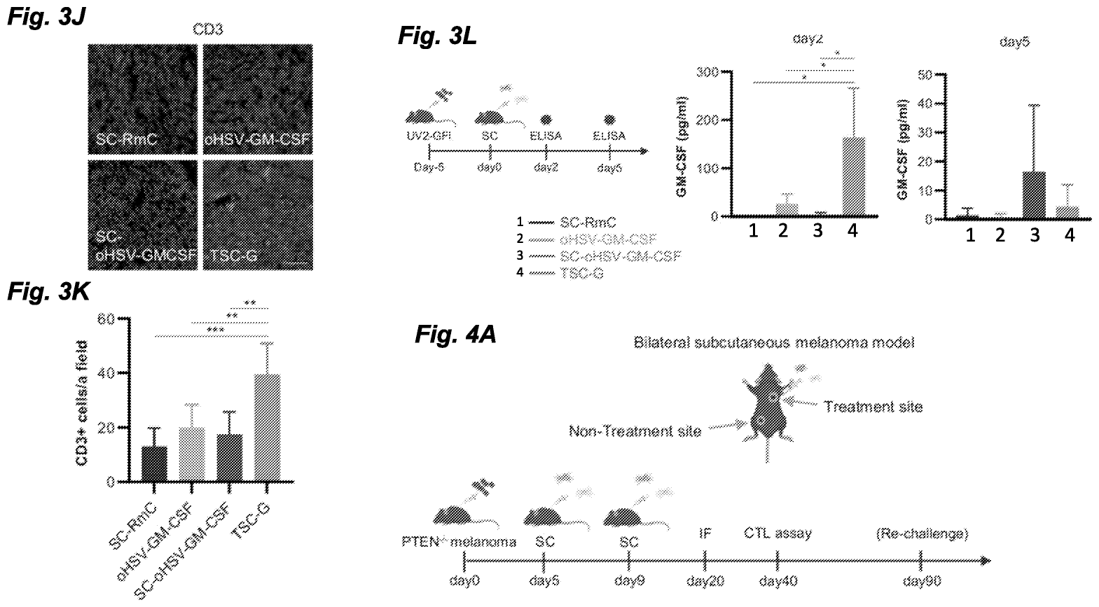

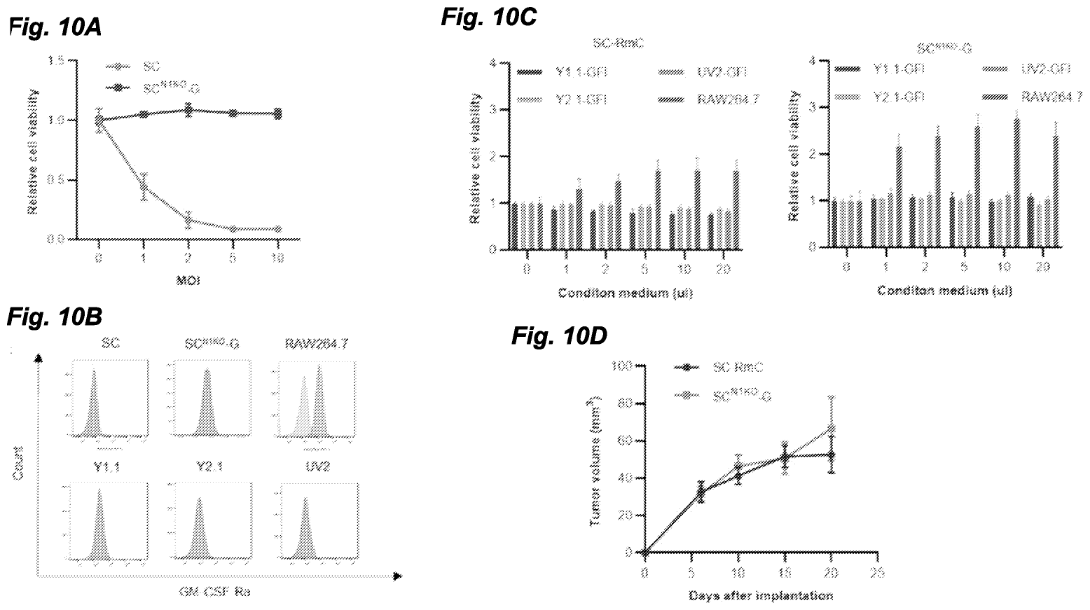

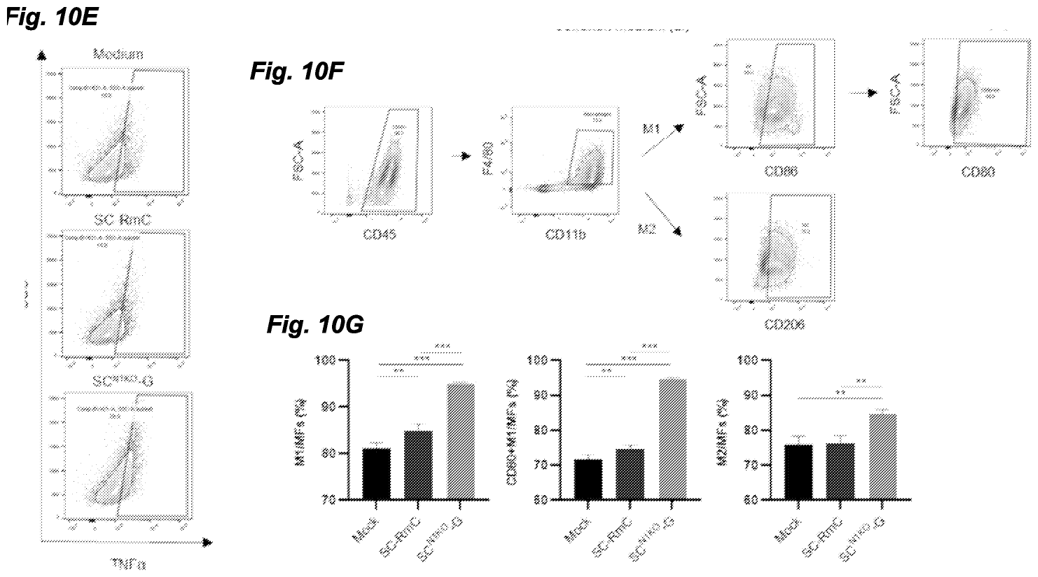

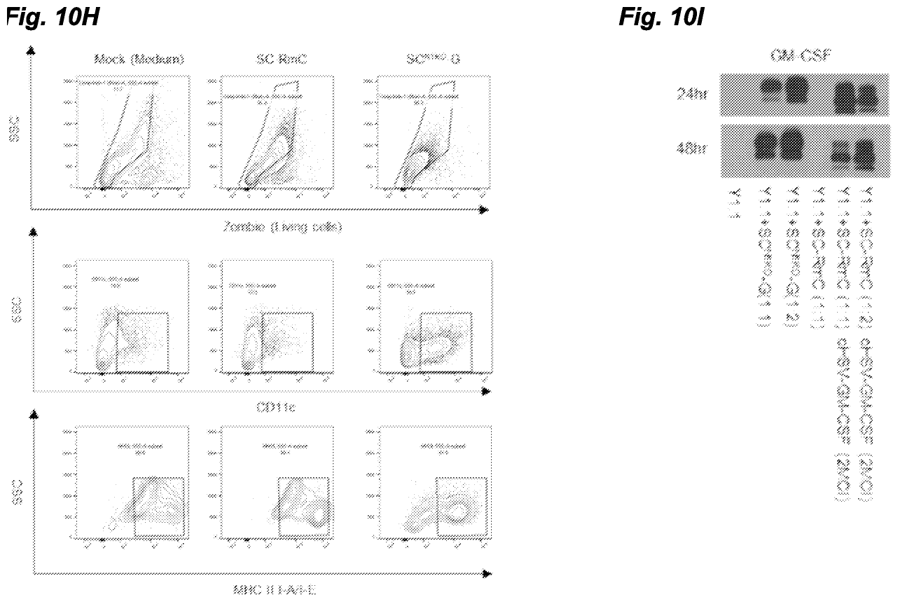

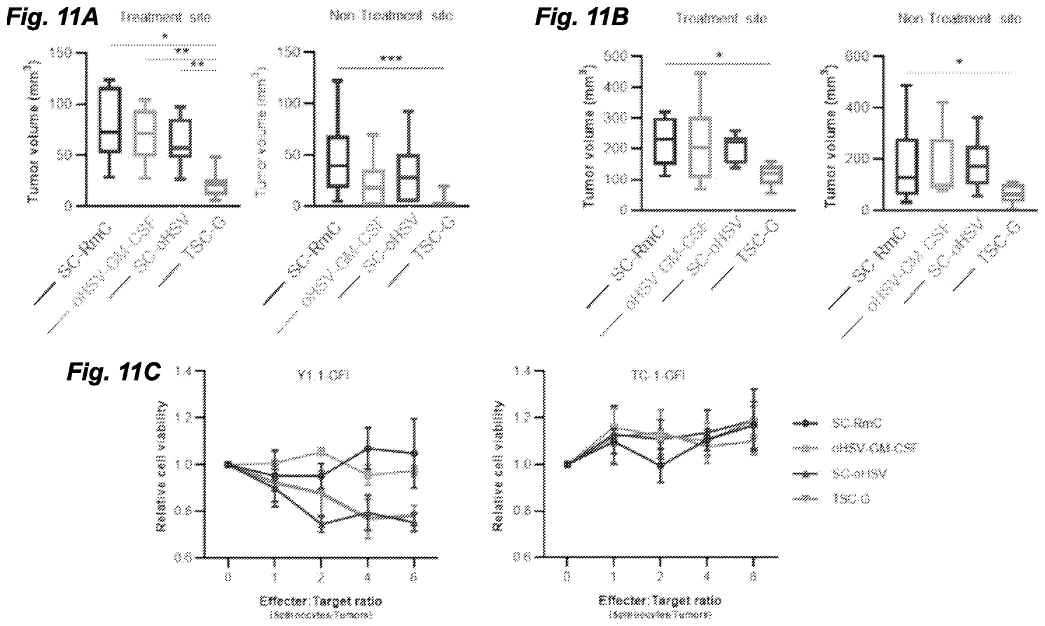

Establishment of oHSV-resistant SCs secreting immunomodulators. The cell surface adhesion molecule Nectin-1 (CD111) is known as the most efficient entry receptor for oHSV (26), and the inventors have previously shown that both mouse and human SC have high expression of this protein (16). To allow prolonged secretion of immunomodulators in the presence of oHSV and enhance the therapeutic efficacy of SC-oHSV for PTEN-deficient melanoma, oHSV-resistant SCs were created by CRISPR/Cas9-mediated knockout of Nectin-1 (SC^{N1KO}) (FIG. 3A). Western blot analysis confirmed the knockout of Nectin-1 in SCs (FIG. 3B), and cell viability assays post-treatment with oHSV confirmed the resistance of SC^{N1KO} to oHSV as compared to unmodified SC (FIG. 3C). SC^{N1KO} were further engineered to secrete IL-12, IL-15, GM-CSF, or 4-1BB ligand (FIG. 3D). To determine which immunomodulator synergically suppressed PTEN-deficient melanoma when combined with SC-oHSV, a mixture comprised of Y1.1-GFl cells, SC-oHSV and SC^{N1KO}-immunomodulator were inoculated into the flank and tumor growth was monitored. In this screening test, a combination therapy of SC^{N1KO}-GM-CSF (SC^{N1KO}-G) and SC-oHSV (TSC-G) showed the most potent therapeutic efficacy compared to the other combination therapies and SC-oHSV-GM-CSF on its own (FIG. 3E). Therefore, the study focused on utilizing of TSC-G therapy for PTEN-deficient melanoma. SC^{N1KO}-G retained the oHSV resistant phenotype of SC^{N1KO}(FIG. 10A). FCM analysis demonstrated the cell surface expression of GM-CSF receptor in a murine macrophage cell line, RAW264.7 cells, but not in Y1.1-GFl, Y2.1-GFl and UV2-GFl cells (FIG. 10B). Furthermore, conditioned medium (CM) derived from SC^{N1KO}-G induced higher growth of RAW264.7 compared to CM from SC-Rluc-mCherry (RmC) (representing control SC), whereas no influence on cell growth was observed for Y1.1-GFl, Y2.1-GFl and UV2-GFl cells in vitro (FIG. 3F, FIG. 10C). Intra-tumoral administration of SC^{N1KO}-G did not influence the growth of Y1.1-GFl flank tumors in vivo (FIG. 10D).

Next, functional assays were performed using RAW264.7 and bone marrow cells to examine the activity of SC^{N1KO}-G-secreted GM-CSF in the differentiation and activation of macrophages and DCs. FCM showed that CM of SC^{N1KO}-GM-CSF significantly induced more TNFα-producing RAW264.7 cells than control medium or CM of SC-RmC (FIG. 3G, FIG. 10E). To explore the ability of the CM to differentiate to M1 (CD11b+F4/80+CD86+) or M2 (CD11b+F4/80+CD206+) macrophages, mouse bone marrow cells were incubated with SC^{N1KO}-G CM for 3 days. FCM analysis of macrophage populations showed that SC^{N1KO}-G CM significantly induced differentiation to M1, activated M1 (CD80+) and M2 macrophages (FIG. 10F, 10G). It was also found that mouse bone marrow cells exposure to SC^{N1KO}-G CM for 4 days significantly induced populations of DCs (CD45+CD11b+CD11c+) and mature DCs (CD45+CD11b+CD11c+ MHCII I-A/I-E+) (FIG. 3H, FIG. 10H). These results confirmed the biological functions of SC^{N1KO}-G secreted GM-CSF.

When co-cultured with Y1.1-GFI cells, SC^{N1KO}-G and oHSV-GM-CSF-infected SC-RmC induced similar levels of GM-CSF in supernatant at 24 h (FIG. 10I). However, at 48 h, GM-CSF levels decreased with oHSV-GM-CSF compared to SC^{N1KO}-G, reflecting cell death of oHSV-GM-CSF-infected cells. Importantly, intra-tumoral injection of TSC-G therapy significantly suppressed tumor growth compared with SC-RmC, oHSV-GM-CSF or SC-oHSV-GM-CSF in the UV2-GFl primary tumor model (FIG. 3I), as seen with the Y1.1-GFl model (FIG. 3E). IF images showed that TSC-G therapy led to the recruitment of more CD3-positive TILs compared to controls in 25 days after therapy (FIGS. 3I and 3K). Interestingly, TSC-G therapy released more GM-CSF than oHSV-GM-CSF or SC-oHSV-GM-CSF 2 days after intra tumoral injection, while the secretion of GM-CSF by SC-oHSV together with SC^{N1KO}-G or oHSV-GM-CSF on its own was drastically reduced 5 days after intra tumoral injection (FIG. 3L). Thus, the efficacy of TSC-G therapy in PTEN-deficient melanoma mouse models was superior to the oHSV-GM-CSF or SC-oHSV-G on their own and was associated with rapid, robust secretion of GM-CSF from SC^{N1KO}-G.

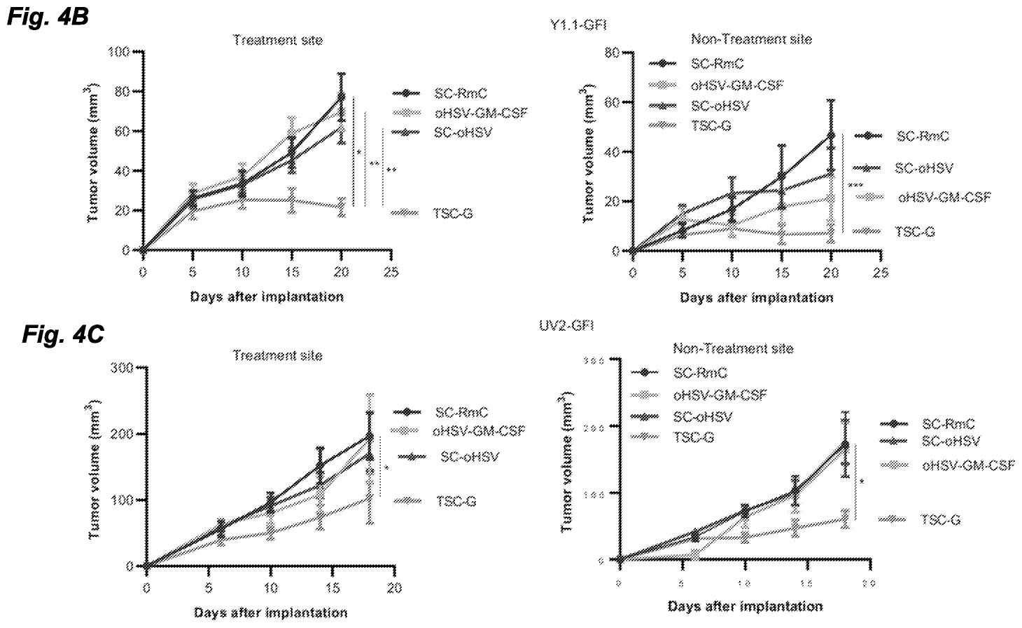

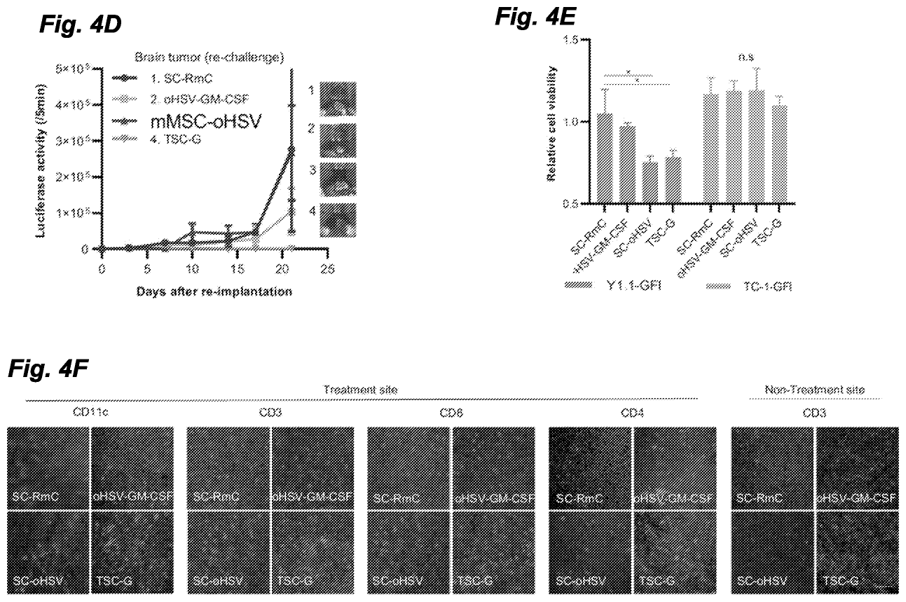

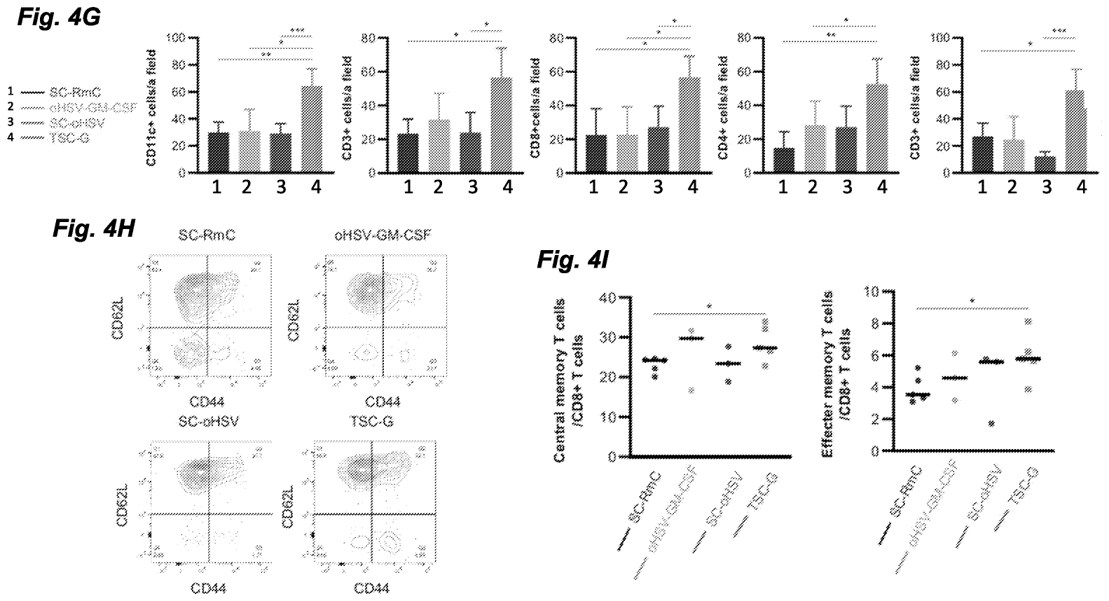

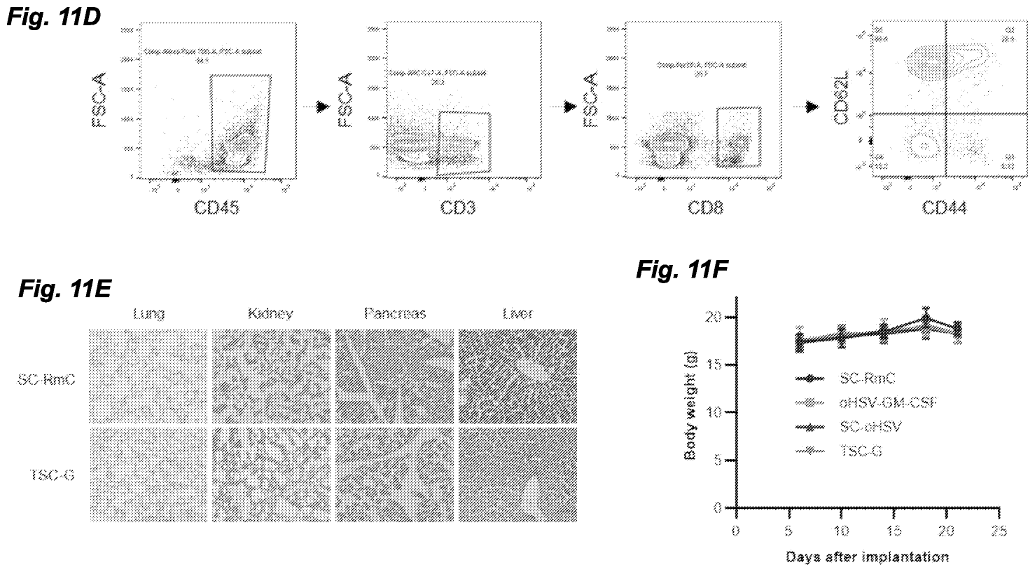

Abscopal effects of twin stem cells releasing oHSV and GM-CSF (TSC-G) in a bilateral subcutaneous tumor model. The abscopal effect is an interesting phenomenon in which tumor shrinkage at metastatic or distant sites is achieved following local therapy of the primary tumor. Next, the antitumor effects, including abscopal effects, of TSC-G therapy in two different bilateral subcutaneous melanoma models, Y1.1-GFl and UV2-GFl were assessed (FIG. 4A). In the Y1.1-GFl tumor model, TSC-G therapy showed significantly increased therapeutic effects compared to SC-RmC, oHSV-GM-CSF or SC-oHSV alone in directly treated tumors (FIG. 4B, FIG. 11A). Furthermore, the suppression of tumor growth by TSC-G therapy was better than SC-RmC at non-treatment site, whereas oHSV-GM-CSF or SC-oHSV alone didn't show efficacy. Similarly, TSC-G therapy decreased tumor growth in injected tumors and mediated abscopal effects in the UV2-GFl tumor model (FIG. 4C, FIG. 11B). Interestingly, when these mice with Y1.1-GFl tumors were re-challenged with Y1.1-GFI cells in the brain after each treatment, all the mice receiving TSC-G therapy (n=4) remained tumor free (FIG. 4D). To understand the mechanism underlying the therapeutic efficacy, cytotoxic T lymphocyte (CTL) assay was performed using splenocytes after treatment. The splenocytes (effector) collected from the spleen of treated mice showed significant cytotoxicity against Y1.1-GFl cells (target) as compared to control SC-RmC treatment, while no cytotoxicity was observed against murine lung TC-1-GFl cells (FIG. 4E, FIG. 10C). These results indicated that the TSC-G therapy induced tumor specific and systemic immune responses in the treated mice.

Next, tumor infiltrating immune cells post-treatment in the UV2-GFl mouse model were profiled. IF analysis demonstrated an increase of CD11c, CD3, CD4, CD8 positive cells at the treatment site and CD3 positive cells at the non-treatment site in the TSC-G group (FIGS. 4F and 4G). FCM was used to characterize memory T cells in the spleen 30 days post-treatment (FIG. 11D). The TSC-G therapy significantly induced effector memory CD8 T cells (CD45+CD3+CD8+CD62L+CD44+ cells), as well as central memory CD8 T cells (CD45+CD3+CD8+CD62L−CD44+ cells) when compared to SC-RmC treatment (FIGS. 4H and 4I). Immunotherapies such as ICIs have been associated with severe adverse effects in several major organs, such as the lung and pancreas (51). No body weight loss was noted and no detectable toxicity was observed in major organs after TSC-G therapy (FIGS. 11E and 11F). These results indicated that the TSC-G therapy described herein drives efficacy through the activation of systemic antitumor T cell immunity.

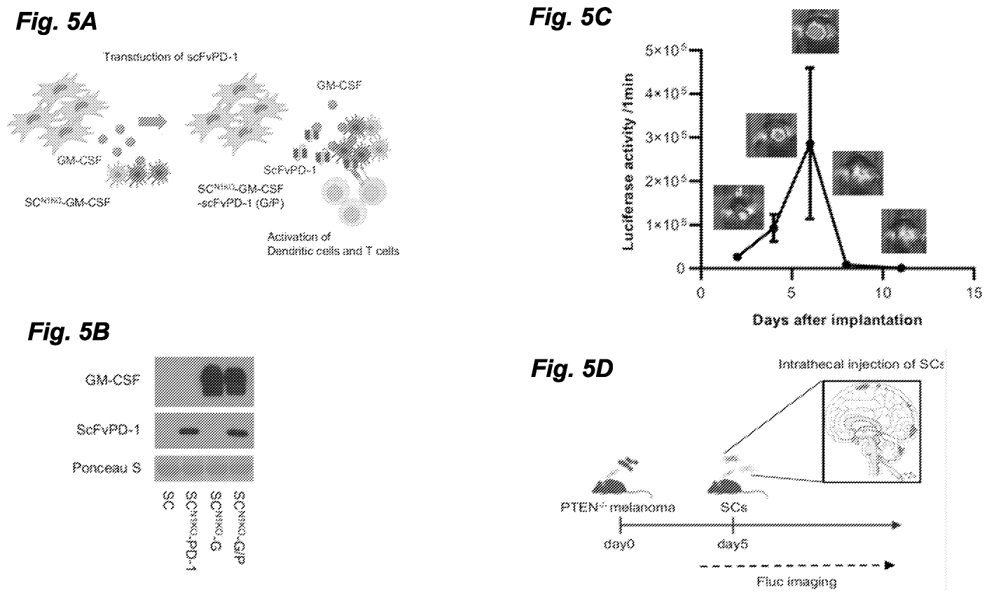

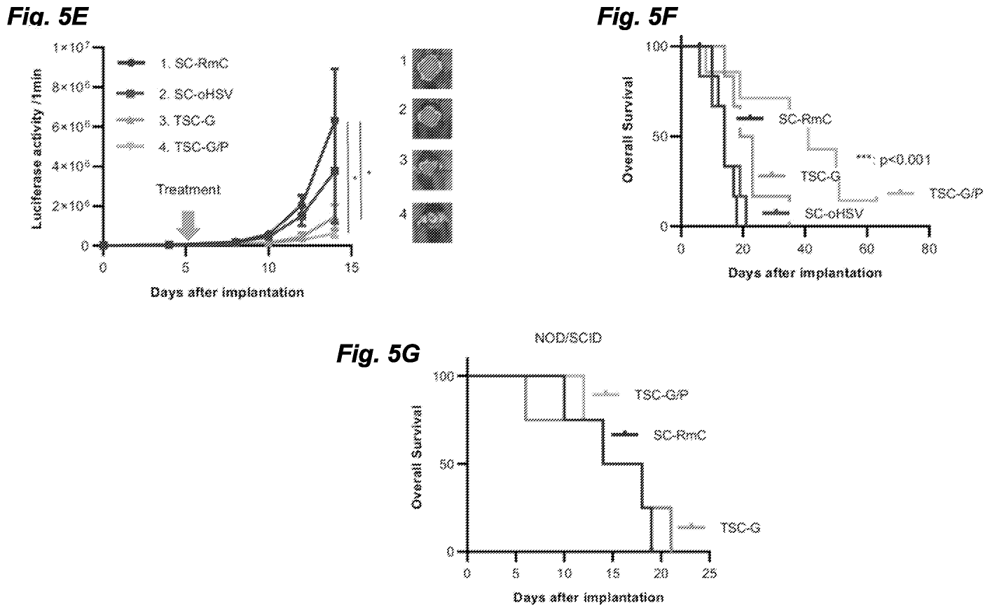

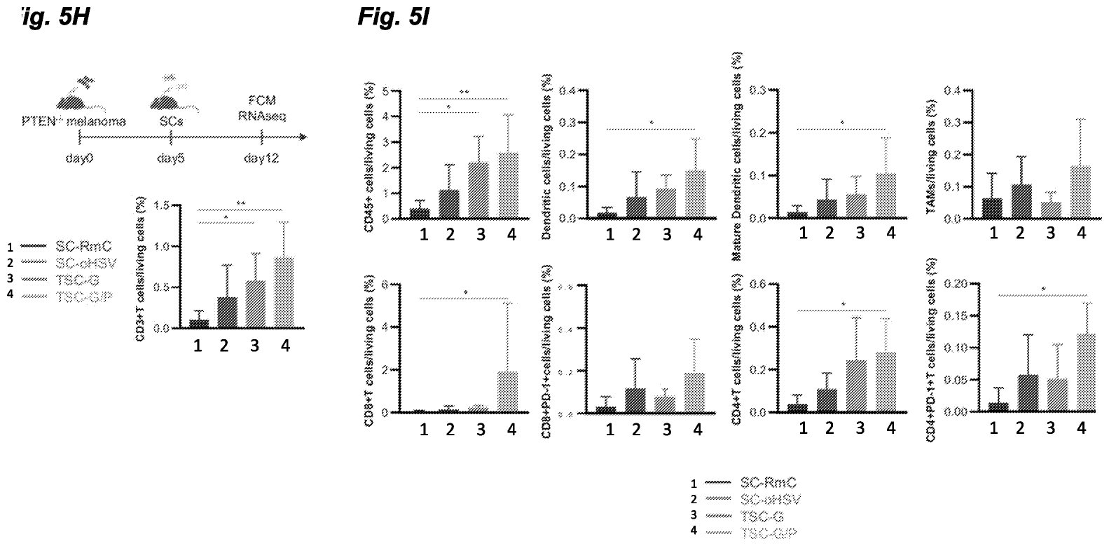

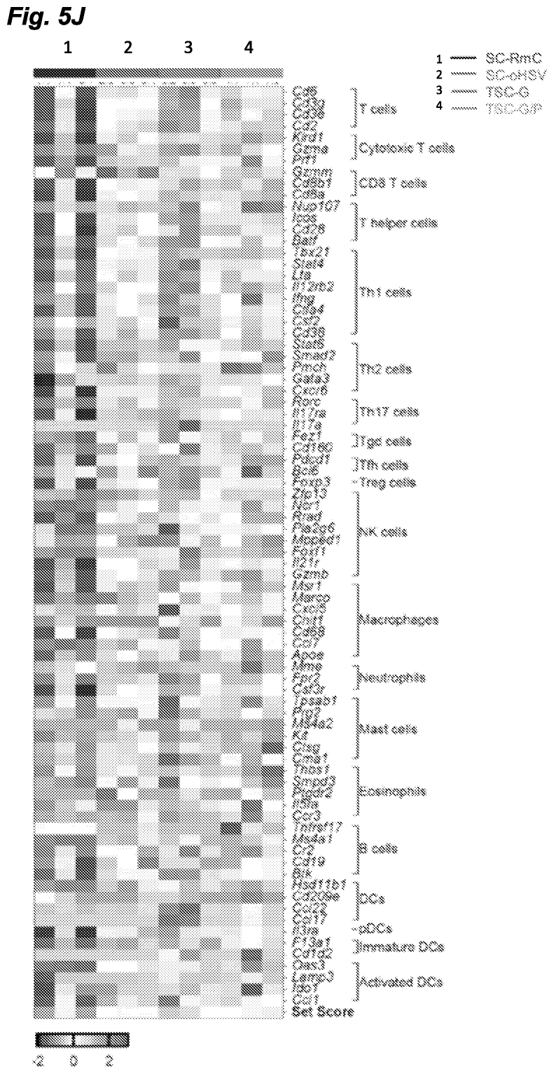

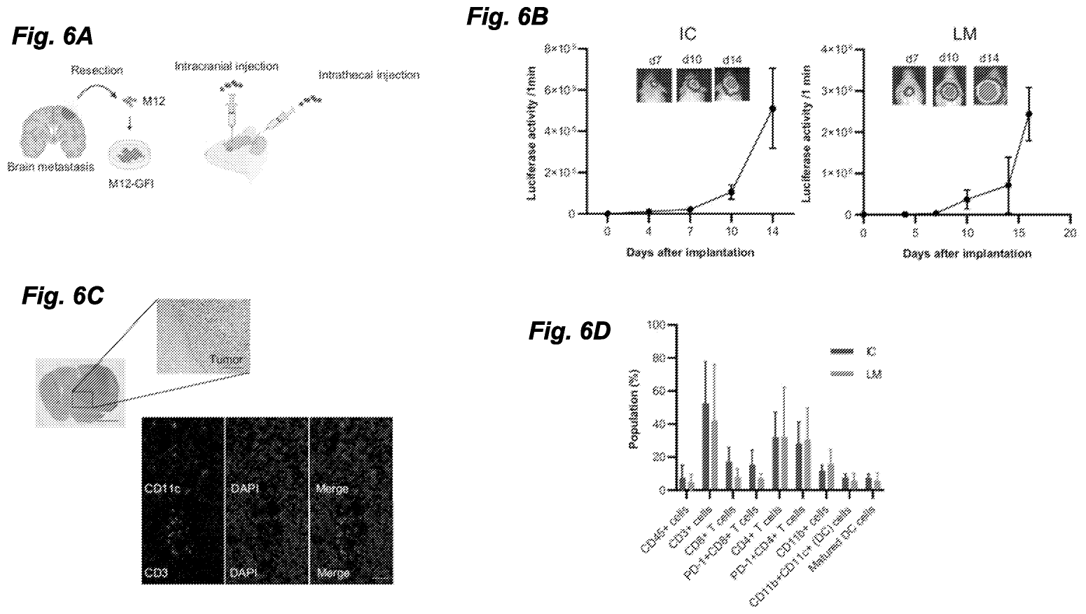

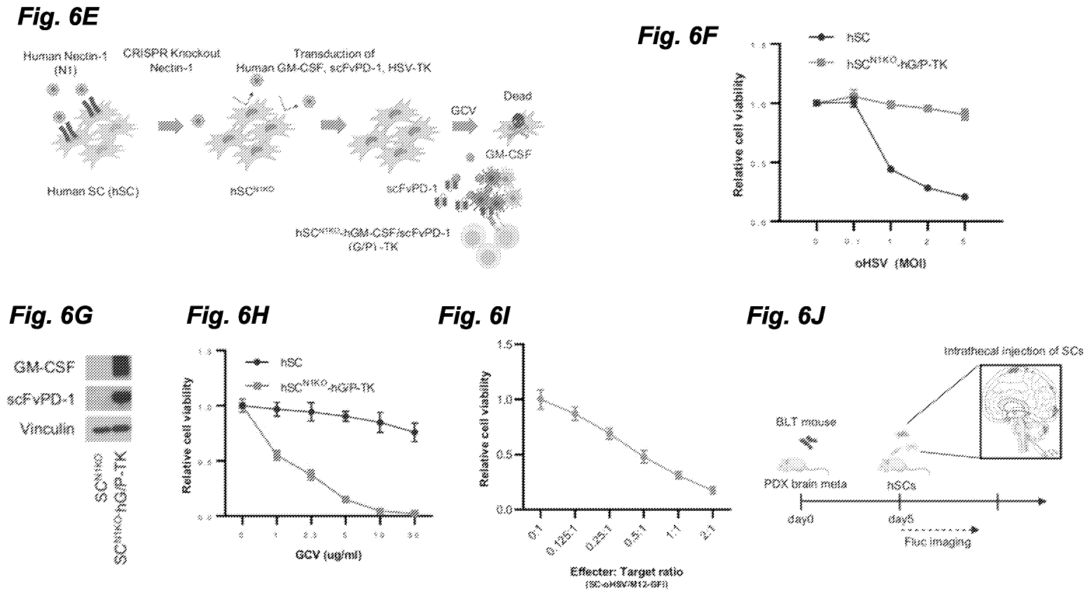

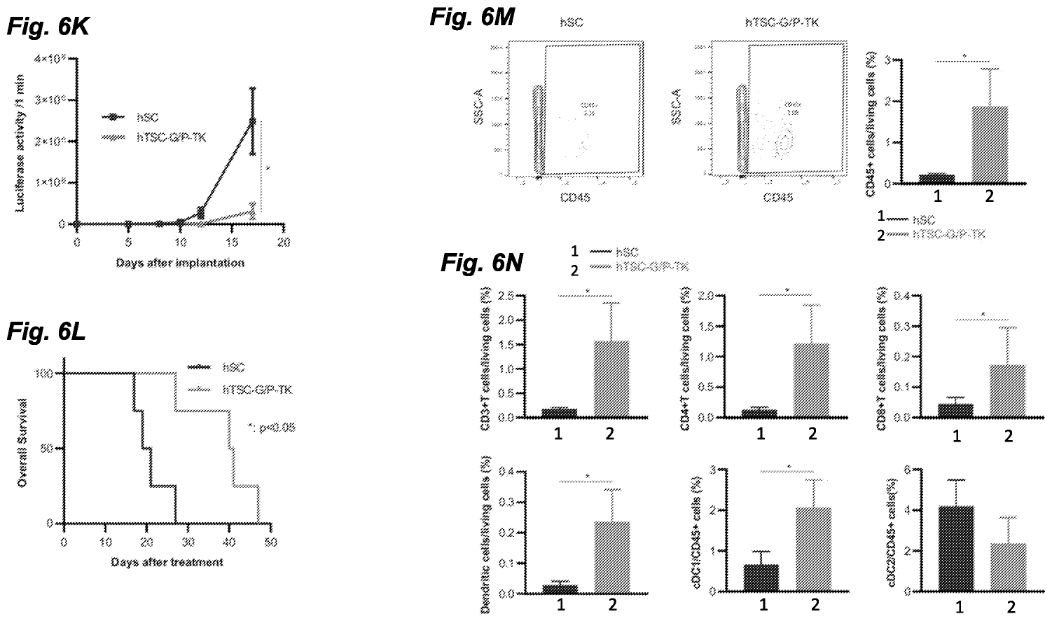

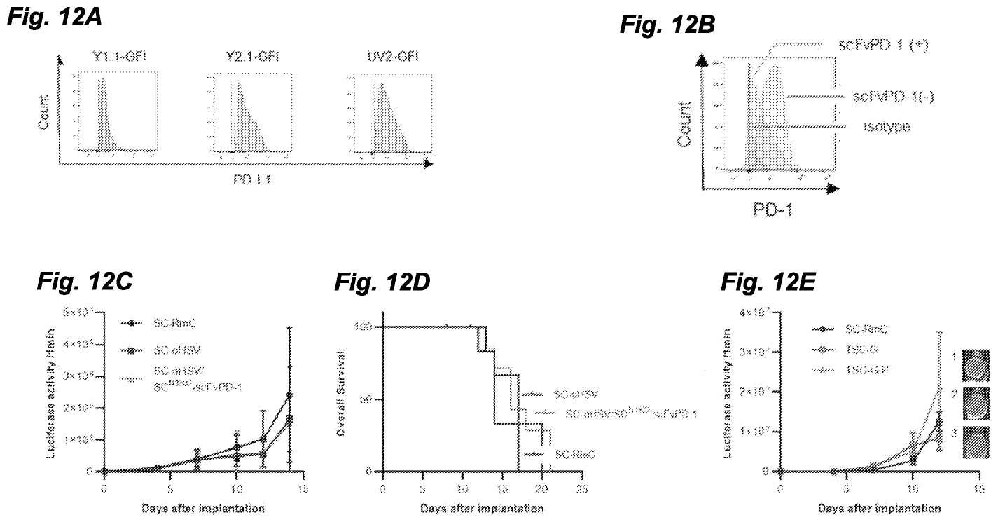

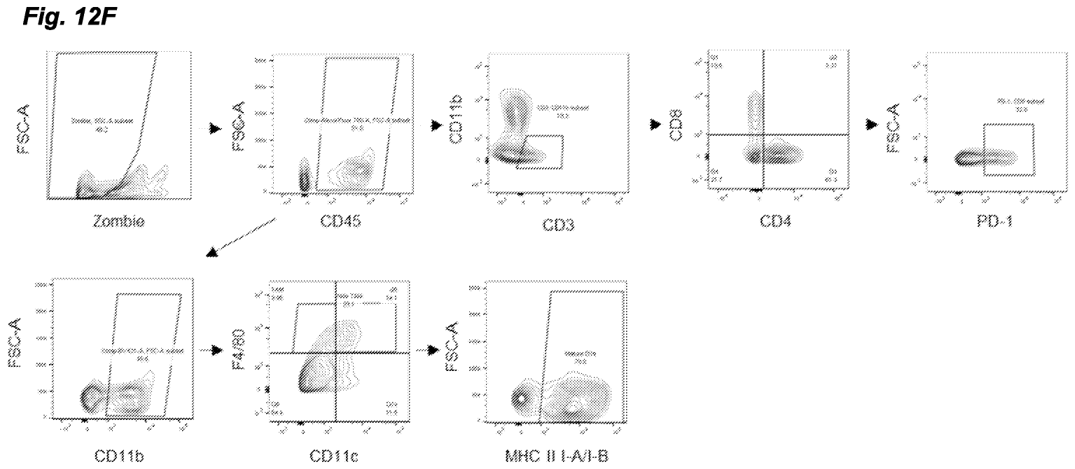

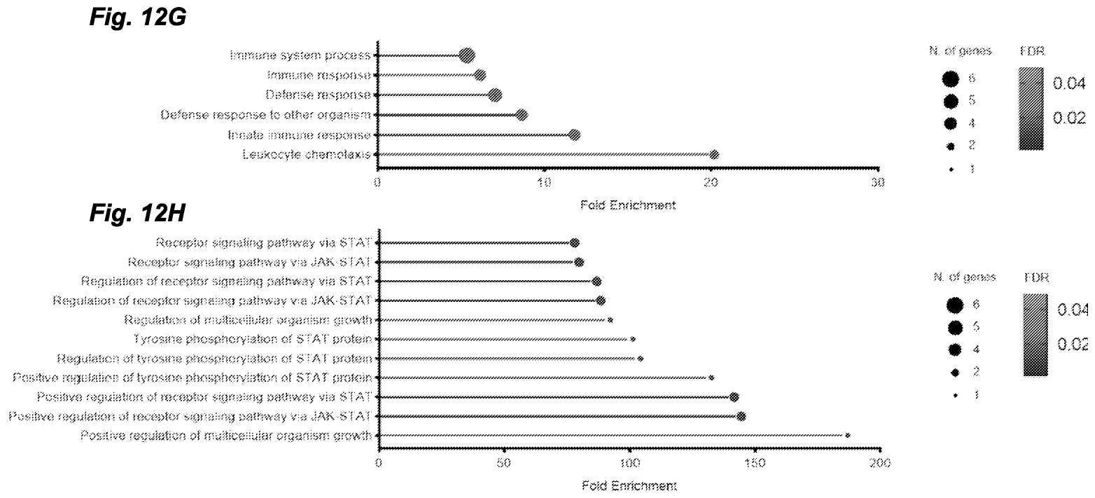

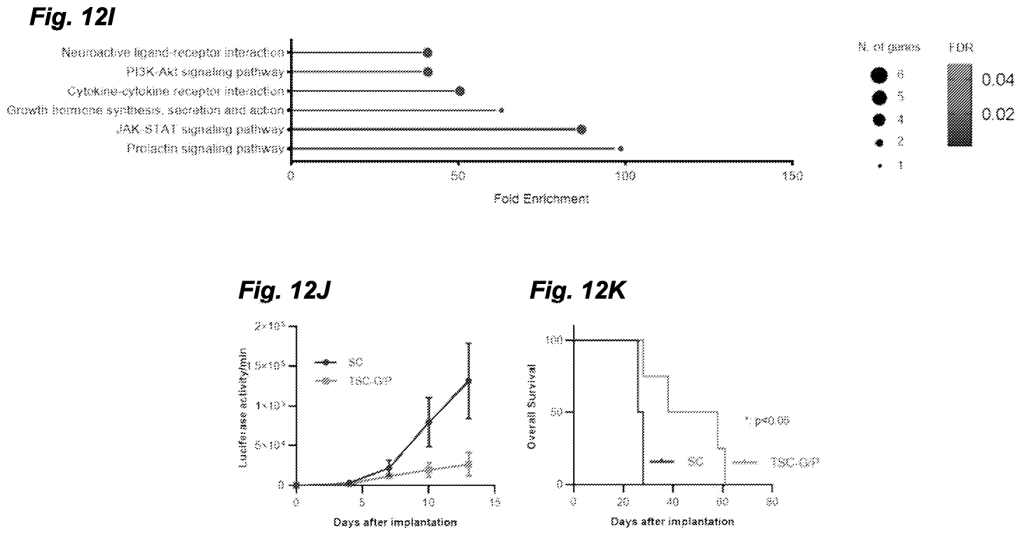

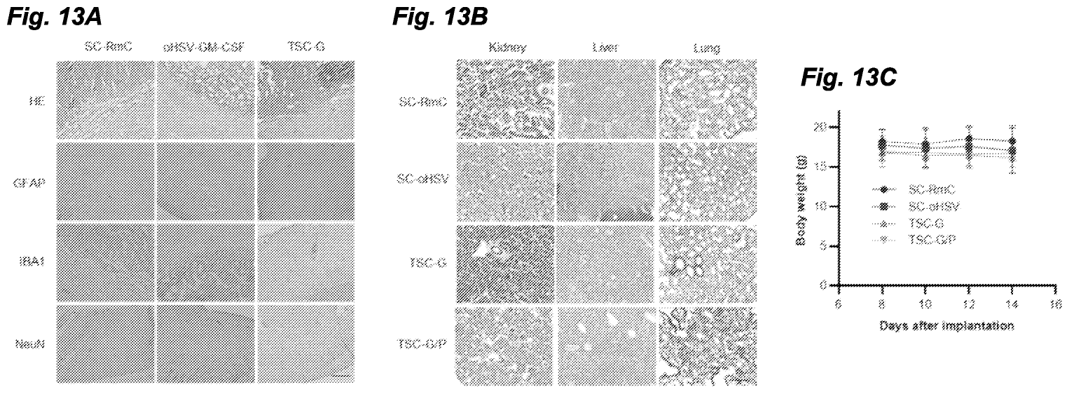

Twin stem cells releasing oHSV, GM-CSF and scFvPD-1 (TSC-G/P) therapy have therapeutic efficacy in immunosuppressive leptomeningeal metastasis. LM is one of the severe disease types in BM most common in patients with breast or lung cancer, or melanoma (52), and manifests through cancer spreading to the membranes lining the brain and spinal cord. Patients with LM have a very poor prognosis (mean survival 8-10 weeks) due to poor performance status and lack of therapeutic options (53). ICIs such as anti PD-1 antibody have shown efficacy in melanomas. Recently, a phase 2 clinical trial with anti PD-1 antibody has been performed for patients with LM (54). To address the immunosuppressive nature of LM that were created with UV2-GFl cells, SC^{N1KO}-G cells were further transduced with a single chain variable fragment (scFv) against PD-1 (FIG. 5A). FCM showed PD-L1 expression on PTEN-deficient melanoma cell lines (Y1.1, Y2.1, UV2) (FIG. 12A). Western blot assay confirmed that SC^{N1KO}-GM-CSF/scFvPD-1 cells expressed both scFvPD-1 and GM-CSF (FIG. 5B). FCM showed that scFvPD-1 blocked anti-PD-1 antibody binding to PD-1 on splenocytes, confirming the function of scFvPD-1 (FIG. 12B). In vivo, it was confirmed that intrathecally injected (IT) SC^{N1KO}-G/P cells survived in the CSF cavity for 7 days and were cleared out afterwards (FIG. 5C). To explore the efficacy of stem cell delivery of oHSV, GM-CSF and scFvPD-1 for LM, IT injection of TSC-G/P was tested in the mouse model of LM (FIG. 5D). A significant suppression of tumor growth was observed when tumors were treated with TSC-G/P or TSC-G compared to control SC-RmC (FIG. 5E), which also translated into a significantly prolonged overall survival (FIG. 5F). On the other hand, treatment with SC-oHSV with and without SC^{N1KO}-scFvPD-1 didn't reduce tumor signal or prolong overall survival compared to SC-RmC alone in this LM mouse model (FIGS. 12C and 12D). Furthermore, the therapeutic efficacy of TSC-G/P or TSC-G was diminished in T cell-deficient NOD/SCID mice (FIG. 5G, FIG. 12E). This result indicated that T cells are critical immune cells for TSC-G/P or TSC-G therapies to be effective. In order to further investigate the mechanism underlying the efficacy of this therapies, FCM immune profiling was performed 7 days post-treatment (FIG. 5H, FIG. 12F). Both TSC-G/P and TSC-G therapies significantly increased CD45+ cells compared to control SC-RmC (FIG. 5I). Additionally, TSC-G/P therapy increased DCs (CD45+CD11b+CD11c+), mature DCs (CD45+CD11b+CD11c+MHC II-A/I-E+), CD3+T cells, CD4+ T cells and CD8+ T cells compared to SC-RmC alone (FIG. 5I). RNA-seq analysis was also performed to study the immune profile and understand the mechanism of action of GM-CSF. The therapy with TSC-G/P or TSC-G upregulated expression levels of immune-cell associated genes including T cells (cytotoxic, helper, Th1 and Th2), macrophages and DCs (FIG. 5J). KEGG analysis showed that treatment with TSC-G/P or TSC-G activated necroptosis signaling, apoptosis signaling and cytokine-cytokine receptor interaction signaling (table Si). Gene Ontology (GO) analysis also indicated activation of immune system after IT injection with TSC-G/P or TSC-G (FIG. 6G). Interestingly, GO and KEGG analysis also revealed that these therapies down-regulated JAK-STAT and PI3K-AKT pathways (FIGS. 12H and S6I). Further, the IT injection with TSC-G/P also suppressed tumor growth and improved overall survival in Y2.1-GFl bearing LM mouse model (FIGS. 6I and 6K). Finally, it was also confirmed that SC^{N1KO}-G or SC^{N1KO}-G/P treatment did not induce any significant toxicity measured as unremarkable histology in major organs and the CNS and body weight loss (FIG. 13A-13C). These results revealed that IT injection with TSC-G/P or TSC-G induced anti-LM activity via the activation of anti-tumor immune system, including DCs and T cells and down-regulated JAK-STAT and PI3K-Akt pathways in immunosuppressive PTEN-deficient LM model.

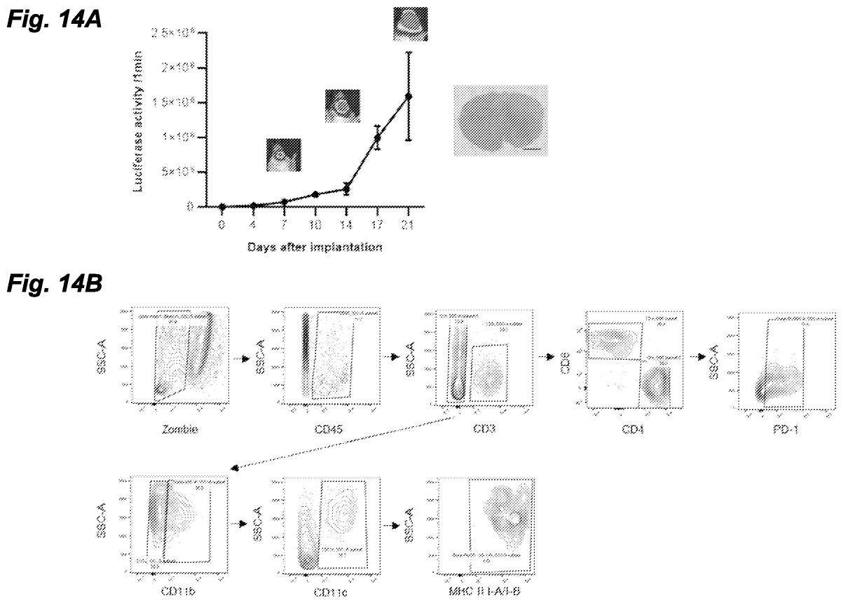

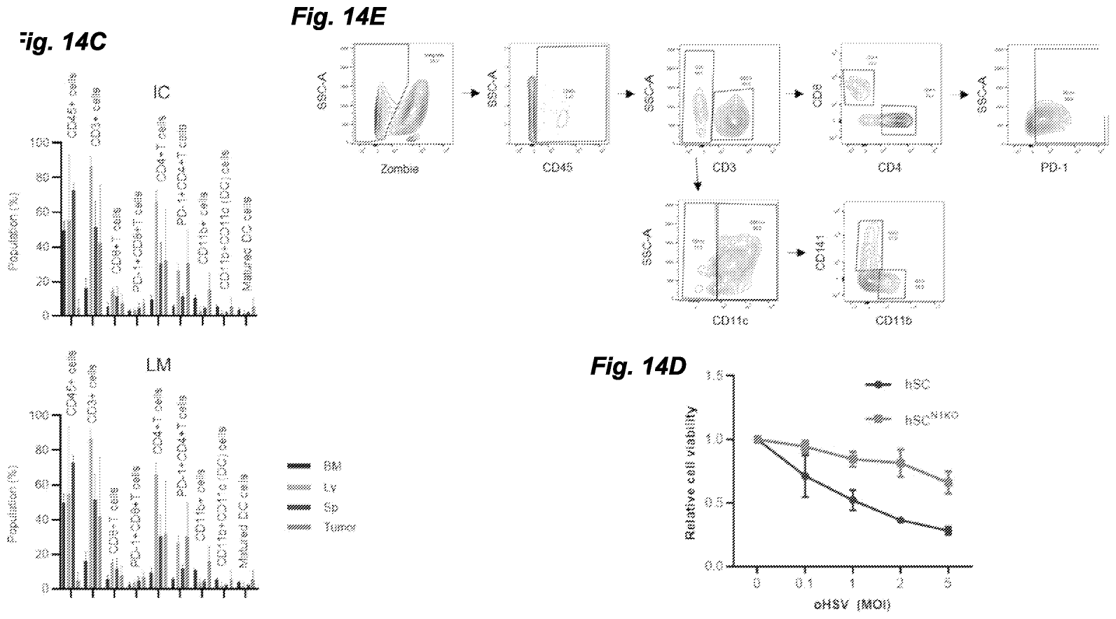

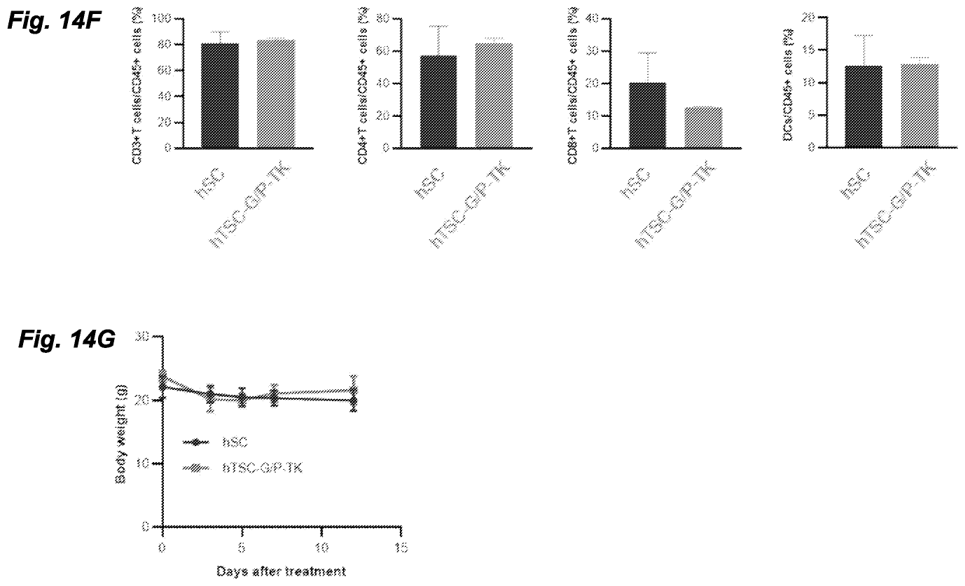

Human twin allogeneic stem cells releasing oHSV, GM-CSF and scFvPD-1 (hTSC-G/P) have therapeutic efficacy in patient derived PTEN-deficient melanoma brain metastasis: To explore the human immune system in preclinical studies of OV therapy, humanized mouse models have been tested (55), with the majority of studies using flank tumor humanized mouse models (56, 57). Patient-derived PTEN-deficient brain metastatic melanoma M12 expressing GFP-Fluc (M12-GFl) cells were first implanted intracranially in NOD-SCID mice and it was confirmed that M12-GFl cells grew well in the brain (FIG. 14A). Next, to establish the M12-GFl bearing humanized mouse model, M12-GFl cells were implanted intracranially in bone marrow-liver-thymic (BLT) humanized mice (FIG. 6A) and it was confirmed that M12-GFl cells grew well in the brain of this model, too (FIG. 6B left). IF of tumor infiltrating immune cells indicated the presence of human immune cells such as CD11c and CD3 positive cells (FIG. 6C). Interestingly, immune profiling of melanoma brain tumors as well as splenocytes, mandibular and cervical lymph nodes, and bone marrow cells all showed the presence of human DCs and T cells (CD4+ cells and CD8+cells) (FIG. 6D, FIGS. 14B and 14C).

In parallel, a M12-GFl LM model was also created using BLT humanized mice (FIG. 6A, 6B right), in which the immune profiling analysis showed human DCs and T cells in LM tumors as well as in splenocytes, mandibular and cervical lymph nodes, and bone marrow cells (FIG. 6D, FIGS. 14B and 14C). Thus, BM and LM melanoma humanized mouse models that allow the investigation of the interaction of the human immune system and human melanoma cells during immunotherapy were successfully established.

To advance the clinical translation of the murine studies described herein, human allogeneic mesenchymal stem cells (hSC) were also created by CRISPR/Cas9 technique (hSC^{N1KO}) (FIG. 6E). The hSC^{N1KO} were resistant to oHSV compared to hSCs (FIG. 8D). Next, hSC^{N1KO} secreting human GM-CSF and scFvPD-1 (hSC^{N1KO}-hG/P) were created, and it was confirmed that these cells were also resistant to oHSV when compared to hSCs (FIG. 6F). Western blot assay showed that hSC^{N1KO}-hG/P cells expressed both scFvPD-1 and GM-CSF (FIG. 6G). A herpes simplex virus thymidine kinase (HSV-TK) gene was also incorporated into hSC^{N1KO}-hG/P as a safety switch and confirmed that Ganciclovir (GCV) killed hSCs^{N1KO}-hG/P-TK in a dose dependent manner (FIGS. 6E and 6H). hSC-oHSV (2 MOI) were also able to kill M12-GFl cells in vitro (FIG. 6I). Finally, IT injection of hSC-oHSV and hSC^{N1KO}-hG/P-TK (hTSC-G/P-TK) were tested to assess the efficacy of stem cell delivery of oHSV, GM-CSF and scFvPD-1 for LM (FIG. 6J). IT injection of hTSC-G/P-TK significantly suppressed tumor growth and resulted in longer overall survival (FIGS. 6K and 6L). Finally, the immune profiling analysis of the tumors showed that hTSC-G/P-TK therapy significantly increased CD45+ cells, T cells (CD4+CD3+, and CD8+CD3+ cells), and DCs (CD11c+ cells) compared to control hSC (FIGS. 6M and 6N, FIGS. 14E and 14F). Interestingly, hTSC-G/P-TK therapy significantly induced an increased ratio of conventional DC1 (cDC1) in CD45+ cells (FIG. 6N) indicating that hGM-CSF and oHSV successfully activated human DCs. In addition, it was also confirmed that the SC-based therapy didn't cause body weight (FIG. 14G). These results showed that IT injection of hTSC-G/P-TK is a safe and effective approach that activates DCs and T cells in a humanized, patient derived melanoma LM model.

### Discussion

In this study, SC mediated delivery of oHSV and immunomodulators was explored to treat primary and metastatic melanomas. Using CRISPR/Cas-9 technology, oHSV resistant Nectin-1 receptor knockout SCs (SC^{N1KO}) were created and it was shown that SC^{N1KO} can be efficiently used to co-deliver immunomodulators with SC-oHSV. While other immunomodulators are contemplated for use in the methods described herein, SC^{N1KO} released GM-CSF was identified as the most potent immunomodulator to partner with SC-oHSV in an in vivo screening test against PTEN-deficient melanoma mouse models. It was shown that SC^{N1KO}-G augmented the therapeutic efficacy of SC-oHSV in orthotopic mouse models of primary and brain metastatic PTEN-deficient melanomas in vivo via activation of the immune system. Furthermore, SC^{N1KO} releasing both GM-SCF and scFvPD-1 effectively boosted SC-oHSV immunotherapy for immunosuppressive PTEN-deficient brain metastasis with both human and mouse immune systems.

PTEN-deficient melanoma is associated with developing BM and poor overall survival in melanoma patients (58), (18). Importantly, the activation of the PI3K pathway through loss of PTEN results in resistance to ICIs (41). The correlation between PTEN expression and BM was compared using TCGA and clinical samples. In accordance with previous reports (59-61), the analysis described herein showed that patients with BM had lower PTEN expression than patients without any metastasis, suggesting PTEN status as a potential biomarker that predicts the development of BM. Moreover, it was found that activation of PI3K/AKT pathway in low-PTEN expression patients was correlated with an immunosuppressive phenotype in melanoma. Some clinical studies showed PTEN-deficient melanoma to be resistant to immunotherapy (41). Therefore, developing novel strategies for PTEN-deficient MBM is crucial.

BM still has critical mortality even though advances in chemotherapy, targeted therapies and immunotherapies have improved survival (62). Further, melanoma leptomeningeal metastasis (MLM) has the poorest prognosis in brain metastasis despite the use of novel BRAF inhibitors or ICIs (53). The lack of understanding of the tumor microenvironment of MLM has limited the development of therapies. However, there are no reports for the establishment of syngeneic MLM mouse models. In a previous study, RNA-seq analysis of CSF from patients with MLM identified the activation of the PI3K/AKT pathway (63). Here, MLM and primary melanoma models were created and characterized using PTEN-deficient melanoma cells and reported, for the first time, immune profiles in mouse MLM models. Immune profiling of UV2 based models revealed major differences in the tumor microenvironment as the LM model was very immunosuppressive compared to flank models, suggesting that the immunosuppressive LM model might be resistant to immunotherapy, as was shown in patients (41), (17).

OV therapy offers a promising strategy to target different cancers, especially melanoma. However, there are still limitations and challenges to translate them for patients with advanced cancers. (64). First, systemic delivery of OVs is problematic for distant metastasis; intravenously administered OVs can invoke antiviral immune responses which result in viral neutralization (65, 66). In brain metastasis, the blood-brain barrier (BBB) further creates a unique challenge for systemic delivery (50). To overcome this, various delivery systems have been developed. In preclinical studies, cells such as SCs and stromal cells have been used as carriers of OVs (25). The inventors have previously explored the advantages of SC as carriers for OV and shown their efficacy against glioblastoma and metastatic melanoma in vivo (16, 25, 67). The inventors have also demonstrated that intracarotid artery administration of SC-oHSV effectively tracked metastatic tumor lesions and significantly prolonged the survival of brain tumor bearing mice (16). However, the mechanism of oHSV delivery by SC was unclear. Importantly, SC-oHSV showed a stealth effect on the immune system in in vivo experiments described herein, as its systemic administration into immune-competent mice did not induce as much anti-oHSV antibody as oHSV alone. Findings presented herein demonstrated that SC is one of the rational carriers for oHSV to overcome the disadvantage of naked oHSV treatment.

T-VEC, an oHSV expressing GM-CSF, was approved by the U.S. FDA for the local treatment of unresectable melanoma and nodule lesions. GM-CSF is recognized as an inflammatory cytokine, which modulates DC differentiation as well as macrophage activation (68). Although T-VEC mediated oncolytic cell death as well as enhanced antitumor immune responses (69), it failed to show an improvement in the overall survival of patients with brain, liver and lung metastasis (13). To overcome the limitation of T-VEC, oHSV-resistant SCs were created by knocking out Nectin-1 receptor using CRISPR/Cas9 and engineered them to secrete immunomodulators to further enhance the activation of anti-tumor immunity. It was found that SC^{N1KO}-G boosted oHSV efficacy by releasing higher levels of GM-CSF at the early phase compared to oHSV-GM-CSF or SC-oHSV-GM-CSF. Early robust expression of GM-CSF from SC^{N1KO}-G compared to oHSV-GM-CSF or SC-oHSV-GM-CSF might underlie the enhanced T cell activation and therapeutic efficacy of SC^{N1KO}-G-facilitated SC-oHSV therapy against primary melanoma and skin metastasis.

LM is a terminal disease condition without effective therapies and the clinical benefit of various systemic or intrathecal treatments remains controversial. Recently, a Phase II clinical trial of pembrolizumab in patients with LM (NCT02886585) (54) revealed safety and limited neurological toxicity. Interestingly, pembrolizumab increased the abundance of CD8+T cells in the CSF compared to pre-treatment in a small fraction of patients (70). Additionally, a Phase II study of ipilimumab and nivolumab revealed an acceptable safety profile and promising efficacy in LM patients (71). However, clinical benefits such as longer survival were limited to patients with LM derived from breast cancer. Therefore, novel treatment options for melanoma derived LM are an unmet need. The inventors previously established LM mouse models using patient derived breast cancer cells and showed that SC delivery of targeted therapeutics is a promising approach for LM (72). Here, SC^{N1KO} secreting GM-CSF and scFvPD-1 were engineered to treat immunosuppressive PTEN-deficient MLM models and investigate the immune profiling as well as therapeutic efficacy following IT injection of TSC-G/P. The findings presented herein indicate that the IT injection of TSC-G/P best prolonged overall survival via the recruitment of mature DCs and T cells even in those immunosuppressive tumors. To the best of the inventors' knowledge, this work is the first to develop IT injection of SC-based OVs and immune profile post-treatment in an LM mouse model. Moreover, these data indicated a role for immunomodulation by TSC-G/P or TSC-G in contributing to the modulation of oncologic pathways and anti-melanoma activities such as JAK-STAT and PI3K-AKT.

Humanized mouse models represent a next generation preclinical oncology platform that enables the study of how human immune cells respond to human tumors in vivo (73). Recent studies used humanized xenografted mice to demonstrate the infiltration of human T, B and NK cells within the tumor following the treatment with oncolytic vaccinia virus (56). The majority of previous reports on humanized mouse models used flank tumors to study immunotherapy for cancers (57, 74). In this study, it was first confirmed that patient derived melanoma cells could grow in the brain of BLT model. Tumor infiltration of DCs and T cells was observed in the melanoma brain tumor humanized model described herein. Furthermore, a patient derived PTEN-deficient MLM BLT mouse model was successfully established, and immune profiling analysis was performed to show human DCs and T cells in MLM tumors as well as in the spleen, mandibular and cervical lymph nodes, and bone marrow. This is the first report to date that describes an approach to establish brain metastasis and MLM BLT models. Towards clinical application, oHSV resistant human SCs were created and transduced with human GM-CSF and scFvPD-1. Additionally, SC^{N1KO}-human GM-CSF/scFvPD-1 was armed with HSV-TK as a safety switch. Finally, it was found that hTSC-G/P therapy resulted in increased overall survival in the patient derived PTEN-deficient MLM BLT mouse model. These results indicate that IT injection of two populations of allogeneic SCs provides therapeutic benefit in the context of a human immune system.

In clinical trials of brain tumors, SC delivery of OV was confirmed to be feasible and safe (NCT03072134) (75). Several clinical trials are testing OV-loaded SC for cancers (NCT02068794, NCT01844661, NCT03896568). A significant point of novelty of the study presented herein is the use of 2 different populations of allogeneic SCs for “off-the-shelf” therapy in advanced melanoma settings. The results support that the allogeneic SC-based OV immunotherapy is widely applicable to varying forms of clinical metastases, demonstrating feasibility and efficacy of intratumoral and IT treatment to improve clinical outcome in patients with advanced melanoma and MBM, respectively.

### Conclusions

oHSV resistant SCs were created using CRISPR/Cas9 and showed the therapeutic efficacy of SC^{N1KO}-GM-CSF and SC-oHSV in a syngeneic bilateral flank PTEN-deficient melanoma model. Moreover, IT injection of TSC-G/P successfully treated immunosuppressive PTEN-deficient LM, via activation of T cell and DC responses in the tumor microenvironment. It is specifically anticipated that the novel immunotherapy described herein will be an effective treatment paradigm for LM, and applicable to tumors with intact PTEN as well. Gene edited and engineered allogeneic SC releasing OV and immunomodulators can be the next breakthrough in cancer immunotherapy for metastatic melanoma as well as MBM.

### Materials and Methods

### Antibodies and Reagents

The following antibodies and reagents were used in this study. Antibodies against CD11c (#97585), β-actin (#4970), phospho-AKT (Ser473, #4060), cleaved caspase-3 (#9661), poly(ADP-ribose) polymerase (PARP; #9541), PI3K P110 alpha (#4254), PTEN (#9188), His Tag (#2365), HA Tag (#3724), GM-CSF (#56712) (CST), CD3 (#5690), CD4 (#183685), CD8 (#22378), CD68 (#125212), GM-CSF (9741) horseradish peroxidase (HRP) anti-rabbit (#7074), HRP anti-mouse (#ab205719), anti-α-tubulin (#T5168), anti-Vinculin (#V4505), (Abcam), NeuN (#MAB377), glial fibrillary acidic protein (GFAP) (#MAB3402), Nectin 1 (37-5900) (Sigma-Aldrich), Alexa Fluor anti-mouse 555 (#A-21422), Alexa Flour anti-rabbit 647 (#A-21244), IBA1 (#019-19741, FUJIFILM). For flowcytometry, Pacific Blue™ anti-mouse I-A/I-E Antibody (M1/70), Brilliant Violet (BV) 605™ anti-mouse CD279 (PD-1) Antibody (29F.1A12), BV711 Rat Anti-Mouse CD4 (RM4-5), PerCP-Cy™5.5 Rat Anti-Mouse CD8a (53-6.7), PE anti-mouse F4/80 Antibody (BM8), Brilliant Violet 711™ anti-mouse F4/80 Antibody (BM8), PE/Cyanine7 anti-mouse/human CD11b Antibody (M1/70), APC anti-mouse CD11c Antibody (N418), APC/Fire™ 750 anti-mouse CD3 Antibody (17A2), Alexa Fluor® 647 anti-mouse FOXP3 Antibody (MF-14), Brilliant Violet 650™ anti-mouse TNF-α Antibody (MP6-XT22), APC/Fire™ 750 anti-mouse NK-1.1 Antibody (PK136), PerCP/Cyanine5.5 anti-mouse I-A/I-E Antibody (M5/114.15.2), Mouse GM-CSF R alpha APC-conjugated Antibody (FAB6130A), Brilliant Violet 650™ anti-mouse CD206 (MMR) Antibody (C068C2), PerCP/Cyanine5.5 anti-mouse CD80 Antibody (16-10A1), APC/Cyanine7 anti-mouse CD86 Antibody (GL-1), Brilliant Violet 421™ anti-mouse CD62L Antibody (MEL-14), APC anti-mouse/human CD44 Antibody (IM7), PE anti-mouse CD274 (B7-H1, PD-L1) Antibody (10F.9G2), BUV395 Mouse Anti-Human CD3 (SK7), Brilliant Violet 421™ anti-human CD11b Antibody (ICRF44), Brilliant Violet 605™ anti-human CD279 (PD-1) Antibody (NAT105), PerCP/Cyanine5.5 anti-human HLA-DR, DP, DQ Antibody (T39), PE anti-human CD45 Antibody (2D1), PE/Cyanine7 anti-human CD8a Antibody (HIT8a), APC anti-human CD11c Antibody (3.9), APC/Cyanine7 anti-human CD4 Antibody (A161A1) were used.

TCGA analysis: mRNA expression profile and patient information of tumor samples were extracted from the R2 Genomics Analysis Visualization Platform (https://hgserverl.amc.nl.cgi-bin/r2/main.cgi). Set-score was determined by subtracting the average z-score of negative regulating genes from average z-score of positive regulating genes, determined by pathway analysis of GO:0014065 (http://www.informatics.jax.org/go/term/GO:0014065). Group determination of PTEN-expression and pathway activation was done based on median and extreme quartiles, respectively. Immune phenotyping data of TCGA patients was based on Thorsson et al study. (76). Extracted data were entered into GraphPad Prism 9 software to generate graphs and heatmaps.

Cell lines and cell cultures: YUMM1.1 (Y1.1), YUMM2.1 (Y2.1), YUMM3.3 (Y3.3), D3UV2 (UV2), D3UV3 (UV3), and B16F10 (B16), murine melanoma cells, and M12 patient-derived melanoma brain metastatic lines (kindly provided by J. Sarkaria, Mayo Clinic, Rochester) were cultured in DMEM supplemented with 10% FBS and 1% penicillin-streptomycin. Mouse adipose tissue-derived mesenchymal SCs were cultured in low-glucose DMEM supplemented with 15% (vol/vol) FBS, 1% (vol/vol) L-Glutamine, 1% (vol/vol) non-essential amino acid solution, and 1% (vol/vol) penicillin/streptomycin. Human adipose tissue-derived mesenchymal SCs were grown in DMEM/F-12 supplemented with 10% (vol/vol) FBS, 1% (vol/vol) L-Glutamine, 1% (vol/vol) penicillin/streptomycin, and recombinant human FGF (40 ng/mL; R & D Systems, 28 Minneapolis, MN). Neither cell line was cultured for more than 2 months following resuscitation. Cell authentication was not performed by the authors.

Lentiviral transductions and engineering of stable cell lines: Lentiviral packaging (LV-mouse GM-CSF-GFP, LV-scFvPD-1-GFP, LV-human GM-CSF/scFvPD-1-mCherry) was performed by transfection of 293T cells and cells were transduced with lentiviral vectors in medium containing protamine sulfate (2 μg/ml). For BLI, cells were transduced with LV-Pico2-GFP-Fluc, or LV-GFP-Fluc. They are selected by fluorescence-activated cell sorting using a BD FACS Aria Fusion cell sorter or by puromycin selection in culture. GFP expression was visualized by fluorescence microscopy.

CRISPR knock out of Nectin-1: To establish human and mouse Nectin-1 knockout lines using CRISPR/Cas9, SCs were transduced with lentiviral Cas9 expression vectors coding for constitutively expressed Cas9 protein and lentiviral single guide RNA (sgRNA) expression vector pLKO.DEST.hygro containing the sgRNA target sequences described above for Nectin-1.

Oncolytic Herpes Simplex Virus: G47Δ BAC contains the genome of G47Δ (γ34.5-, ICP6-, ICP47-) and a cytomegalovirus promoter driven enhanced green fluorescent protein (EGFP) in place of lacZ in G47Δ. Recombinant oHSV vectors, G47Δ-empty (oHSV), and G47Δ-mChery-Fluc (oHSV-FmC), were generated using the methods described previously (ref). G47Δ-mouse GM-CSF (oHSV-GM-CSF) is also a BAC-based recombinant oHSV vector with the genomic backbone of G47Δ (γ34.5-, ICP6-, ICP47-). Briefly, the respective shuttle plasmid carrying mouse GM-CSF was integrated into G47Δ BAC using Cre-mediated recombination in DH10B Escherichia coli, and proper recombination confirmed by restriction analysis of BAC clones. Next, the resulting BAC and an Flpe-expressing plasmid were co-transfected to Vero cells, to remove the BAC-derived sequences and the EGFP gene and allow virus to be produced. Recombinant virus oHSV-GM-CSF was plaque purified and expanded. oHSV-GM-CSF express E. coli lacZ driven by endogenous ICP6 promoter and GM-CSF driven by the herpes simplex virus immediate early 4/5 promoter. GM-CSF secretion from oHSV-GM-CSF-infected Vero cells was confirmed by ELISA (365 ng/ml/1×10⁵ cells/48 h). Multiplicity of infection (MOI) and plaque-forming units (PFU) were used as virus units in vitro and in vivo, respectively. Titers of infectious oHSV were determined by plaque assay on Vero cells (American Type Culture Collection, Manassas, MA).

Cell viability assay: Cells were seeded in 96-well plates (1×10³ cells/well) (n=5) and treated with oHSV at the indicated MOI. Cell viability was determined 2-3 days after treatment using a Cell Titer Glo (Promega, Madison, WI, USA) according to the manufacturer's protocol. For co-cultures of SC and melanoma cells, SCs were infected with oHSV (MOI=2, 5) for 6 h, washed with PBS 2 times and then co-cultured with Y1.1-GFl, Y2.1-GFI and UV2-GFI cells at indicated ratio on 96-well plate (1×10³ cells/well). Cell viability assay was performed by measuring the in vitro Firefly luciferase bioluminescence.

Immunofluorescence: Tissues were extracted and fixed in 4% paraformaldehyde in PBS overnight at 4° C., followed by further fixation at 4° C. in 20% sucrose in PBS overnight and 30% sucrose in PBS overnight. 8-30 μm sections were cut at the halfway points of the tumor diameters. Fluorescent staining of untreated tumors and spleens was performed without antigen retrieval according to standard protocol (Cell Signaling Technology, MA, USA) with an additional methanol permeabilization step added after thawing sections to room temperature. Tumor sections were incubated with primary antibody against CD3, CD8, CD4, CD68 (Abcam, Cambridge, MA, USA), CD11c (Cell Signaling Technology, MA, USA), IBA1 (FUJIFILM, Tokyo, Japan) and probed with Alexa Fluor© 647, or Alexa Fluor© 555 conjugated secondary antibody (Abcam). The number of cells expressing CD3, CD8, CD4, CD68, CD11c and IBA1 was determined from three randomly selected fields.

Multi-cytokine and chemokine assays: SCs were treated with oHSV (0, and 2 MOI) for 24 h, after which various cytokines and chemokines in the supernatants were measured using a mouse cytokine array (R&D Systems, Minneapolis, MN, USA), according to the manufacturers' protocols.

ATP assays: Y1.1, Y2.1, and UV2 cells were treated with oHSV (0, 2, and 5 MOI) for 24 h, and 48 h (n=5), after which levels of extracellular ATP in the supernatants were measured using an ENLITEN ATP assay (Promega, Madison, WI, USA) according to the manufacturers' protocols.

Western blot analysis: Proteins extracted from whole-cell lysates were electrophoresed on 10-20% SDS-polyacrylamide gels and transferred to PVDF membrane (Merck Millipore). The membranes were incubated with primary antibodies against GM-CSF (Abcam, Cambridge, MA, USA), HA-Tag, His-Tag, phospho-AKT, phospho-mammalian target of rapamycin (mTOR), Phosphoinositide 3-kinase (p-PI3K), Caspase-8, poly (ADP ribose) polymerase (PARP), phospho-MLKL, LC3B, β-Actin, Phosphatase and tensin homologue deleted on chromosome ten (PTEN) (Cell Signaling Technology, Danvers, MA, USA), and Vinculin (Sigma), followed by peroxidase-linked secondary antibody. The membrane was probed with secondary antibodies and developed with ECL (Thermo Fisher Scientific, Waltham, MA, USA). Equal loading of samples was confirmed using Vinculin, or β-Actin.

In vivo mouse experiments: All in vivo experiments were approved by the Subcommittee on Research Animal Care at Brigham and Women's Hospital. Mice that died or were euthanized for ethical reasons before defined experimental end points were excluded. Animals were randomly allocated to cages and experimental groups.

Bilateral primary melanoma model: Y1.1-GFl cells (2×10⁶ cells/per mouse), or UV2-GFl (1×10⁶ cells/per mouse) were subcutaneously implanted into the bilateral flanks of 6-8-week-old female C57BL/6 mice (vendor info). Treatment was initiated 7 days after implantation. One side was treated with SC-RmC (4×10⁵ cells), SC-oHSV (2MOI, 6 h)+SC-RmC (2×10⁵ cells), oHSV-GM-CSF (2×10⁵ PFU)+SC-RmC (4×10⁵ cells), and combination therapy with SC-oHSV (2×10⁵ cells) and SC^{N1KO}-G (2×10⁵ cells) intratumorally on day 7, and day 11. The perpendicular diameter of each tumor was then measured every 3-5 days, and tumor volume was calculated using the following formula: tumor volume (mm³)=a×b²×0.5, where a represents the longest diameter, b represents the shortest diameter and 0.5 is a constant used to calculate the volume of an ellipsoid. In Y1.1-GFl melanoma mouse model, these mice were further re-challenged with intracranially Y1.1-GFl cells (2×10⁵ cells/per mouse) on day 40, and the tumor growth was monitored by IVIS system.

Leptomeningeal metastasis model: IT injection of tumor was performed based on previous report (72). Female C57BL/6, or NOD/SCID mice (6 to 10 weeks of age) were immobilized on a surgical platform after anesthesia with ketamine-xylazine. Midline skin incision was made behind the neck and occipital muscles were dissected. The dura mater between skull and atlas vertebra was exposed. Under the observation of cerebellum and brainstem through the dura mater, a catheter connected to microsyringe (Hamilton) was inserted into cisterna magna. UV2-GFl (5×10⁴ cells per mouse) in 4 μl was injected slowly through the catheter. The hole in the dura mater was closed with a small muscle piece immediately after removing catheter. On day 5, SC-RmC (4×10⁵ cells/per mouse), SC-oHSV (2MOI, 6 h/per mouse)+SC-RmC (2×10⁵ cells/per mouse), combination therapy with SC-oHSV (2×10⁵ cells/per mouse)+SC^{N1KO}-G (2×10⁵ cells/per mouse), and combination therapy with SC-oHSV (2×10⁵ cells/per mouse)+SC^{N1KO}-G/P (2×10⁵ cells/per mouse) was injected in a similar manner via the same hole from the previous injection. Then, the tumor growth was monitored by IVIS system.

Patient-derived melanoma humanized BLT mouse model: BLT mice were generated as previously described (77). Briefly, NOD/SCID mice or NOD/SCID/c/mice at 6 to 8 weeks of age were conditioned with sublethal (2 Gy) whole-body irradiation. They were anesthetized the same day and fragments of human fetal thymus and liver were implanted under the recipient kidney capsules bilaterally. Then CD34+ cells were injected intravenously. After 8 weeks, the human immune cell engraftment was monitored by FCM by determining the percentages of human CD45+ cells in peripheral blood. Then, BLT mice with over 25% of human CD45/human and mouse CD45 ratio (mean:60%) were used in this study. To establish patient-derived melanoma humanized BLT mouse model, M12-GFl cells were intracranially implanted into the brain. Next, M12-GFl cells (5×10⁴ cells/per mouse) were intrathecally implanted as patient derived melanoma leptomeningeal metastasis model. To explore the therapeutic efficacy of IT injection of hSC, hSC (4×10⁵ cells/per mouse), or hSC-oHSV (2×10⁵ cells/per mouse) and hSC^{N1KO}-hG/P-TK (2×10⁵ cells/per mouse) were intrathecally injected on day 5. Then, the tumor growth was monitored by IVIS system. Further, brain tumors, splenocytes, mandibular and cervical lymph nodes and bone marrow cells were collected to make sure the immune profiling by FCM.

Bone marrow derived dendritic cells: Femurs and tibias were collected from C57BL/6 mice (6 to 10 weeks of age). Bone marrows were harvested by flashing DMEM media (DMEM containing 10% FBS and 1% penicillin-streptomycin) using a 23 G needle. Bone marrow cells were centrifuged, resuspended in the DMEM medium and seeded in a 6 well. They were incubated with SC-RmC or SC^{N1KO}-G conditioned medium for 4 days. Then, the population of dendritic cells (CD45+CD11b+CD11c+ cells) and mature dendritic cells (CD45+CD11b+CD11c+MHC II+ cells) by FCM.

CTL assay: Mice were sacrificed about 60 days after initiation of treatment (n=4). Splenocytes, collected by homogenization of the spleen, were treated by RBC lysis buffer (Biolegend). Then, splenocytes were co-incubated with Y1.1-GFl and TC-1-GFl cells (Target) were incubated with splenocytes for 24 h at 37° C. at different ratios of Effector:Target (0:1, 1:1, 2:1, 4:1 and 8:1). Cell viability assay was performed by measuring the in vitro Flue bioluminescence.

Immune profile experiments: C57BL/6 mice or BLT mice were intrathecally implanted with UV2-GFl (5×10⁴ cells per mouse) or M12-GFl (5×10⁴ cells per mouse), respectively and treated with intrathecal injection of SC based therapy on day 5. On day 12 or 15, mice were euthanized and tumors were collected. Tumor tissues or spleens were harvested from mice and mashed through a 100 μm strainer. For splenocytes, red blood cells were lysed using Mouse RBC Lysis Buffer (Boston BioProducts, IBB-198). Live/dead cell discrimination was performed using Zombie UV Fixable Viability Kit (BioLegend). Cells were incubated with FcR Blocking Reagent (Miltenyi Biotec) or Human TruStain FcX™ (Fc Receptor Blocking Solution) (BioLegend), followed by cell surface staining with fluorochrome-conjugated anti-mouse antibodies or anti-human antibodies. Stained cells were fixed with 4% paraformaldehyde prior to running them on BD Fortessa. FCS files were analyzed on FlowJo (version 10.3.1).

RNA-sequencing: C57BL/6 mice were intrathecally implanted with UV2-GFl (5×10⁴ cells per mouse), and treated with intrathecal injection of SC based therapy on days 5. On day 12, mice were euthanized and tumors were collected. Total RNA was extracted from tumor tissues using RNeasy Mini Kit (Qiagen, 74104) following the manufacturer's protocol and kept at −80° C. until analysis. Sample quality was checked using Agilent 2100 Bioanalyzer (Agilent). Library preparation and sequencing were performed by BGI Genomics using DNBseq platform (BGI) at a total of 4.47 Gb bases per sample. After sequencing, the raw reads were filtered using Soapnuke (BGI). After getting clean reads, hierarchical indexing for spliced alignment of transcripts (HISAT2) was used to align the clean reads to the reference genome (Mus musculus, GCF_000001635.9_GRCm39) (78). The average mapping ratio with reference genome is 95.60%, the average mapping ratio with gene is 90.45%, and 55417 genes were identified. For differential gene expression analysis, pairwise comparisons between the RNA-seq counts between different experimental groups were performed using DESeq2 in RNAdetector (79). Genes with an adjusted p value less than 0.05 were considered to be differentially expressed. For gene ontology pathway enrichment analysis, differentially expressed genes between groups were analysed using ShinyGO v0.75 (80). Pathways with an adjusted enrichment p value less than 0.05 were considered to be significantly enriched.

Statistical analysis: Statistical analysis was performed using JMP software (SAS Institute, Cary, NC, USA). Student's t test was used to assess the significance of differences in most continuous variables. The log-rank test was used for Kaplan-Meier survival analyses. P values<0.05 were considered significant.

### REFERENCES

1. M. C. Chamberlain, Brain metastases: a medical neuro-oncology perspective. Expert Rev Neurother 10, 563-573 (2010).
2. J. Zakrzewski et al., Clinical variables and primary tumor characteristics predictive of the development of melanoma brain metastases and post-brain metastases survival. Cancer 117, 1711-1720 (2011).
3. A. Y. Bedikian et al., Predictive factors for the development of brain metastasis in advanced unresectable metastatic melanoma. Am J Clin Oncol 34, 603-610 (2011).
4. D. G. Coit et al., Cutaneous Melanoma, Version 2.2019, NCCN Clinical Practice Guidelines in Oncology. Journal of the National Comprehensive Cancer Network: JNCCN 17, 367-402 (2019).
5. T. Gummadi et al., Impact of BRAF mutation and BRAF inhibition on melanoma brain metastases. Melanoma Res 25, 75-79 (2015).
6. M. Aghi, R. L. Martuza, Oncolytic viral therapies—the clinical experience. Oncogene 24, 7802-7816 (2005).
7. L. A. Selznick et al., Molecular strategies for the treatment of malignant glioma—genes, viruses, and vaccines. Neurosurg Rev 31, 141-155; discussion 155 (2008).
8. P. R. Buijs, J. H. Verhagen, C. H. van Eijck, B. G. van den Hoogen, Oncolytic viruses: From bench to bedside with a focus on safety. Hum Vaccin Immunother, 0 (2015).
9. T. C. Liu, E. Galanis, D. Kim, Clinical trial results with oncolytic virotherapy: a century of promise, a decade of progress. Nat Clin Pract Oncol 4, 101-117 (2007).
10. J. M. Markert et al., Phase Ib trial of mutant herpes simplex virus G207 inoculated pre-and post-tumor resection for recurrent GBM. Mol Ther 17, 199-207 (2009).
11. J. M. Markert et al., A phase 1 trial of oncolytic HSV-1, G207, given in combination with radiation for recurrent GBM demonstrates safety and radiographic responses. Mol Ther 22, 1048-1055 (2014).
12. A. L. Pecora et al., Phase I trial of intravenous administration of PV701, an oncolytic virus, in patients with advanced solid cancers. J Clin Oncol 20, 2251-2266 (2002).
13. R. H. Andtbacka et al., Talimogene Laherparepvec Improves Durable Response Rate in Patients With Advanced Melanoma. Journal of clinical oncology: official journal of the American Society of Clinical Oncology 33, 2780-2788 (2015).
14. K. J. Harrington et al., Clinical development of talimogene laherparepvec (T-VEC): a modified herpes simplex virus type-1-derived oncolytic immunotherapy. Expert Rev Anticancer Ther 15, 1389-1403 (2015).
15. S. J. Russell, K. W. Peng, J. C. Bell, Oncolytic virotherapy. Nat Biotechnol 30, 658-670 (2012).
16. W. Du et al., Stem cell-released oncolytic herpes simplex virus has therapeutic efficacy in brain metastatic melanomas. Proc Natl Acad Sci USA 114, E6157-E6165 (2017).
17. G. M. Fischer et al., Molecular Profiling Reveals Unique Immune and Metabolic Features of Melanoma Brain Metastases. Cancer discovery 9, 628-645 (2019).
18. A. D. Bucheit et al., Complete loss of PTEN protein expression correlates with shorter time to brain metastasis and survival in stage IIIB/C melanoma patients with BRAFV600 mutations. Clin Cancer Res 20, 5527-5536 (2014).
19. B. R. Champion, K. Fisher, L. Seymour, A PTENtial cause for the selectivity of oncolytic viruses? Nat Immunol 17, 225-226 (2016).
20. S. Li et al., The tumor suppressor PTEN has a critical role in antiviral innate immunity. Nat Immunol 17, 241-249 (2016).
21. A. Koski et al., Treatment of cancer patients with a serotype 5/3 chimeric oncolytic adenovirus expressing GMCSF. Mol Ther 18, 1874-1884 (2010).
22. S. Parviainen et al., GMCSF-armed vaccinia virus induces an antitumor immune response. Int J Cancer 136, 1065-1072 (2015).
23. J. C. Hu et al., A phase I study of OncoVEXGM-CSF, a second-generation oncolytic herpes simplex virus expressing granulocyte macrophage colony-stimulating factor. Clin Cancer Res 12, 6737-6747 (2006).
24. S. Malhotra et al., Use of an oncolytic virus secreting GM-CSF as combined oncolytic and immunotherapy for treatment of colorectal and hepatic adenocarcinomas. Surgery 141, 520-529 (2007).
25. M. Duebgen et al., Stem cells loaded with multimechanistic oncolytic herpes simplex virus variants for brain tumor therapy. J Natl Cancer Inst 106, dju090 (2014).
26. C. Krummenacher et al., Comparative usage of herpesvirus entry mediator A and nectin-1 by laboratory strains and clinical isolates of herpes simplex virus. Virology 322, 286-299 (2004).
27. J. R. Brahmer et al., Safety and activity of anti-PD-L1 antibody in patients with advanced cancer. N Engl J Med 366, 2455-2465 (2012).
28. N. Cancer Genome Atlas, Genomic Classification of Cutaneous Melanoma. Cell 161, 1681-1696 (2015).
29. S. E. Lawler, M. C. Speranza, C. F. Cho, E. A. Chiocca, Oncolytic Viruses in Cancer Treatment: A Review. JAMA Oncol 3, 841-849 (2017).
30. A. Ribas et al., Oncolytic Virotherapy Promotes Intratumoral T Cell Infiltration and Improves Anti-PD-1 Immunotherapy. Cell 170, 1109-1119 e1110 (2017).
31. S. S. Wheeler, W. M. Boyce, W. K. Reisen, Field Methods and Sample Collection Techniques for the Surveillance of West Nile Virus in Avian Hosts. Methods Mol Biol 1435, 207-220 (2016).
32. A. Marabelle, L. Tselikas, T. de Baere, R. Houot, Intratumoral immunotherapy: using the tumor as the remedy. Ann Oncol 28, xii33-xii43 (2017).
33. D. Saha, R. L. Martuza, S. D. Rabkin, Macrophage Polarization Contributes to Glioblastoma Eradication by Combination Immunovirotherapy and Immune Checkpoint Blockade. Cancer Cell 32, 253-267 e255 (2017).
34. G. Gampa, S. Vaidhyanathan, J. N. Sarkaria, W. F. Elmquist, Drug delivery to melanoma brain metastases: Can current challenges lead to new opportunities? Pharmacol Res 123, 10-25 (2017).
35. N. M. Rochet et al., Melanoma brain metastases and vemurafenib: need for further investigation. Mayo Clin Proc 87, 976-981 (2012).
36. D. Pluim et al., Enzyme linked immunosorbent assay for the quantification of nivolumab and pembrolizumab in human serum and cerebrospinal fluid. J Pharm Biomed Anal 164, 128-134 (2019).
37. R. Kim et al., Differences in tumor microenvironments between primary lung tumors and brain metastases in lung cancer patients: therapeutic implications for immune checkpoint inhibitors. BMC Cancer 19, 19 (2019).
38. A. Garcia-Diaz et al., Interferon Receptor Signaling Pathways Regulating PD-L1 and PD-L2 Expression. Cell Rep 19, 1189-1201 (2017).
39. M. Thorn et al., Tumor-associated GM-CSF overexpression induces immunoinhibitory molecules via STAT3 in myeloid-suppressor cells infiltrating liver metastases. Cancer Gene Ther 23, 188-198 (2016).
40. M. Song et al., PTEN loss increases PD-L1 protein expression and affects the correlation between PD-L1 expression and clinical parameters in colorectal cancer. PLoS One 8, e65821 (2013).
41. W. Peng et al., Loss of PTEN Promotes Resistance to T Cell-Mediated Immunotherapy. Cancer Discov 6, 202-216 (2016).
42. H. Tian, Y. Lyu, Y. G. Yang, Z. Hu, Humanized Rodent Models for Cancer Research. Frontiers in oncology 10, 1696 (2020).
43. G. Hoxhaj, B. D. Manning, The PI3K-AKT network at the interface of oncogenic signalling and cancer metabolism. Nature reviews. Cancer 20, 74-88 (2020).
44. K. Meeth, J. X. Wang, G. Micevic, W. Damsky, M. W. Bosenberg, The YUMM lines: a series of congenic mouse melanoma cell lines with defined genetic alterations. Pigment cell & melanoma research 29, 590-597 (2016).
45. M. H. Jenkins et al., Multiple murine BRaf(V600E) melanoma cell lines with sensitivity to PLX4032. Pigment Cell Melanoma Res 27, 495-501 (2014).
46. K. Meeth, J. X. Wang, G. Micevic, W. Damsky, M. W. Bosenberg, The YUMM lines: a series of congenic mouse melanoma cell lines with defined genetic alterations. Pigment Cell Melanoma Res 29, 590-597 (2016).
47. B. Homet Moreno et al., Response to Programmed Cell Death-1 Blockade in a Murine Melanoma Syngeneic Model Requires Costimulation, CD4, and CD8 T Cells. Cancer immunology research 4, 845-857 (2016).
48. I. Comerford, S. R. McColl, Mini-review series: focus on chemokines. Immunol Cell Biol 89, 183-184 (2011).
49. A. Zlotnik, O. Yoshie, Chemokines: a new classification system and their role in immunity. Immunity 12, 121-127 (2000).
50. S. K. Totsch et al., Oncolytic herpes simplex virus immunotherapy for brain tumors: current pitfalls and emerging strategies to overcome therapeutic resistance. Oncogene 38, 6159-6171 (2019).
51. J. Naidoo et al., Toxicities of the anti-PD-1 and anti-PD-L1 immune checkpoint antibodies. Ann Oncol 26, 2375-2391 (2015).
52. S. Taillibert, M. C. Chamberlain, Leptomeningeal metastasis. Handbook of clinical neurology 149, 169-204 (2018).
53. K. S. Smalley, I. V. Fedorenko, R. S. Kenchappa, S. Sahebjam, P. A. Forsyth, Managing leptomeningeal melanoma metastases in the era of immune and targeted therapy. International journal of cancer 139, 1195-1201 (2016).
54. P. K. Brastianos et al., Single-arm, open-label phase 2 trial of pembrolizumab in patients with leptomeningeal carcinomatosis. Nature medicine 26, 1280-1284 (2020).
55. J. J. Morton, G. Bird, Y. Refaeli, A. Jimeno, Humanized Mouse Xenograft Models: Narrowing the Tumor-Microenvironment Gap. Cancer research 76, 6153-6158 (2016).
56. D. Tsoneva et al., Humanized Mice with Subcutaneous Human Solid Tumors for Immune Response Analysis of Vaccinia Virus-Mediated Oncolysis. Molecular therapy oncolytics 5, 41-61 (2017).
57. S. Nakao et al., Intratumoral expression of IL-7 and IL-12 using an oncolytic virus increases systemic sensitivity to immune checkpoint blockade. Science translational medicine 12, (2020).
58. R. Cabrita et al., The Role of PTEN Loss in Immune Escape, Melanoma Prognosis and Therapy Response. Cancers 12, 742 (2020).
59. A. D. Bucheit et al., Complete loss of PTEN protein expression correlates with shorter time to brain metastasis and survival in stage IIIB/C melanoma patients with BRAFV600 mutations. Clin Cancer Res 20, 5527-5536 (2014).
60. H. Wikman et al., Relevance of PTEN loss in brain metastasis formation in breast cancer patients. Breast Cancer Res 14, R49 (2012).
61. L. Zhang et al., Microenvironment-induced PTEN loss by exosomal microRNA primes brain metastasis outgrowth. Nature 527, 100-104 (2015).
62. S. Taillibert, É. Le Rhun, [Epidemiology of brain metastases]. Cancer radiotherapie: journal de la Societe francaise de radiotherapie oncologique 19, 3-9 (2015).
63. I. Smalley et al., Proteomic Analysis of CSF from Patients with Leptomeningeal Melanoma Metastases Identifies Signatures Associated with Disease Progression and Therapeutic Resistance. Clin Cancer Res 26, 2163-2175 (2020).
64. K. Twumasi-Boateng, J. L. Pettigrew, Y. Y. E. Kwok, J. C. Bell, B. H. Nelson, Oncolytic viruses as engineering platforms for combination immunotherapy. Nature reviews. Cancer 18, 419-432 (2018).
65. N. Jayawardena, J. T. Poirier, L. N. Burga, M. Bostina, Virus-Receptor Interactions and Virus Neutralization: Insights for Oncolytic Virus Development. Oncolytic virotherapy 9, 1-15 (2020).
66. K. Harrington, D. J. Freeman, B. Kelly, J. Harper, J. C. Soria, Optimizing oncolytic virotherapy in cancer treatment. Nat Rev Drug Discov 18, 689-706 (2019).
67. J. Martinez-Quintanilla, D. He, H. Wakimoto, R. Alemany, K. Shah, Encapsulated stem cells loaded with hyaluronidase-expressing oncolytic virus for brain tumor therapy. Molecular therapy: the journal of the American Society of Gene Therapy 23, 108-118 (2015).
68. B. Becher, S. Tugues, M. Greter, GM-CSF: From Growth Factor to Central Mediator of Tissue Inflammation. Immunity 45, 963-973 (2016).
69. B. L. Liu et al., ICP34.5 deleted herpes simplex virus with enhanced oncolytic, immune stimulating, and anti-tumour properties. Gene Ther 10, 292-303 (2003).
70. S. M. Prakadan et al., Genomic and transcriptomic correlates of immunotherapy response within the tumor microenvironment of leptomeningeal metastases. Nat Commun 12, 5955 (2021).
71. P. K. Brastianos et al., Phase II study of ipilimumab and nivolumab in leptomeningeal carcinomatosis. Nat Commun 12, 5954 (2021).
72. Y. Kitamura et al., Anti-EGFR VHH-armed death receptor ligand-engineered allogeneic stem cells have therapeutic efficacy in diverse brain metastatic breast cancers. Science advances 7, (2021).
73. A. K. Wege et al., Humanized tumor mice—a new model to study and manipulate the immune response in advanced cancer therapy. Int J Cancer 129, 2194-2206 (2011).
74. M. Wang et al., Humanized mice in studying efficacy and mechanisms of PD-1-targeted cancer immunotherapy. Faseb j 32, 1537-1549 (2018).
75. J. Fares et al., Neural stem cell delivery of an oncolytic adenovirus in newly diagnosed malignant glioma: a first-in-human, phase 1, dose-escalation trial. The Lancet. Oncology 22, 1103-1114 (2021).
76. V. Thorsson et al., The Immune Landscape of Cancer. Immunity 48, 812-830.e814 (2018).
77. D. M. Brainard et al., Induction of robust cellular and humoral virus-specific adaptive immune responses in human immunodeficiency virus-infected humanized BLT mice. Journal of virology 83, 7305-7321 (2009).
78. D. Kim, J. M. Paggi, C. Park, C. Bennett, S. L. Salzberg, Graph-based genome alignment and genotyping with HISAT2 and HISAT-genotype. Nature biotechnology 37, 907-915 (2019).
79. A. La Ferlita et al., RNAdetector: a free user-friendly stand-alone and cloud-based system for RNA-Seq data analysis. BMC Bioinformatics 22, 298 (2021).
80. S. X. Ge, D. Jung, R. Yao, ShinyGO: a graphical gene-set enrichment tool for animals and plants. Bioinformatics 36, 2628-2629 (2020).

### Example 2: CRISPR Gene Editing for Prolonged Survival and Improved Anticancer Efficacy of Therapeutic Stem Cells

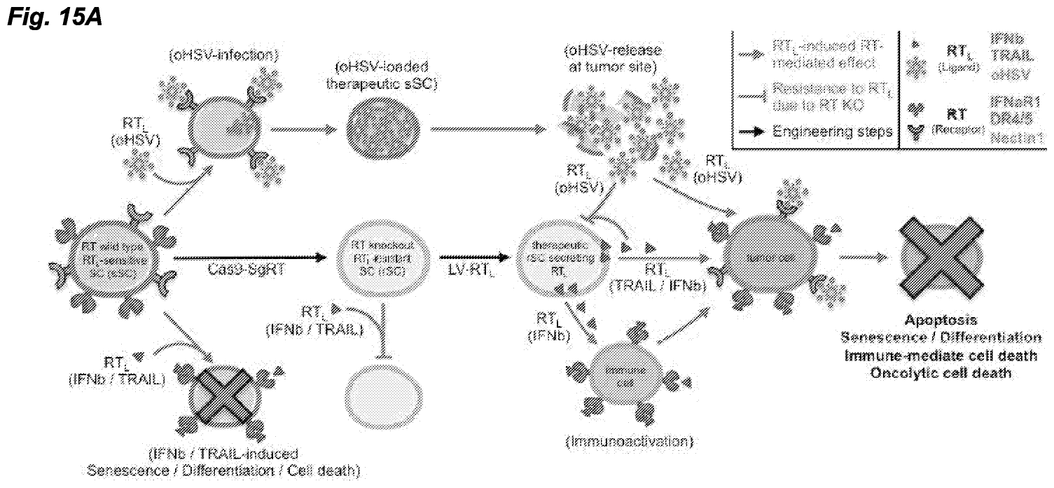

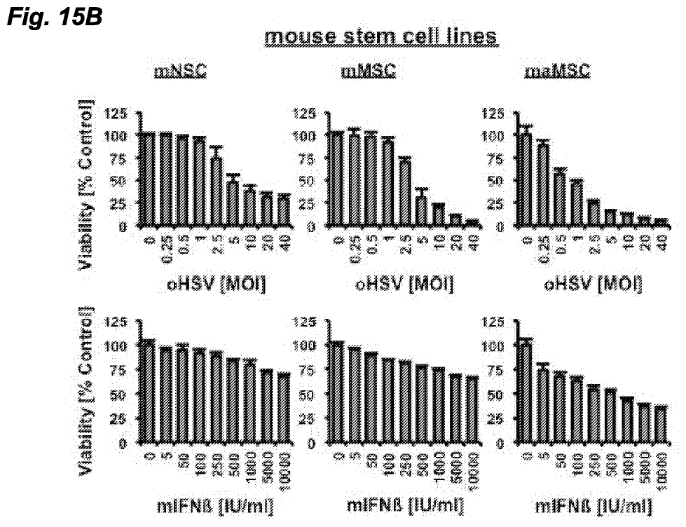

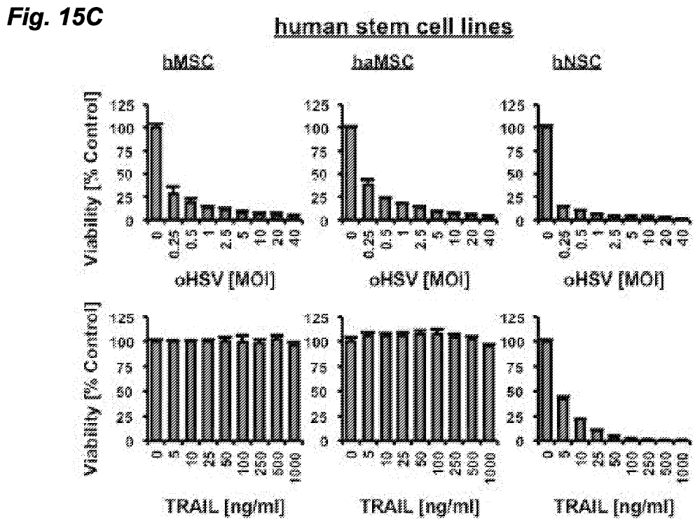

The pathotropic capabilities of stem cells (SC) and the readiness to be engineered have rendered them promising tools in the setting of both primary and metastatic cancer treatments. However, their sensitivity to endogenous cytokines in the host's microenvironment or autocrine toxicity to self-expressed transgenes has yet to be systematically addressed. In this study a panel of both human- and mouse-derived SC types were tested for their sensitivity to different cell surface receptor-targeted anticancer agents/ligands (RTL). It was demonstrated that while some SC lines are inherently resistant to these ligands, others exhibit a dose-dependent sensitivity, which in some cases prohibits stable SC-based expression of their secretable variants. Using CRISPR/Cas9 technology to knockout therapy-specific surface receptors, a roadmap towards the sensitivity-independent expression of receptor-targeted therapeutics from SCs was establish (FIG. 15A). Moreover, in the case of oncolytic virus (OV)-based therapies, it was demonstrated that this approach also allows the establishment of OV-resistant SCs with the ability to continuously secrete therapeutics even when administered in an admixture together with OV-loaded (OV-releasing) tSCs. The findings presented in this study extend SC-based therapies to a broader patient base by allowing the use of autologous SCs independent of their sensitivity to therapeutic transgenes and offers the potential to further increase therapeutic efficacy in cases of oHSV-combined SC-based therapies.

### Introduction

Research on stem cells (SCs) has provided valuable knowledge about their role during development as well as their potential in regenerative and oncologic medicine. Besides SCs intrinsic self-renewing properties and their ability to differentiate into functional, lineage-specific cell types, their pathotropic capabilities and the possibility to utilize them as site-specific delivery vehicles for various therapeutic agents have rendered them promising tools in the setting of both primary and metastatic cancer treatments (1-3). As a result, therapeutic stem cells (tSCs) are currently under investigation in numerous preclinical and clinical studies (4). In the field of cancer therapy specifically, engineering SCs ex-vivo to express receptor-targeted cytotoxic, proapoptotic or antiproliferative agents followed by local or system application has resulted in promising therapy effects (5, 6). SCs ability to stably express transgenes in combination with their tumor homing properties may be particularly valuable in cases where secreted agents are otherwise known to induce side effects/toxicity or have demonstrated limited efficacy upon systemic application due to their short biologic half-life or inability to cross the blood-brain-barrier. In addition to protein-based therapeutics, SCs have also successfully been employed for targeted delivery of oncolytic viruses (OVs) towards primary and metastatic tumor sites (7-11).

Besides promising research findings, several factors are currently still preventing a more widespread clinical translation of SC-based therapies. In addition to inherent restraints, such as issues of large-scale cell production (12), the limited survival and engraftment of tSCs, due to a lack of pro-survival signals from the local microenvironment or as a result of immunorejection (HLA incompatibility of non-autologous SCs), remain major obstacles (12-18). Moreover, tSC survival may additionally be restricted by their sensitivity to cytokines naturally expressed in the host's microenvironment or autocrine toxicity to therapeutically expressed transgenes (19-24). While combined research efforts have led to progress in overcoming some of the above-mentioned hurdles over the last decades (15, 25, 26), issues of auto- or paracrine toxicity due to SCs-released therapeutic agents have, to the best of the inventors' knowledge, not yet been systematically addressed. In this study a panel of both human- and mouse-derived SC types were tested for their sensitivity to three receptor-targeted anticancer agents (RTL): (1) the death receptor (DR) targeted pro-apoptotic tumor necrosis factor related apoptosis-inducing ligand (TRAIL), (2) the interferon-α surface receptor complex (IFNaR1 and IFNaR2) targeted cytokine interferon-ß (IFNß), and (3) Nectin1 receptor targeted oncolytic herpes simplex virus (oHSV). It is demonstrated herein that while some SC lines are inherently resistant to these ligands, others exhibit a dose-dependent sensitivity, which in case of TRAIL and IFNß may prohibit stable SC-based expression of their secretable variants. Using CRISPR/Cas9 technology to knockout therapy-specific surface receptors, a roadmap towards sensitivity-independent expression of receptor-targeted therapeutics from SCs was establish (FIG. 15A). Moreover, in case of OV-based therapies, it is demonstrated that this approach also allows establishment of oHSV-resistant SCs with the ability to continuously secrete therapeutics even when administered in an admixture together with oHSV-loaded (oHSV-releasing) tSCs.

### Results

### Different Stem Cell Types Demonstrate a Range of Responses to Receptor-Targeted Therapies

To test the hypothesis that not all stem cell types can be readily engineered to continuously release receptor-targeted therapeutics without inflicting autocrine toxicity, different mouse and human stem cell lines were first tested for their sensitivity to a panel of promising anticancer receptor ligands (RTL). Exposure of the mouse neuro-progenitor cell line (NPC, herein referred to as mNSCs), as well as mouse bone marrow-derived and mouse adipose-derived mesenchymal stem cells (herein referred to as mMSCs and maMSCs respectively) to interferon-α/ß receptor (IFNaR1/2) targeted recombinant murine IFNß (IFNß) and to nectin cell adhesion molecule 1 (Nectin1) targeted oncolytic herpes simplex virus (oHSV) identified that maMSCs as the most sensitive mouse-derived stem cell line to both therapeutics (FIG. 15B). In addition, exposure of human neural stem cells (hNS1, herein referred to as hNSCs) as well as human bone marrow-derived and human adipose-derived mesenchymal stem cells (herein referred to as hMSCs and haMSCs respectively) to death receptor (DR) targeted human TRAIL and to oHSV identified all tested human SCs as sensitive to oHSV and hNSCs as sensitive to TRAIL (FIG. 15C). Together these results demonstrate that both mouse- and human-derived SCs may demonstrate sensitivity to receptor-targeted anticancer agents, with all of the tested cell lines showing dose-dependent sensitivity to oHSV, partial or complete response to IFNß in all mouse SC lines, and viability reduction in response to TRAIL exposure in one out of the three tested human SCs.

### TCGA Analysis Identifies the Receptors Nectin1, IFNaR1 2 and DR4 5 as Promising Targets in a Variety of Cancers and Screening of Mouse and Human Cancer Cell Lines Confirms Broad Therapeutic Applicability

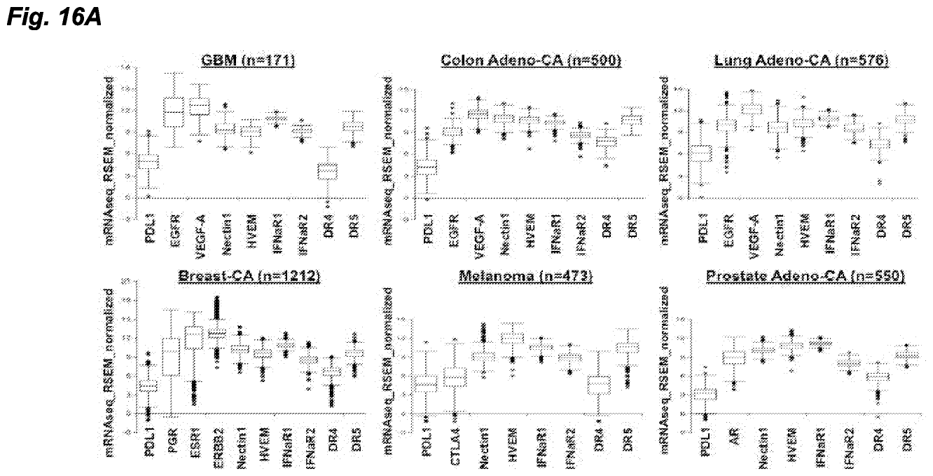

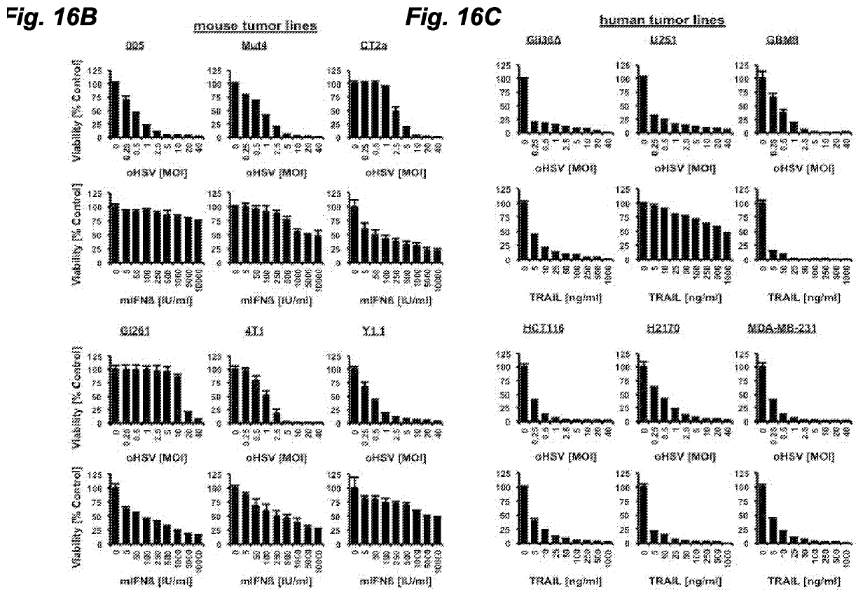

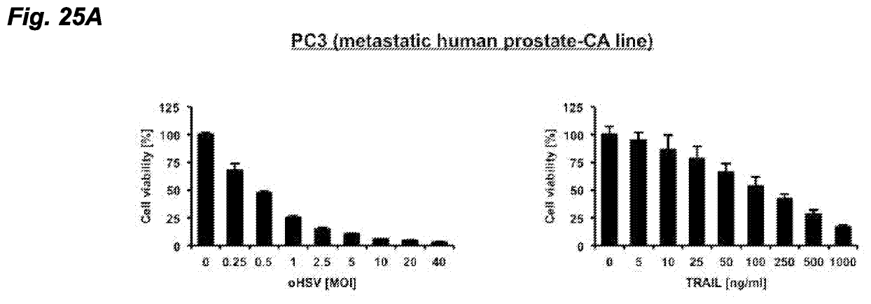

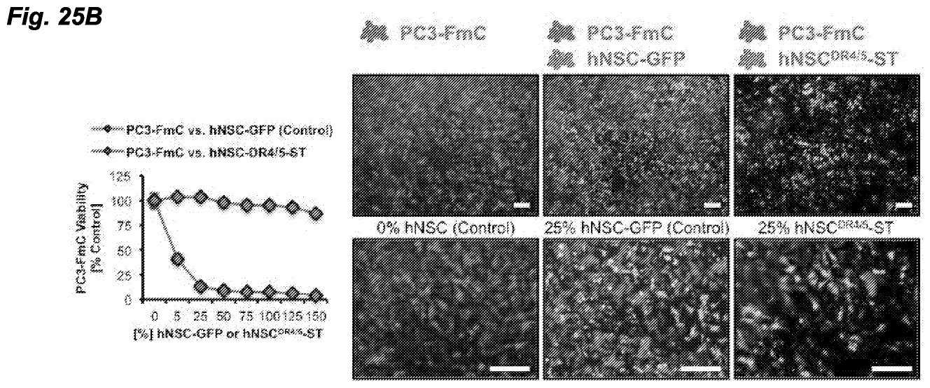

To investigate the clinical relevance of anticancer therapeutics targeting the receptors Nectin1, IFNaR1/2 and DR4/5 a TCGA query was run to determine relative RNA expression levels for some of the most common cancer types, including glioblastoma (GBM), colon, lung and prostate adenocarcinoma as well as melanoma (FIG. 16A). For comparison, receptors commonly targeted in current clinical practice were also queried. Overexpression of Nectin1, IFNaR1/2 and DR4/5 was confirmed for all cancer types with varying expression levels, however, mostly comparable to clinically established receptor target controls, such as VEGF-A receptors in colon cancer or androgen receptor (AR) in prostate cancer. To confirm these findings and to identify cell lines suitable for preclinical xenograft/syngeneic model development, a panel of both mouse and human cancer cell lines were further tested for their sensitivity to oHSV (both mouse and human), IFNß (mouse cancer cell lines only) and TRAIL (human cancer cell lines only). All of the human cancer cell lines demonstrated high sensitivity to oHSV and, although less sensitive in general, a good response was observed in most mouse-derived cancer cell lines as well, with only G1261 (C57/BL6 derived GBM) showing partial resistance (FIG. 16B, 16C). IFNß treatment resulted in a dose-dependent viability decrease in all of the tested mouse cancer cell lines with the best efficacy seen in CT2a and G1261 (both GBM). In the tested human cell lines, only U251 (GBM) demonstrated partial resistance to TRAIL. In addition, the human metastatic prostate cancer cell line PC3 demonstrated a good response to both oHSV and TRAIL (FIG. 25A). Together these data confirm that oHSV, IFNß and TRAIL are promising targets for future SC-based therapy of a wide variety of cancers.

### CRISPR Cas9 Engineering of Therapy-Sensitive Stem Cells Allows Targeted Receptor Knockout

SC lines previously identified as sensitive to receptor-targeted therapeutics (FIG. 15B, 15C) were engineered with lentivirus (LV) expressing CRISPR-associated protein 9 (Cas9) RNA-guided DNA endonuclease and SgRNAs targeting exonic gene sequences of DR4, DR5, Nectin1 or IFNaR1 as previously described (27). Cas9 expression was confirmed by western blotting (FIG. 16A). Previously identified TRAIL sensitive hNSCs engineered with Cas9 targeting DR4, DR5 or both receptors were clonally selected, and single clone receptor expression was assessed via western blotting to identify a DR4/5 double knockout clone (FIG. 16B). In a similar fashion, the oHSV sensitive maMSCs were engineered with Cas9 and SgRNAs targeting mouse Nectin1 followed by screening for Nectin1 expression against non-SgRNA engineered control via western blotting (FIG. 16C). In parallel maMSCs, previously identified as IFNß sensitive, were engineered with Cas9 targeting IFNaR1 followed by sub-clonal selection for identification of IFNaR1 knockout clones. Due to lack of mouse-specific IFNaR1 western blotting antibody, maMSCs clones were screened for IFNaR1-KO status via treatment with and without recombinant mouse IFNß followed by probing for downstream pathway activation (STAT1 and phospho-STAT1) 6 h post IFNß exposure in comparison to non-CRISPR-engineered control via western blotting (FIG. 16D). To further confirm CRISPR-mediated receptor knockout, genomic DNA (gDNA) of identified knockout SCs was extracted followed by Sanger sequencing of SgRNA-targeted gene sequences for identification of site-specific indel mutations (FIG. 16E). Together these findings demonstrate successful CRISPR engineering of RTL-sensitive SCs to knock out DR4, DR5, Nectin1 and IFNaR1 receptors.

DR Knockout hNSCs Engineered to Secrete TRAIL Show Anticancer Efficacy Against a Broad Panel of Cancers

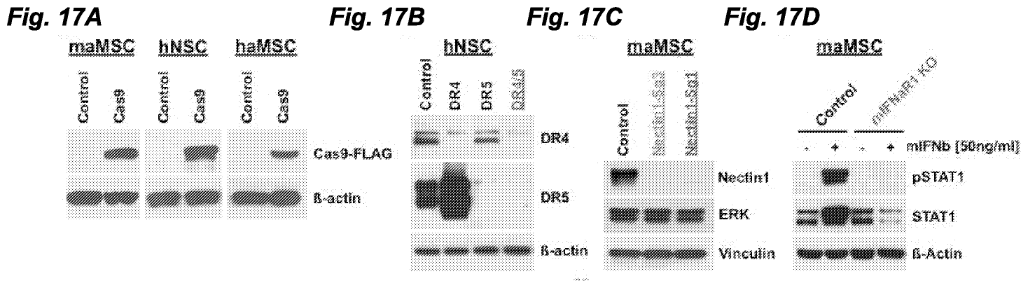

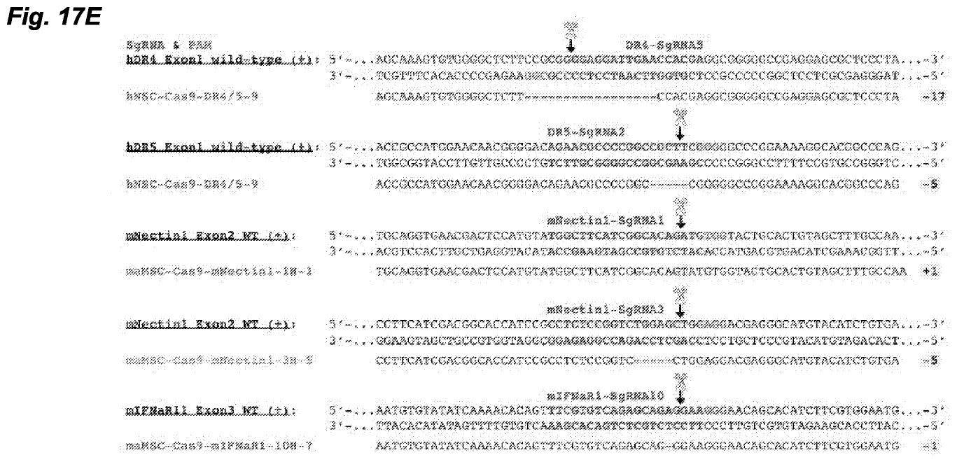

Following the confirmation of the CRISPR-mediated receptor knockout strategy, whether the identified hNSC DR4/5 double knockout clone (herein referred to as hNSC^{DR4/5}) could serve as a cell-based platform for continuous S-TRAIL delivery was determined. Titration with increasing concentrations of TRAIL confirmed complete TRAIL resistance of hNSC^{DR4/5} in comparison to DR wild type hNSCs (FIG. 17A). Moreover, in comparison to hNSC wild type cells, treatment with TRAIL did not result in apoptosis induction in hNSCDR4/5 as indicated by lack of cleaved PARP and cleaved caspase 8 expressions (FIG. 28). Next, hNSC^{DR4/5} were transduced with LV coding for S-TRAIL (ST) and GFP. Following transduction, hNSC^{DR4/5} turned green without showing signs of apoptosis, indicating stable ST expression (FIG. 17B, top). S-TRAIL expression from hNSC^{DR4/5} (herein referred to as hNSC^{DR4/5}-ST) was further confirmed via western blotting of cell lysates and conditioned media (CM) harvested 5 days post transduction (FIG. 4B, bottom). To investigate S-TRAIL expression in more detail, hNSC^{DR4/5} were transduced with LV encoding a fusion variant of S-TRAIL with the optical reporter Renilla luciferase (Rluc(o), RI) (21). Bioluminescent imaging (BLI) following transduction of hNSC^{DR4/5} demonstrated continued secretion of S-TRAIL into culture media without autocrine-toxicity of therapeutic cells, as indicated by a time-dependent increase of BLI intensity (FIG. 17C).

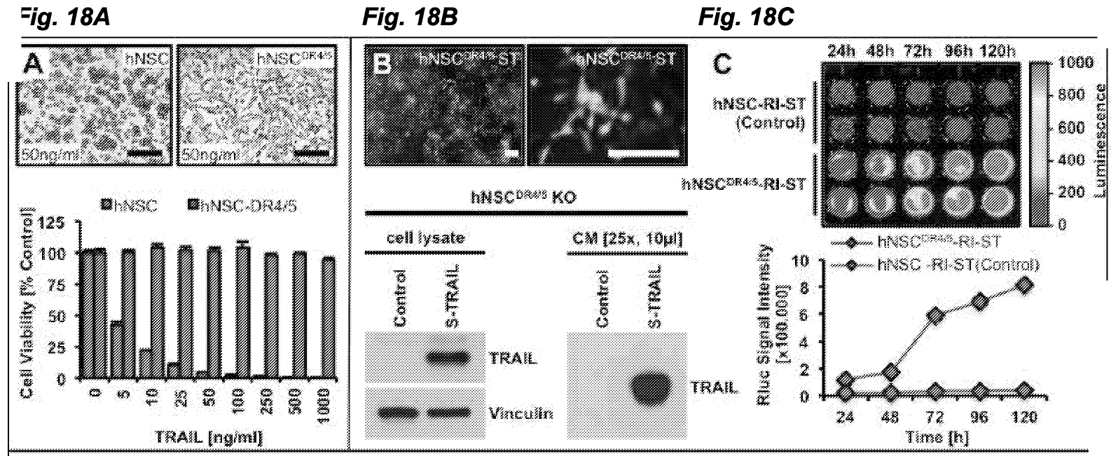

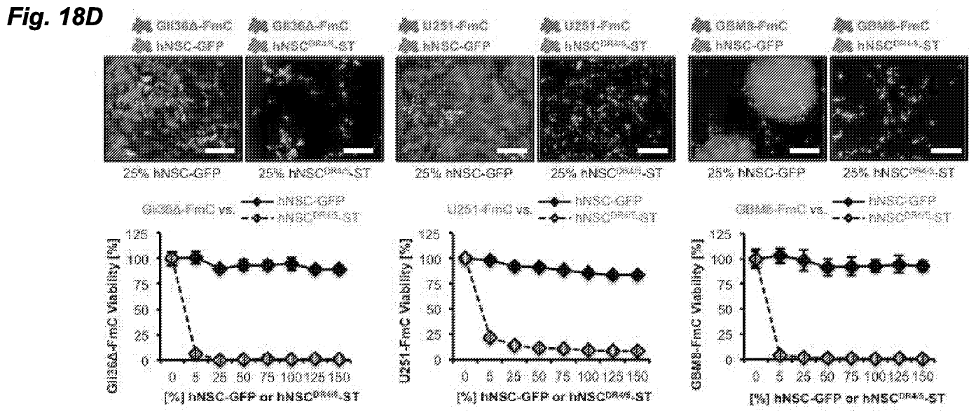

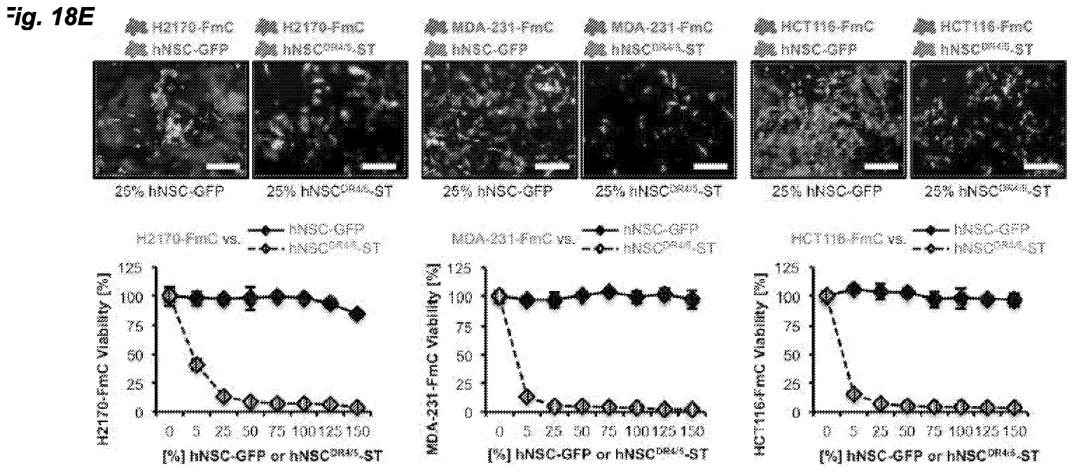

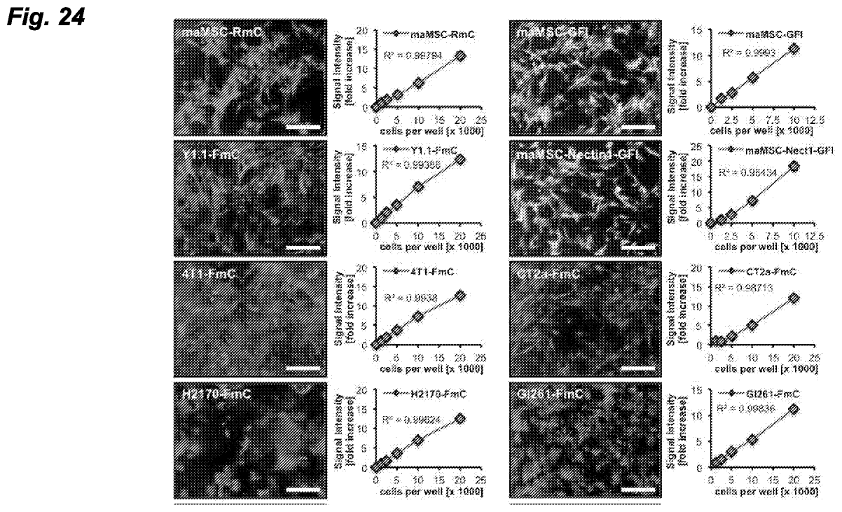

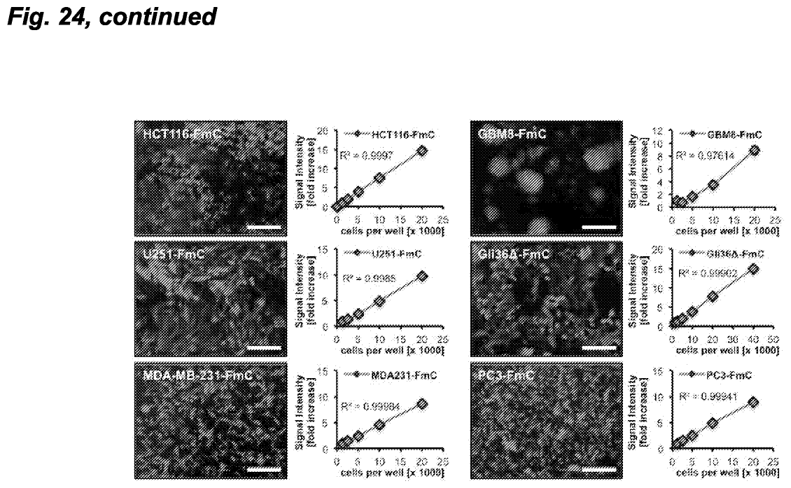

Next, to test whether hNSC^{DR4/5}-ST can serve as a vehicle for S-TRAIL delivery towards TRAIL-sensitive tumors, previously TRAIL-tested human tumor cell lines (FIG. 16C) were transduced with LV to express Flue and mCherry (FmC) (FIG. 24), followed by their coculture with hNSC^{DR4/5}-ST or hNSC-GFP (control). Established GBM cell lines Gli36Δ-FmC (TRAIL-sensitive) and U251-FmC (TRAIL semi-sensitive) as well as the primary patient-derived GBM cell line GBM8-FmC demonstrated a dose-dependent response to coculture with increasing percentages of hNSC^{DR4/5}-ST, but not hNSC-GFP (FIG. 18D). A similar response was observed when coculturing hNSC^{DR4/5}-ST with FmC-engineered established lung cancer cell line H2170-FmC, breast cancer cell line MDA-MB-231-FmC, colon cancer cell line HCT116-FmC (FIG. 18E) and the metastatic prostate cancer cell line PC3-FmC (FIG. 25B).

Together these results indicate that hNSC^{DR4/5} can be engineered to secrete S-TRAIL and that S-TRAIL released from hNSC^{DR4/5}-ST can bind to neighboring cells and induce apoptosis in vitro in a variety of cancers cell types including metastatic cancers.

IFNaR1 Knockout maMSCs are Resistant to IFNβ, can be Engineered to Secrete IFNβ and Show In Vitro Anticancer Efficacy Against Mouse-Derived Glioblastoma and Breast Cancer Cell Lines

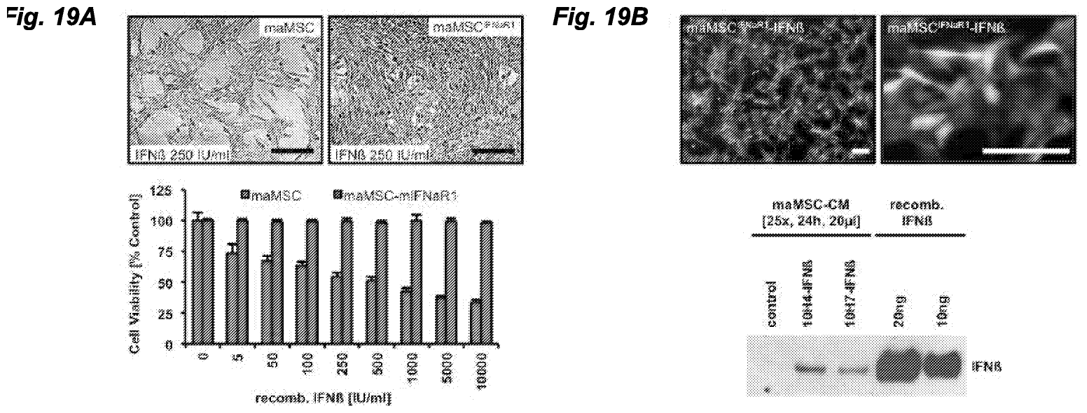

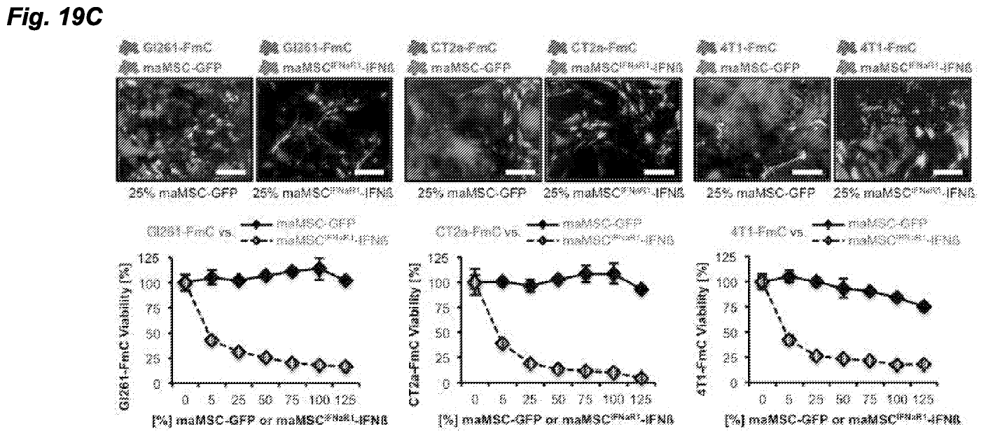

After confirmation of successful IFNaR1 receptor knockout, the identified IFNaR1 knockout clone (herein referred to as maMSC^{IFNaR1}) was to tested to determine whether it could serve as a cell-based platform for continuous IFNß delivery towards cancers in a similar fashion as achieved for hNSC^{DR4/5} with TRAIL. Titration with increasing concentrations of IFNß confirmed complete IFNß-resistance of maMSC^{IFNaR1} in comparison to IFNaR1 wild type maMSCs (FIG. 19A). Next, maMSC^{IFNaR1} were transduced with retrovirus (RV) coding for secretable IFNß and GFP. Following transduction, maMSC^{IFNaR1} turned green without showing signs of senescence/apoptosis, indicating stable IFNß expression (FIG. 19B, top). IFNß expression from maMSC^{IFNaR1} (herein referred to as maMSC^{IFNaR1}-IFNß) was further confirmed via western blotting of concentrated CM harvested 24 h post plating of maMSC^{IFNaR1}-IFNß in comparison to a known amount of recombinant IFNß (FIG. 19B, bottom). To test whether maMSC^{IFNaR1}-IFNß can serve as a vehicle for continuous IFNß delivery, previously IFNß-tested mouse tumor cell lines (FIG. 16B) were transduced with LV to express Flue and mCherry (FmC) (FIG. 24), followed by their coculture with maMSC^{IFNaR1}-IFNß or maMSC-GFP (control). Syngeneic C57BL/6 mouse-derived GBM cell lines G1261-FmC and CT2a-FmC as well as the BALB/c mouse-derived breast cancer cell 4T1-FmC all demonstrated dose-dependent response to coculture with increasing percentages of maMSC^{IFNaR1}-IFNß, but not maMSC-GFP (FIG. 19C). Together these results indicate that maMSC^{IFNaR1} can be engineered to continuously secrete IFNß without inducing autocrine effects and that IFNß released from maMSC^{IFNaR1}-IFNß can bind to neighboring cancer cells and reduce their viability in vitro.

Nectin1 Knockout Confers Resistance to oHSV

To explore the possibility of using oHSV-resistant SCs, able to continuously deliver cytokines or other secretable anticancer agents when administered in admixture with oHSV-loaded tSCs, Nectin1 knockout strategy was tested to determine if it would protect SCs from oHSV infection and oncolysis.

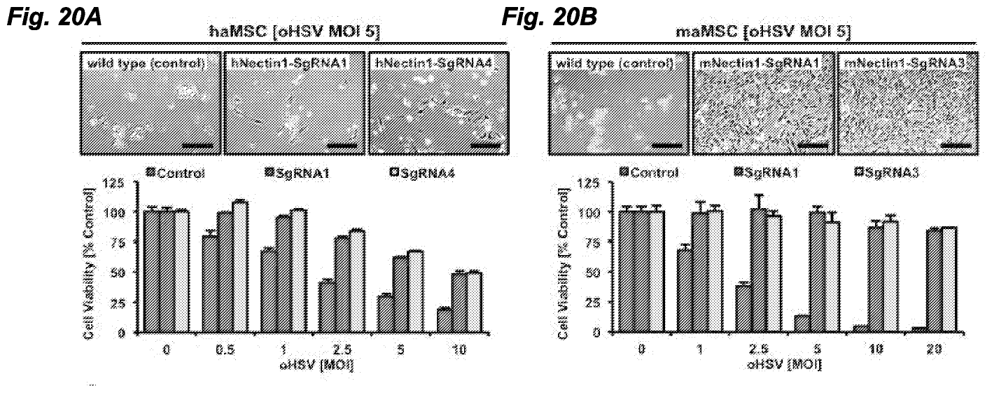

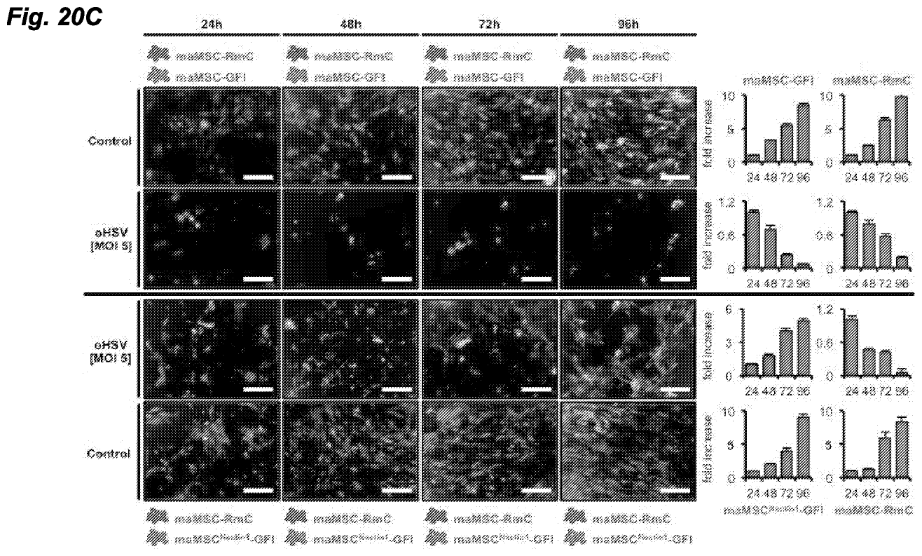

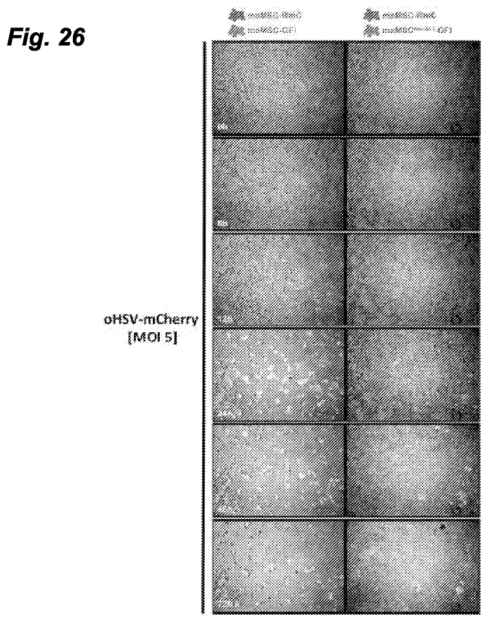

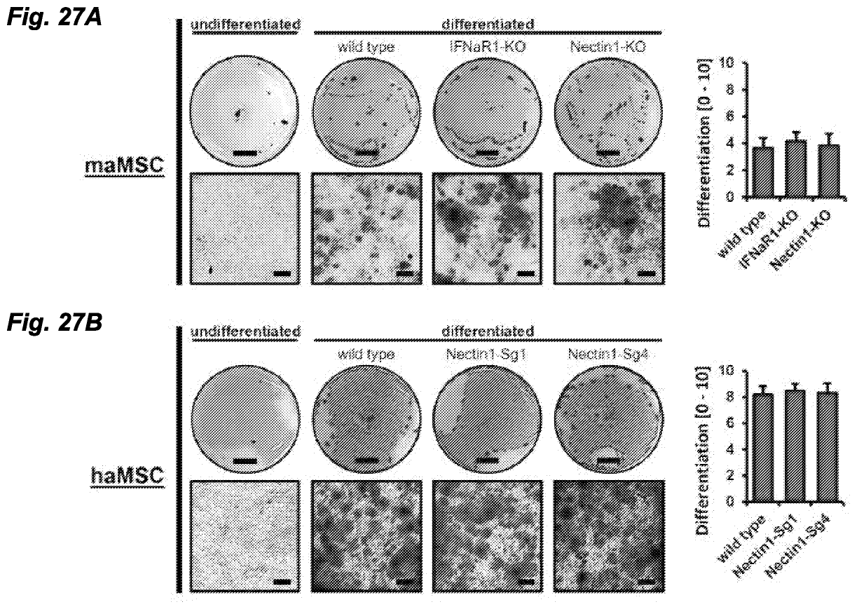

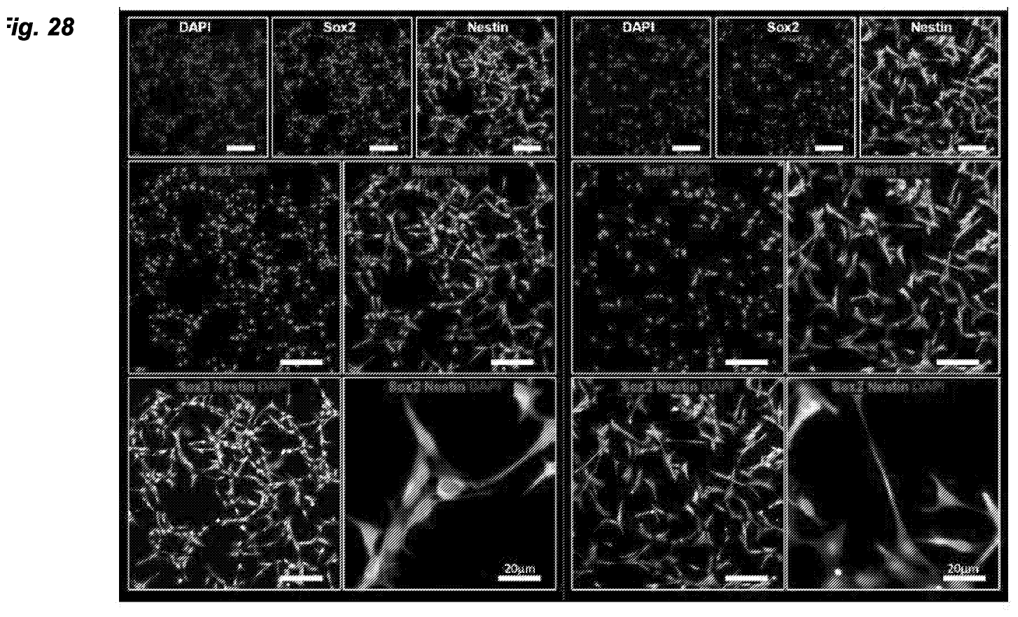

First, Cas9-expressing haMSCs co-engineered with SgRNAs targeting the human Nectin1 gene were titrated with oHSV and cell viability was compared to Nectin1 wild type control haMSCs (FIG. 20A). Interestingly, although a significant reduction of oHSV-sensitivity was observed, a stable oHSV-resistant Nectin1 knockout haMSC line was unable to be established due to observed latent oHSV infection even in post-oHSV treatment surviving cells. In contrast, the previously identified maMSC Nectin1 KO cell lines demonstrated complete resistance to oHSV treatment and showed no sign of latent infection after in vitro selection with oHSV MOI 5 (FIG. 6B). Following the confirmation of oHSV resistance in CRISPR-modified maMSCs, the oHSV-resistant maMSC-Nectin1 knockout cell line engineered with SgRNA3 (herein referred to as maMSC^{Nectin1}) was tested to determine if it could survive direct coculture with maMSC wild type cells following oHSV infection. The maMSC^{Nectin1} line was engineered with LV to express Firefly luciferase and GFP (herein referred to as maMSC^{Nectin1}-GFl, FIG. 24) and in parallel maMSC wild type cells were transduced with LV to express either Renilla luciferase (Rluc) and mCherry (RmC) or GFl (herein referred to as maMSC-RmC and maMSC-GFl respectively, FIG. 24). Next, maMSC-RmC (Nectin1 wild type) were plated in 1:1 ratio together with either maMSC-GFl (Nectin1 wild type) or maMSC^{Nectin1}-GFl (Nectin1 KO) followed by infection with or without oHSV at MOI 5 (FIG. 20C). Cell fate/viability of individual cell populations was then followed by adding luciferin (Fluc-expressing cells) or coelenterazine (Rluc-expressing cells) at different time points post oHSV treatment. A significant decrease of both maMSC-RmC and maMSC-GFl cell populations was observed following oHSV treatment in comparison to non-oHSV treated control (FIG. 20C). In contrast, maMSC^{Nectin1}-GFl showed continuous cell-expansion, even if cocultured with maMSC-RmC actively dying due to oHSV infection/oncolysis (FIG. 20C). To further observe the oHSV infection of different cell populations in real-time, maMSC-RmC were cocultured in 1:10 ratio together with either maMSC-GFl or maMSC^{Nectin1}-GFl followed by adding oHSV co-expressing mCherry (oHSV-mCherry) at MOI 5 (FIG. 26). Infection and viral replication (indicated by the appearance of red color) and following oncolysis were only observed for Nectin1 wild type maMSCs (left side of the video), but not for maMSC^{Nectin1}-GFl (right side of the video). Together these results indicate that maMSC^{Nectin1} are resistant to oHSV-infection and can keep proliferating upon oHSV exposure, even when in direct contact with oHSV-infected/oncolytic maMSCs.

### CRISPR-Modified Therapeutic Stem Cells Demonstrate In Vivo Anticancer Efficacy

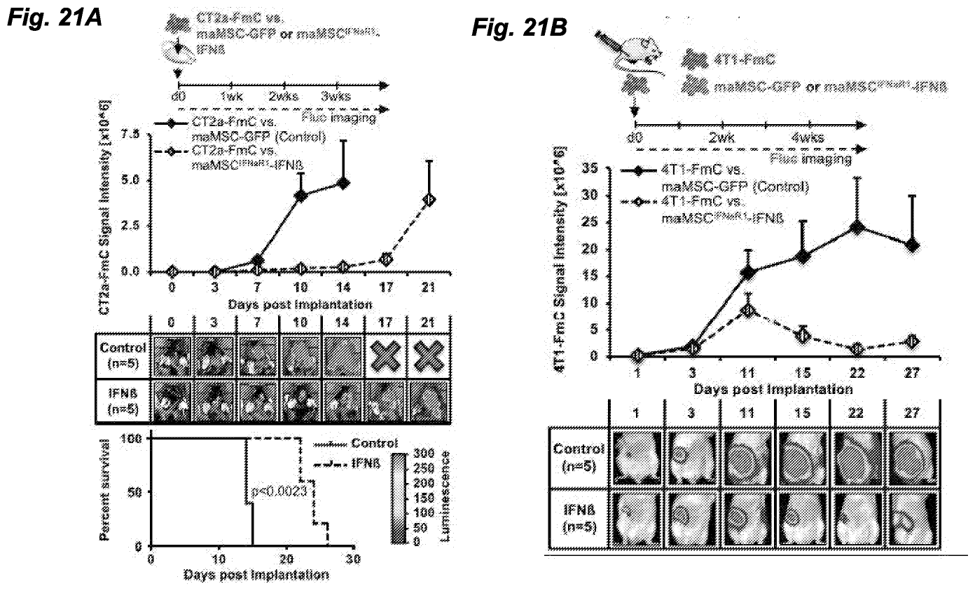

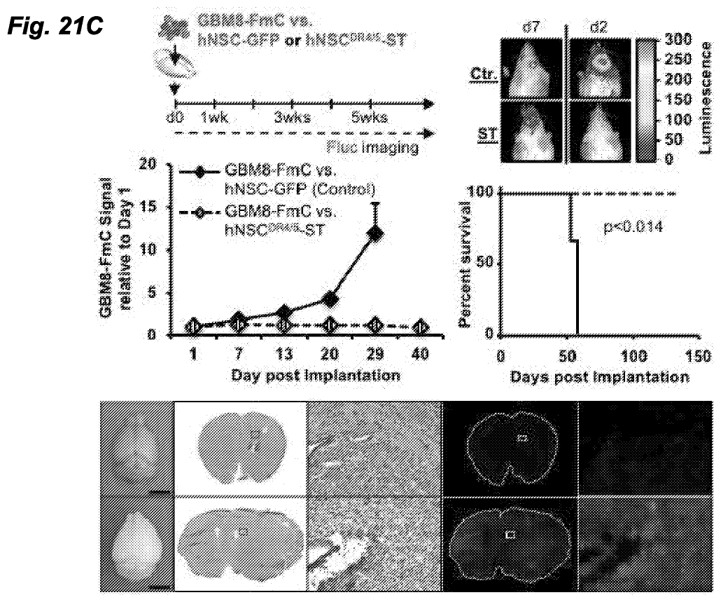

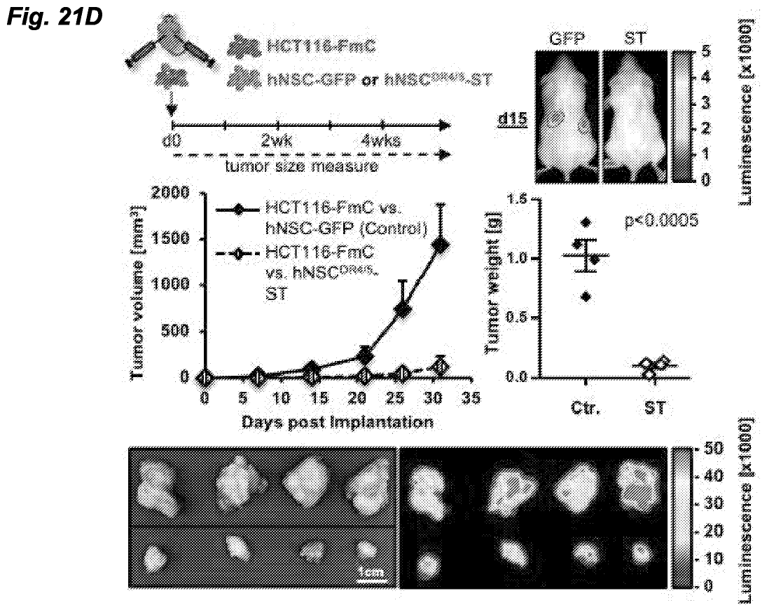

After exploring the effects of CRISPR-modification in vitro, therapeutic knockout SCs were investigated to determine if they could be used for anticancer therapy in vivo. To test the efficacy of maMSC-delivered IFNß against GBM in a syngeneic setting, CT2a-FmC were mixed 1:1 with either maMSC-GFP (control) or maMSC^{IFNaR1}-IFNß (1.5×10\hat{ }5 cells each) followed by implantation of the admixture into the right hemisphere of C57BL/6 mice (n=5 per group). After implantation, CT2a-FmC tumor growth was non-invasively monitored via BLI (FIG. 21A). In comparison to maMSC-GFP admixed control, the maMSC^{IFNaR1}-IFNß group showed marked delay in tumor growth and significantly prolonged survival (p<0.01). To further explore the in vivo efficacy of maMSC-released IFNß against other cancer types, 4T1-FmC were admixed 1:1 with either maMSC-GFP (control) or maMSC^{IFNaR1}-IFNß and implanted orthotopically into the mammary fat pad of BALB/c mice (n=5 per group). Again, 4T1-FmC tumor growth was markedly delayed in maMSC^{IFNaR1}-IFNß co-injected mice and tumor size was significantly smaller in comparison to the maMSC-GFP group (FIG. 21B). To test the in vivo efficacy of hNSC^{DR4/5} delivered S-TRAIL, hNSC^{DR4/5}-ST or hNSC-GFP (control) were admixed 1:1 with either GBM8-FmC (1.5×10\hat{ }5 cells each) followed by intracranial implantation (control n=3, S-TRAIL n=4 mice) or admixed 1:1 with HCT116-FmC (5×10\hat{ }5 cells each) followed by injection into the flanks of SCID mice (2 injections per mouse, control n=2, S-TRAIL n=3 mice). In comparison to hNSC-GFP coinjected control, GBM8-FmC admixed with hNSC^{DR4/5}-ST completely suppressed intracranial tumor formation, leading to the survival of all mice (FIG. 21C top, p<0.05). Elimination of GBM8-FmC tumors in hNSC^{DR4/5}-ST coinjected mice was additionally confirmed via cryosectioning of mouse brains followed by HE-staining and fluorescence microscopy imaging (FIG. 21C bottom). Similarly, coinjection of HCT116-FmC with hNSC^{DR4/5}-ST markedly delayed tumor formation and led to significantly smaller tumors in comparison to control (p<0.0005) when evaluated via weighing (FIG. 21D). Moreover, tumor formation was completely suppressed in 2 out of the 6 hNSC^{DR4/5}-ST coinjected implantation sites.

Nectin1 Knockout Increases Survival of maMSCs when Injected in Admixture with oHSV-Infected Wild Type maMSCs In Vivo

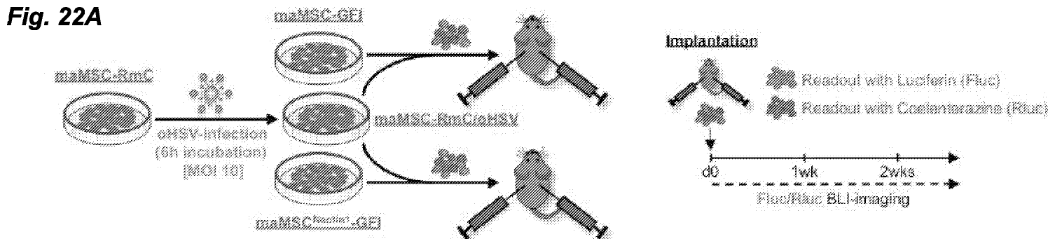

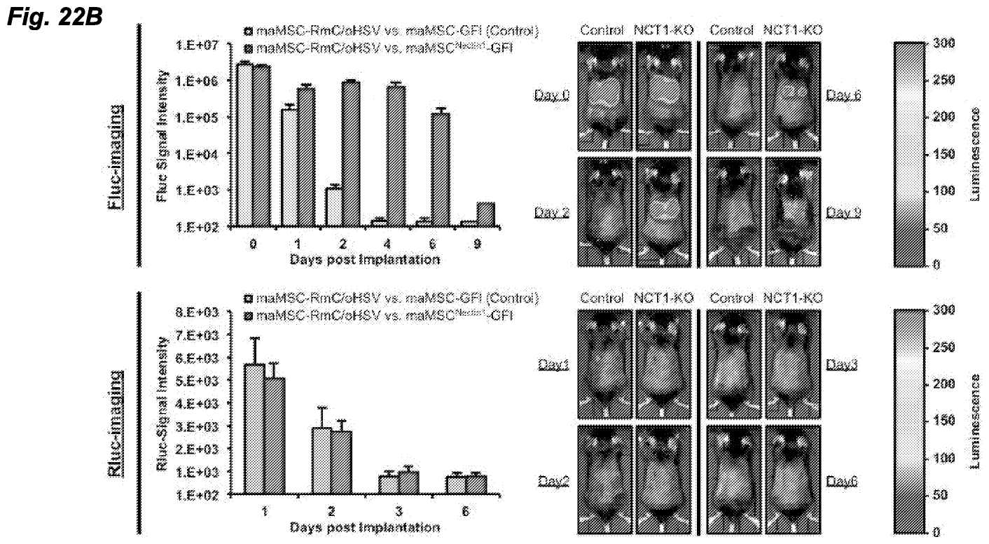

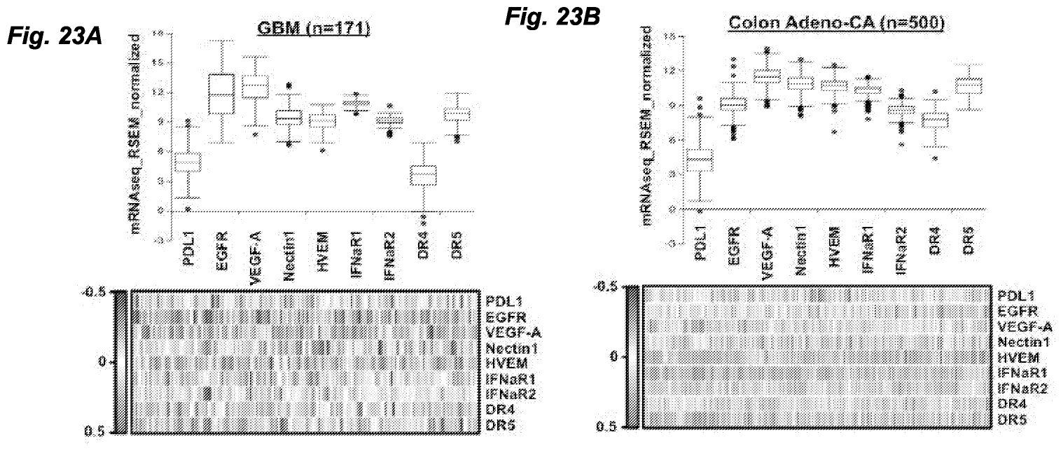

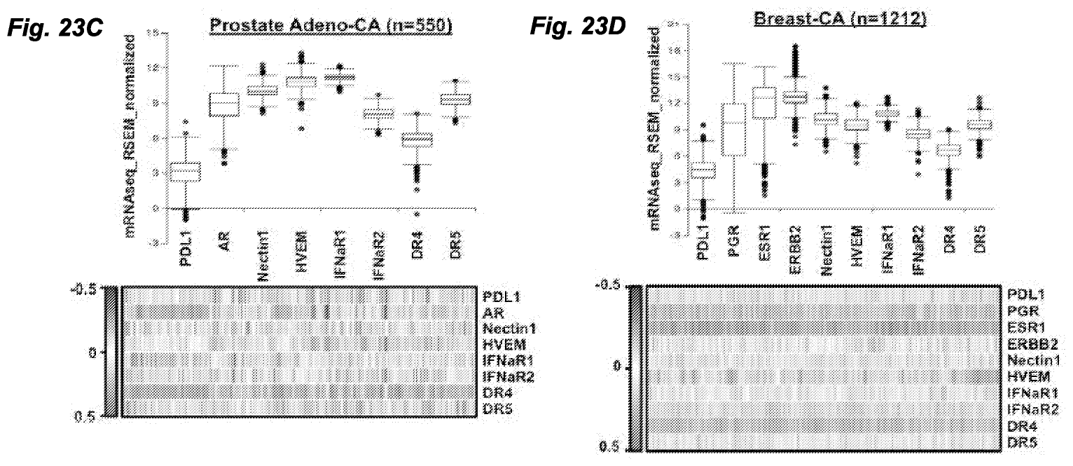

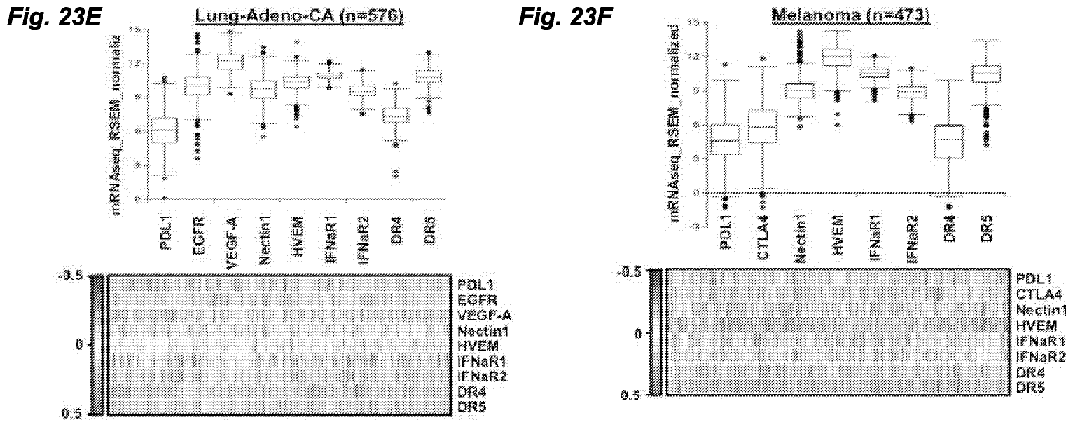

To explore the effect of Nectin1 knockout on the in vivo survival of maMSCs during exposure to oHSV, maMSC-RmC (Nectin1 wild type) were incubated with oHSV at MOI 10 for 6 hours (maMSC-RmC/oHSV) followed by subcutaneous implantation in 1:1 ratio (5×10\hat{ }5 cells each) with either maMSC-GFl (Nectin1 wild type control) or maMSC^{Nectin1}-GFl into the flanks of C57BL/6 mice (2 injections per mouse, n=2 mice for control, n=3 mice for maMSC^{Nectin1}-GFl). Following implantation, the fate of individual cell populations was non-invasively monitored via injection of Luciferin or Coelanterazine to monitor in vivo survival of GFl- or RmC-engineered maMSC cell populations respectively (FIG. 22A). In both groups, Rluc-imaging revealed a rapid decrease of maMSC-RmC/oHSV cell populations following implantation, with no significant difference observed in maMSC-GFl or maMSC^{Nectin1}-GFl coinjected populations and no visible BLI signal post day 3 in both groups (FIG. 22B). Fluc-imaging on the other hand revealed significantly increased in vivo survival of maMSC^{Nectin1}-GFl cells, with visible signal up to day 9 post implantation in comparison to visible signal only up to day 2 for maMSC-GFl control cells (FIG. 22C). Together these results demonstrate that Nectin1 knockout significantly increases the survival of maMSCs in vivo when injected in admixture with oHSV-infected maMSCs.

### Discussion

In this study it is demonstrated that CRISPR-mediated knockout of SC surface receptors allows for the sensitivity-independent expression of receptor-targeted ligands from tSCs. CRISPR engineered tSCs were resistant to autocrine toxicity when engineered with previously self-toxic receptor-targeted agents and demonstrated enhanced survival and anticancer efficacy in vitro and in vivo. Additionally, in the case of oHSV-based therapies, it is demonstrated that this approach allows the establishment of oHSV-resistant SCs which can permit continuous secretion of therapeutics when administered in an admixture together with oHSV-releasing SCs.

The recent discovery of the CRISPR/Cas9 system along with its vast array of possible use cases has inspired a multitude of novel diagnostic and therapeutic approaches. However, while a number of studies have demonstrated CRISPR/Cas9's translational potential for gene therapy of rare diseases, screening purposes as well as disease modeling, practical examples of using CRISPR/Cas9 to improve anticancer effects remain scarce (28). Therapeutic SCs and other cell-based therapies have shown promise in the treatment of cancer and are currently being investigated in numerous preclinical and clinical studies (4, 29). Recently, Deuse et al. have demonstrated how CRISPR gene editing of SCs may enhance their survival post transplantation via a reduction of immune-mediated cell death (30). In the inventors' previous work, they were able to demonstrate the anticancer potential of utilizing stem cells as local delivery platforms of TRAIL and mIFNb (31, 32). Hereby, continuous local delivery of TRAIL and mIFNb via SCs holds particular promise for tumor treatment since both cytokines feature very short half-lives (33, 34) and have been associated with toxicity upon systemic application (34, 35).

Nevertheless, as mentioned in one of the inventors' previous studies, they had experienced a case of significant decrease in anticancer efficacy due to autocrine toxicity of TRAIL secreted from engineered NSCs, leading to rapid NSC cell death within 24 h of TRAIL transgene introduction (21). Similarly, a decrease in cell viability of mIFNb engineered maMSCs, usually occurring over a period of 4-5 days post transduction, was also observed, prompting the use of freshly engineered maMSC-mIFNb for therapeutic studies. While pre-screening allows for identification of TRAIL-resistant SC lines which are able to stably secrete TRAIL, the current study presented herein shows that continuous mIFNb secretion inflicts autocrine toxicity in all tested SC lines, most likely as a result of mIFNb-induced cell cycle arrest and ultimately apoptosis (36). Considering clinical translation, being unrestricted by autocrine sensitivity to secretable anticancer agents would be desirable and would allow choosing optimal SC lines regardless of their sensitivity profile.

Similar to TRAIL (DR4 and DR5), two receptors (IFNaR1 and IFNaR2) are involved in mediating mIFNb's downstream effects. However, while DR4 and DR5 both engage the same apoptotic mechanism, IFNaR1 and IFNaR2 first require heterodimeric complex formation (IFNaR1/IFNaR2) to allow mIFNb binding and downstream pathway activation. Previous studies have identified that in some cases mIFNb activity may be further transmitted via IFNaR1/IFNaR1, but not IFNaR2/IFNaR2, homodimeric complexes (37, 38). Research presented herein confirms these findings, as IFNaR1 knockout alone was sufficient to induce mIFNb resistance and allowed engineering of stably mIFNb secreting maMSCs. In contrast, TRAIL-resistance of NSCs could only be achieved by using the inventors' previously established DR4/DR5 double KO strategy (27).

In recent years, immunotherapies have gained clinical traction, with the main focus being on systemic treatment with checkpoint inhibitors and the first clinical applications for CAR-T's. In addition, OV-based immunotherapies involving direct injection of OV into tumors or application of OV-loaded tumor-tropic SCs have shown promise in a number of preclinical and clinical studies (7, 42, 43). The inventors and others have further shown that combining local OV-based therapies with systemic checkpoint inhibition may improve outcomes (7, 44). In this study it was demonstrated that Nectin1 knockout leads to improved survival of SCs following exposure to oHSV, thus establishing feasibility for sustained intratumoral combinational treatments.

In conclusion, this study demonstrates the feasibility of using CRISPR to enhance SC's therapeutic efficacy and survival via the knockout of therapeutically targetable surface receptors.

### Materials and Methods

Cell lines: The mouse neural stem cell line (mNSC) was provided by Dr. E. Snyder (Burnham Cancer Institute, La Jolla, CA) and was established via v-myc immortalization of mouse neuroprogenitor cells derived from the C17.2 cell line; mNSCs stably expresses B-galactosidase and firefly luciferase as previously described (45). The human fetal neural stem cell line hNSC100 (hNSC) was provided by Dr. Alberto Martinez-Serrano (Autonomous University of Madrid). hNSC's were derived from the human diencephalic and telencephalic regions of 10-10.5 weeks gestational age from an aborted human Caucasian embryo. hNSCs were previously immortalized using retrovirally transduced v-myc (Villa et al., 2000 (46). in vitro and in vivo properties (including the absence of transformation, clonality, multipotency, stability, and survival) have been described in detail previously (47-49). Mouse adipose-derived mesenchymal stem cells (maMSCs) were obtained from iXCells Biotechnologies (available on the world wide web at www.ixcellsbiotech.com). The bone marrow-derived mouse mesenchymal stem cells (mMSC) were kindly provided by Dr. Darwin Prockop (University of Texas). The hASC-TS cell line is an immortalized human adipose-derived mesenchymal stem cell (haMSC) line that was kindly provided to us by Dr. Luigi Balducci and has been described in detail previously (50).

The human breast cancer cell line MDA-MB-231 was kindly provided by Dr. Joan Massagué (Memorial Sloan Kettering Cancer Center, NY). The established glioblastoma cell line Gli36Δ-EGFR was previously generated from parental Gli36 cells (a gift from Anthony Capanogni, UCLA, CA) by retroviral transduction with a cDNA coding for a mutant EGFR (a gift from Drs. H. J. Huang and Webster K. Cavenee). Patient-derived primary invasive glioblastoma cell line GBM8, was provided by Dr. Hiroaki Wakimoto (Massachusetts General Hospital, Boston) and grown in neurobasal medium (Invitrogen) supplemented with 3 mM L-glutamine, B27 supplement, N2 supplement, 2 μg/ml heparin, 20 ng/ml EGF, and 20 ng/ml FGF as described previously (51, 52). Human colorectal cancer cell line HCT116 and human metastatic prostate cancer cell line PC3 were kindly provided by Dr. Umar Mahmood (Massachusetts General Hospital, Boston).

The mouse GBM cell line CT2A was kindly provided by Dr. I. Verma (Salk Institute, San Diego, CA). The mouse GBM cell line 005 was kindly provided by Dr. Martuza and Dr. Rabkin (Massachusetts General Hospital, Boston, MA). All mouse tumor cells were cultured at 37° C. in a humidified atmosphere with 5% CO2 and 1% penicillin/streptomycin (#15140122, Invitrogen) and grown in high glucose Dulbecco's modified Eagle's medium (#11965118, Invitrogen) supplemented with 10% v/v fetal bovine serum (#A4766801, Invitrogen). Cell lines were regularly tested for mycoplasma using a mycoplasma polymerase chain reaction kit (#30-1012K, ATCC).

All cells were cultured at 37° C. in a humidified atmosphere with 5% CO2 and 1% penicillin/streptomycin (Invitrogen). maMSC were grown in low-glucose DMEM supplemented with 15% (vol/vol) FBS, 1% (vol/vol) L-Glutamine, 1% (vol/vol) non-essential amino acid solution. mMSC were grown in high glucose DMEM supplemented with 10% (vol/vol) horse serum and 10% (vol/vol) FBS, 1% (vol/vol) L-Glutamine. hNSC were cultured in 4:1 culturing medium [DMEM/F-12 (Invitrogen, Carlsbad, CA), 0.6% D-glucose (Sigma-Aldrich, St. Louis, MO), 0.5% albumax (Invitrogen), 0.5% glutamine (Invitrogen), recombinant human FGF (40 ng/ml; R & D Systems, Minneapolis, MN), recombinant human EGF (40 ng/ml; R & D Systems), N2 supplements (Invitrogen), and 1% non-essential amino acids (Cellgro; Mediatech, Manassas, VA)] and growth medium [DMEM with 5% fetal bovine serum (Sigma-Aldrich), 1 mM sodium pyruvate (Cellgro; Mediatech), and 26 mM sodium bicarbonate]. haMSC were grown in DMEM/F-12 supplemented with 10% (vol/vol) FBS, 1% (vol/vol) L-Glutamine and recombinant human FGF (40 ng/ml; R&D Systems, Minneapolis, MN). hMSC were grown in Alpha-MEM supplemented with 16.5% (vol/vol) FBS, 1% (vol/vol) L-Glutamine and 1% (vol/vol) penicillin/streptomycin. GBM8, 005 and Mut4 were cultured as ‘neurospheres” in neurobasal medium (Invitrogen/GIBCO) supplemented with 3 mM L-glutamine (Mediatech), 1× B27 (Invitrogen/GIBCO), 2 μg/mL heparin (Sigma), 20 ng/ml human EGF (R and DSystems), and 20 ng/ml human FGF-2 (Peprotech). HCT116 were grown in McCoy's 5a medium supplemented with 10% FBS. PC3 was cultured in F-12K medium supplemented with 10% FBS. 4T1 cells were grown in RPMI-1640 supplemented with 10% (vol/vol) FBS. All other cell lines were cultured in high glucose DMEM supplemented with 10% (vol/vol) FBS, 1% (vol/vol) L-Glutamine and 1% (vol/vol) penicillin/streptomycin.

Lentiviral transduction and stable cell line generation: Lentiviral vectors for GFP and S-TRAIL (ST) were described previously (27, 56). Lentiviral packaging was performed by transfection of 293T cells as previously described (57), and cells were transduced with lentiviral vectors at a multiplicity of infection (M.O.I) of 2 in the medium containing protamine sulfate (2 μg/mL). For BLI, cells were transduced with LV-Pico2-Fluc-mCherry, LV-Pico2-Rluc-mCherry, LV-Pico2-Fluc-GFP, or LV-Pico2-Rluc-GFP. They are selected either via FACS using a BD FACS Aria Fusion cell sorter or by puromycin selection (1 μg/mL) in culture. GFP or mCherry expression was confirmed with fluorescence microscopy.

Immunohistochemistry of stem cell markers and osteogenic differentiation of adipose-derived stem cells: To confirm that CRISPR engineered hNSCs retain their neuronal stem cell markers, hNSC (control) and hNSC^{DR4/5} were plated on a Geltrex™ precoated 24-well plate, followed by immunohistochemical analysis using a human neural stem cell immunohistochemistry kit as per manufacturers recommendation (Life Technologies, #A24354). For osteogenic differentiation, maMSCs and haMSCs were grown to 80% confluency followed by the change of the culture medium to osteogenic differentiation medium (iXCells Biotechnologies USA, LLC). Osteogenic differentiation medium was changed every 3 days for 3 weeks followed by detection of extracellular calcium deposits via staining with Alizarin Red S (Sigma, #A5533).

Cell viability assays: Cells were plated in 96-well plates and treated with different doses of TRAIL, recombinant mouse IFNß and oHSV as indicated for 72 h. Cell viability was measured using an ATP-dependent luminescent reagent (CellTiter-Glo, #G755A, Promega; Glomax, Promega) according to the manufacturer's instructions for non-Fluc expressing cells, or with D-luciferin (#122799, PerkinElmer) and Coelenterazine h (#760506, PerkinElmer) for Flue- and Rluc-expressing cells, respectively. Experiments were performed in triplicate.

Coculture experiments: Fluc-mCherry engineered tumor cells (2×10³ per well) were cocultured with control cells expressing GFP (control) or therapeutic cells expressing GFP and IFNß or TRAIL in 96-well plates. For evaluation of therapeutic effects, the relative number of Fluc-mCherry-expressing tumor cells was determined by Flue bioluminescence 72 hours later. In the case for coculture of Nectin1 wild type or knockout maMSC, wild type maMSCs were transduced with either RmC or GFI and maMSC^{Nectin1} (mNectin1 knockout) were transduced with GFl (maMSC^{Nectin1}-GFl). Stably transduced maMSC-Rmc (mNectin1 wild type) were then coculture in 1:1 ratio in a 96 well plate in triplicate with either maMSC-GFl (mNectin1 wild type) or maMSC^{Nectin1}-GFl. 12 h later cells were treated with oHSV at MOI5 and viability of Rmc- and GFl-expressing cells was assessed overtime via Rluc- and Flue-bioluminescent imaging using a Glomax device (Promega).

Bioluminescence imaging of ST secretion: Control hNSC and hNSC^{DR4/5} were transduced in parallel with LV encoding a fusion variant of ST with the optical reporter Renilla luciferase [Rluc(o) (RI)] as previously described (21) and plated with 2.5×10⁴ per well into 24-well culture plates. ST secretion was evaluated via daily BLI up to 5 d post transduction by adding Coelenterazine h to the culture medium in 1:100 dilution.

Western blot analysis: After treatment, cells were washed with cold PBS twice, and then lysed with cold RIPA buffer (20 mM Tris-HCl pH 8.0, 137 mM NaCl, 10% glycerol, 1% NP-40, 0.1% SDS, 0.5% Na-deoxycholate, 2 EDTA pH 8.0) supplemented with protease and phosphatase inhibitors (Roche protease inhibitor cocktail; Phosphatase Inhibitor Cocktail I and Phosphatase Inhibitor Cocktail II from Sigma-Aldrich). Cells were then centrifuged at 4° C., 16,000 g for 10 minutes. Supernatant protein concentrations were determined using a Bio-Rad protein assay kit. 6×SDS-sample buffer was added samples, which were then boiled for 3 minutes and resolved on SDS-PAGE gel. 10-40 μg of protein was resolved on SDS-PAGE gel, transferred to nitrocellulose membrane, and probed with primary antibodies.

Conditioned media: To obtain conditioned media containing ST or IFNß, cells were engineered with LV as described and medium was collected at indicated time-points followed by concentration using a centrifugal filter (#UFC901024, MilliporeSigma). Samples were stored at −80° C. until future use.

Cloning of lentiviral CRISPR SgRNA expression plasmids and establishment of knockout cell lines: Top and bottom strands of SgRNA oligos were aligned as previously described (53) followed by cloning into PX459 plasmid (Addgene plasmid 48139) using restriction enzyme BbsI. U6-sgRNA regions of sequencing-confirmed PX459-sgRNA clones were PCR-amplified with the following primers containing flanking Gateway-attB1 and -attB2 sequences, respectively: attB1-forward: 5′-GGGGACAAGTTTGTACAAAAAAGCAGGGTCCGAGGGCCTATTTCCCATGATT-3′ (SEQ ID NO: 5), attB2-reverse: 5′-GGGGACCACTTTGTACAAGAAAGCTGGGTCTCTAGAGCCATTTGTCTGCAG-3′ (SEQ ID NO: 6). To prepare Gateway Entry clones, the amplified PCR products were gel-extracted, purified, and cloned into pDONR201 vector (Invitrogen) using the Gateway BP reaction. The lentiviral cDNA/shRNA gateway vectors pLKO.DEST.egfp (Addgene plasmid 32684) or pLKO.DEST.hygro (Addgene plasmid 32685) served as destination vectors after Gateway LR reaction. All destination SgRNA-expression vectors were sequenced to confirm correct U6-sgRNA inserts before proceeding with 3^{rd} generation lentiviral packaging. The following sequences served for human DR4 targeting (5′-3′; PAM underlined): either CGTGGTTCAATCCTCCCCGCGG (SEQ ID NO: 7) or TCTTGTGGACCCGGAGCCGAGGG (SEQ ID NO: 8), and for human DR5 targeting: either AGAACGCCCCGGCCGCTTCGGGG (SEQ ID NO: 9) or CCTACCGCCATGGAACAACGGGG (SEQ ID NO: 10). For targeting of mouse IFNaR1: either CACTGCCCATTGACTCTCCGTGG (SEQ ID NO: 11) or TTCGTGTCAGAGCAGAGGAAGGG (SEQ ID NO: 12). For targeting of human Nectin1 the sequences TGGCTTCATCGGCACAGACGTGG (SEQ ID NO: 13) or GCAGGATGACAAGGTCCTGGTGG (SEQ ID NO: 14) and for targeting of mouse Nectin1 the sequences TGGCTTCATCGGCACAGATGTGG (SEQ ID NO: 15) or CCTCTCCGGTCTGGAGCTGGAGG (SEQ ID NO: 16) were used.

To establish knockout lines, cells were transduced with lentiviral Cas9 expression vectors coding for either tetracycline-inducible or constitutively expressed Cas9 protein as previously described (54, 55). Confirmed Cas9 lines were engineered with lentiviral SgRNA expression vector pLKO.DEST.hygro containing the SgRNA target sequences described above, followed by selection with hygromycin (200-500 μg/ml). For creation of DR4/5 double knockout lines, confirmed Cas9 lines were co-engineered with pLKO.DEST.hygro and pLKO.DEST.egfp lentiviral expression vectors to express both DR4 and DR5 targeting SgRNAs followed by treatment of mixed knockout populations with high-dose TRAIL (1000 ng/ml) to positively select for knockout clones. To screen for knockout efficacy, whole cell lysates of mixed populations were analyzed for sensitivity to respective ligands in comparison to non-engineered controls. Candidate populations were then clonally selected followed by screening of individual clones for DR-KO status with western blotting of cell lysates. To analyze KO clones for targeted indel formation, genomic DNA was isolated from clonal cell populations as previously described (56), and the following primers were used for sequencing of target regions: DR4-forward: TCAGGGTTAGCCAACAGGAGCC (SEQ ID NO: 17); DR4-reverse: TTCTTCCTCCGACTCCGACGAC (SEQ ID NO: 18). DR5-forward: AGGCAGTGAAAGTACAGCCGCG (SEQ ID NO: 19); DR5-reverse: ATTCCCTCCTTGTCGCCCTCCC (SEQ ID NO: 20). IFNaR1 Exon 2: CTTTCTGTACCGTACTGGTCATT (SEQ ID NO: 21) (forward); TCTCAGCTCAGTCTCCACGG (SEQ ID NO: 22) (reverse). IFNaR1 Exon 3: AACACGTTTTAAAAGCCCATGTAT (SEQ ID NO: 23) (forward); GGACCTGCTAAAAGGCTCTTGA (SEQ ID NO: 24) (reverse). Human Nectin1 Exon 2: CTGAGCGGAAGGATCATGGGAT (SEQ ID NO: 25) (forward); GGTCATTGAGGCATCCTGAGGA (SEQ ID NO: 26) (reverse). Human Nectin1 Exon 3: CTTCCTGCAAGAGGTTCTGGGA (SEQ ID NO: 27) (forward); GGGAGGAGAAAGGAGAGGAGGA (SEQ ID NO: 28) (reverse). Mouse Nectin1 Exon2: TAAAGGTCAAGGGCAGAGGACG (SEQ ID NO: 29) (forward); TTGGGTAGTCTCGCTCGTCAAG (SEQ ID NO: 30) (reverse).

Antibodies: Antibodies against cleaved PARP (#9541), caspase 8 (#9746), B-Actin (#4970), p44/42 (MAPK, ERK) (#9102), STAT1 (#9172), phospho-STAT1 (#9167), HRP anti-rabbit (#7074) (Cell Signaling Technologies), anti-Vinculin (#V4505), anti-FLAG (#F7425) (Sigma), anti-DR4 (#1139), anti-DR5 (#2019) (ProSci), anti-TRAIL (#ab9959), anti-Nectin1 (#ab66985), HRP anti-mouse (#ab205719) (Abcam), anti-IFNß (#sc-57201) (Santa Cruz Biotechnology) and were used for western blotting. Anti-human CD261 (DR4)PE (eBioscience), and anti-human CD262 (DR5)PE (eBioscience) were used for flow cytometry analysis.

Mouse models: Female SCID mice, 6-8 weeks of age (Charles River Laboratories), were used for all in vivo xenograft models. Female C57BL/6 mice or Balb/c, 6-8 weeks of age, were used for syngeneic models. BLI was used to follow in vivo growth of Flue- or Rluc-engineered implanted tumor cells over time using a Perkin-Elmer IVIS Lumina system. All in vivo procedures were approved by the Subcommittee on Research Animal Care at Brigham and Women's Hospital. Animals were randomly allocated to cages and experimental groups.

To assess the therapeutic efficacy of ligand secreting knockout SCs, CT2a-FmC were co-injected in 1:1 ratio with either maMSC-GFP (control) or maMSC^{IFNaR1}-IFNß intracranially into the right hemisphere of C57BL/6 mice (1.5×10⁵ each), followed by Flue imaging to monitor in vivo CT2a-FmC tumor growth. GBM8-FmC were co-injected in similar fashion with either hNSC-GFP (control) or hNSC^{DR4/5}-ST. For immunohistochemical and fluorescence analysis of intracranial tumor sections, one control and one hNSC^{DR4/5}-ST co-injected mouse was sacrificed at day 40 post implantation via anesthetization with ketamine/xylazine followed by transcardial perfusion with phosphate-buffered saline (PBS) and subsequently with 4% formaldehyde. 4T1-FmC cells were orthotopically co-injected in 1:1 ration with either maMSC-GFP or maMSC^{IFNaR1}-IFNß (5×10⁵ cells each) into the right mammary fat pat of Balb/c mice followed by BLI overtime. HCT116-FmC were co-injected bilaterally into the flanks of SCID mice (n=2 injections per mouse) in 1:1 ratio with either hNSC-GFP (control) or hNSC^{DR4/5}-ST (5×10⁵ each) and HCT116-FmC tumor volume was followed by calliper measurements over time. In addition, BLI was performed at day 15 post implantation. Tumors were harvested at day 31 post implantation to assess tumor weight. To assess the survival of wild type and Nectin1 knockout maMSCs co-injected with oHSV-infected maMSC-RmC, maMSC-GFl (control) or maMSC^{Nectin1}-GFl were mixed in 1:1 ratio with maMSC-RmC (5×10⁵ each) which had been incubated with oHSV at MOI 10 for 6 h, followed by co-injection into the flanks (n=2 per mouse) of C57/BL6 mice.

In vivo BLI imaging: The viability of Flue- or Rluc-engineered implanted tumor or stem cells was assessed over time using a PerkinElmer IVIS Lumina system. For Flue imaging, mice were imaged 7 min after intraperitoneal injection of D-luciferin (#122799, PerkinElmer) or Coelenterazine h (#760506, PerkinElmer). Tissue processing and HE staining: Tumor-bearing mice were perfused and brains were harvested as described above, followed by coronal sectioning for histological analysis. Brain sections on slides were washed in PBS and mounted with aqueous mounting medium (Vectashield) to be visualized with fluorescence microscopy. For HE staining, sections were incubated with hematoxylin and eosin Y dye (1% alcohol), dehydrated with 70%, 95% and 100% EtOH, and mounted in xylene-based mounting medium (Permount, Fisher Scientific).

Statistical Analysis: Data were expressed as mean±SD for in vitro studies and ±SEM for in vivo studies and analyzed by Student's t test when comparing two groups. Survival times of mouse groups were analyzed and compared using log-rank test. GraphPad Prism 5 Software was used for all statistical analysis and also to generate Kaplan-Meier survival plots. Differences were considered significant at P<0.05 (*), P<0.01 (**), P<0.001 (***), P<0.0001 (****).

### REFERENCES

Bagó J R, et al. (2017) Tumor-homing cytotoxic human induced neural stem cells for cancer therapy. Science translational medicine 9(375).
Park J S, Suryaprakash S, Lao Y-H H, & Leong K W (2015) Engineering mesenchymal stem cells for regenerative medicine and drug delivery. Methods (San Diego, Calf) 84:3-16.
Zhang C-L L, Huang T, Wu B-L L, He W-X X, & Liu D (2017) Stem cells in cancer therapy: opportunities and challenges. Oncotarget 8(43):75756-75766.
Trounson A & McDonald C (2015) Stem Cell Therapies in Clinical Trials: Progress and Challenges. Cell stem cell 17(1):11-22.
Shah K (2016) Stem cell-based therapies for tumors in the brain: are we there yet? Neuro-oncology 18(8):1066-1078.
Stuckey D W & Shah K (2014) Stem cell-based therapies for cancer treatment: separating hope from hype. Nature reviews. Cancer 14(10):683-691.
Du W, et al. (2017) Stem cell-released oncolytic herpes simplex virus has therapeutic efficacy in brain metastatic melanomas. Proceedings of the National Academy of Sciences of the United States of America 114(30).
Hoyos V, et al. (2015) Mesenchymal Stromal Cells for Linked Delivery of Oncolytic and Apoptotic Adenoviruses to Non-small-cell Lung Cancers. Molecular therapy: the journal of the American Society of Gene Therapy 23(9):1497-1506.
Kaczorowski A, et al. (2016) Delivery of improved oncolytic adenoviruses by mesenchymal stromal cells for elimination of tumorigenic pancreatic cancer cells. Oncotarget 7(8):9046-9059.
Parker Kerrigan B C, Shimizu Y, Andreeff M, & Lang F F (2017) Mesenchymal stromal cells for the delivery of oncolytic viruses in gliomas. Cytotherapy 19(4):445-457.
Yuan X, et al. (2016) Mesenchymal stem cells deliver and release conditionally replicative adenovirus depending on hepatic differentiation to eliminate hepatocellular carcinoma cells specifically. Cancer letters 381(1):85-95.
Abraham E, Ahmadian B B, Holderness K, Levinson Y, & McAfee E (2017) Platforms for Manufacturing Allogeneic, Autologous and iPSC Cell Therapy Products: An Industry Perspective. Advances in biochemical engineering biotechnology.
Allan J S & Madsen J C (2002) Recent advances in the immunology of chronic rejection. Current opinion in nephrology and hypertension 11(3):315-321.
Gomalusse G G G, et al. (2017) HLA-E-expressing pluripotent stem cells escape allogeneic responses and lysis by NK cells. Nature biotechnology 35(8):765-772.
Lee A S, et al. (2018) Prolonged survival of transplanted stem cells after ischaemic injury via the slow release of pro-survival peptides from a collagen matrix. Nature biomedical engineering 2(2):104-113.
Nguyen P K, Riegler J, & stem cell W-J C (2014) Stem cell imaging: from bench to bedside. Cell stem cell.
Rosado-de-Castro P H, et al. (2013) Biodistribution of bone marrow mononuclear cells after intra-arterial or intravenous transplantation in subacute stroke patients. Regenerative medicine 8(2):145-155.
Zhang X-M M, Huang G-W W, Tian Z-H H, Ren D-L L, & X Wilson J (2009) Folate deficiency induces neural stem cell apoptosis by increasing homocysteine in vitro. Journal of clinical biochemistry and nutrition 45(1): 14-19.
Adelita T, Stilhano R S, Han S W, Justo G Z, & Porcionatto M (2017) Proteolytic processed form of CXCL12 abolishes migration and induces apoptosis in neural stem cells in vitro. Stem cell research 22:61-69.
Chen N-N N, et al. (2016) Tumor Necrosis Factor Alpha Induces Neural Stem Cell Apoptosis Through Activating p38 MAPK Pathway. Neurochemical research 41(11):3052-3062.
Hingtgen S D, Kasmieh R, van de Water J, Weissleder R, & Shah K (2010) A novel molecule integrating therapeutic and diagnostic activities reveals multiple aspects of stem cell-based therapy. Stem cells (Dayton, Ohio) 28(4):832-841.
Hou Y S, et al. (2015) Lipopolysaccharide pretreatment inhibits LPS-induced human umbilical cord mesenchymal stem cell apoptosis via upregulating the expression of cellular FLICE-inhibitory protein. Molecular medicine reports 12(2):2521-2528.
Shimozato O, Ortaldo J R, Komschlies K L, & Young H A (2002) Impaired NK cell development in an IFN-gamma transgenic mouse: aberrantly expressed IFN-gamma enhances hematopoietic stem cell apoptosis and affects NK cell differentiation. Journal of immunology (Baltimore, Md.: 1950) 168(4):1746-1752.
Stuckey D W, Hingtgen S D, Karakas N, Rich B E, & Shah K (2015) Engineering toxin-resistant therapeutic stem cells to treat brain tumors. Stem cells (Dayton, Ohio) 33(2):589-600.
Naderi-Meshkin H, Bahrami A R, Bidkhori H R, Mirahmadi M, & Ahmadiankia N (2015) Strategies to improve homing of mesenchymal stem cells for greater efficacy in stem cell therapy. Cell biology international 39(1):23-34.
Nowakowski A, Walczak P, Lukomska B, & Janowski M (2016) Genetic Engineering of Mesenchymal Stem Cells to Induce Their Migration and Survival. Stem cells international 2016:4956063.
Reinshagen C, et al. (2018) CRISPR-enhanced engineering of therapy-sensitive cancer cells for self-targeting of primary and metastatic tumors. Science Translational Medicine 10(449).
Mollanoori H & Teimourian S (2018) Therapeutic applications of CRISPR/Cas9 system in gene therapy. Biotechnology letters 40(6):907-914.
June C H, O'Connor R S, Kawalekar O U, Ghassemi S, & Milone M C (2018) CAR T cell immunotherapy for human cancer. Science (New York, N. Y.) 359(6382):1361-1365.
Deuse T, et al. (2019) Hypoimmunogenic derivatives of induced pluripotent stem cells evade immune rejection in fully immunocompetent allogeneic recipients. Nat Biotechnol 37(3):252-258.
Choi S H, et al. (2017) Tumor Resection Recruits Effector T Cells and Boosts Therapeutic Efficacy of Encapsulated Stem Cells Expressing IFN13 in Glioblastomas. Clinical cancer research: an official journal of the American Association for Cancer Research 23(22):7047-7058.
Kauer T M, Figueiredo J-L L, Hingtgen S, & Shah K (2011) Encapsulated therapeutic stem cells implanted in the tumor resection cavity induce cell death in gliomas. Nature neuroscience 15(2):197204.
Ashkenazi A, Pai R C, Fong S, & Leung S (1999) Safety and antitumor activity of recombinant soluble Apo2 ligand. The Journal of . . . .
Salmon P, Le Cotonnec J Y, Galazka A, Abdul-Ahad A, & Darragh A (1996) Pharmacokinetics and pharmacodynamics of recombinant human interferon-beta in healthy male volunteers. Journal of interferon & cytokine research: the official journal of the International Society for Interferon and Cytokine Research 16(10):759-764.
Trinchieri G (2010) Type I interferon: friend or foe? The Journal of experimental medicine 207(10):2053-2063.
Sangfelt O, Erickson S, & Grander D (2000) Mechanisms of interferon-induced cell cycle arrest. Frontiers in bioscience: a journal and virtual library 5:87.
de Weerd N A, et al. (2013) Structural basis of a unique interferon-13 signaling axis mediated via the receptor IFNAR1. Nature immunology 14(9):901-907.
Zitvogel L, Galluzzi L, Kepp O, Smyth M J, & Kroemer G (2015) Type I interferons in anticancer immunity. Nature reviews. Immunology 15(7):405-414.
Sedý J R, Spear P G, & Ware C F (2008) Cross-regulation between herpesviruses and the TNF superfamily members. Nature reviews. Immunology 8(11):861-873.
Ran F A, et al. (2013) Double nicking by RNA-guided CRISPR Cas9 for enhanced genome editing specificity. Cell 154(6):1380-1389.
Jones B S, Lamb L S, Goldman F, & Di Stasi A (2014) Improving the safety of cell therapy products by suicide gene transfer. Frontiers in pharmacology 5:254.
Bayan C-A Y, et al. (2018) The Role of Oncolytic Viruses in the Treatment of Melanoma. Current oncology reports 20(10):80.
Duebgen M, et al. (2014) Stem cells loaded with multimechanistic oncolytic herpes simplex virus variants for brain tumor therapy. Journal of the National Cancer Institute 106(6).
Bommareddy P K, Shettigar M, & Kaufman H L (2018) Integrating oncolytic viruses in combination cancer immunotherapy. Nature reviews. Immunology 18(8):498-513.
Tang Y, et al. (2003) In vivo tracking of neural progenitor cell migration to glioblastomas. Human gene therapy 14(13):1247-1254.
Villa A, Snyder E Y, Vescovi A, & Martinez-Serrano A (2000) Establishment and properties of a growth factor-dependent, perpetual neural stem cell line from the human CNS. Experimental neurology 161(1):67-84.
Villa A, et al. (2004) Long-term molecular and cellular stability of human neural stem cell lines. Experimental cell research 294(2):559-570.
Navarro-Galve B, et al. (2005) Gene marking of human neural stem/precursor cells using green fluorescent proteins. The journal of gene medicine 7(1):18-29.
Rubio F J, Bueno C, Villa A, Navarro B, & Martinez-Serrano A (2000) Genetically perpetuated human neural stem cells engraft and differentiate into the adult mammalian brain. Molecular and cellular neurosciences 16(1):1-13.
Balducci L, et al. (2014) Immortalization of human adipose-derived stromal cells: production of cell lines with high growth rate, mesenchymal marker expression and capability to secrete high levels of angiogenic factors. Stem cell research & therapy 5(3):63.
Wakimoto H, et al. (2009) Human glioblastoma-derived cancer stem cells: establishment of invasive glioma models and treatment with oncolytic herpes simplex virus vectors. Cancer research 69(8):3472-3481.
Wakimoto H, et al. (2012) Maintenance of primary tumor phenotype and genotype in glioblastoma stem cells. Neuro-oncology 14(2):132-144.
Ran F A, et al. (2013) Genome engineering using the CRISPR-Cas9 system. Nature protocols 8(11):2281-2308.
Sanjana N E, Shalem O, & Zhang F (2014) Improved vectors and genome-wide libraries for CRISPR screening. Nature methods 11(8):783-784.
Wang T, Wei J J, Sabatini D M, & Lander E S (2014) Genetic screens in human cells using the CRISPR-Cas9 system. Science 343(6166):80-84.
Strauss W M (2001) Preparation of genomic DNA from mammalian tissue. Current protocols in molecular biology Chapter 2.

### Example 3: Developing Enhanced Stem Cell Based Combined Oncolytic Virus Therapies for Cancer

Cytokines are considered as potent immunomodulatory molecules and have been successfully used as adjuvants in OV therapy for cancer. Among the cytokines that have been successfully used as adjuvants in OV therapy for cancer, IL-12 and GMCSF have been extensively explored as candidates for tumor immunotherapy, due to their ability to activate both innate and adaptive immunity 1. However, their short half-life, severe side effects associated, particularly with systemic administration of IL-12^{42, 431, 2} and a very narrow therapeutic benefit has prompted local delivery of IL-12 and GMCSF directly into a tumor microenvironment. Numerous studies have shown constitutive expression of IL-12 via armed OVs such as oHSV³⁻⁶, adenovirus⁷⁻¹⁰, newcastle disease virus¹¹, and semliki forest virus¹² result in anti-tumor efficacy in a variety of different tumor models. Although promising, however, OV mediated expression of IL-12 leads to its release during active propagation of OVs in tumor cells thus inducing anti-viral immune response and premature clearance of OVs.

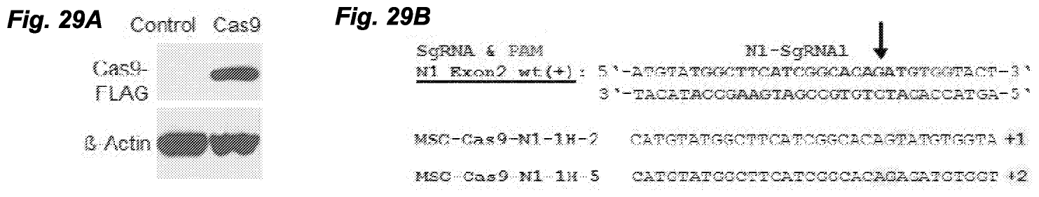

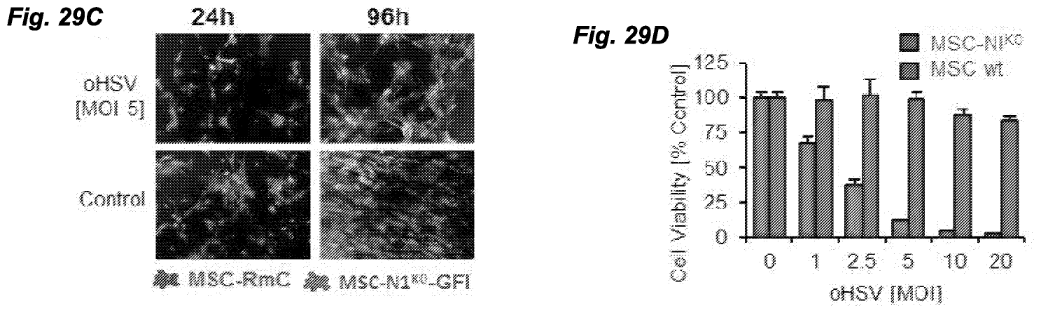

The inventors have previously shown that MSC loaded oHSV undergo viral oncolysis and progeny release within 72 hrs of their loading¹³. Therefore, it is not feasible to co-deliver oHSV and immune-modulators from the same MSC. Nectin-1 (CD111), a cell surface adhesion molecule is known as the most efficient entry receptor for oHSV¹⁴ and the inventors have previously shown that both mouse and human MSC have high expression of nectin-1¹⁵. To circumvent issues related to the delivery of oHSV and immunomodulators to GBM tumors, a cell-based strategy to simultaneously co-deliver oHSV and regulatable immunomodulators was developed. Specifically, using CRISPR/Cas9 technology, nectin-1 receptor (N1) knockout MSC (MSC-N1^{KO}) were created and it was shown herein that MSC-N1^{KO} are resistant to oHSV mediated oncolysis (FIG. 29) and thus can be utilized to co-deliver immune-modulators in combination with MSC-oHSV. It is shown herein that MSC-N1^{KO} are resistant to oHSV mediated oncolysis and thus can be utilized to co-deliver cytokines that act as adjuvants to OV mediated therapy for cancer (FIG. 29).

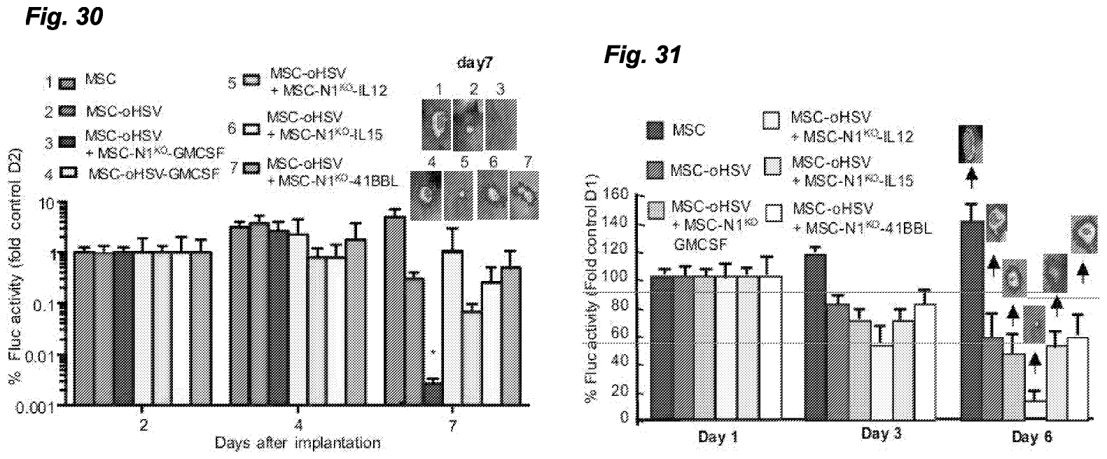

Based on these findings, MSC-N1^{KO} were further engineered to release immunomodulators: interleukin (IL)-12, granulocyte macrophage colony stimulating factor (GM-CSF), 41BB ligand (L) and IL-15 and performed an in vivo screen to test the efficacy of MSC-oHSV in combination with MSC-N1^{KO} releasing immunomodulators in a brain seeking syngeneic mouse melanoma cell model. The data indicated that GM-CSF release from MSC significantly boosts oHSV efficacy (FIG. 30) and therefore is an ideal candidate to be used in combination with MSC-oHSV for treating metastatic melanomas. Using a similar screen, it was shown that IL-12 released from MSC significantly boosts oHSV efficacy (FIG. 31) and therefore is an ideal candidate to be used in combination with MSC-oHSV for treating primary brain tumors, GBMs.

### Data

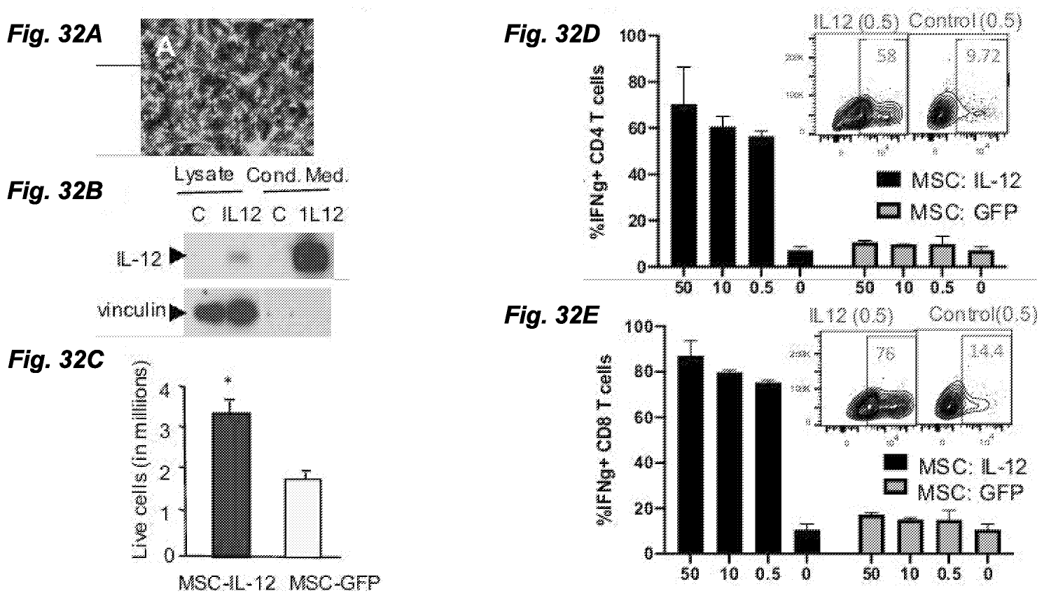

Creating and Characterizing oHSV resistant MSC-N1^{KO} expressing IL-12. Previous preclinical studies have shown that IL-12 expressing oHSV mediate superior anti-tumor effects when compared to other non-cytokine HSVs¹⁶ specifically by enhancing the influx of both CD4⁺ and CD8⁺ T-cells in murine models of brain tumors^{17, 18}. A number of studies have shown that the presence of IL-12 in the tumor microenvironment induces IFN-γ production, cytolytic activity of natural killer (NK) cells and cytotoxic T cells and promotes the development of a T helper 1 (Th1)-type immune response^{19, 20}. Recent studies have shown that oHSV-IL-12 results in a significantly higher CD8:MDSC and CD8:T regulatory cell (Treg) ratios, indicating that oHSV-IL-12 creates a more favorable immune tumor microenvironment^{3, 21, 22}. Mouse IL-12 were used and constructed a dual promoter vector bearing secretable murine p35 and p40 subunits of IL-12 separated by a linker sequence that the inventors have optimized previously²³. Data presented herein indicate that MSCN1^{KO} can be readily engineered to express functional mouse IL-12 (FIG. 32A-32E).

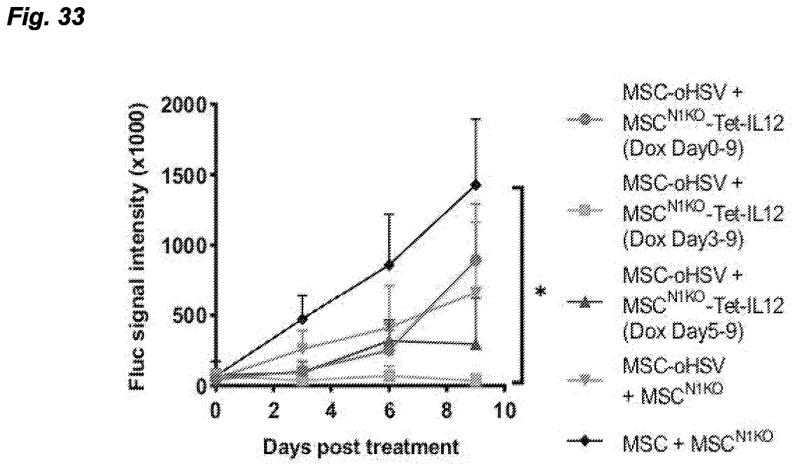

Assessing the influence of the timing of regulatable IL-12 release on MSC-oHSV efficacy: The inventors have previously generated recombinant oHSV vectors, G47Δ-empty (referred to oHSV), G47Δ-firefly luciferase (oHSV-Flue) and G47Δ-mCherry (oHSV-mCh)²⁴ and recently oHSV-Fluc-mCh (oHSV-FmC). In parallel utilizing the pCW57.1 Dox-inducible lentiviral vector (Addgene)²⁵, double promoter lentiviral vectors for Tet regulatable GFP-Fluc, mouse p40 and p35 subunits of IL-12 were created. Data regarding CT2A-FmC GBM tumors treated with intratumoral injections of MSC-oHSV and MSC^{N1KO}-Tet-IL-12 indicated that presence of IL-12 at the beginning of oHSV infection of tumor cells does not influence the efficacy MSC-oHSV and MSC^{N1KO}-Tet-IL-12 combination therapy. However, the expression of IL-12 post-day 3 enhances the efficacy of MSC-oHSV and MSC^{N1KO}-Tet-IL-12 treatment (FIG. 33).

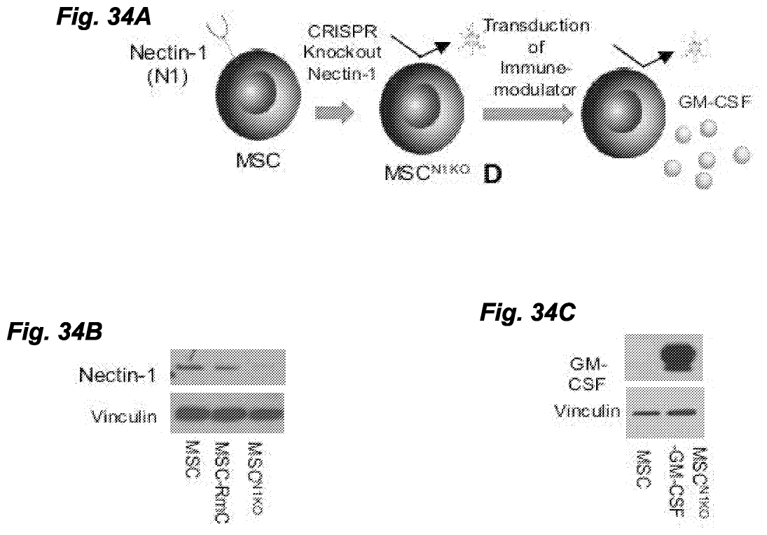

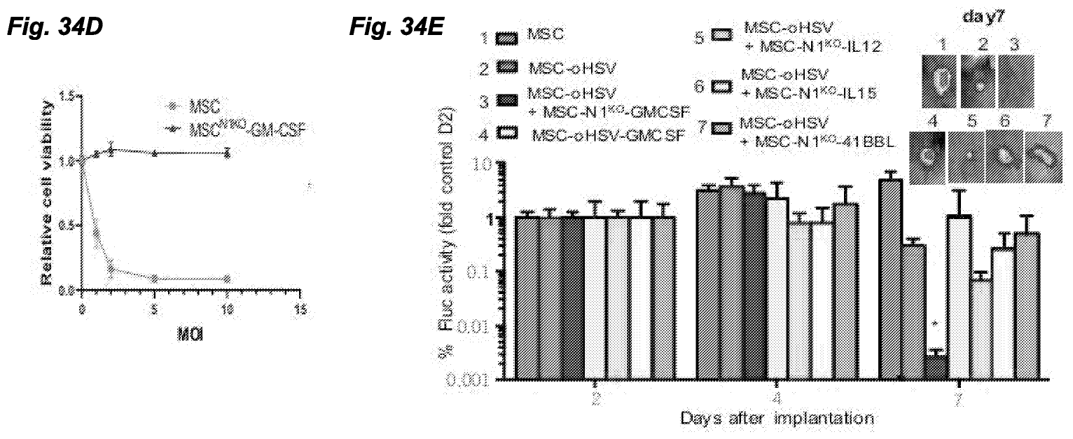

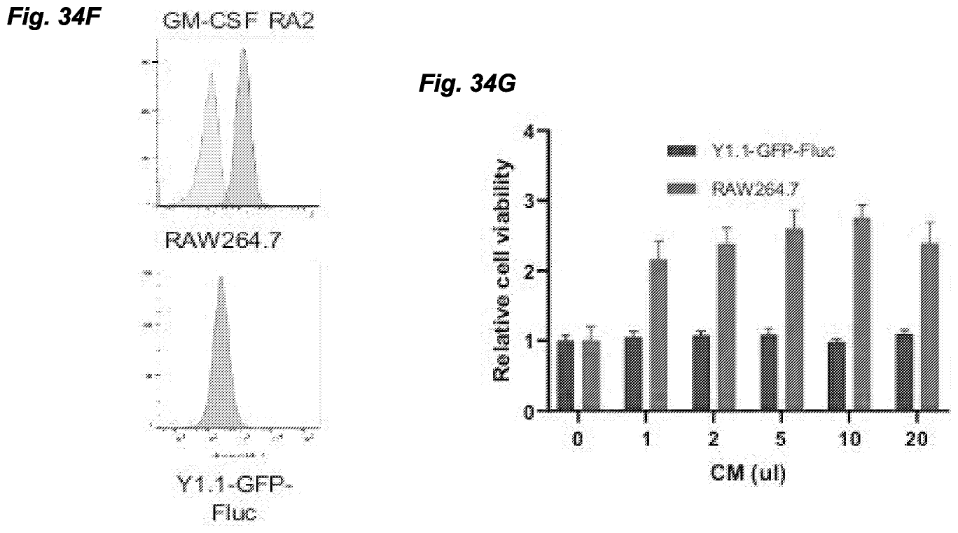

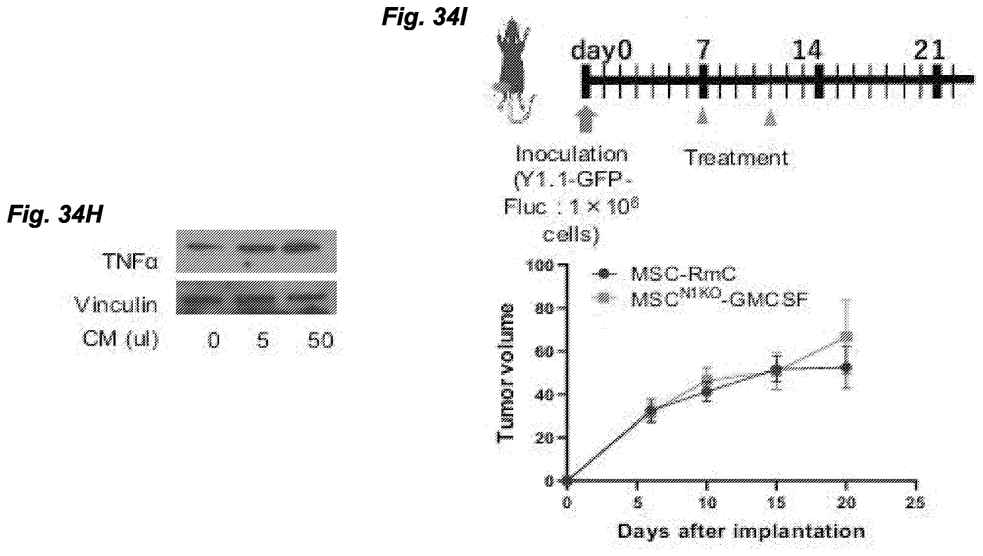

GM-CSF is known for its ability to recruit dendritic cells and natural killer cells, mediate induction of tumor-specific CD8+ cytotoxic T-lymphocytes and stimulate a systemic and adaptive antitumor immune response^{26, 27}. In an effort to overcome the short half-life of GM-CSF, numerous pre-clinical and clinical studies have explored constitutive expression of GM-CSF via armed OVs such as oHSV 28, adenovirus s, and vaccinia virus³⁰ in different cancer types. Although promising, however, OV mediated expression of GM-CSF leads to its prolonged uncontrolled release in the tumor microenvironment and previous studies have shown that prolonged release results in the development of neutralizing antibodies in melanoma patients thus abrogating the potential clinical benefit of GM-CSF³¹. Mouse MSC-N1^{KO} (oHSV resistant nectin-1 knockout MSC) were engineered to express GMCSF and the cells have been characterized extensively in vitro. Specifically, it was shown that MSC-N1^{KO} are resistant to oHSV and express GMCSF when transduced with lentiviral vector bearing GMCSF (FIG. 34A-34D). Next, it was shown that MSC^{N1KO} mediated expression of GMCSF enhances the antitumor activity of oHSV-loaded MSC in vivo (FIG. 6E). Furthermore, it was shown that MSC-N1^{KO}-GMCSF conditioned medium induces murine macrophage, RAW264.7 growth but not melanoma, Y1.1-GFP-Fluc growth in vitro and results in TNF-α release from RAW264.7 (FIG. 34H). To assess the influence of MSC^{N1KO}-GMCSF on tumor growth, Y1.1-GFP-Fluc (GFl) bearing tumors were treated with MSC^{N1KO}-GMCSF and control MSC-RmC intratumorally. Flue imaging data indicated that MSC^{N1KO}-GMCSF did not directly influence melanoma tumor growth in vivo. These data indicate that oHSV resistant MSC^{N1KO} expressing GMCSF enhance the function of MSC-oHSV via macrophage activation.

### Abscopal Effects of Combination Therapy in a Bilateral Subcutaneous Tumor Model

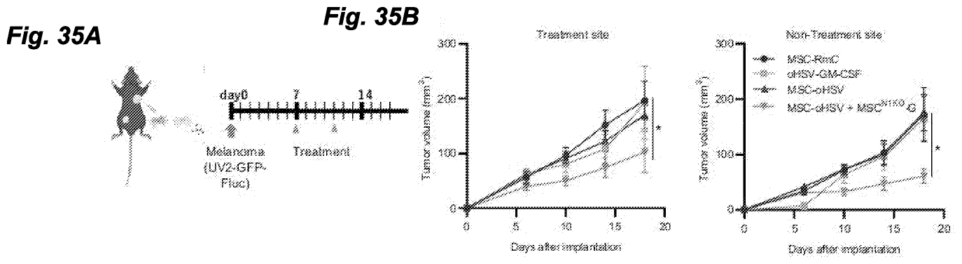

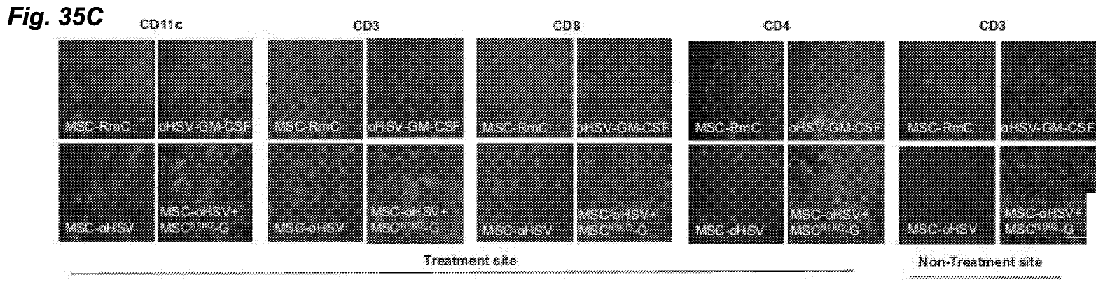

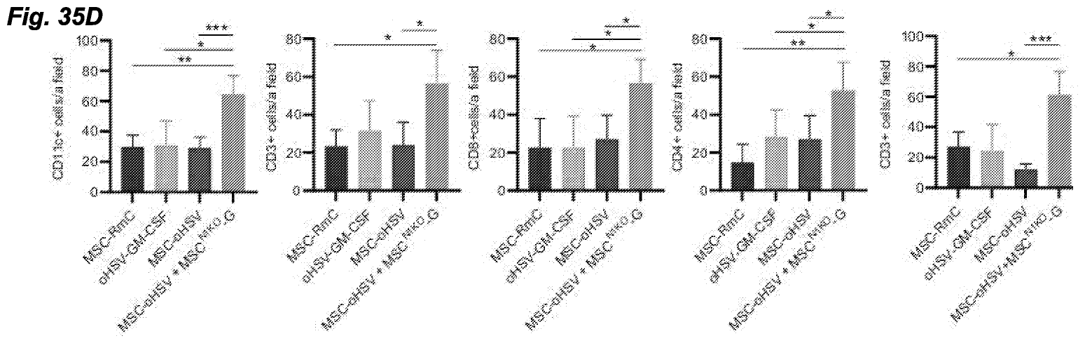

The abscopal effects as well as the antitumor effects of combination therapy with MSC-oHSV, and MSC^{N1KO}-GM-CSF in bilateral subcutaneous PTEN mutant line derived UV2-GFP-Fluc melanoma model were assessed (FIG. 35A). Treatment with MSC-oHSV and MSC^{N1KO}-GM-CSF resulted in significant therapeutic effects compared to control MSC-RmC treatment (FIG. 35B). Further, the combined therapy suppressed tumor growth compared to control treatment whereas oHSV-GM-CSF, or MSC-oHSV alone did not show efficacy. Next, tumor infiltrating immune cells post-treatment were assessed by immunofluorescence (IF) in UV2-GFP-Fluc mouse model. IF analysis indicated the increase of CD11c, CD3, CD4, CD8 positive cells at the treatment site and CD3 positive cells in non-treatment site were in the combination therapy (FIG. 35C-35D).

### Therapeutic Efficacy of MSC Secreting Dual Immunomodulators and MSC-oHSV Against Immunosuppressive Leptomeningeal Metastasis

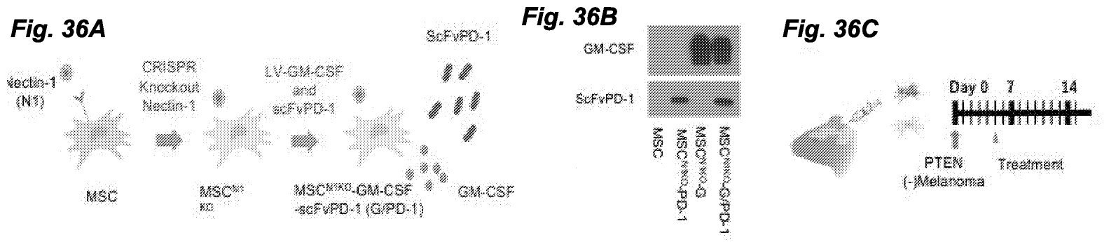

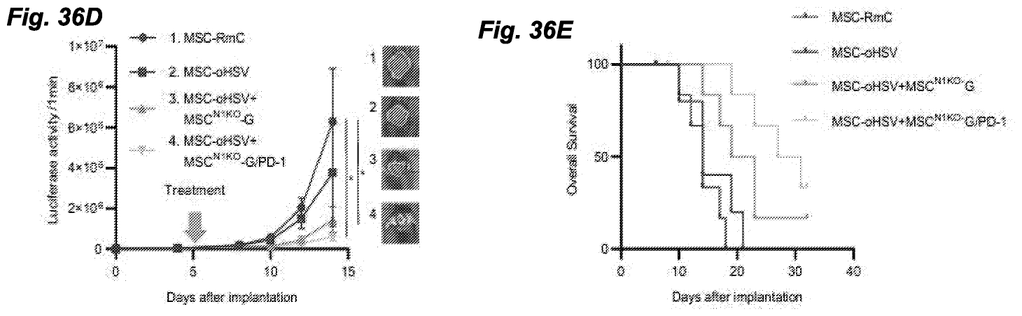

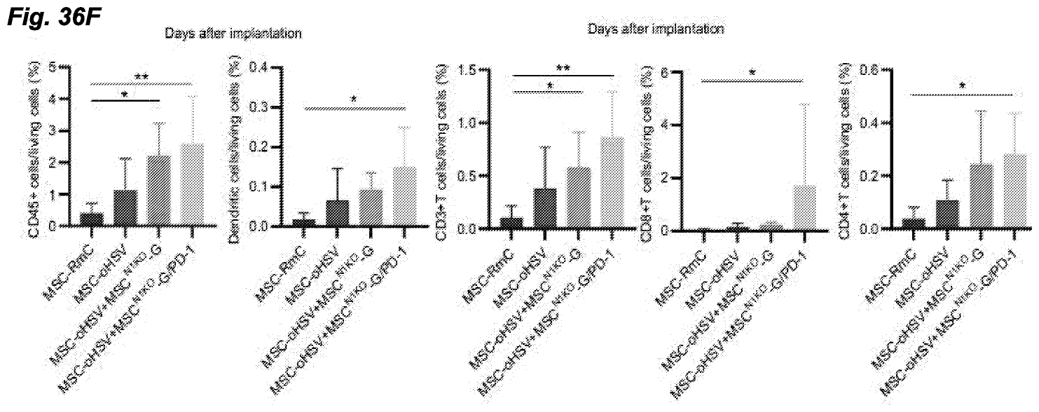

An immunosuppressive leptomeningeal metastasis (LM) model was established by intrathecally (IT) injecting brain seeking melanoma tumor cells, UV2-GFP-Fluc into the cisterna magna. To combat immunosuppressive LM model, MSC^{N1KO}-GM-SCF were further transduced with LV bearing cDNA for a single chain variable fragment (scFv) PD-1 to cells. Functional assay showed scFvPD-1 blocked PD-1 expression on splenocytes by Flowcytometry (FIG. 36A). To explore the efficacy of stem cell delivery of oHSV, GM-CSF, and scFvPD-1 for LM, intrathecal (IT) injection of MSC-oHSV and MSC^{N1KO}-GM-CSF-scFvPD-1 (G/PD-1) were tested. A significant difference in tumor volumes was seen when tumors were treated with MSC-oHSV and MSC^{N1KO}-GM-CSF and MSC^{N1KO}-G/PD-1 compared to control MSC-RmC (FIG. 36C-36D) resulting in a significantly prolonged overall survival (FIG. 36E).

### MSC Releasing Human GM-CSF and MSC-oHSV Against Patient Derived PTEN-Deficient Melanoma Brain Metastasis

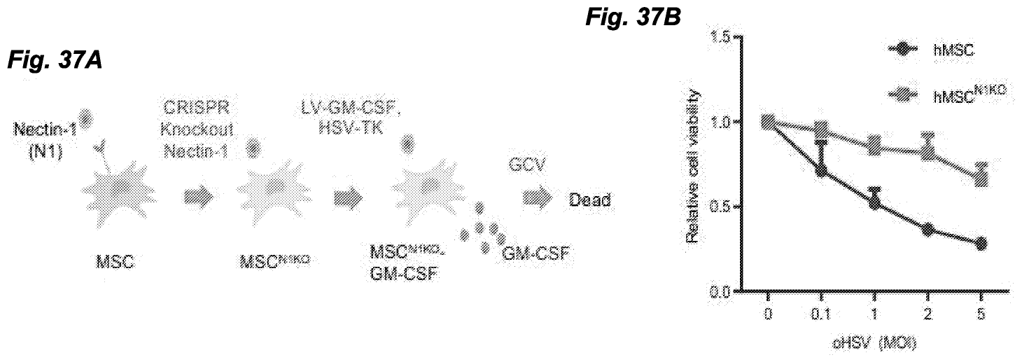

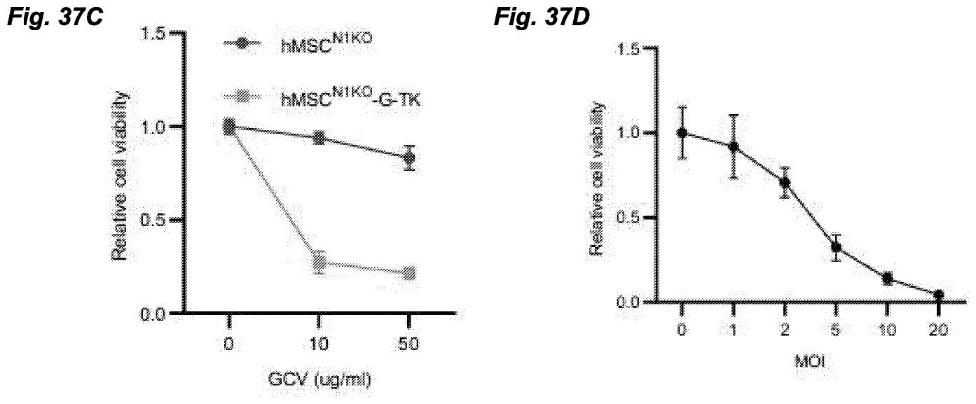

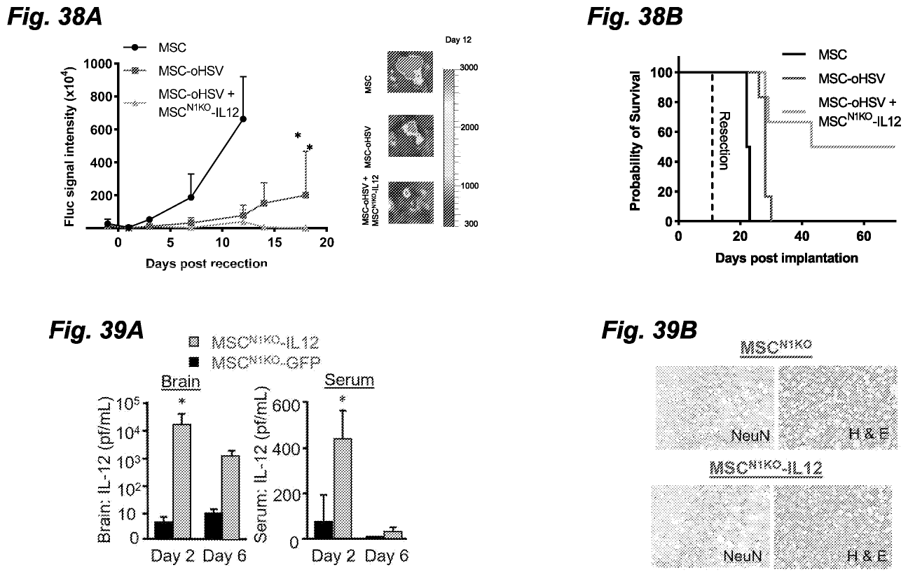

To set up clinical setting, human MSC^{N1KO} secreting human GM-CSF were created (FIG. 37A) and it was shown that human MSC^{N1KO} are resistant to oHSV compared to naïve human MSCs (FIG. 37B). HSV-TK into MSC^{N1KO}-GM-CSF were also engineered as a safety switch, and confirmed that activation of HSV-TK via ganciclovir (GCV) killed MSCs^{N1KO}-GM-CSF in a dose dependent manner (FIG. 37C). In addition, cell viability assay revealed that oHSV treatment resulted in killing of M12-GFP Flue cells in vitro (FIG. 37D).

### REFERENCES

Lasek, W., Zagozdzon, R. & Jakobisiak, M. Interleukin 12: still a promising candidate for tumor immunotherapy? Cancer Immunol Immunother 63, 419-435 (2014).
Alkayyal, A. A., et al. NK-Cell Recruitment Is Necessary for Eradication of Peritoneal Carcinomatosis with an IL-12-Expressing Maraba Virus Cellular Vaccine. Cancer Immunol Res 5, 211-221 (2017).
Cheema, T. A., et al. Multifaceted oncolytic virus therapy for glioblastoma in an immunocompetent cancer stem cell model. Proc Natl Acad Sci USA 110, 12006-12011 (2013).
Zhang, W., et al. Combination of oncolytic herpes simplex viruses armed with angiostatin and IL-12 enhances antitumor efficacy in human glioblastoma models. Neoplasia 15, 591-599 (2013).
Patel, D. M., et al. Design of a Phase I Clinical Trial to Evaluate M032, a Genetically Engineered HSV-1 Expressing IL-12, in Patients with Recurrent/Progressive Glioblastoma Multiforme, Anaplastic Astrocytoma, or Gliosarcoma. Hum Gene Ther Clin Dev 27, 69-78 (2016).
Thomas, E. D., et al. IL-12 Expressing oncolytic herpes simplex virus promotes anti-tumor activity and immunologic control of metastatic ovarian cancer in mice. J Ovarian Res 9, 70 (2016).
Choi, I. K., et al. Oncolytic adenovirus co-expressing IL-12 and IL-18 improves tumor-specific immunity via differentiation of T cells expressing IL-12Rbeta2 or IL-18Ralpha. Gene Ther 18, 898-909 (2011).
El-Shemi, A. G., et al. Combined therapy with oncolytic adenoviruses encoding TRAIL and IL-12 genes markedly suppressed human hepatocellular carcinoma both in vitro and in an orthotopic transplanted mouse model. J Exp Clin Cancer Res 35, 74 (2016).
Huang, J. H., et al. Therapeutic and tumor-specific immunity induced by combination of dendritic cells and oncolytic adenovirus expressing IL-12 and 4-1BBL. Mol Ther 18, 264-274 (2010).
Poutou, J., et al. Safety and antitumor effect of oncolytic and helper-dependent adenoviruses expressing interleukin-12 variants in a hamster pancreatic cancer model. Gene Ther 22, 696-706 (2015).
Ren, G., et al. Recombinant Newcastle Disease Virus Encoding IL-12 and/or IL-2 as Potential Candidate for Hepatoma Carcinoma Therapy. Technol Cancer Res Treat 15, NP83-94 (2016).
Quetglas, J. I., et al. Virotherapy with a Semliki Forest Virus-Based Vector Encoding IL-12 Synergizes with PD-1/PD-L1 Blockade. Cancer Immunol Res 3, 449-454 (2015).
Duebgen, M., et al. Stem cells loaded with multimechanistic oncolytic herpes simplex virus variants for brain tumor therapy. Journal of the National Cancer Institute 106, dju090 (2014).
Krummenacher, C., et al. Comparative usage of herpesvirus entry mediator A and nectin-1 by laboratory strains and clinical isolates of herpes simplex virus. Virology 322, 286-299 (2004).
Du, W., et al. Stem cell-released oncolytic herpes simplex virus has therapeutic efficacy in brain metastatic melanomas. Proc Natl Acad Sci USA 114, E6157-E6165 (2017).
Hellums, E. K., et al. Increased efficacy of an interleukin-12-secreting herpes simplex virus in a syngeneic intracranial murine glioma model. Neuro Oncol 7, 213-224 (2005).
Markert, J. M., et al. Preclinical evaluation of a genetically engineered herpes simplex virus expressing interleukin-12. J Virol 86, 5304-5313 (2012).
Parker, J. N., et al. Engineered herpes simplex virus expressing IL-12 in the treatment of experimental murine brain tumors. Proc Natl Acad Sci USA 97, 2208-2213 (2000).
Kishima, H., et al. Systemic interleukin 12 displays anti-tumour activity in the mouse central nervous system. Br J Cancer 78, 446-453 (1998).
Meko, J. B., Tsung, K. & Norton, J. A. Cytokine production and antitumor effect of a nonreplicating, noncytopathic recombinant vaccinia virus expressing interleukin-12. Surgery 120, 274-282; discussion 282-273 (1996).
Ring, E. K., et al. Newly Characterized Murine Undifferentiated Sarcoma Models Sensitive to Virotherapy with Oncolytic HSV-1 M002. Mol Ther Oncolytics 7, 27-36 (2017).
Saha, D., Martuza, R. L. & Rabkin, S. D. Macrophage Polarization Contributes to Glioblastoma Eradication by Combination Immunovirotherapy and Immune Checkpoint Blockade. Cancer Cell 32, 253-267 e255 (2017).
van de Water, J. A., et al. Therapeutic stem cells expressing variants of EGFR-specific nanobodies have antitumor effects. Proc Natl Acad Sci USA 109, 16642-16647 (2012).
Tamura, K., et al. Multimechanistic tumor targeted oncolytic virus overcomes resistance in brain tumors. Mol Ther 21, 68-77 (2013).
Wang, T., Wei, J. J., Sabatini, D. M. & Lander, E. S. Genetic screens in human cells using the CRISPR-Cas9 system. Science 343, 80-84 (2014).
Mastrangelo, M. J., et al. Intratumoral recombinant GM-CSF-encoding virus as gene therapy in patients with cutaneous melanoma. Cancer Gene Ther 6, 409-422 (1999).
Dranoff, G. GM-CSF-secreting melanoma vaccines. Oncogene 22, 3188-3192 (2003).
Andtbacka, R. H., et al. Talimogene Laherparepvec Improves Durable Response Rate in Patients With Advanced Melanoma. J Clin Oncol 33, 2780-2788 (2015).
Koski, A., et al. Treatment of cancer patients with a serotype 5/3 chimeric oncolytic adenovirus expressing GMCSF. Mol Ther 18, 1874-1884 (2010).
Parviainen, S., et al. GMCSF-armed vaccinia virus induces an antitumor immune response. Int J Cancer 136, 1065-1072 (2015).
Spitler, L. E., et al. Biological Effects of Anti-Granulocyte-Macrophage Colony-Stimulating Factor (GM-CSF) Antibody Formation in Patients Treated With GM-CSF (Sargramostim) as Adjuvant Therapy of Melanoma. American journal of clinical oncology 40, 207-213 (2017).

1. A method of treating cancer, the method comprising administering to a subject in need thereof a first stem cell (SC) modified to release an oncolytic virus and a second SC which is gene edited to inactivate a receptor for the oncolytic virus, thereby generating a SC resistant to the virus, wherein the second SC is also engineered to express an immunomodulatory polypeptide agent.

2. The method of claim 1, wherein the second SC is gene edited to inactivate the receptor for the oncolytic virus before the second SC is engineered to express the immunomodulatory polypeptide agent.

3. The method of claim 1 or claim 2, wherein the first and/or second SC is a mesenchymal stem cell (MSC) or a neuronal stem cell (NSC).

4. The method of any one of claims 1-3, wherein the first and/or second SC is autologous to the subject.

5. The method of any one of claims 1-3, wherein the first and/or second SC is allogeneic to the subject.

6. The method of any one of claims 1-5, wherein the oncolytic virus is an oncolytic herpes simplex virus (oHSV).

7. The method of claim 6, wherein the oHSV is or is derived from G47Δ oHSV.

8. The method of claim 6 or claim 7, wherein the receptor is nectin-1.

9. The method of any one of claims 1-8, wherein the oncolytic virus encodes a heterologous polypeptide.

10. The method of claim 9, wherein the heterologous polypeptide is a tumor necrosis factor related apoptosis-inducing ligand (TRAIL) polypeptide or a cytokine that promotes an anti-tumor immune response.

11. The method of claim 10, wherein the cytokine that promotes an anti-tumor immune response is one or more of GM-CSF, IL-2, IL-12, Flt3L, IL-5 and IL-15.

12. The method of any one of claims 1-11, wherein the immunomodulatory polypeptide agent expressed by the second SC comprises a cytokine that promotes an anti-tumor immune response.

13. The method of claim 12, wherein the cytokine is one or more of GM-CSF, IL-2, IL-12, Flt3L, IL-5 and IL-15.

14. The method of claim 12 or 13, wherein the cytokine is a GM-CSF polypeptide.

15. The method of any one of claims 1-11, wherein the immunomodulatory polypeptide agent comprises a modulator of an immune checkpoint molecule.

16. The method of claim 15, wherein the modulator of an immune checkpoint molecule is an agonist of a stimulatory checkpoint molecule or an antagonist of an inhibitory checkpoint molecule.

17. The method of claim 15 or 16, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.

18. The method of claim 15 or 16, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.

19. The method of any one of claims 15-18, wherein the modulator of an immune checkpoint molecule comprises an antibody or an antigen-binding fragment thereof that specifically binds the immune checkpoint molecule.

20. The method of any one of claims 1-19, wherein the second SC further expresses a second therapeutic polypeptide.

21. The method of claim 20, wherein the second therapeutic polypeptide comprises a cytokine or a modulator of an immune checkpoint molecule.

22. The method of claim 21, wherein the cytokine is one or more of GM-CSF, IL-2, IL-12, Flt3L, IL-5 and IL-15.

23. The method of claim 21, wherein the modulator of an immune checkpoint molecule is an agonist of a stimulatory checkpoint molecule or an antagonist of an inhibitory checkpoint molecule.

24. The method of claim 23, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.

25. The method of claim 23, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.

26. The method of any one of claims 21 or 23-25, wherein the modulator of an immune checkpoint molecule comprises an antibody or an antigen-binding fragment thereof that specifically binds the immune checkpoint molecule.

27. A method of treating cancer, the method comprising administering to a subject in need thereof a stem cell (SC) modified to express and secrete a receptor-targeted cytotoxic agent, wherein the SC is gene edited to inactivate a receptor for the receptor-targeted cytotoxic agent.

28. The method of claim 27, wherein the receptor-targeted cytotoxic agent is a cytokine or a death receptor-targeted pro-apoptotic factor.

29. The method of claim 28, wherein the cytokine is IFNβ.

30. The method of claim 28 or 29, wherein the receptor for the receptor-targeted cytotoxic agent is IFNaR1 or IFNaR2.

31. The method of claim 28, wherein the death receptor-targeted pro-apoptotic factor is tumor necrosis factor related apoptosis-inducing ligand (TRAIL).

32. The method of claim 31, wherein the receptor for the receptor-targeted cytotoxic agent is death receptor (DR) 4 or DR5.

33. The method of any one of claims 1-32, wherein the receptor for the oncolytic virus or the receptor-targeted cytotoxic agent is inactivated by targeted gene editing.

34. The method of any one of claims 27-33, wherein the SC is further engineered to express an immunomodulator polypeptide.

35. The method of claim 34, wherein the immunomodulator polypeptide is a cytokine that promotes an anti-tumor immune response.

36. The method of claim 35, wherein the cytokine is one or more of GM-CSF, IL-12, IL-2, IL-12, Flt3L, IL-5 and IL-15.

37. The method of claim 35 or claim 36, wherein the cytokine is a GM-CSF polypeptide.

38. The method of claim 34, wherein the immunomodulator polypeptide comprises a modulator of an immune checkpoint molecule.

39. The method of claim 38, wherein the modulator of an immune checkpoint molecule is an agonist of a stimulatory checkpoint molecule or an antagonist of an inhibitory checkpoint molecule.

40. The method of claim 39, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.

41. The method of claim 39, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.

42. The method of any one of claims 38-41, wherein the modulator of an immune checkpoint molecule comprises an antibody or an antigen-binding fragment thereof that specifically binds the immune checkpoint molecule.

43. The method of any one of claims 27-42, further comprising administering a second SC engineered to express an immunomodulatory polypeptide agent.

44. The method of any one of claims 1-43, wherein the cancer comprises a solid tumor cancer.

45. The method of any one of claims 1-44, wherein the cancer is selected from melanoma, lung cancer, breast cancer and glioblastoma.

46. The method of any one of claims 1-45, wherein the cancer comprises a primary tumor or a metastatic tumor.

47. The method of claim 46, wherein the metastatic tumor comprises a metastasis to the brain.

48. The method of any one of claims 1-47, wherein the cancer is PTEN-deficient.

49. The method of any one of claims 1-48, wherein the administering comprises intratumor administration.

50. The method of any one of claims 1-48, wherein the administering comprises systemic administration.

51. The method of any one of claims 1-48, wherein the administering comprises administration of any or all of the SCs to a tumor resection cavity.

52. A composition comprising
a) a first stem cell (SC) modified to release an oncolytic virus, and
b) a second SC which is gene edited to inactivate a receptor for the oncolytic virus, thereby generating a SC resistant to the virus,
wherein the second SC is also engineered to express an immunomodulatory polypeptide agent.

53. The composition of claim 52, wherein the second SC is gene edited to inactivate the receptor for the oncolytic virus before the second SC is engineered to express the immunomodulatory polypeptide agent.

54. The composition of claim 52 or claim 53, wherein the first and/or second SC is a mesenchymal stem cell (MSC) or a neuronal stem cell (NSC).

55. The composition of any one of claims 52-54, wherein the first and/or second SC is autologous to the subject.

56. The composition of any one of claims 52-54, wherein the first and/or second SC is allogeneic to the subject.

57. The composition of any one of claims 52-56, wherein the oncolytic virus is an oncolytic herpes simplex virus (oHSV).

58. The composition of claim 57, wherein the oHSV is or is derived from G47Δ oHSV.

59. The composition of claim 57 or claim 58, wherein the receptor is nectin-1.

60. The composition of any one of claims 52-59, wherein the oncolytic virus encodes a heterologous polypeptide.

61. The composition of claim 60, wherein the heterologous polypeptide is a tumor necrosis factor related apoptosis-inducing ligand (TRAIL) polypeptide or a cytokine that promotes an anti-tumor immune response.

62. The composition of claim 61, wherein the cytokine that promotes an anti-tumor immune response is one or more of GM-CSF, IL-2, IL-12, Flt3L, IL-5 and IL-15.

63. The composition of any one of claims 52-62, wherein the immunomodulatory polypeptide agent expressed by the second SC comprises a cytokine that promotes an anti-tumor immune response.

64. The composition of claim 63, wherein the cytokine is one or more of GM-CSF, IL-2, IL-12, Flt3L, IL-5 and IL-15.

65. The composition of claim 63 or 64, wherein the cytokine is a GM-CSF polypeptide.

66. The composition of any one of claims 52-62, wherein the immunomodulatory polypeptide agent comprises a modulator of an immune checkpoint molecule.

67. The composition of claim 66, wherein the modulator of an immune checkpoint molecule is an agonist of a stimulatory checkpoint molecule or an antagonist of an inhibitory checkpoint molecule.

68. The composition of claim 66 or 67, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.

69. The composition of claim 66 or 67, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.

70. The composition of any one of claims 66-69, wherein the modulator of an immune checkpoint molecule comprises an antibody or an antigen-binding fragment thereof that specifically binds the immune checkpoint molecule.

71. The composition of any one of claims 52-70, wherein the second SC further expresses a second therapeutic polypeptide.

72. The composition of claim 71, wherein the second therapeutic polypeptide comprises a cytokine or a modulator of an immune checkpoint molecule.

73. The composition of claim 72, wherein the cytokine is one or more of GM-CSF, IL-2, IL-12, Flt3L, IL-5 and IL-15.

74. The composition of claim 72, wherein the modulator of an immune checkpoint molecule is an agonist of a stimulatory checkpoint molecule or an antagonist of an inhibitory checkpoint molecule.

75. The composition of claim 74, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.

76. The composition of claim 74, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.

77. The composition of any one of claims 72 or 74-76, wherein the modulator of an immune checkpoint molecule comprises an antibody or an antigen-binding fragment thereof that specifically binds the immune checkpoint molecule.

78. A composition comprising a stem cell (SC) modified to express and secrete a receptor-targeted cytotoxic agent, wherein the SC is gene edited to inactivate a receptor for the receptor-targeted cytotoxic agent.

79. The composition of claim 78, wherein the receptor-targeted cytotoxic agent is a cytokine or a death receptor-targeted pro-apoptotic factor.

80. The composition of claim 79, wherein the cytokine is IFNβ.

81. The composition of claim 78 or 79, wherein the receptor for the receptor-targeted cytotoxic agent is IFNaR1 or IFNaR2.

82. The composition of claim 78, wherein the death receptor-targeted pro-apoptotic factor is tumor necrosis factor related apoptosis-inducing ligand (TRAIL).

83. The composition of claim 81, wherein the receptor for the receptor-targeted cytotoxic agent is death receptor (DR) 4 or DR5.

84. The composition of any one of claims 78-83, wherein the receptor for the oncolytic virus or the receptor-targeted cytotoxic agent is inactivated by targeted gene editing.

85. The composition of any one of claims 78-84, wherein the SC is further engineered to express an immunomodulator polypeptide.

86. The composition of claim 85, wherein the immunomodulator polypeptide is a cytokine that promotes an anti-tumor immune response.

87. The composition of claim 86, wherein the cytokine is one or more of GM-CSF, IL-12, IL-2, IL-12, Flt3L, IL-5 and IL-15.

88. The composition of claim 86 or claim 87, wherein the cytokine is a GM-CSF polypeptide.

89. The composition of claim 85, wherein the immunomodulator polypeptide comprises a modulator of an immune checkpoint molecule.

90. The composition of claim 89, wherein the modulator of an immune checkpoint molecule is an agonist of a stimulatory checkpoint molecule or an antagonist of an inhibitory checkpoint molecule.

91. The composition of claim 90, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.

92. The met composition of claim 90, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.

93. The composition of any one of claims 78-92, wherein the modulator of an immune checkpoint molecule comprises an antibody or an antigen-binding fragment thereof that specifically binds the immune checkpoint molecule.

94. The composition of any one of claims 78-93, further comprising at a least a second SC, wherein the second SC is different from the first SC.

95. The composition of any preceding claim, further comprising a pharmaceutically acceptable carrier.

96. Use of a composition of any one of claims 52-95 for the treatment of cancer in a subject in need thereof.

97. The use of claim 96, wherein the cancer comprises a solid tumor cancer.

98. The use of claim 96, wherein the cancer is selected from melanoma, lung cancer, breast cancer and glioblastoma.

99. The use of any one of claim 96-98, wherein the cancer comprises a primary tumor or a metastatic tumor.

100. The use of claim 99, wherein the metastatic tumor comprises a metastasis to the brain.

101. The use of any one of claims 96-100, wherein the cancer is PTEN-deficient.

102. The method of any one of claims 1-11, wherein the immunomodulatory polypeptide agent of the second SC comprises a modulator of an immune checkpoint molecule.

103. The method of claim 102, wherein the modulator of an immune checkpoint molecule is an agonist of a stimulatory checkpoint molecule or an antagonist of an inhibitory checkpoint molecule.

104. The method of claim 102 or 103, wherein the modulator of an immune checkpoint molecule is an agonist of a stimulatory checkpoint molecule selected from OX40, 4-1BB, GITR, CD28, ICOS, LIGHT, CD27, DNAM-1, 2B4, DC-SIGN, DR3, and CD40.

105. The method of claim 102 or 103, wherein the modulator of an immune checkpoint molecule is an antagonist of an inhibitory checkpoint molecule selected from PD-1, PD-L1, CTLA4, B7-H3, B7-H4, VISTA, TMIGD2, B7-H7, BTLA, HVEM, CD160, LAG3, TIGIT, CD96, CD155 and TIM-3.

106. The method of any one of the preceding claims, wherein the immunomodulatory polypeptide agent of the first and second SC are the same.

107. The method of any one of the preceding claims, wherein the immunomodulatory polypeptide agent of the first and second SC are different.

108. The composition of any one of the preceding claims, wherein the immunomodulatory polypeptide agent of the first and second SC are the same.

109. The composition of any one of the preceding claims, wherein the immunomodulatory polypeptide agent of the first and second SC are different.
